(19) United States
(12) Patent Application Publication
(10) Pub. No.: US 2020/0055817 A1
(43) Pub. Date: Feb. 20, 2020

(54) SPIRO-COMPOUNDS AS SIP MODULATORS

(71) Applicant: AbbVie Deutschland GmbH & Co. KG, Wiesbaden (DE)

(72) Inventors: Wilhelm AMBERG, Ludwigshafen (DE); Jeroen VAN BERGEIJK, Ludwigshafen (DE); Herve GENESTE, Ludwigshafen (DE); Wilfried HORNBERGER, Ludwigshafen (DE); Elizabeth Louise VAN DER KAM, Ludwigshafen (DE); Udo LANGE, Ludwigshafen (DE); Mario MEZLER, Ludwigshafen (DE); Michael OCHSE, Ludwigshafen (DE); Frank OELLIEN, Ludwigshafen (DE); Patrick WALLESER, Kriens (CH); Sean C. TURNER, Ludwigshafen (DE)

(21) Appl. No.: 16/346,642

(22) PCT Filed: Nov. 2, 2017
(86) PCT No.: PCT/EP2017/078043

§ 371 Date: May 1, 2019

(30) Foreign Application Priority Data
Nov. 2, 2016 (CN) PCT/CN2016/104349

(51) Int. Cl. C07D 205/12; C07D 405/12; C07D 401/10; C07D 403/12; C07D 491/107; C07D 221/20

(52) U.S. Cl. CPC C07D 205/12; C07D 405/12; C07D 221/20; C07D 403/12; C07D 491/107; C07D 401/10

(57) ABSTRACT

The invention relates to heterocyclic compounds of formula (I) as SIP modulators, pharmaceutical compositions comprising such compounds, and uses thereof in the treatment or alleviation of diseases or disorders mediated by an SIP receptor.

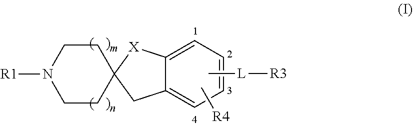

The invention relates to spiro compounds with affinity to S1P receptors, pharmaceutical compositions comprising such compounds, the use of such compounds in the treatment or alleviation of diseases and disorders in which an S1P receptor is involved or in which modulation of the endogenous S1P signaling system via an S1P receptor is involved and the preparation of a medicament for treating or alleviating such diseases and disorders.

### BACKGROUND OF THE INVENTION

Sphingosine-1-phosphate (S1P) is part of the sphingolipid class of molecules. S1P is a bioactive sphingolipid that mediates a wide variety of cellular responses, such as proliferation, autophagy, blockade of apoptosis, cell differentiation, blockade of cell senescence, cytoskeletal organization and migration, adherence- and tight junction assembly, and morphogenesis. Moreover, S1P is a modulator of APP processing via BACE1 regulation as well as lipid raft formation and can interact with ABC transporters thereby modulating cellular in- and efflux. S1P can bind with members of the endothelial cell differentiation gene family (EDG receptors) of plasma membrane-localized G protein-coupled receptors. To date, five members of this family have been identified as S1P receptors in different cell types, S1P1 (EDG-1), S1P2 (EDG-5), S1P3 (EDG-3), S1P4 (EDG-6) and S1P5 (EDG-8). S1P can produce cytoskeletal re-arrangements in many cell types to regulate immune cell trafficking, vascular homeostasis and cell communication in the central nervous system (CNS) and in peripheral organ systems. The above mentioned actions of S1P are mediated by interaction with its receptors. Therefore, S1P receptors are therapeutic targets for the treatment of, for example, neoplastic diseases, diseases of the central and peripheral nervous system, autoimmune disorders and tissue rejection in transplantation.

It is known that S1P is secreted by vascular endothelium and is present in blood at concentrations of 200-900 nanomolar and is bound by albumin and other plasma proteins. This provides both a stable reservoir in extracellular fluids and efficient delivery to high-affinity cell-surface receptors. S1P binds with low nanomolar affinity to the five receptors S1P1-5. In addition, platelets also contain S1P and may be locally released to cause e.g. vasoconstriction. The receptor subtypes S1P1, S1P2 and S1P3 are widely expressed and represent dominant receptors in the cardiovascular system. Further, S1P1 is also a receptor on lymphocytes. S1P4 receptors are almost exclusively in the haematopoietic and lymphoid system. S1P5 is primarily (though not exclusively) expressed in central nervous system (CNS; brain and spinal cord). Other tissues with S1P5 expression are skin and spleen. Moreover, S1P5 is expressed on NK cells. Early study showed that the CNS expression in mice appeared restricted to oligodendrocytes, while in men and rats expression was more diverse. Recent evidence has shown a broader distribution in all species: S1P5 expression is shown at the level of astrocytes, endothelial cells, glial cells, oligodendrocytes and to a lesser extent neurons.

The present invention relates to modulators of the S1P5 receptor, in particular agonists, and preferably to agonists with selectivity over S1P1, S1P3 and/or S1P4 receptors, in view of unwanted cardiovascular and/or peripheral immune-modulatory effects. It has now been found that S1P5 agonists can be used in the treatment of cognitive disorders, in particular age-related cognitive decline. Moreover, evidence has shown an impact on amyloid 6 (protein) processing, ABC transporter expression, blood-brain-barrier integrity, neuro-inflammatory processes, and (sphingo)lipid content in the CNS.

The latter is of high relevance as an altered sphingolipid metabolism is strongly implicated in several neurodegenerative and cognitive diseases. A comparison of CNS gene expression profiles of normal and Alzheimer's Disease (AD) patients indicated that genes responsible for S1P degradation were strongly upregulated, including the phosphatidic acid phosphatase PPAP2A and S1P lyase genes, while genes for ceramide production (apoptotic sphingolipid) were upregulated (Katsel et al, 2007, Neurochem Res, 32, 845-856). These gene expression data are predictive of actual changes in enzyme and lipid levels in the brain and cerebrospinal fluid (CSF): compared to normal subjects, AD brain are characterized by higher levels of ceramide and cholesterol as well as decreased levels of S1P. These changes also correlate with disease severity of the patients and are related to levels of Amyloid ß and Tau, two hallmarks of Alzheimer's Disease (Cutler et al, 2004, PNAS, 101, 2070-2075; He et al, 2010, Neurobiol. Aging, 31, 398-408; Koal et al, 2015, J. Alz Disease, 44, 1193-1201). The same changes have been reported in brain tissues (and CSF) from patients suffering HIV dementia, Amyotrophic Lateral Sclerosis (ALS), Parkinson's Disease, Parkison's Disease with Lewy Bodies, Multiple Sclerosis, Huntington's Disease, and several sphingolipdidosis disorders (Lysosomal Storage Disorders) such as Niemann Pick Disease and Gauchers (Cutler et al, 2002, Ann Neurol, 52, 448-457; Haughey et al, 2004, Ann Neurol, 55, 257-267; Cutler et al, 2010, Neurol, 63, 636-630; Mielke et al, 2013, PLOS ONE, 8; Bras et al, 2008, FEBS Journal, 275, 5767-5773; Vidaurre et al, 2014, Brain, 137, 2271-2286; Fan et al, 2013, J Lipid Research, 54, 2800-2814). Modulating the activity of the S1P5 receptor in the central nervous system may be a therapeutic method for such neurodegenerative or cognitive disorders by shifting the ceramide/S1P balance towards S1P effects and away from ceramide-mediated cell death.

Soluble ß-amyloid (Aß) oligomers are considered the proximate effectors of synaptic injury and neuronal death occurring in AD. Aß induces increased ceramide levels and oxidative stress in neuronal cultures, leading to apoptosis and cell death. S1P is a potent neuroprotective factor against this Aß-induced damage, consistent with its role as ceramide's counterpart (Cutler et al, 2004, PNAS, 101, 2070-2075, Malaplate-Armand, 2006, Neurobiol. Dis, 23, 178-189). Aß is also pro-inflammatory, inducing the migration of monocytes to sites of injury, and the S1P1, S1P3, S1P4, S1P5 agonist FTY720/Fingolimod inhibits such migration. Aß is known to induce expression of S1P2 and S1P5, but not of S1P1, S1P3 and S1P4 (Kaneider et al, 2004, FASEB). The actions of FTY720/FIngolimod and those expressed by monocytes suggest these effects are mediated by the S1P5 receptor. The same applies to more recent findings that FTY720/Fingolimod is able to modulate Aß-induced memory deficits (Fukumoto et al, 2014, Beh Brain Res, 268, 88-93).

Additional studies suggest a role for S1P in modulating pain signals. In example, S1P modulates action potentials in capsaicin-sensitive sensory neurons (Zhang et al, 2006, J Physiol, 575, 101-113) and S1P levels are known to be decreased in CSF in acute and inflammatory pain models (Coste et al, 2008, J Biol Chem, 283, 32442-32451). The S1P1, S1P3, S1P4, S1P5 receptor agonist FTY720/Fingolimod is indeed able to reduce nociceptive behavior in neuropathic pain models (Coste et al, 2008, 12, 995-1004), while the selective S1P1 agonist SEW2817 fails to have an effect. Given the high CNS expression of S1P5 and lack of effects of S1P1 agonism, the effects can be contributed to effects on the S1P5 receptor.

In summary, potent and selective agents that are agonists of the S1P5 receptor will be beneficial for the treatment of cognitive disorders, neurodegenerative disorders and pain. In particular, S1P5-selective ligands would be beneficial for these diseases by not engaging the S1P1, S1P3 and/or S1P4 receptor ensuring a lack of peripheral immune suppression and cardiovascular side-effects.

WO 2012/004373 describes S1P receptor modulators containing a fused heterocyclic core. This fused heterocyclic core structurally differs from the compounds of the present invention in the size and position of the rings constituting the core and the type and number of heteroatoms present in the rings.

WO 2012/004378 also describes S1P receptor modulators containing a core comprising a fused bicyclic ring structure. These compounds structurally differ from the compounds of the present invention in the position of the ring structures relative to each other, resulting in differences in overall three-dimensional configuration of the chemical structure. Other differences are in the size of the ring structures and the heteroatoms present in the ring structure.

Currently, there is still a need for new, potent S1P receptor modulators, in particular selective S1P5 receptor modulators.

### SUMMARY OF THE INVENTION

It is an object of the present invention to provide S1P5 receptor modulators, in particular agonists, preferably to agonists with selectivity over in particular S1P1, S1P3 and/or S1P4 receptors to avoid unwanted cardiovascular and/or immunomodulatory effects. It is a further object of the invention to provide a method for treatment or alleviation of a variety of CNS disorders, such as cognitive disorders, in particular age-related cognitive decline. The invention therefor provides a compound of formula (I):

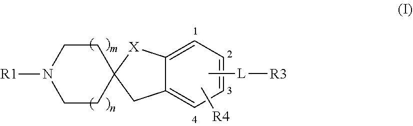

or a pharmaceutically acceptable salt thereof, wherein

### X is CH₂ or O;

m and n are independently 0 or 1;
R1 is selected from the group consisting of —(C1-4)alkylene-R2, —(C3-6)cycloalkylene-R2, —(C1-3)alkylene-(C3-6)cycloalkylene-R2 and —(C3-6)cycloalkylene-(C1-3)alkylene-R2, wherein the (C1-4)alkylene is optionally substituted with up to 3 carbon atoms, with (CH₂)₂ to form a cyclopropyl moiety or with (CH₂)₃ to form a cyclobutyl moiety and wherein R2 is selected from the group consisting of —COOH, —OH, —OPO₃H₂, —PO₃H₂, —COO(C1-4)alkyl and tetrazol-5-yl;
L is attached to atom 1, 2, 3 or 4 and is a group —W—(CH₂)p-T- wherein:
W is attached to the phenylene moiety and selected from the group consisting of —O—, —CO—, —S—, —SO—, —SO₂—, —NH—, —CH₂—CH₂—, —CF₂—CH₂—, —CH₂—CF₂—, —CH═CH—, —C(CF₃)═CH—, —CH═C(CF₃)—, —C≡C—, phenyl, —(C3-7)cycloalkyl-, -pyridyl-, -thienyl- and -thiazolyl-, wherein the phenyl, (C3-7)cycloalkyl, pyridyl, thienyl or thiazolyl is optionally substituted with one or more substituents independently selected from the group consisting of a halogen atom, hydroxy, (C1-4)alkyl optionally substituted with one or more halogen atoms, and (C1-4)alkoxy optionally substituted with one or more halogen atoms;
p is 0 or an integer from 1 to 4; and
T is absent or attached to R3 and selected from the group consisting of —O—, —O—(C1-4)alkyl-, —S—, —SO—, —SO₂—, —NH—, —CO—, —CH═CH—, —C≡C— and cyclopropylene;
   R3 is selected from the group consisting of (C3-6)cycloalkyl, (C4-6)cycloalkenyl, phenyl, biphenyl, naphthyl, a monocyclic heterocycle and a 8-10 membered fused bicyclic group, each optionally substituted with one or more substituents independently selected from the group consisting of:
halogen,
cyano,
(C1-6)alkyl optionally substituted with one or more fluoro atoms,
(C1-4)alkoxy optionally substituted with one or more fluoro atoms or with (C3-6)cycloalkyl,
(C3-6)cycloalkoxy optionally substituted with one or more fluoro atoms;
—S—(C1-4)-alkyl,
—SF₅,
phenoxy, benzyl or benzyloxy, and
(C3-8)cycloalkyl optionally substituted with phenyl, (C1-4)alkyl, (C1-4)alkoxy or a halogen atom;
   R4 is absent or selected from the group consisting of a halogen atom, (C1-4)alkyl optionally substituted with one or more halogen atoms, and (C1-4)alkoxy optionally substituted with one or more halogen atoms.

In a further aspect the invention provides a pharmaceutical composition comprising a compound according to the invention or a pharmaceutically acceptable salt thereof and at least one pharmaceutically acceptable auxiliary.

In a still further aspect the invention provides a method of treatment or alleviation of a disease or disorder in which an S1P receptor is involved or in which modulation of the endogenous S1P signaling system via an S1P receptor is involved, preferably S1P5, comprising administering to a patient in need thereof a compound according to the invention or a pharmaceutically acceptable salt thereof.

In a still further aspect the invention provides a use of a compound according to the invention or a pharmaceutically acceptable salt thereof for the manufacture of a medicament for the treatment or alleviation of a disease or disorder in which an S1P receptor is involved or in which modulation of the endogenous S1P signaling system via an S1P receptor is involved, preferably S1P5 receptor.

In a still further aspect the invention provides a compound according to the invention or a pharmaceutically acceptable salt thereof for use in therapy.

In a still further aspect the invention provides a compound according to the invention or a pharmaceutically acceptable salt thereof for use in the treatment or alleviation of a disease or disorder in which an S1P receptor is involved or in which modulation of the endogenous S1P signaling system via an S1P receptor is involved, preferably S1P5.

### DETAILED DESCRIPTION OF THE INVENTION

The compounds of the invention are modulators of the S1P receptor, in particular of the S1P5 receptor. More specifically, the compounds of the invention are S1P5 receptor agonists. The compounds of the invention and their pharmaceutically acceptable salts are in particular suitable for agonizing S1P5 in a subject suffering from a disorder in which modulation of S1P5 activity and the subsequent ceramide/S1P axis is beneficial. Administration of such compound to a subject is preferably such that S1P5 activity in the subject is altered and treatment is achieved. The compounds of the present invention are particularly suitable to treat or alleviate diseases and disorder s in which an S1P receptor is involved or in which modulation of the endogenous S1P signaling system via an S1P receptor is involved. In particular the compounds of the present invention are suitable to treat or alleviate a disorder or disorder selected from the group consisting of Alzheimer's Disease (AD) and associated dementia's, amyloid 6-associated disorders, Mild Cognitive Impairment (MCI), Parkinson's Disease (PD), Lewy Body Dementia (LBD), Progressive Supranuclear Palsy (PSP), Cerebral Palsy (CP), Amyotrophic Lateral Sclerosis (ALS), Frontal Temporal Lobe Dementia (FTLD), multiple sclerosis, Huntington's Disease, neurological symptoms of sphingolipidosis disorders, a lysosomal storage disorder including Tay Sachs Disease, Sandhoff Disease, Fabry's Disease, Krabbe Disease, Gaucher's Disease, Niemann Pick A, B or C, and Batten's Disease, stroke, HIV-associated Dementia (HAD), HIV-associate Neurocognitive Disorder (HAND), HIV-associated neuropathy, schizophrenia, cognitive deficits in Schizophrenia, an attention deficit disorder including Anxiety Attention Deficit Disorder and Attention Deficit Hyperactivity Disorder (ADHD), a bipolar disorder, Obsessive-Compulsive Behavior, pain including neuropathic, back pain and pain-associated with multiple sclerosis, spinal cord injury, Parkinson's Disease, epilepsy, diabetes and cancer, cancer-induced peripheral neuropathy (CIPN), depression, treatment-resistant depression, Creutzfeld-Jakob Disease and other Prion-related Disorders, Down's Syndrome, autism, age-related cognitive decline or memory impairment, cognitive deficits associated with diabetes, dementia, dementia associated with Down's Syndrome, cognitive deficits in psychiatric disorders, dementia associated with Lewy Body pathology, diminished CNS function associated with traumatic brain injury, Pick's Disease, spinal cord injury, a demyelinating disorder, a disorder of basal ganglia and AIDS-associated dementia. Given the neuro-inflammatory actions of S1P receptors, and S1P5 in specific, as well as the peripheral localization of S1P5 in skin tissue and a role in endothelial function and NK cells, the compounds of the invention are further suitable to treat or alleviate a disease with a neuro-inflammatory component, in particular a disease or disorder selected from the group consisting of Psoriasis type 1 and type 2, atopic dermatitis, dermatitis scleroderma, insulin-dependent diabetes mellitus, ulcerative colitis, atherosclerosis, sepsis syndrome, septic shock, Dengue hemorrhagic fever, Dengue, atopic allergy, HIV/AIDS, barrier-integrity associated lung diseases, leukemia, contact dermatitis, encephalomyelitis, Epstein Barr virus infection and other virus infections requiring cell-cell fusion.

In the compounds of the invention, or pharmaceutically acceptable salts thereof, m is 0 or 1 and n is 0 or 1. The nitrogen containing ring preferably is a 4-membered or 6-membered ring. Hence, preferably m and n are both 0 or both 1. In one embodiment, m and n are both 1 and the compound has the formula (Ib):

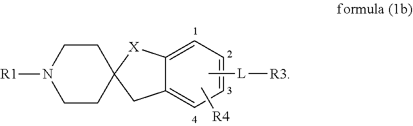

Most preferably m and n are both 0 and the compound has the formula (Ia):

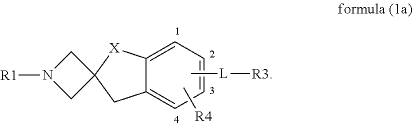

In one embodiment X is O. In a preferred embodiment, X is CH₂.

In the compounds of the invention, or pharmaceutically acceptable salts thereof, L is attached to one of the atoms numbered 1, 2, 3 or 4. Preferably, the group L-R3 is attached to one of the carbon atoms numbered 2 or 3. Hence, formula (I) is preferably selected from formula (Ic) and formula (Id):

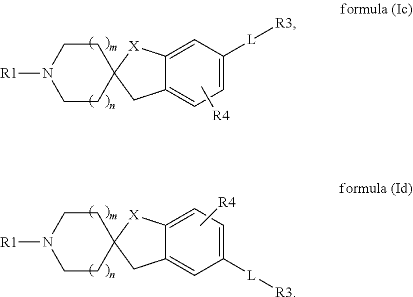

In a further preferred embodiment, formula (I) is selected from formula (Ie) and (If):

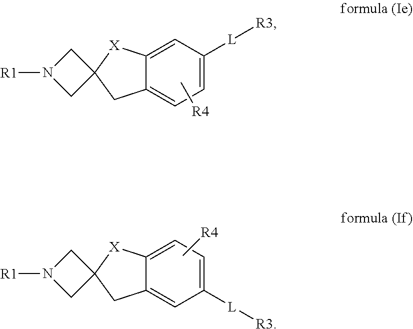

In a particularly preferred embodiment, a compound of the invention is a compound of formula (Ie).

L is a group —W—(CH₂)p-T- wherein:

W is attached to the phenylene moiety and selected from the group consisting of —O—, —CO—, —S—, —SO—, —SO₂—, —NH—, —CH₂—CH₂—, —CF₂—CH₂—, —CH₂—CF₂—, —CH═CH—, —C(CF₃)═CH—, —CH═C(CF₃)—, —C≡C—, phenyl, —(C3-7)cycloalkyl-, -pyridyl-, -thienyl- and -thiazolyl-, wherein the phenyl, (C3-7)cycloalkyl, pyridyl, thienyl or thiazolyl is optionally substituted with one or more substituents independently selected from the group consisting of a halogen atom, hydroxy, (C1-4)alkyl optionally substituted with one or more halogen atoms, and (C1-4)alkoxy optionally substituted with one or more halogen atoms;
p is 0 or an integer from 1 to 4; and
T is absent or attached to R3 and selected from the group consisting of —O—, —O—(C1-4)alkyl-, —S—, —SO—, —SO₂—, —NH—, —CO—, —CH═CH—, —C≡C— and cyclopropylene;

Preferably, W is selected from the group consisting of —O—, —CO—, —S—, —SO—, —SO₂—, —CH₂—CH₂—, —CF₂—CH₂—, —CH₂—CF₂—, —CH═CH—, —C(CF₃)═CH—, —CH═C(CF₃)—, —C≡C—, and -phenyl-, p is 0, or an integer from 1 to 4 and T is absent or selected from the group consisting of —O— and —O—(C1-4)alkyl-.

In a particular embodiment, T is —O— or —O—(C1-4)alkyl-, preferably —O— or —O—CH₂—, if W is selected from the group consisting of —O— and optionally substituted-phenyl, —(C3-7)cycloalkyl-, -pyridyl-, -thienyl- or -thiazolyl-. Otherwise, i.e. if W is —CO—, —S—, —SO—, —SO₂—, —NH—, —CH₂—CH₂—, —CF₂—CH₂—, —CH₂—CF₂—, —CH═CH—, —C(CF₃)═CH—, —CH═C(CF₃)— or —C≡C—, T is preferably absent.

In a further preferred embodiment, L is selected from the group consisting of —(C2-4)alkyl-, —O—, —O—(C1-3)alkyl-, —O—(C1-4)alkyl-O—, -phenyl-(C1-4)alkyl-, -phenyl-O—(C1-4)alkyl-, —CH═CH— and —C≡C—, more preferably from the group consisting of —(C2-4)alkyl-, —O—, —O—(C1-3)alkyl-, —O—(CH₂)₂—O—, -phenyl-O—CH₂— and —C≡C—.

R1 is selected from the group consisting of —(C1-4)alkylene-R2, —(C3-6)cycloalkylene-R2, —(C1-3)alkylene-(C3-6)cycloalkylene-R2 and —(C3-6)cycloalkylene-(C1-3)alkylene-R2, wherein the (C1-4)alkylene is optionally substituted with up to 3 carbon atoms, with (CH₂)₂ to form a cyclopropyl moiety or with (CH₂)₃ to form a cyclobutyl moiety. Preferably R1 is —(C1-3)alkylene-R2 or —(C3-6)cycloalkylene-R2, wherein the (C1-3)alkylene is optionally substituted with up to two CH₃ groups, with (CH₂)₂ to form a cyclopropyl moiety or with (CH₂)₃ to form a cyclobutyl moiety.

In a preferred embodiment, R1 is selected from the group consisting of —CH₂—R2, —(CH₂)₂—R2, —CH(CH₃)—CH₂—R2, —CH₂—C(CH₃)—CH₂—R2,

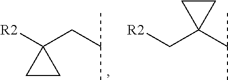

and -1,3-cyclobutylene-R2.

In a further preferred embodiment, R1 is selected from the group consisting of -1,3-cyclobutylene-R2 and —(C1-3)alkylene-R2, wherein the (C1)alkyl is unsubstituted and the (C2)alkyl and the (C3)alkyl are substituted with up to two CH₃ groups, with (CH₂)₂ to form a cyclopropyl moiety or with (CH₂)₃ to form a cyclobutyl moiety.

In a further preferred embodiment, R1 is selected from the group consisting of —CH₂—R2, —C2-alkylene-R2 wherein the C2-alkylene is substituted with up to two CH₃ groups, with (CH₂)₂ to form a cyclopropyl moiety or with (CH₂)₃ to form a cyclobutyl moiety, —C3-alkylene-R2 wherein the alkylene is substituted with one CH₃ group, and —(C3-6)cycloalkylene-R2. In a particularly preferred embodiment, R1 is selected from the group consisting of —CH₂—R2, —CH₂—C(CH₃)—CH₂—R2

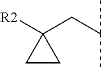

and -1,3-cyclobutylene-R2, wherein R2 is preferably COOH. In a further particularly preferred embodiment, R1 is —CH₂—R2,

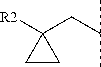

or -1,3-cyclobutylene-R2, wherein R2 is COOH.

R2 is selected from the group consisting of —COOH, —OH, —OPO₃H₂, —PO₃H₂, —COO(C1-4)alkyl and tetrazol-5-yl. R2 is preferably selected from the group consisting of —OH, —COOH and —COO(C1-4)alkyl. In a particularly preferred embodiment, R2 is —COOH.

R3 is selected from the group consisting of (C3-6)cycloalkyl, (C4-6)cycloalkenyl, phenyl, biphenyl, naphthyl, a monocyclic heterocycle and a 8-10 membered fused bicyclic group, each optionally substituted with one or more substituents independently selected from the group consisting of:
halogen,
cyano,
(C1-6)alkyl optionally substituted with one or more fluoro atoms,
(C1-4)alkoxy optionally substituted with one or more fluoro atoms or with (C3-6)cycloalkyl,
(C3-6)cycloalkoxy optionally substituted with one or more fluoro atoms;
—S—(C1-4)-alkyl,
—SF₅,
phenoxy, benzyl or benzyloxy, and
(C3-8)cycloalkyl optionally substituted with phenyl, (C1-4)alkyl, (C1-4)alkoxy or a halogen atom.

Preferably, R3 is selected from the group consisting of (C3-6)cycloalkyl, (C4-6)cycloalkenyl, phenyl, a monocyclic heterocycle and a 8-10 membered fused bicyclic group, each optionally substituted with one or more substituents, preferably 1 to 3 substituents, independently selected from the group consisting of:
halogen,
cyano,
(C1-4)alkyl optionally substituted with one or more fluoro atoms,
(C1-4)alkoxy optionally substituted with one or more fluoro atoms or with (C3-6)cycloalkyl,
(C3-6)cycloalkoxy optionally substituted with one or more fluoro atoms; and
(C3-8)cycloalkyl optionally substituted with (C1-4)alkyl, (C1-4)alkoxy or a halogen atom.

More preferably, R3 is selected from the group consisting of:
(C3-6)cycloalkyl, preferably cyclohexyl, optionally substituted with (C1-4)alkyl;
monocyclic heterocycle, preferably oxanyl or pyridyl, most preferably pyridyl, optionally substituted with 1 or 2 substituents independently selected from the group consisting of:
   halogen,
   cyano,
   (C1-4)alkyl optionally substituted with one or more fluoro atoms,
   (C1-4)alkoxy optionally substituted with one or more fluoro atoms or with (C3-6)cycloalkyl,
   (C3-5)cycloalkoxy optionally substituted with one or more fluoro atoms; and
   (C3-6)cycloalkyl optionally substituted with (C1-4)alkyl, (C1-4)alkoxy or a halogen atom;
indanyl, optionally substituted with one or two halogen atoms;
8-10 membered fused bicyclic group, preferably 2,3-dihydrobenzofuranyl, or indanyl, optionally substituted with one or two halogen atoms;
phenyl optionally substituted with 1 to 3 substituents independently selected from the group consisting of:
   halogen,
   cyano,
   (C1-4)alkyl optionally substituted with one or more fluoro atoms,
   (C1-4)alkoxy optionally substituted with one or more fluoro atoms or with (C3-6)cycloalkyl,
   (C3-5)cycloalkoxy optionally substituted with one or more fluoro atoms; and
   (C3-6)cycloalkyl optionally substituted with (C1-4)alkyl, (C1-4)alkoxy or a halogen atom.

In a further preferred embodiment, R3 is selected from the group consisting of:
phenyl, indanyl, indolyl, oxanyl or pyridyl, optionally substituted with one or more substituents independently selected from the group consisting of:
   a halogen atom,
   (C1-4)alkyl optionally substituted with one or more fluoro atoms,
   (C1-4)alkoxy optionally substituted with one or more fluoro atoms or with (C3-6)cycloalkyl, (C3-6)cycloalkoxy optionally substituted with one or more fluoro atoms, and
2,3-dihydrobenzofuranyl or 1,3-dioxaindanyl.

A particularly preferred R3 is pyridyl, optionally substituted with 1 or 2 substituents independently selected from the group consisting of halogen, cyano, (C1-4)alkyl optionally substituted with one or more fluoro atoms, (C1-4)alkoxy optionally substituted with one or more fluoro atoms or with (C3-6)cycloalkyl, (C3-5)cycloalkoxy optionally substituted with one or more fluoro atoms, and (C3-6)cycloalkyl optionally substituted with (C1-4)alkyl, (C1-4)alkoxy or a halogen atom. Such optionally substituted pyridyl is further preferably attached to the cyclic core via linker —O—CH₂—, whereby the —CH₂— is attached to R3.

R4 is absent or is attached to atom 1, 2, 3 or 4 and selected from the group consisting of a halogen atom, (C1-4)alkyl optionally substituted with one or more halogen atoms, and (C1-4)alkoxy optionally substituted with one or more halogen atoms. Preferably, R4 is absent or a halogen atom. In a particularly preferred embodiment, R4 is absent.

Further particularly preferred compounds of the invention are compounds of formula (I), preferably formula (Ic) or (Id), or pharmaceutically acceptable salts thereof, wherein

X is CH₂ or O;

m and n are both 0 or both 1, preferably both 0;

R1 is selected from the group consisting of —CH₂—R2, —C2-alkylene-R2 wherein the C2-alkylene is substituted with up to two carbon atoms, with (CH₂)₂ to form a cyclopropyl moiety or with (CH₂)₃ to form a cyclobutyl moiety, and —(C3-6)cycloalkylene-R2, wherein R2 is selected from the group consisting of —COOH, —OH, —OPO₃H₂, —PO₃H₂, —COO(C1-4)alkyl and tetrazol-5-yl, preferably from the group consisting of —OH, —COOH and —COO(C1-4)alkyl;

L is attached to atom 1, 2, 3 or 4, preferably to atom 2 or 3, and is a group —W—(CH₂)p-T- wherein:

W is attached to the phenylene moiety and selected from the group consisting of —O—, —CO—, —S—, —SO—, —SO₂—, —CH₂—CH₂—, —CF₂—CH₂—, —CH₂—CF₂—, —CH═CH—, —C(CF₃)═CH—, —CH═C(CF₃)—, —C≡C—, and -phenyl-,
p is 0, or an integer from 1 to 4 and

T is absent or selected from the group consisting of —O— and —O—(C1-4)alkyl-;

R3 is selected from the group consisting of (C3-6)cycloalkyl, (C4-6)cycloalkenyl, phenyl, a monocyclic heterocycle and a 8-10 membered fused bicyclic group, each optionally substituted with one or more substituents, preferably 1 to 3 substituents, independently selected from the group consisting of:
halogen,
cyano,
(C1-4)alkyl optionally substituted with one or more fluoro atoms,
(C1-4)alkoxy optionally substituted with one or more fluoro atoms or with (C3-6)cycloalkyl,
(C3-6)cycloalkoxy optionally substituted with one or more fluoro atoms; and
(C3-8)cycloalkyl optionally substituted with (C1-4)alkyl, (C1-4)alkoxy or a halogen atom;

R4 is absent or selected from the group consisting of a halogen atom, (C1-4)alkyl optionally substituted with one or more halogen atoms, and (C1-4)alkoxy optionally substituted with one or more halogen atoms.

Further particularly preferred compounds of the invention are compounds of formula (Ie), or pharmaceutically acceptable salts thereof,

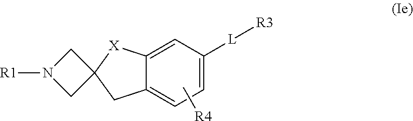

wherein

X is CH₂ or O;

R1 is selected from the group consisting of —CH₂—R2, —C2-alkylene-R2 wherein the C2-alkylene is substituted with up to two carbon atoms, with (CH₂)₂ to form a cyclopropyl moiety or with (CH₂)₃ to form a cyclobutyl moiety, —C3-alkylene-R2 wherein the 3 alkylene is substituted with one carbon atom, and —(C3-6)cycloalkylene-R2, wherein R2 is selected from the group consisting of —COOH, —OH, —OPO₃H₂, —PO₃H₂, —COO(C1-4)alkyl and tetrazol-5-yl, preferably from the group consisting of —OH, —COOH and —COO(C1-4)alkyl;

L is a group —W—(CH₂)p-T- wherein:

W is attached to the phenylene moiety and selected from the group consisting of —O—, —CO—, —S—, —SO—, —SO₂—, —CH₂—CH₂—, —CF₂—CH₂—, —CH₂—CF₂—, —CH═CH—, —C(CF₃)═CH—, —CH═C(CF₃)—, —C≡C—, and -phenyl-,

p is 0, or an integer from 1 to 4 and

T is absent or selected from the group consisting of —O— and —O—(C1-4)alkyl-;

R3 is selected from the group consisting of:
(C3-6)cycloalkyl, preferably cyclohexyl, optionally substituted with (C1-4)alkyl;
monocyclic heterocycle, preferably oxanyl or pyridyl, optionally substituted with 1 or 2 substituents independently selected from the group consisting of:
   halogen,
   cyano,
   (C1-4)alkyl optionally substituted with one or more fluoro atoms,
   (C1-4)alkoxy optionally substituted with one or more fluoro atoms or with (C3-6)cycloalkyl,
   (C3-5)cycloalkoxy optionally substituted with one or more fluoro atoms; and
   (C3-6)cycloalkyl optionally substituted with (C1-4)alkyl, (C1-4)alkoxy or a halogen atom,
indanyl, optionally substituted with one or two halogen atoms
8-10 membered fused bicyclic group, preferably 2,3-dihydrobenzofuranyl, indanyl, indoly or 1,3-dioxaindanyl, optionally substituted with one or two halogen atoms;
phenyl optionally substituted with 1 to 3 substituents independently selected from the group consisting of:
   halogen,
   cyano,
   (C1-4)alkyl optionally substituted with one or more fluoro atoms,
   (C1-4)alkoxy optionally substituted with one or more fluoro atoms or with (C3-6)cycloalkyl,
   (C3-5)cycloalkoxy optionally substituted with one or more fluoro atoms; and
   (C3-6)cycloalkyl optionally substituted with (C1-4)alkyl, (C1-4)alkoxy or a halogen atom; and

R4 is absent.

Further particularly preferred compounds of the invention are compounds of formula (Ie), or pharmaceutically acceptable salts thereof,

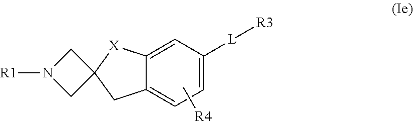

wherein

X is CH₂ or O;

R1 is selected from the group consisting of —CH2-R2, —CH2-C(CH3)-CH2-R2, and -1,3-cyclobutylene-R2, wherein R2 is selected from the group consisting of —OH, —COOH and —COO(C1-4)alkyl, and preferably is —OH or —COOH, more preferably —COOH;

L is selected from the group consisting of —(C2-4)alkyl-, —O—, —O—(C1-3)alkyl-, —O—(C1-4)alkyl-O—, -phenyl-(C1-4)alkyl-, -phenyl-O—(C1-4)alkyl-, —CH═CH— and —C≡C—, more preferably from the group consisting of —(C2-4)alkyl-, —O—, —O—(C1-3)alkyl-, —O—(CH₂)₂—O—, -phenyl-O—CH₂- and —C≡C—;

R3 is selected from the group consisting of:
(C3-6)cycloalkyl, preferably cyclohexyl, optionally substituted with (C1-4)alkyl;
monocyclic heterocycle, preferably oxanyl or pyridyl, optionally substituted with one or two substituents independently selected from the group consisting of:
   halogen,
   cyano,
   (C1-4)alkyl optionally substituted with one or more fluoro atoms,
   (C1-4)alkoxy optionally substituted with one or more fluoro atoms or with (C3-6)cycloalkyl,
   (C3-5)cycloalkoxy optionally substituted with one or more fluoro atoms; and
   (C3-6)cycloalkyl optionally substituted with (C1-4)alkyl, (C1-4)alkoxy or a halogen atom,
indanyl, optionally substituted with one or two halogen atoms
8-10 membered fused bicyclic group, preferably 2,3-dihydrobenzofuranyl, indanyl, indoly or 1,3-dioxaindanyl, optionally substituted with one or two halogen atoms;
phenyl optionally substituted with 1 to 3 substituents independently selected from the group consisting of:
   halogen,
   cyano,
   (C1-4)alkyl optionally substituted with one or more fluoro atoms,
   (C1-4)alkoxy optionally substituted with one or more fluoro atoms or with (C3-6)cycloalkyl,
   (C3-5)cycloalkoxy optionally substituted with one or more fluoro atoms; and
   (C3-6)cycloalkyl optionally substituted with (C1-4)alkyl, (C1-4)alkoxy or a halogen atom; and

R4 is absent.

Further particularly preferred compounds of the invention are compounds of formula (Ie), or pharmaceutically acceptable salts thereof,

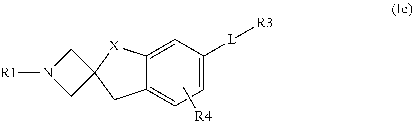

wherein

X is CH₂ or O;

R1 is selected from the group consisting of —CH2-R2, —CH2-C(CH3)-CH2-R2, and -1,3-cyclobutylene-R2, wherein R2 is selected from the group consisting of —OH, —COOH and —COO(C1-4)alkyl, and preferably is —OH or —COOH, more preferably —COOH;

L is —O—(C1-3)alkyl-, preferably —O—CH₂—;

R3 is a monocyclic heterocycle, preferably oxanyl or pyridyl, optionally substituted with one or two substituents independently selected from the group consisting of
halogen,
(C1-4)alkyl optionally substituted with one or more fluoro atoms,
(C1-4)alkoxy optionally substituted with one or more fluoro atoms or with (C3-6)cycloalkyl,
(C3-5)cycloalkoxy optionally substituted with one or more fluoro atoms; and
(C3-6)cycloalkyl optionally substituted with (C1-4)alkyl, (C1-4)alkoxy or a halogen atom; and

R4 is absent.

Further particularly preferred compounds of the invention are compounds of formula (I), or pharmaceutically acceptable salts thereof, wherein

X is CH₂;

m and n are both 0 or both 1, preferably both 0;

R1 is selected from the group consisting of -1,3-cyclobutylene-R2 and —(C₁₋₃)alkylene-R2 wherein the (C1)alkylene is unsubstituted, and the (C2)-alkylene and the (C3)-alkylene are substituted with up to two carbon atoms, with (CH₂)₂ to form a cyclopropyl moiety or with (CH₂)₃ to form a cyclobutyl moiety, wherein R2 is selected from the group consisting of —COOH, —OH, —OPO₃H₂, —PO₃H₂, —COO(C1-4)alkyl and tetrazol-5-yl, preferably from the group consisting of —OH, —COOH and —COO(C1-4)alkyl;

L is attached to atom 2 or 3, and is a group —W—(CH₂)p-T- wherein:

W is attached to the phenylene moiety and selected from the group consisting of —O—, —CO—, —S—, —SO—, —SO₂—, —CH₂—CH₂—, —CF₂—CH₂—, —CH₂—CF₂—, —CH═CH—, —C(CF₃)═CH—, —CH═C(CF₃)—, —C≡C—, and -phenyl-,

p is 0, or an integer from 1 to 4 and

T is absent or selected from the group consisting of —O— and —O—(C1-4)alkyl-;

R3 is selected from the group consisting of:
(C3-6)cycloalkyl, preferably cyclohexyl, optionally substituted with (C1-4)alkyl;
monocyclic heterocycle, preferably oxanyl or pyridyl, optionally substituted with 1 or 2 substituents independently selected from the group consisting of:
   halogen,
   cyano,
   (C1-4)alkyl optionally substituted with one or more fluoro atoms,
   (C1-4)alkoxy optionally substituted with one or more fluoro atoms or with (C3-6)cycloalkyl,
   (C3-5)cycloalkoxy optionally substituted with one or more fluoro atoms; and
   (C3-6)cycloalkyl optionally substituted with (C1-4)alkyl, (C1-4)alkoxy or a halogen atom,
indanyl, optionally substituted with one or two halogen atoms
8-10 membered fused bicyclic group, preferably preferably 2,3-dihydrobenzofuranyl, indanyl, indoly or 1,3-dioxaindanyl, optionally substituted with one or two halogen atoms;
phenyl optionally substituted with 1 to 3 substituents independently selected from the group consisting of:
   halogen,
   cyano,
   (C1-4)alkyl optionally substituted with one or more fluoro atoms,
   (C1-4)alkoxy optionally substituted with one or more fluoro atoms or with (C3-6)cycloalkyl,
   (C3-5)cycloalkoxy optionally substituted with one or more fluoro atoms; and
   (C3-6)cycloalkyl optionally substituted with (C1-4)alkyl, (C1-4)alkoxy or a halogen atom;

R4 is absent or selected from the group consisting of a halogen atom, (C1-4)alkyl optionally substituted with one or more halogen atoms, and (C1-4)alkoxy optionally substituted with one or more halogen atoms.

Further preferred compounds of the invention are depicted in table 1, and include their pharmaceutically acceptable salts.

Particularly preferred compounds depicted in table 1 are compounds having a EC50 for the S1P5 receptor of 100 nM or less, as shown in table 1, i.e. compounds having an S1P5 EC50 range A or B in table 1. Such compounds having an EC50 for the S1P5 receptor of 100 nM or less further preferably have an EC50 for at least one of the S1P1 receptor, the S1P3 receptor and the S1P4 receptor of more than 1 μM. Hence, in a preferred embodiment are provided compounds depicted in table 1 having an S1P5 EC50 of 100 nM or less (indicated with range A or B in table 1) and EC50 of more than 1 μM for at least one of the S1P1 receptor, the S1P3 receptor and the S1P4 receptor.

Further particularly preferred compounds depicted in table 1 are compounds having a EC50 for the S1P5 receptor of 10 nM or less, as shown in table 1, i.e. compounds having an S1P5 EC50 range A in table 1. Hence, a preferred compound according to the invention is selected from the group consisting of:
3-(5′-((2-chloro-6-ethylbenzyl)oxy)-1′,3′-dihydrospiro[azetidine-3,2′-inden]-1-yl)propanoic acid;
2-(5′-((2-chloro-6-cyclopropylbenzyl)oxy)-1′,3′-dihydrospiro[azetidine-3,2′-inden]-1-yl)acetic acid;
2-(5′-((3-chlorobenzyl)oxy)-1′,3′-dihydrospiro[azetidine-3,2′-inden]-1-yl)acetic acid compound with acetic acid;
2-(5′-((2-fluoro-6-methylbenzyl)oxy)-1′,3′-dihydrospiro[azetidine-3,2′-inden]-1-yl)acetic acid;
2-(5′-((2-chloro-6-fluorobenzyl)oxy)-1′,3′-dihydrospiro[azetidine-3,2′-inden]-1-yl)acetic acid;
2-(5′-((2-fluoro-6-(trifluoromethyl)benzyl)oxy)-1′,3′-dihydrospiro[azetidine-3,2′-inden]-1-yl)acetic acid;
2-(5′-((2-chloro-6-(trifluoromethyl)benzyl)oxy)-1′,3′-dihydrospiro[azetidine-3,2′-inden]-1-yl)acetic acid;
2-(5′-((2-ethyl-6-fluorobenzyl)oxy)-1′,3′-dihydrospiro[azetidine-3,2′-inden]-1-yl)acetic acid;
2-(5′-((2,5-dichlorobenzyl)oxy)-1′,3′-dihydrospiro[azetidine-3,2′-inden]-1-yl)acetic acid;
2-(5′-((2-chloro-6-ethylbenzyl)oxy)-1′,3′-dihydrospiro[azetidine-3,2′-inden]-1-yl)acetic acid;
2-(5′-((5-fluoro-2-(trifluoromethyl)benzyl)oxy)-1′,3′-dihydrospiro[azetidine-3,2′-inden]-1-yl)acetic acid;
1-((5′-((2-chloro-6-ethylbenzyl)oxy)-1′,3′-dihydrospiro[azetidine-3,2′-inden]-1-yl)methyl)cyclopropanecarboxylic acid;
1-((5′-((2,6-dimethylbenzyl)oxy)-1′,3′-dihydrospiro[azetidine-3,2′-inden]-1-yl)methyl)cyclopropanecarboxylic acid;
1-((5′-((2-chloro-6-cyclopropylbenzyl)oxy)-1′,3′-dihydrospiro[azetidine-3,2′-inden]-1-yl)methyl)cyclopropanecarboxylic acid;
1-((5′-((2,6-dichlorobenzyl)oxy)-1′,3′-dihydrospiro[azetidine-3,2′-inden]-1-yl)methyl)cyclopropanecarboxylic acid;
1-((5′-((2-chloro-6-(trifluoromethyl)benzyl)oxy)-1′,3′-dihydrospiro[azetidine-3,2′-inden]-1-yl)methyl)cyclopropanecarboxylic acid;
1-((5′-((2-ethyl-6-fluorobenzyl)oxy)-1′,3′-dihydrospiro[azetidine-3,2′-inden]-1-yl)methyl)cyclopropanecarboxylic acid;
1-((5′-((2-fluoro-6-(trifluoromethyl)benzyl)oxy)-1′,3′-dihydrospiro[azetidine-3,2′-inden]-1-yl)methyl)cyclopropanecarboxylic acid;
1-((5′-(4-((3-fluorobenzyl)oxy)phenyl)-1′,3′-dihydrospiro[azetidine-3,2′-inden]-1-yl)methyl)cyclopropanecarboxylic acid;
1-((5′-(2,6-dichlorophenethoxy)-1′,3′-dihydrospiro[azetidine-3,2′-inden]-1-yl)methyl)cyclopropanecarboxylic acid;
2-(5′-(2-chlorophenethoxy)-1′,3′-dihydrospiro[azetidine-3,2′-inden]-1-yl)acetic acid;
2-(5′-(2,6-dichlorophenethoxy)-1′,3′-dihydrospiro[azetidine-3,2′-inden]-1-yl)acetic acid;
4-(5′-((2-chloro-6-ethylbenzyl)oxy)-1′,3′-dihydrospiro[azetidine-3,2′-inden]-1-yl)-3-methylbutanoic acid;
2-(5′-((2,3-dihydro-1H-inden-1-yl)oxy)-1′,3′-dihydrospiro[azetidine-3,2′-inden]-1-yl)acetic acid;
2-(5′-((7-chloro-2,3-dihydro-1H-inden-1-yl)oxy)-1′,3′-dihydrospiro[azetidine-3,2′-inden]-1-yl)acetic acid;
2-(5′-((5-bromo-7-fluoro-2,3-dihydro-1H-inden-1-yl)oxy)-1′,3′-dihydrospiro[azetidine-3,2′-inden]-1-yl)acetic acid;
2-(5′-((5,7-dichloro-2,3-dihydro-1H-inden-1-yl)oxy)-1′,3′-dihydrospiro[azetidine-3,2′-inden]-1-yl)acetic acid;
2-(5′-((2,3-dihydro-1H-inden-4-yl)methoxy)-1′,3′-dihydrospiro[azetidine-3,2′-inden]-1-yl)acetic acid;
2-(5′-((6-isopropoxypyridin-3-yl)ethynyl)-1′,3′-dihydrospiro[azetidine-3,2′-inden]-1-yl)acetic acid;
2-(5′-((4-methylcyclohexyl)ethynyl)-1′,3′-dihydrospiro[azetidine-3,2′-inden]-1-yl)acetic acid;
2-(5′-((4-ethoxyphenyl)ethynyl)-1′,3′-dihydrospiro[azetidine-3,2′-inden]-1-yl)acetic acid;
2-(5′-((6-(cyclopentyloxy)pyridin-3-yl)ethynyl)-1′,3′-dihydrospiro[azetidine-3,2′-inden]-1-yl)acetic acid;
2-(5′-((6-((1,1,1-trifluoropropan-2-yl)oxy)pyridin-3-yl)ethynyl)-1′,3′-dihydrospiro[azetidine-3,2′-inden]-1-yl)acetic acid;
2-(5′-((2-chlorophenyl)ethynyl)-1′,3′-dihydrospiro[azetidine-3,2′-inden]-1-yl)acetic acid;
3-(5′-((2-cyclopropyl-6-fluorobenzyl)oxy)-1′,3′-dihydrospiro[azetidine-3,2′-inden]-1-yl)cyclobutanecarboxylic acid;
3-(5′-((2-chloro-6-cyclopropylbenzyl)oxy)-1′,3′-dihydrospiro[azetidine-3,2′-inden]-1-yl)cyclobutanecarboxylic acid;
3-(5′-((2,6-dichlorobenzyl)oxy)-1′,3′-dihydrospiro[azetidine-3,2′-inden]-1-yl)cyclobutanecarboxylic acid;
3-(5′-((2-chloro-6-ethylbenzyl)oxy)-1′,3′-dihydrospiro[azetidine-3,2′-inden]-1-yl)cyclobutanecarboxylic acid;
3-(5′-((6-(cyclopentyloxy)pyridin-3-yl)ethynyl)-1′,3′-dihydrospiro[azetidine-3,2′-inden]-1-yl)cyclobutanecarboxylic acid;
3-(5′-((4-isopropoxyphenyl)ethynyl)-1′,3′-dihydrospiro[azetidine-3,2′-inden]-1-yl)cyclobutanecarboxylic acid;
3-(5′-((4-ethoxy-3-fluorophenyl)ethynyl)-1′,3′-dihydrospiro[azetidine-3,2′-inden]-1-yl)cyclobutanecarboxylic acid;
3-(5′-((4-methylcyclohexyl)ethynyl)-1′,3′-dihydrospiro[azetidine-3,2′-inden]-1-yl)cyclobutanecarboxylic acid;
3-(5′-((6-((1,1,1-trifluoropropan-2-yl)oxy)pyridin-3-yl)ethynyl)-1′,3′-dihydrospiro[azetidine-3,2′-inden]-1-yl)cyclobutanecarboxylic acid;
3-(5′-((2,3-dihydro-1H-inden-1-yl)oxy)-1′,3′-dihydrospiro[azetidine-3,2′-inden]-1-yl)cyclobutanecarboxylic acid;
3-(5′-((7-chloro-2,3-dihydro-1H-inden-1-yl)oxy)-1′,3′-dihydrospiro[azetidine-3,2′-inden]-1-yl)cyclobutanecarboxylic acid;
2-(5′-((2,6-dichlorobenzyl)oxy)-1′,3′-dihydrospiro[azetidine-3,2′-inden]-1-yl)acetic acid;
2-(5′-((2-cyclopropyl-6-fluorobenzyl)oxy)-1′,3′-dihydrospiro[azetidine-3,2′-inden]-1-yl)acetic acid;
2-(5′-((2-cyclopropylbenzyl)oxy)-1′,3′-dihydrospiro[azetidine-3,2′-inden]-1-yl)acetic acid;
1-((5′-((4-isopropoxyphenyl)ethynyl)-1′,3′-dihydrospiro[azetidine-3,2′-inden]-1-yl)methyl)cyclopropanecarboxylic acid;
1-((5′-((4-methylcyclohexyl)ethynyl)-1′,3′-dihydrospiro[azetidine-3,2′-inden]-1-yl)methyl)cyclopropanecarboxylic acid;
1-((5′-((6-((1,1,1-trifluoropropan-2-yl)oxy)pyridin-3-yl)ethynyl)-1′,3′-dihydrospiro[azetidine-3,2′-inden]-1-yl)methyl)cyclopropanecarboxylic acid;
1-((5′-((7-chloro-2,3-dihydro-1H-inden-1-yl)oxy)-1′,3′-dihydrospiro[azetidine-3,2′-inden]-1-yl)methyl)cyclopropanecarboxylic acid;
1-((5′-((2-cyclopropyl-6-fluorobenzyl)oxy)-1′,3′-dihydrospiro[azetidine-3,2′-inden]-1-yl)methyl)cyclopropanecarboxylic acid;
2-(6′-(cyclohexylethynyl)-3′H-spiro[azetidine-3,2′-benzofuran]-1-yl)acetic acid;
2-(6′-(phenylethynyl)-3′H-spiro[azetidine-3,2′-benzofuran]-1-yl)acetic acid;
2-(6′-((3-chlorophenyl)ethynyl)-3′H-spiro[azetidine-3,2′-benzofuran]-1-yl)acetic acid;
2-(6′-((2-chlorophenyl)ethynyl)-3′H-spiro[azetidine-3,2′-benzofuran]-1-yl)acetic acid;
1-((6′-((2-chloro-6-ethylbenzyl)oxy)-3′H-spiro[azetidine-3,2′-benzofuran]-1-yl)methyl)cyclopropanecarboxylic acid;
4-(6′-((2-chloro-6-ethylbenzyl)oxy)-3′H-spiro[azetidine-3,2′-benzofuran]-1-yl)-3-methylbutanoic acid;
2-(6′-((2-chloro-6-ethylbenzyl)oxy)-3′H-spiro[azetidine-3,2′-benzofuran]-1-yl)acetic acid;
2-(5-((2-ethyl-6-fluorobenzyl)oxy)-1,3-dihydrospiro[indene-2,4′-piperidin]-1′-yl)acetic acid;
2-(5-((2,6-dichlorobenzyl)oxy)-1,3-dihydrospiro[indene-2,4′-piperidin]-1′-yl)acetic acid and
2-(5-((2-chloro-6-ethylbenzyl)oxy)-1,3-dihydrospiro[indene-2,4′-piperidin]-1′-yl)acetic acid,
   or a pharmaceutically acceptable salt of any of these compounds.

Such compounds having an EC50 for the S1P5 receptor of 10 nM or less further preferably have an EC50 for at least one of the S1P1 receptor, the S1P3 receptor and the S1P4 receptor of more than 1 μM. Thus, in a preferred embodiment are provided compounds depicted in table 1 having an S1P5 EC50 of 10 nM or less (indicated with range A in table 1) and a EC50 of more than 1 μM for at least one of the S1P1 receptor, the S1P3 receptor and the S1P4 receptor. Hence, a preferred compound according to the invention is selected from the group consisting of:
3-(5′-((2-chloro-6-ethylbenzyl)oxy)-1′,3′-dihydrospiro[azetidine-3,2′-inden]-1-yl)propanoic acid;
2-(5′-((2-chloro-6-cyclopropylbenzyl)oxy)-1′,3′-dihydrospiro[azetidine-3,2′-inden]-1-yl)acetic acid;
2-(5′-((3-chlorobenzyl)oxy)-1′,3′-dihydrospiro[azetidine-3,2′-inden]-1-yl)acetic acid compound with acetic acid;
2-(5′-((2-fluoro-6-methylbenzyl)oxy)-1′,3′-dihydrospiro[azetidine-3,2′-inden]-1-yl)acetic acid;
2-(5′-((2-chloro-6-fluorobenzyl)oxy)-1′,3′-dihydrospiro[azetidine-3,2′-inden]-1-yl)acetic acid;
2-(5′-((2-fluoro-6-(trifluoromethyl)benzyl)oxy)-1′,3′-dihydrospiro[azetidine-3,2′-inden]-1-yl)acetic acid;
2-(5′-((2-chloro-6-(trifluoromethyl)benzyl)oxy)-1′,3′-dihydrospiro[azetidine-3,2′-inden]-1-yl)acetic acid;
2-(5′-((2-ethyl-6-fluorobenzyl)oxy)-1′,3′-dihydrospiro[azetidine-3,2′-inden]-1-yl)acetic acid;
2-(5′-((2,5-dichlorobenzyl)oxy)-1′,3′-dihydrospiro[azetidine-3,2′-inden]-1-yl)acetic acid;
2-(5′-((2-chloro-6-ethylbenzyl)oxy)-1′,3′-dihydrospiro[azetidine-3,2′-inden]-1-yl)acetic acid;
2-(5′-((5-fluoro-2-(trifluoromethyl)benzyl)oxy)-1′,3′-dihydrospiro[azetidine-3,2′-inden]-1-yl)acetic acid;
1-((5′-((2-chloro-6-ethylbenzyl)oxy)-1′,3′-dihydrospiro[azetidine-3,2′-inden]-1-yl)methyl)cyclopropanecarboxylic acid;
1-((5′-((2,6-dimethylbenzyl)oxy)-1′,3′-dihydrospiro[azetidine-3,2′-inden]-1-yl)methyl)cyclopropanecarboxylic acid;
1-((5′-((2-chloro-6-cyclopropylbenzyl)oxy)-1′,3′-dihydrospiro[azetidine-3,2′-inden]-1-yl)methyl)cyclopropanecarboxylic acid;
1-((5′-((2,6-dichlorobenzyl)oxy)-1′,3′-dihydrospiro[azetidine-3,2′-inden]-1-yl)methyl)cyclopropanecarboxylic acid;
1-((5′-((2-chloro-6-(trifluoromethyl)benzyl)oxy)-1′,3′-dihydrospiro[azetidine-3,2′-inden]-1-yl)methyl)cyclopropanecarboxylic acid;
1-((5′-((2-ethyl-6-fluorobenzyl)oxy)-1′,3′-dihydrospiro[azetidine-3,2′-inden]-1-yl)methyl)cyclopropanecarboxylic acid;
1-((5′-((2-fluoro-6-(trifluoromethyl)benzyl)oxy)-1′,3′-dihydrospiro[azetidine-3,2′-inden]-1-yl)methyl)cyclopropanecarboxylic acid;
1-((5′-(4-((3-fluorobenzyl)oxy)phenyl)-1′,3′-dihydrospiro[azetidine-3,2′-inden]-1-yl)methyl)cyclopropanecarboxylic acid;
1-((5′-(2,6-dichlorophenethoxy)-1′,3′-dihydrospiro[azetidine-3,2′-inden]-1-yl)methyl)cyclopropanecarboxylic acid;
2-(5′-(2-chlorophenethoxy)-1′,3′-dihydrospiro[azetidine-3,2′-inden]-1-yl)acetic acid;
2-(5′-(2,6-dichlorophenethoxy)-1′,3′-dihydrospiro[azetidine-3,2′-inden]-1-yl)acetic acid;
4-(5′-((2-chloro-6-ethylbenzyl)oxy)-1′,3′-dihydrospiro[azetidine-3,2′-inden]-1-yl)-3-methylbutanoic acid;
2-(5′-((2,3-dihydro-1H-inden-1-yl)oxy)-1′,3′-dihydrospiro[azetidine-3,2′-inden]-1-yl)acetic acid;
2-(5′-((7-chloro-2,3-dihydro-1H-inden-1-yl)oxy)-1′,3′-dihydrospiro[azetidine-3,2′-inden]-1-yl)acetic acid;
2-(5′-((5-bromo-7-fluoro-2,3-dihydro-1H-inden-1-yl)oxy)-1′,3′-dihydrospiro[azetidine-3,2′-inden]-1-yl)acetic acid;
2-(5′-((5,7-dichloro-2,3-dihydro-1H-inden-1-yl)oxy)-1′,3′-dihydrospiro[azetidine-3,2′-inden]-1-yl)acetic acid;
2-(5′-((2,3-dihydro-1H-inden-4-yl)methoxy)-1′,3′-dihydrospiro[azetidine-3,2′-inden]-1-yl)acetic acid;
2-(5′-((6-isopropoxypyridin-3-yl)ethynyl)-1′,3′-dihydrospiro[azetidine-3,2′-inden]-1-yl)acetic acid;
2-(5′-((4-methylcyclohexyl)ethynyl)-1′,3′-dihydrospiro[azetidine-3,2′-inden]-1-yl)acetic acid;
2-(5′-((2-chlorophenyl)ethynyl)-1′,3′-dihydrospiro[azetidine-3,2′-inden]-1-yl)acetic acid;
3-(5′-((2-cyclopropyl-6-fluorobenzyl)oxy)-1′,3′-dihydrospiro[azetidine-3,2′-inden]-1-yl)cyclobutanecarboxylic acid;
3-(5′-((2,6-dichlorobenzyl)oxy)-1′,3′-dihydrospiro[azetidine-3,2′-inden]-1-yl)cyclobutanecarboxylic acid;
3-(5′-((4-isopropoxyphenyl)ethynyl)-1′,3′-dihydrospiro[azetidine-3,2′-inden]-1-yl)cyclobutanecarboxylic acid;
3-(5′-((4-ethoxy-3-fluorophenyl)ethynyl)-1′,3′-dihydrospiro[azetidine-3,2′-inden]-1-yl)cyclobutanecarboxylic acid;
3-(5′-((4-methylcyclohexyl)ethynyl)-1′,3′-dihydrospiro[azetidine-3,2′-inden]-1-yl)cyclobutanecarboxylic acid;
3-(5′-((2,3-dihydro-1H-inden-1-yl)oxy)-1′,3′-dihydrospiro[azetidine-3,2′-inden]-1-yl)cyclobutanecarboxylic acid;
2-(5′-((2,6-dichlorobenzyl)oxy)-1′,3′-dihydrospiro[azetidine-3,2′-inden]-1-yl)acetic acid;
2-(5′-((2-cyclopropyl-6-fluorobenzyl)oxy)-1′,3′-dihydrospiro[azetidine-3,2′-inden]-1-yl)acetic acid;
2-(5′-((2-cyclopropylbenzyl)oxy)-1′,3′-dihydrospiro[azetidine-3,2′-inden]-1-yl)acetic acid;
1-((5′-((4-isopropoxyphenyl)ethynyl)-1′,3′-dihydrospiro[azetidine-3,2′-inden]-1-yl)methyl)cyclopropanecarboxylic acid;
1-((5′-((4-methylcyclohexyl)ethynyl)-1′,3′-dihydrospiro[azetidine-3,2′-inden]-1-yl)methyl)cyclopropanecarboxylic acid;
1-((5′-((6-((1,1,1-trifluoropropan-2-yl)oxy)pyridin-3-yl)ethynyl)-1′,3′-dihydrospiro[azetidine-3,2′-inden]-1-yl)methyl)cyclopropanecarboxylic acid;
1-((5′-((7-chloro-2,3-dihydro-1H-inden-1-yl)oxy)-1′,3′-dihydrospiro[azetidine-3,2′-inden]-1-yl)methyl)cyclopropanecarboxylic acid;
1-((5′-((2-cyclopropyl-6-fluorobenzyl)oxy)-1′,3′-dihydrospiro[azetidine-3,2′-inden]-1-yl)methyl)cyclopropanecarboxylic acid;
2-(6′-(cyclohexylethynyl)-3′H-spiro[azetidine-3,2′-benzofuran]-1-yl)acetic acid;
2-(6′-(phenylethynyl)-3′H-spiro[azetidine-3,2′-benzofuran]-1-yl)acetic acid;
2-(6′-((3-chlorophenyl)ethynyl)-3′H-spiro[azetidine-3,2′-benzofuran]-1-yl)acetic acid;
2-(6′-((2-chlorophenyl)ethynyl)-3′H-spiro[azetidine-3,2′-benzofuran]-1-yl)acetic acid;
1-((6′-((2-chloro-6-ethylbenzyl)oxy)-3′H-spiro[azetidine-3,2′-benzofuran]-1-yl)methyl)cyclopropanecarboxylic acid;
4-(6′-((2-chloro-6-ethylbenzyl)oxy)-3′H-spiro[azetidine-3,2′-benzofuran]-1-yl)-3-methylbutanoic acid;
2-(5-((2-ethyl-6-fluorobenzyl)oxy)-1,3-dihydrospiro[indene-2,4′-piperidin]-1′-yl)acetic acid;
2-(5-((2,6-dichlorobenzyl)oxy)-1,3-dihydrospiro[indene-2,4′-piperidin]-1′-yl)acetic acid and
2-(5-((2-chloro-6-ethylbenzyl)oxy)-1,3-dihydrospiro[indene-2,4′-piperidin]-1′-yl)acetic acid,
   or a pharmaceutically acceptable salt of any of these compounds.

In a preferred embodiment, a compound of the invention has a EC50 for the S1P5 receptor of 10 nM or less, i.e. compounds having an S1P5 EC50 range A in table 1, and an EC50 of more than 1 μM for the S1P1 receptor. Hence, a preferred compound according to the invention is selected from the group consisting of:
3-(5′-((2-chloro-6-ethylbenzyl)oxy)-1′,3′-dihydrospiro[azetidine-3,2′-inden]-1-yl)propanoic acid;
2-(5′-((2-chloro-6-cyclopropylbenzyl)oxy)-1′,3′-dihydrospiro[azetidine-3,2′-inden]-1-yl)acetic acid;
2-(5′-((3-chlorobenzyl)oxy)-1′,3′-dihydrospiro[azetidine-3,2′-inden]-1-yl)acetic acid compound with acetic acid;
2-(5′-((2-fluoro-6-methylbenzyl)oxy)-1′,3′-dihydrospiro[azetidine-3,2′-inden]-1-yl)acetic acid;
2-(5′-((2-chloro-6-fluorobenzyl)oxy)-1′,3′-dihydrospiro[azetidine-3,2′-inden]-1-yl)acetic acid;
2-(5′-((2-fluoro-6-(trifluoromethyl)benzyl)oxy)-1′,3′-dihydrospiro[azetidine-3,2′-inden]-1-yl)acetic acid;
2-(5′-((2-chloro-6-(trifluoromethyl)benzyl)oxy)-1′,3′-dihydrospiro[azetidine-3,2′-inden]-1-yl)acetic acid;
2-(5′-((2-ethyl-6-fluorobenzyl)oxy)-1′,3′-dihydrospiro[azetidine-3,2′-inden]-1-yl)acetic acid;
2-(5′-((2,5-dichlorobenzyl)oxy)-1′,3′-dihydrospiro[azetidine-3,2′-inden]-1-yl)acetic acid;
2-(5′-((2-chloro-6-ethylbenzyl)oxy)-1′,3′-dihydrospiro[azetidine-3,2′-inden]-1-yl)acetic acid;
2-(5′-((5-fluoro-2-(trifluoromethyl)benzyl)oxy)-1′,3′-dihydrospiro[azetidine-3,2′-inden]-1-yl)acetic acid;
1-((5′-((2-chloro-6-ethylbenzyl)oxy)-1′,3′-dihydrospiro[azetidine-3,2′-inden]-1-yl)methyl)cyclopropanecarboxylic acid;
1-((5′-((2,6-dimethylbenzyl)oxy)-1′,3′-dihydrospiro[azetidine-3,2′-inden]-1-yl)methyl)cyclopropanecarboxylic acid;
1-((5′-((2-chloro-6-cyclopropylbenzyl)oxy)-1′,3′-dihydrospiro[azetidine-3,2′-inden]-1-yl)methyl)cyclopropanecarboxylic acid;
1-((5′-((2,6-dichlorobenzyl)oxy)-1′,3′-dihydrospiro[azetidine-3,2′-inden]-1-yl)methyl)cyclopropanecarboxylic acid;
1-((5′-((2-chloro-6-(trifluoromethyl)benzyl)oxy)-1′,3′-dihydrospiro[azetidine-3,2′-inden]-1-yl)methyl)cyclopropanecarboxylic acid;
1-((5′-((2-ethyl-6-fluorobenzyl)oxy)-1′,3′-dihydrospiro[azetidine-3,2′-inden]-1-yl)methyl)cyclopropanecarboxylic acid;
1-((5′-((2-fluoro-6-(trifluoromethyl)benzyl)oxy)-1′,3′-dihydrospiro[azetidine-3,2′-inden]-1-yl)methyl)cyclopropanecarboxylic acid;
1-((5′-(4-((3-fluorobenzyl)oxy)phenyl)-1′,3′-dihydrospiro[azetidine-3,2′-inden]-1-yl)methyl)cyclopropanecarboxylic acid;
1-((5′-(2,6-dichlorophenethoxy)-1′,3′-dihydrospiro[azetidine-3,2′-inden]-1-yl)methyl)cyclopropanecarboxylic acid;
2-(5′-(2-chlorophenethoxy)-1′,3′-dihydrospiro[azetidine-3,2′-inden]-1-yl)acetic acid;
2-(5′-(2,6-dichlorophenethoxy)-1′,3′-dihydrospiro[azetidine-3,2′-inden]-1-yl)acetic acid;
2-(5′-((2,3-dihydro-1H-inden-1-yl)oxy)-1′,3′-dihydrospiro[azetidine-3,2′-inden]-1-yl)acetic acid;
2-(5′-((7-chloro-2,3-dihydro-1H-inden-1-yl)oxy)-1′,3′-dihydrospiro[azetidine-3,2′-inden]-1-yl)acetic acid;
2-(5′-((5-bromo-7-fluoro-2,3-dihydro-1H-inden-1-yl)oxy)-1′,3′-dihydrospiro[azetidine-3,2′-inden]-1-yl)acetic acid;
2-(5′-((5,7-dichloro-2,3-dihydro-1H-inden-1-yl)oxy)-1′,3′-dihydrospiro[azetidine-3,2′-inden]-1-yl)acetic acid;
2-(5′-((2,3-dihydro-1H-inden-4-yl)methoxy)-1′,3′-dihydrospiro[azetidine-3,2′-inden]-1-yl)acetic acid;
2-(5′-((4-methylcyclohexyl)ethynyl)-1′,3′-dihydrospiro[azetidine-3,2′-inden]-1-yl)acetic acid;
2-(5′-((2-chlorophenyl)ethynyl)-1′,3′-dihydrospiro[azetidine-3,2′-inden]-1-yl)acetic acid;
3-(5′-((2-cyclopropyl-6-fluorobenzyl)oxy)-1′,3′-dihydrospiro[azetidine-3,2′-inden]-1-yl)cyclobutanecarboxylic acid;
3-(5′-((2,6-dichlorobenzyl)oxy)-1′,3′-dihydrospiro[azetidine-3,2′-inden]-1-yl)cyclobutanecarboxylic acid;
3-(5′-((4-methylcyclohexyl)ethynyl)-1′,3′-dihydrospiro[azetidine-3,2′-inden]-1-yl)cyclobutanecarboxylic acid;
3-(5′-((2,3-dihydro-1H-inden-1-yl)oxy)-1′,3′-dihydrospiro[azetidine-3,2′-inden]-1-yl)cyclobutanecarboxylic acid;
2-(5′-((2,6-dichlorobenzyl)oxy)-1′,3′-dihydrospiro[azetidine-3,2′-inden]-1-yl)acetic acid;
2-(5′-((2-cyclopropyl-6-fluorobenzyl)oxy)-1′,3′-dihydrospiro[azetidine-3,2′-inden]-1-yl)acetic acid;
2-(5′-((2-cyclopropylbenzyl)oxy)-1′,3′-dihydrospiro[azetidine-3,2′-inden]-1-yl)acetic acid;
1-((5′-((4-methylcyclohexyl)ethynyl)-1′,3′-dihydrospiro[azetidine-3,2′-inden]-1-yl)methyl)cyclopropanecarboxylic acid;
1-((5′-((7-chloro-2,3-dihydro-1H-inden-1-yl)oxy)-1′,3′-dihydrospiro[azetidine-3,2′-inden]-1-yl)methyl)cyclopropanecarboxylic acid;
1-((5′-((2-cyclopropyl-6-fluorobenzyl)oxy)-1′,3′-dihydrospiro[azetidine-3,2′-inden]-1-yl)methyl)cyclopropanecarboxylic acid;
2-(6′-(cyclohexylethynyl)-3′H-spiro[azetidine-3,2′-benzofuran]-1-yl)acetic acid;
2-(6′-(phenylethynyl)-3′H-spiro[azetidine-3,2′-benzofuran]-1-yl)acetic acid;
2-(6′-((3-chlorophenyl)ethynyl)-3′H-spiro[azetidine-3,2′-benzofuran]-1-yl)acetic acid;
2-(6′-((2-chlorophenyl)ethynyl)-3′H-spiro[azetidine-3,2′-benzofuran]-1-yl)acetic acid;
1-((6′-((2-chloro-6-ethylbenzyl)oxy)-3′H-spiro[azetidine-3,2′-benzofuran]-1-yl)methyl)cyclopropanecarboxylic acid;
4-(6′-((2-chloro-6-ethylbenzyl)oxy)-3′H-spiro[azetidine-3,2′-benzofuran]-1-yl)-3-methylbutanoic acid;
2-(5-((2-ethyl-6-fluorobenzyl)oxy)-1,3-dihydrospiro[indene-2,4′-piperidin]-1′-yl)acetic acid;
2-(5-((2,6-dichlorobenzyl)oxy)-1,3-dihydrospiro[indene-2,4′-piperidin]-1′-yl)acetic acid and
2-(5-((2-chloro-6-ethylbenzyl)oxy)-1,3-dihydrospiro[indene-2,4′-piperidin]-1′-yl)acetic acid,
   or a pharmaceutically acceptable salt of any of these compounds.

In another preferred embodiment, a compound of the invention has a EC50 for the S1P5 receptor of 10 nM or less, i.e. compounds having an S1P5 EC50 range A in table 1, and a EC50 of more than 1 μM for the S1P3 receptor. Hence, a preferred compound according to the invention is selected from the group consisting of:
3-(5′-((2-chloro-6-ethylbenzyl)oxy)-1′,3′-dihydrospiro[azetidine-3,2′-inden]-1-yl)propanoic acid;
2-(5′-((2-chloro-6-cyclopropylbenzyl)oxy)-1′,3′-dihydrospiro[azetidine-3,2′-inden]-1-yl)acetic acid;
2-(5′-((3-chlorobenzyl)oxy)-1′,3′-dihydrospiro[azetidine-3,2′-inden]-1-yl)acetic acid compound with acetic acid;
2-(5′-((2,5-dichlorobenzyl)oxy)-1′,3′-dihydrospiro[azetidine-3,2′-inden]-1-yl)acetic acid;
2-(5′-((2-chloro-6-ethylbenzyl)oxy)-1′,3′-dihydrospiro[azetidine-3,2′-inden]-1-yl)acetic acid;
1-((5′-((2-chloro-6-ethylbenzyl)oxy)-1′,3′-dihydrospiro[azetidine-3,2′-inden]-1-yl)methyl)cyclopropanecarboxylic acid;
1-((5′-((2,6-dimethylbenzyl)oxy)-1′,3′-dihydrospiro[azetidine-3,2′-inden]-1-yl)methyl)cyclopropanecarboxylic acid;
1-((5′-((2-chloro-6-cyclopropylbenzyl)oxy)-1′,3′-dihydrospiro[azetidine-3,2′-inden]-1-yl)methyl)cyclopropanecarboxylic acid;
1-((5′-((2,6-dichlorobenzyl)oxy)-1′,3′-dihydrospiro[azetidine-3,2′-inden]-1-yl)methyl)cyclopropanecarboxylic acid;
1-((5′-(4-((3-fluorobenzyl)oxy)phenyl)-1′,3′-dihydrospiro[azetidine-3,2′-inden]-1-yl)methyl)cyclopropanecarboxylic acid;
1-((5′-(2,6-dichlorophenethoxy)-1′,3′-dihydrospiro[azetidine-3,2′-inden]-1-yl)methyl)cyclopropanecarboxylic acid;
2-(5′-(2-chlorophenethoxy)-1′,3′-dihydrospiro[azetidine-3,2′-inden]-1-yl)acetic acid;
2-(5′-(2,6-dichlorophenethoxy)-1′,3′-dihydrospiro[azetidine-3,2′-inden]-1-yl)acetic acid;
4-(5′-((2-chloro-6-ethylbenzyl)oxy)-1′,3′-dihydrospiro[azetidine-3,2′-inden]-1-yl)-3-methylbutanoic acid;
2-(5′-((2,6-dichlorobenzyl)oxy)-1′,3′-dihydrospiro[azetidine-3,2′-inden]-1-yl)acetic acid;
2-(5′-((2-cyclopropyl-6-fluorobenzyl)oxy)-1′,3′-dihydrospiro[azetidine-3,2′-inden]-1-yl)acetic acid;
2-(5′-((2-cyclopropylbenzyl)oxy)-1′,3′-dihydrospiro[azetidine-3,2′-inden]-1-yl)acetic acid;
2-(5-((2-ethyl-6-fluorobenzyl)oxy)-1,3-dihydrospiro[indene-2,4′-piperidin]-1′-yl)acetic acid;
2-(5-((2,6-dichlorobenzyl)oxy)-1,3-dihydrospiro[indene-2,4′-piperidin]-1′-yl)acetic acid and
2-(5-((2-chloro-6-ethylbenzyl)oxy)-1,3-dihydrospiro[indene-2,4′-piperidin]-1′-yl)acetic acid,
   or a pharmaceutically acceptable salt of any of these compounds.

In another preferred embodiment, a compound of the invention has a EC50 for the S1P5 receptor of 10 nM or less, i.e. compounds having an S1P5 EC50 range A in table 1, and a EC50 of more than 1 μM for the S1P4 receptor. Hence, a preferred compound according to the invention is selected from the group consisting of:
2-(5′-((6-isopropoxypyridin-3-yl)ethynyl)-1′,3′-dihydrospiro[azetidine-3,2′-inden]-1-yl)acetic acid;
3-(5′-((4-isopropoxyphenyl)ethynyl)-1′,3′-dihydrospiro[azetidine-3,2′-inden]-1-yl)cyclobutanecarboxylic acid;
3-(5′-((4-ethoxy-3-fluorophenyl)ethynyl)-1′,3′-dihydrospiro[azetidine-3,2′-inden]-1-yl)cyclobutanecarboxylic acid;
1-((5′-((4-isopropoxyphenyl)ethynyl)-1′,3′-dihydrospiro[azetidine-3,2′-inden]-1-yl)methyl)cyclopropanecarboxylic acid and
1-((5′-((6-((1,1,1-trifluoropropan-2-yl)oxy)pyridin-3-yl)ethynyl)-1′,3′-dihydrospiro[azetidine-3,2′-inden]-1-yl)methyl)cyclopropanecarboxylic acid, or a pharmaceutically acceptable salt of any of these compounds.

In another preferred embodiment are provided compounds depicted in table 1 having an S1P5 EC50 of 10 nM or less (indicated with range A in table 1) and a EC50 of more than 1 μM for at least two of the S1P1 receptor, the S1P3 receptor and the S1P4 receptor. Hence, a preferred compound according to the invention is selected from the group consisting of:
3-(5′-((2-chloro-6-ethylbenzyl)oxy)-1′,3′-dihydrospiro[azetidine-3,2′-inden]-1-yl)propanoic acid;
2-(5′-((2-chloro-6-cyclopropylbenzyl)oxy)-1′,3′-dihydrospiro[azetidine-3,2′-inden]-1-yl)acetic acid;
2-(5′-((3-chlorobenzyl)oxy)-1′,3′-dihydrospiro[azetidine-3,2′-inden]-1-yl)acetic acid compound with acetic acid;
2-(5′-((2,5-dichlorobenzyl)oxy)-1′,3′-dihydrospiro[azetidine-3,2′-inden]-1-yl)acetic acid;
2-(5′-((2-chloro-6-ethylbenzyl)oxy)-1′,3′-dihydrospiro[azetidine-3,2′-inden]-1-yl)acetic acid;
1-((5′-((2-chloro-6-ethylbenzyl)oxy)-1′,3′-dihydrospiro[azetidine-3,2′-inden]-1-yl)methyl)cyclopropanecarboxylic acid;
1-((5′-((2,6-dimethylbenzyl)oxy)-1′,3′-dihydrospiro[azetidine-3,2′-inden]-1-yl)methyl)cyclopropanecarboxylic acid;
1-((5′-((2-chloro-6-cyclopropylbenzyl)oxy)-1′,3′-dihydrospiro[azetidine-3,2′-inden]-1-yl)methyl)cyclopropanecarboxylic acid;
1-((5′-((2,6-dichlorobenzyl)oxy)-1′,3′-dihydrospiro[azetidine-3,2′-inden]-1-yl)methyl)cyclopropanecarboxylic acid;
1-((5′-(4-((3-fluorobenzyl)oxy)phenyl)-1′,3′-dihydrospiro[azetidine-3,2′-inden]-1-yl)methyl)cyclopropanecarboxylic acid;
1-((5′-(2,6-dichlorophenethoxy)-1′,3′-dihydrospiro[azetidine-3,2′-inden]-1-yl)methyl)cyclopropanecarboxylic acid;
2-(5′-(2-chlorophenethoxy)-1′,3′-dihydrospiro[azetidine-3,2′-inden]-1-yl)acetic acid;
2-(5′-(2,6-dichlorophenethoxy)-1′,3′-dihydrospiro[azetidine-3,2′-inden]-1-yl)acetic acid;
2-(5′-((2,6-dichlorobenzyl)oxy)-1′,3′-dihydrospiro[azetidine-3,2′-inden]-1-yl)acetic acid;
2-(5′-((2-cyclopropyl-6-fluorobenzyl)oxy)-1′,3′-dihydrospiro[azetidine-3,2′-inden]-1-yl)acetic acid;
2-(5′-((2-cyclopropylbenzyl)oxy)-1′,3′-dihydrospiro[azetidine-3,2′-inden]-1-yl)acetic acid;
2-(5-((2-ethyl-6-fluorobenzyl)oxy)-1,3-dihydrospiro[indene-2,4′-piperidin]-1′-yl)acetic acid;
2-(5-((2,6-dichlorobenzyl)oxy)-1,3-dihydrospiro[indene-2,4′-piperidin]-1′-yl)acetic acid and
2-(5-((2-chloro-6-ethylbenzyl)oxy)-1,3-dihydrospiro[indene-2,4′-piperidin]-1′-yl)acetic acid
   or a pharmaceutically acceptable salt of any of these compounds.

As used herein, the term “halogen” or “a halogen atom” refers to fluoro, chloro, bromo, or iodo. Preferred halogen atoms are fluoro and chloro.

As used herein, the term “(Cx-y)alkyl” refers to a branched or unbranched alkyl group having x-y carbon atoms. For instance, (C1-4)alkyl means a branched or unbranched alkyl group having 1-4 carbon atoms, for example methyl, ethyl, propyl, isopropyl or butyl. Similarly, the term “(C1-2) alkyl” refers to an alkyl group having 1 or 2 carbon atoms. Preferred alkyl groups are methyl and ethyl.

As used herein, the term (Cx-y)alkoxy refers to an alkoxy group having x-y carbon atoms, wherein the alkyl moiety is as defined above. For instance, the term (C1-4)alkoxy means an alkoxy group having a (C1-4)-alkyl moiety. Examples of alkoxy groups are methoxy, ethoxy, and —O—CH(CH₃)—CF₃.

As used herein, the term “(Cx-y)alkylene” refers to a branched or unbranched saturated alkylene group having x-y carbon atoms. For instance, the term “(C1-4)alkylene” means a saturated alkylene group having 1-4 carbon atoms, for example methylene, (CH₂)₃—CHCH₃—, —C(CH₃)₂—, —CHCH₃CH₂—. In the definition of R1 as —(C1-4)alkylene-R2, one or more carbon atoms in the alkylene group may independently be substituted with (CH₂)₂ to form a cyclopropyl moiety, for instance to form an R1 group

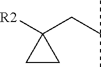

or with (CH₂)₃ to form a cyclobutyl moiety.

As used herein a dashed line in a partial structure, such as

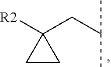

means that the partial structure is attached to the remainder of the structure at the site of the dashed line. For instance, if R1 is

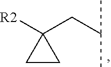

the compound of formula (I) is

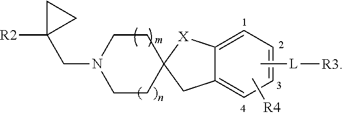

As used herein the term “(Cx-y)alkenyl” means a branched or unbranched alkenyl group having x-y carbon atoms, wherein the double bond may be present at various positions in the group. Examples are ethenyl, propenyl, 1-butenyl, 2-butenyl. For instance, the term “(C2-4)alkenyl” means a branched or unbranched alkenyl group having 2-4 carbon atoms.

As used herein, the term “(Cx-y)alkynyl” refers to a branched or unbranched alkynyl group having x-y carbon atoms, wherein the triple bond may be present at different positions in the group, for example ethynyl, propanyl, 1-butynyl, 2-butynyl. For instance, the term “(C2-4)alkynyl” refers to a branched or unbranched alkynyl group having 2-4 carbon atoms.

As used herein the term “(Cx-y)cycloalkyl” refers to a cyclic alkyl group having x-y carbon atoms. For instance, the term “(C3-6)cycloalkyl” refers to a cyclic alkyl group having 3-6 carbon atoms, i.e. cyclopropyl, cyclobutyl, cyclopentyl or cyclohexyl. A preferred (C3-8)cycloalkyl in the definition of R3 is cyclopentyl, cyclohexyl or cycloheptyl, more preferably cyclohexyl.

As used herein the term “(Cx-y)cycloalkoxy” means an alkoxy as defined above wherein the alkyl moiety is a Cx-y-cycloalkyl, e.g.

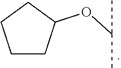

As used herein the term “(Cx-y)cycloalkenyl” means a cyclic alkenyl group having x-y carbon atoms. For instance, the term “(C4-6)cycloalkenyl” means a cyclic alkenyl group having 4-6 carbon atoms and comprising one or two double bonds, for example cyclohexenyl. Preferably a cycloalkenyl as used herein has one carbon-carbon double bond, e.g. cyclobutene, cyclopentene, cyclohexene and cycloheptene.

As used herein the term “(Cx-y)cycloalkylene” means a saturated cyclic group having x-y carbon atoms. For instance, the term “(C3-7)cycloalkylene” means a saturated cyclic group having 3-7 carbon atoms, e.g. cyclobutylene, cyclopentylene, cyclohexylene and cycloheptane.

As used herein the term “8-10 membered fused bicyclic group” for R3 means a fused ring system of two ring structures together having 8-10 atoms. The rings can be either aromatic or non-aromatic ring structures, preferably the fused bicyclic group contains at least one aromatic ring. Preferred 8-10 membered fused bicyclic groups in the definition of R3 contain up to two heteroatoms, preferably O, S and/or N. Preferred 8-10 membered bused bicyclic groups are indane, tetralin, benzofuran, isobenzofuran, dihydrobenzofuran, dihydroisobenzofuran, tetrahydrobenzofuran, tetrahydroisobenzofuran, indoline, isoindoline, indole, isoindole, dihydroindole, dihydroisoindole, tetrahydroindole, tetrahydroisoindole, quinolone, isoquinoline, tetrahydroquinoline, tetrahydroisoquinoline, quinoxaline, dihydroquinoxaline, tetrahydroquinoxaline, quinazoline, dihydroquinazoline, tetrahydroquinazoline, dihydrobenzopyran, benzothiophene, benzo[c]thiophene, dihydrobenzothiophene, dihydrobenzo[c]thiophene, tetrahydrobenzothiophene, tetrahydroquinoxaline, indazole, dihydroindazole, tetrahydroindazole, benzimidazole, dihydrobenzimidazole and tetrahydrobenzimidazole, benzoxazole, dihydrobenzoxazole, tetrahydrobenzoxazole, benzisoxazole, dihydrobenzisoxazole and tetrahydrobenzisoxazole. More preferred 8-10 membered fused bicyclic groups in the definition of R3 are indanyl, benzofuranyl, isobenzofuranyl, indolyl, isoindolyl, indazolyl, benzimidazolyl, benzothiophenyl, benzo[c]thiophenyl benzoxazolyl, 2,3-dihydrobenzofuranyl and 1,3-dioxaindanyl, more preferably indanyl, indolyl, 2,3-dihydrobenzofuranyl and 1,3-dioxaindanyl.

As used herein the term “monocyclic heterocycle” means a heteroatom-containing cyclic group. The term “monocyclic heterocycle” encompasses monocyclic heteroaryl groups and non-aromatic heteromonocyclic groups. Preferred monocyclic heterocycles are furanyl, thienyl, pyrrolyl, oxanyl, oxazolyl, thiazolyl, imidazolyl, pyrazolyl, isoxazolyl, isothiazolyl, triazolyl, oxadiazolyl, pyridyl, pyridazinyl, pyrimidinyl, pyrazinyl, triazinyl, tetrahydrofuranyl, tetrahydropyranyl, dioxanyl, morpholinyl. Particularly preferred monocyclic heterocycles in the definition of R3 are pyridyl, piperidyl, oxanyl, pyranyl, thianyl and thiopyranyl, more preferably pyridyl and oxanyl.

With respect to substituents, the term “optionally substituted” indicates a group may be unsubstituted or substituted with the indicated number and type of the substituent(s). The term “one or more substituents independently selected from . . . ” means that if a group that is substituted with more than one substituent, these substituents may be the same or different from each other. Similarly, if multiple variables are independently chosen from more than one definition, such as m and n in present formula (I) which can be 0 or 1, the term “independently” means that each variables may have the same or different definition as the other variable(s).

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the compound. Each such asymmetric center will independently produce two optical isomers and it is intended that all of the possible optical isomers and diastereomers in mixtures and as pure or partially purified compounds are included within the ambit of this invention. The present invention encompasses all such isomeric forms of these compounds. The independent syntheses of these diastereomers or their chromatographic separations may be achieved with any method known in the art, for instance as described in the Examples. The absolute stereochemistry of a compound may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as chiral HPLC or SFC (Supercritical Fluid Chromatography) techniques. In the Examples, two suitable SFC methods are described.

Salts of compounds according to the invention are also provided. Such salts include, but are not limited to, acid addition salts and base addition salts. The term “pharmaceutically acceptable salt” as used herein refers to those salts retain the pharmacological activity of the compounds and that are, within the scope of sound medical judgment, suitable for use in humans or animals without undue toxicity, irritation, allergic response, and the like, and are commensurate with a reasonable benefit/risk ratio. Pharmaceutically acceptable salts are well-known in the art. They can be prepared in situ when isolating and purifying the compounds of the invention, or separately by reacting them with pharmaceutically acceptable non-toxic bases or acids, including inorganic or organic bases and inorganic or organic acids, for instance by reacting the free acid or free base forms of the product with one or more equivalents of the appropriate acid or base in a solvent or medium in which the salt is insoluble, or in a solvent such as water or an organic solvent which is then removed in vacuo or by freeze-drying, or by exchanging the cations of an existing salt for another cation on a suitable ion exchange resin. Examples of pharmaceutically acceptable acids and bases include organic and inorganic acids such as acetic acid, trifluoroacetic acid, hydrochloric acid, and bases.

Compounds may exist as polymorphs and as such are intended to be included in the present invention.

The compounds of the invention may be prepared by methods known in the art and to a skilled person. Suitable methods to prepare the compounds are described in the experimental section of this description.

Compounds according to the invention are useful in counteracting diseases or disorders mediated by an S1P receptor, preferably S1P5. They are preferably mixed with pharmaceutically suitable auxiliaries, e.g. as described in the standard reference “Remington, The Science and Practice of Pharmacy” (21st edition, Lippincott Williams & Wilkins, 2005, see especially Part 5: Pharmaceutical Manufacturing). The compounds together with pharmaceutically suitable auxiliaries may be compressed into solid dosage units, such as pills or tablets, or be processed into capsules or suppositories. By means of pharmaceutically suitable liquids the compounds can also be applied in the form of a solution, suspension or emulsion.

Provided is therefore a pharmaceutical composition comprising a compound according to the invention or a pharmaceutically acceptable salt thereof, and at least one pharmaceutically acceptable carrier, diluent and/or excipient. By “pharmaceutically acceptable” it is meant that the carrier, diluent or excipient must be compatible with the other ingredients of the formulation and not deleterious to the recipient thereof. In general, any pharmaceutically suitable additive which does not interfere with the function of the active compounds can be used. A pharmaceutical composition according to the invention is preferably suitable for human use.

Examples of suitable carriers comprise a solution, lactose, starch, cellulose derivatives and the like, or mixtures thereof. In a preferred embodiment said suitable carrier is a solution, for example saline. For making dosage units, e.g. tablets, the use of conventional additives such as fillers, colorants, polymeric binders and the like, is contemplated. Examples of excipients which can be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. When the dosage unit form is a capsule, it may contain, in addition to materials of the above type, a liquid carrier such as fatty oil. Various other materials may be present as coatings or to otherwise modify the physical form of the dosage unit. For instance, tablets may be coated with shellac, sugar or both. A syrup or elixir may contain the active compound, sucrose as a sweetening agent, methyl and propyl parabens as preservatives, a dye and a flavoring such as cherry or orange flavor. Compositions for intravenous administration may for example be solutions of the compounds of the invention in sterile isotonic aqueous buffer. Where necessary, the intravenous compositions may include for instance solubilizing agents, stabilizing agents and/or a local anesthetic to ease the pain at the site of the injection.

The compounds of the invention may be administered enterally or parenterally. The exact dose and regimen of these compounds and compositions thereof will be dependent on the biological activity of the compound per se, the age, weight and sex of the individual, the needs of the individual subject to whom the medicament is administered, the degree of affliction or need and the judgment of the medical practitioner. In general, parenteral administration requires lower dosages than other methods of administration which are more dependent upon adsorption. However, the dosages for humans are preferably 0.001-10 mg per kg body weight. In general, enteral and parenteral dosages will be in the range of 0.1 to 1.000 mg per day of total active ingredients.

In an embodiment of the invention, a pharmaceutical kit or kit of parts is provided comprising one or more containers filled with one or more pharmaceutical compositions of the invention and optionally one or more pharmaceutically acceptable excipients as described herein. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration. Preferably, a pharmaceutical kit or kit of parts comprises instructions for use.

The compounds of the invention are modulators of the S1P receptor, in particular of the S1P5 receptor. More specifically, the compounds of the invention are S1P5 receptor agonists. The compounds are useful in the treatment or alleviation of diseases or disorders mediated by an S1P receptor, preferably S1P5. The compounds of the present invention are particularly suitable to treat or alleviate diseases and disorder s in which an S1P receptor is involved or in which modulation of the endogenous S1P signaling system via an S1P receptor is involved, preferably S1P5.

Provided is therefore a method of treatment or alleviation of a disease or disorder in which an S1P receptor is involved or in which modulation of the endogenous S1P signaling system via an S1P receptor is involved, preferably S1P5, comprising administering to a patient in need thereof a compound according to the invention or a pharmaceutically acceptable salt thereof. Said patient is preferably a human patient.

Further provided is a use of a compound according to the invention or a pharmaceutically acceptable salt thereof for the manufacture of a medicament for the treatment or alleviation of a disease or disorder in which an S1P receptor is involved or in which modulation of the endogenous S1P signaling system via an S1P receptor is involved, preferably S1P5 receptor.

Further provided is a compound according to the invention, or a pharmaceutically acceptable salt thereof for use in therapy, preferably for use as a medicament.

Further provided is a compound according to the invention or a pharmaceutically acceptable salt thereof, or a pharmaceutical composition comprising such compound or a pharmaceutically acceptable salt thereof, for use in the treatment or alleviation of a disease or disorder in which an S1P receptor is involved or in which modulation of the endogenous S1P signaling system via an S1P receptor is involved, preferably S1P5.

Said diseases or disorder is preferably selected from the group consisting of Alzheimer's Disease (AD), multiple sclerosis, Huntington's Disease and Parkinson's Disease.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described in this document.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

References described herein are incorporated by reference. Neither these, nor any other documents or citations to any references, are admitted to be prior art documents or citations.

The invention will be explained in more detail in the following, non-limiting examples.

### EXAMPLES

### Abbreviations

AIBN azobisisobutyronitril
ACN acetonitrile
DBU diazabicyclo[5.4.0]undec-7-ene
Bn benzyl
DCM dichloromethane
DEAD diethyl azodicarboxylate
DIPEA N,N-Diisopropylethylamine
DMA N,N-dimethylacetamide
DMF N,N-dimethylformamide
DMSO dimethyl sulfoxide
EA ethyl acetate
Eq molar equivalent
EtOAc ethyl acetate
EtOH ethanol
HEPES 2-[4-(2-hydroxyethyl)piperazin-1-yl]ethanesulfonic acid
HPLC high-performance liquid chromatography
LAH lithium aluminium hydride
LC-MS Liquid Chromatography—Mass Spectrometry
LDA lithium diisopropyl amine
MeOH methanol
NaOH sodium hydroxide
NEt3 triethylamine
NBS N-bromosuccinimide
NMR nuclear magnetic resonance
Pd—C palladium-on-carbon
PG protection group
Ph3P triphenylphosphine
PS-Triphenylphosphine polystyrene-triphenylphosphine (resin-bound triphenylphosphine)
RT room temperature
SFC supercritical fluid chromatography
TEA triethylamine
Tf2O trifluoromethanesulfonic anhydride
TFA trifluoroacetic acid
THF tetradydrofuran
TLC thin layer chromatography
v/v volume/volume

### 1. General Methods

Chemicals

Chemicals were purchased from Sigma-Aldrich, Alfa, Acros and SCRC.

Liquid Chromatography—Mass Spectrometry (LC-MS)

Generally, LC-MS measurements were run on Agilent 1200 HPLC/6100 SQ System controlled by Agilent ChemStation Software using one of the follow conditions:

Method A: Mobile Phase: A: Water (0.01% TFA), B: ACN (0.01% TFA)

Gradient: 5% B for 0.2 min, increase to 95% B within 1.7 min, 95% B for 1.3 min, back to 5% B within 0.01 min.

Flow Rate: 2.3 ml/min.

Column: XBridge C18, 4.6*50 mm, 3.5 um

Column Temperature: 50° C.

Method B: Mobile Phase: A: Water (10 mM NH₄HCO₃), B: ACN

Gradient: 5% for 0.2 min, increase to 95% B within 1.7 min, 95% B for 1.4 min, back to 5% B within 0.01 min.

Flow Rate: 2.1 ml/min.

Column: XBridge C18, 4.6*50 mm, 3.5 um

Column Temperature: 50° C.

Nuclear Magnetic Resonance (NMR)

The compounds were either characterized via proton-NMR in d6-dimethylsulfoxide,d-chloroform, d-methanol or d-pyridine on a 400 MHz (Bruker AVM 400) or 500 MHz NMR instrument (Bruker Avance 500 MHz with 5 mm BBFo-z-Grd) or a 600 MHz (Bruker Avance 600 MHz with 5 mm Cryoprobe CPTCI (1H-13C/15N z-Grd), and/or by mass spectrometry.

The magnetic nuclear resonance spectral properties (NMR) refer to the chemical shifts (6) expressed in parts per million (ppm). The relative area of the shifts in the 1H-NMR spectrum corresponds to the number of hydrogen atoms for a particular functional type in the molecule. The nature of the shift, as regards multiplicity, is indicated as singlet (s), broad singlet (s. br.), doublet (d), broad doublet (d br.), triplet (t), broad triplet (t br.), quartet (q), quintet (quint.) and multiplet (m).

Separation of the Pure Enantiomers of Chiral Compounds.

Two Supercritical Fluid Chromatography (SFC) methods were used to separate enantiomers from racemates of chiral compounds, referred to as “analytical SFC” and “preparative SFC”. The former is in particular suitable for small scale and the latter for larger scale.

Analytical SFC

Samples were run on an Agilent 1260 Infinity Hybrid SFC System, controlled by Agilent OpenLab CDS ChemStation Edition. The system consists of an injector, a heated column compartment including a switch for 15 columns, a CO₂-booster pump and a binary pump module for CO₂ and modifier flow. Detection was done with an UV-detector and Agilent 1100 series quadrupole mass spectrometer (ESI ionization). The backpressure regulator was set to 160 bar and heated to 60° C. If not stated otherwise, the columns were 100 mm in length, 4.6 mm in diameter and packed with 5 μm material. They were kept at RT during analysis. As mobile phase, a mixture of liquefied CO₂ and organic modifier with additive was used as indicated for each sample. The flow rate was kept at 3.5 mL/min.

Preparative SFC

Preparative separations were carried out on a Waters Prep 100 q SFC System, controlled by Waters MassLynx Software. The system consists of an open bed injector/collector, a heated column compartment including a switch for 6 columns, a CO₂-booster pump, a pump module for modifier flow. Detection was done by UV and a quadrupole mass spectrometer (Waters Aquity QDa, ESI-ionization). To enable quantitative collection, the gas liquid separator was driven with a make-up flow of 30 mL/min methanol. The backpressure regulator was set to 120 bar and heated to 60° C. If not stated otherwise, the columns were 250 mm in length, 20 mm in diameter and packed with 5 μm material. They were kept at 30° C. during the separation. As mobile phase, a mixture of liquefied CO₂ and organic modifier with additive was used as indicated for each sample. The flow rate was kept at 100 g/min.

### 2. General Synthesis Methods and Synthesis of Intermediates

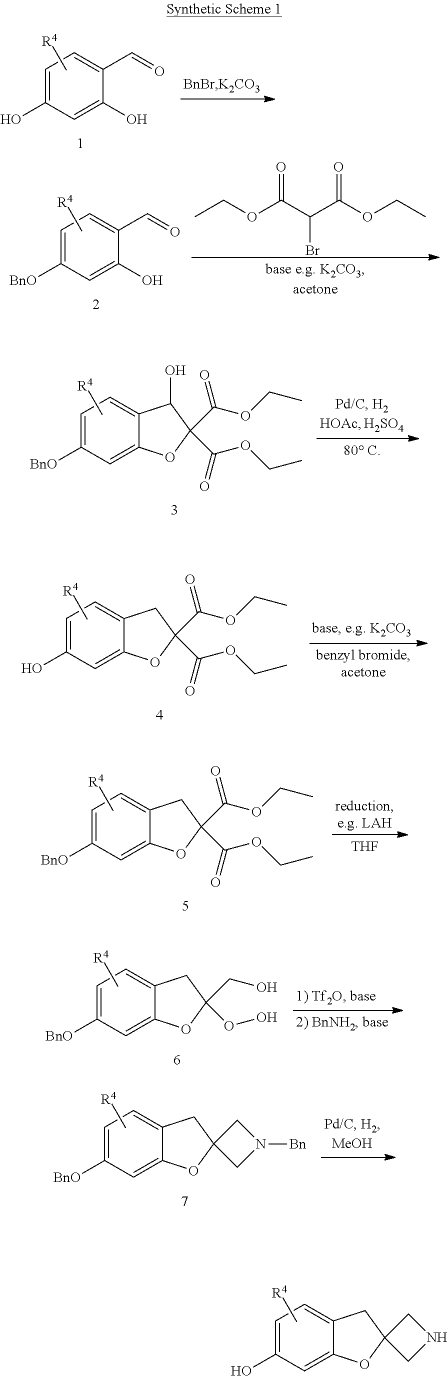

Procedures of Synthetic Scheme 1:

Below the procedure of synthetic scheme 1 is described in detail for compounds wherein R4 is H. Appropriately substituted starting compound 1 can be used to prepare the corresponding substituted 3′H-spiro[azetidine-3,2′-benzofuran]-6′-ol. In brief, R4-substituted 2,4-dihydroxybenzaldehyde derivatives can be converted to the corresponding parabenzyl ether derivatives with benzyl bromide under basic conditions. Reaction with diethyl 2-bromomalonate under basic conditions gives the corresponding diethyl R4-substituted 6-(benzyloxy)-3-hydroxybenzofuran-2,2(3H)-dicarboxylate. Hydrogenation under acidic conditions affords the R4-substituted diethyl 6-hydroxybenzofuran-2,2(3H)-dicarboxylate. Reaction of the phenol derivative under basic conditions with benzyl bromide yields the corresponding R4-substituted diethyl 6-(benzyloxy)benzofuran-2,2(3H)-dicarboxylate. Reduction with e.g. lithium aluminium hydride can afford the corresponding (6-(benzyloxy)-2,3-dihydrobenzofuran-2,2-diyl)dimethanol. Treatment with trifluoromethanesulfonic anhydride gives the corresponding bis-triflate which upon reaction with benzyl amine could afford the corresponding R4-substituted 1-benzyl-6′-(benzyloxy)-3′H-spiro[azetidine-3,2′-benzofuran]. Hydrogenation using e.g. Pd/C yields the corresponding R4-substituted 3′H-spiro[azetidine-3,2′-benzofuran]-6′-ol.

### Detailed Procedure of Synthetic Scheme 1 for Compounds Wherein R4 is H

### 1. Synthesis of Compound 2

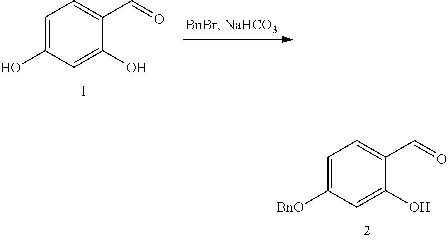 [SMILES: O=Cc1ccc(O)cc1O ; O=Cc1ccc(OCc2ccccc2)cc1O]

A mixture of sodium bicarbonate (1.216 g, 14.48 mmol), benzyl bromide (2.477 g, 14.48 mmol) and 2,4-dihydroxybenzaldehyde (2 g, 14.48 mmol)) in acetonitrile (50 mL) was stirred at 85° C. for 20 hours. The solvent was removed to give a brown oil. The residue was diluted with 80 mL of water, extracted with ethyl acetate (3×75 mL). The combined organic layers were dried over Na₂SO₄, filtered through glass funnel and concentrated to give an orange oil. The crude material was purified by silica gel column chromatography and eluted with 5% ethyl acetate/heptane. The following fractions were collected and concentrated to give the title compound 4-(benzyloxy)-2-hydroxybenzaldehyde (2.2 g, 8.67 mmol, 59.9% yield) as a white solid.

LCMS(ESI-MS): m/z: 312.1 [M+H]⁺; Rt=2.09 min. (Method A)

### 2. Synthesis of Compound 3

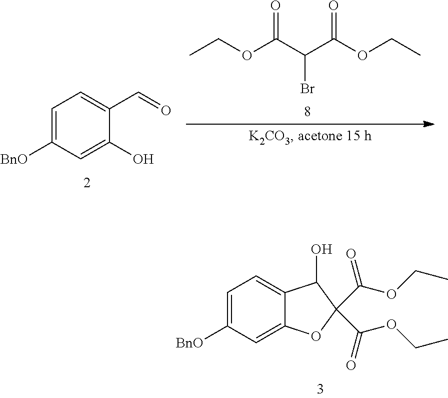 [SMILES: CCOC(=O)C(Br)C(=O)OCC ; O=Cc1ccc(OCc2ccccc2)cc1O ; CCOC(=O)C1(C(=O)OCC)Oc2cc(OCc3ccccc3)ccc2C1O]

To a solution of 4-(benzyloxy)-2-hydroxybenzaldehyde (1.0 g, 4.38 mmol) in acetone (15 mL) was added K₂CO₃ (1.817 g, 13.14 mmol) and followed by addition of diethyl 2-bromomalonate (1.047 g, 4.38 mmol). The mixture was stirred at RT for 10 hours. The mixture was filtered. The filtrate was concentrated under reduced pressure. The crude material was purified by silica gel column chromatography and eluted with 50% ethyl acetate/hexane to give the title compound 3 (1.4 g, 3.08 mmol, 70.3% yield) as a yellow solid.

LCMS(ESI-MS): m/z: 309 [M+Na]⁺; Rt=2.04 min. (Method A)

### 3. Synthesis of Compound 4

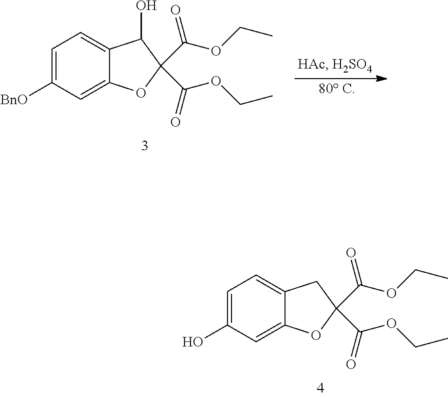 [SMILES: CCOC(=O)C1(C(=O)OCC)Oc2cc(OCc3ccccc3)ccc2C1O ; CCOC(=O)C1(C(=O)OCC)Cc2ccc(O)cc2O1]

To a solution of compound 3 (1.0 g, 2.59 mmol) in acetic acid (50 mL) was added Pd—C (1.0 g, 0.940 mmol) and a drop of H₂SO₄. The mixture was attached to a hydrogenation apparatus. The system was evacuated and then refilled with hydrogen several times. The mixture was stirred and hydrogenated at 20° C. for 15 hours. The solid was filtered off. The filtrate was concentrated. The residue was purified by silica gel column chromatography and eluted with ethyl acetate/hexane (1:1) to give the title compound diethyl 6-hydroxybenzofuran-2,2(3H)-dicarboxylate (0.5 g, 1.695 mmol, 65.5% yield) as a white solid.

LCMS(ESI-MS): m/z: 281 [M+H]⁺; Rt=1.56 min. (Method B)

### 4. Synthesis of Compound 5

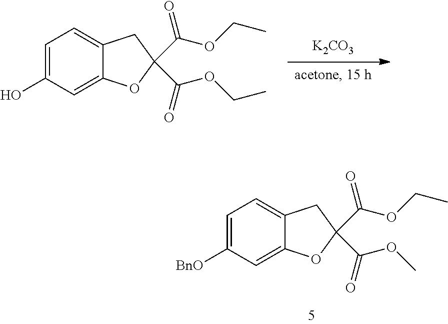

To a solution of diethyl 6-hydroxybenzofuran-2,2(3H)-dicarboxylate (500 mg, 1.784 mmol) in acetone (5 mL) was added K₂CO₃ (616 mg, 4.46 mmol) and benzyl bromide (0.275 mL, 2.319 mmol). The mixture was stirred at 25° C. for 2 hours. LCMS showed the reaction was complete. The mixture was concentrated in vacuum. The residue was purified by silica gel column chromatography and eluted with ethyl acetate/petroleum ether=5:1) to give title compound diethyl 6-(benzyloxy)benzofuran-2,2(3H)-dicarboxylate (600 mg, 1.458 mmol, 82% yield).

LCMS(ESI-MS): m/z: 371 [M+H]⁺; Rt=2.19 min. (Method A)

### 5. Synthesis of Compound 6

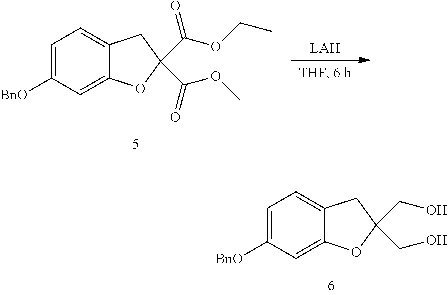 [SMILES: CCOC(=O)C1(C(=O)OC)Cc2ccc(OCc3ccccc3)cc2O1 ; "LAH" ; "THF, 6 h" ; OCC1(CO)Cc2ccc(OCc3ccccc3)cc2O1]

To a solution of diethyl 6-(benzyloxy)benzofuran-2,2(3H)-dicarboxylate (1.2 g, 3.24 mmol) in THF (5 mL) was added LiAlH₄ (0.492 g, 12.96 mmol) at −5° C. The mixture was stirred at −5° C. for 2 hours. LCMS showed the reaction was complete. The reaction was quenched by addition of water (1 mL) and 15% NaOH aqueous solution (1 mL). Then 20 g of sodium sulfate was added to the mixture. The solid was filtered off. The filtrate was concentrated in vacuum. The residue was purified by silica gel column chromatography and eluted with ethyl acetate/petroleum ether=1:3) to give the title compound (6-(benzyloxy)-2,3-dihydrobenzofuran-2,2-diyl)dimethanol (0.6 g, 1.886 mmol, 58.2% yield) as a white solid

LCMS: ESI-MS: m/z: 287 [M+H]+; Rt=2.87 min. (Method B)

### 6. Synthesis of Compound 7

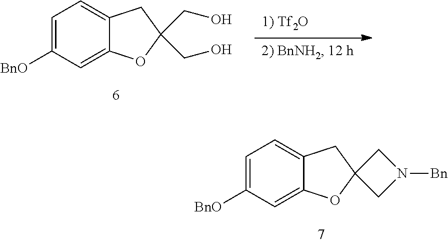

To a solution of (6-(benzyloxy)-2,3-dihydrobenzofuran-2,2-diyl)dimethanol (3.0 g, 10.48 mmol) in acetonitrile (10 mL) was added trifluoromethanesulfonic anhydride (3.72 mL, 22.00 mmol) at −20° C., followed by DIPEA (4.57 mL, 26.2 mmol). After stirred for 0.5 hour, benzylamine (2.005 mL, 18.34 mmol) was added at −20° C. The mixture was stirred at 70° C. for 2 hours. The mixture was diluted with 100 mL of ethyl acetate and 100 mL of saturated NaCl. The organic layer was dried over Na₂SO₄, filtered and concentrated. The residue was purified by silica gel chromatography column and eluted with ethyl acetate/petroleum ether=1:3 to give the title compound 1-benzyl-6′-(benzyloxy)-3′H-spiro[azetidine-3,2′-benzofuran] (2 g, 4.48 mmol, 42.7% yield) as a yellow solid.

LCMS: ESI-MS: m/z: 358 [M+H]⁺; Rt=2.19 min. (Method B)

### 7. Synthesis of 3′H-spiro[azetidine-3,2′-benzofuran]-6′-ol

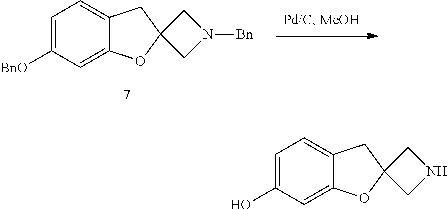 [SMILES: c1ccc(COc2ccc3c(c2)OC2(C3)CN(Cc3ccccc3)C2)cc1 ; "Pd/C, MeOH" ; Oc1ccc2c(c1)OC1(CNC1)C2]

To a solution of 1-benzyl-6′-(benzyloxy)-3′H-spiro[azetidine-3,2′-benzofuran] (9.0 g, 25.2 mmol) in ethyl acetate (500 mL) was added Pd—C (2.68 g, 25.2 mmol). The mixture was attached to a hydrogenation apparatus. The system was evacuated and then refilled with hydrogen several times. The mixture was stirred and hydrogenated at 26° C. for 10 hours. The mixture was filtered and concentrated in vacuum. The residue was purified by silica gel column chromatography and eluted with ethyl acetate/petroleum ether=1:3 to give title compound 3′H-spiro[azetidine-3,2′-benzofuran]-6′-ol (1.35 g, 7.01 mmol, 27.8% yield).

LCMS: ESI-MS: m/z: 178[M+H]+; Rt=0.55 min. (Method A)

NMR (400 MHz, MeOD-d₄): δ 6.971-6.950 (d, J=8 Hz, 1H), 6.314-6.294 (d, J=8 Hz, 1H), 6.229 (s, 1H), 3.939-3.914 (d, J=10 Hz, 2H), 3.645-3.620 (d, J=10 Hz, 2H) 3.306 (s, 2H)

Reference: WO2006/40178

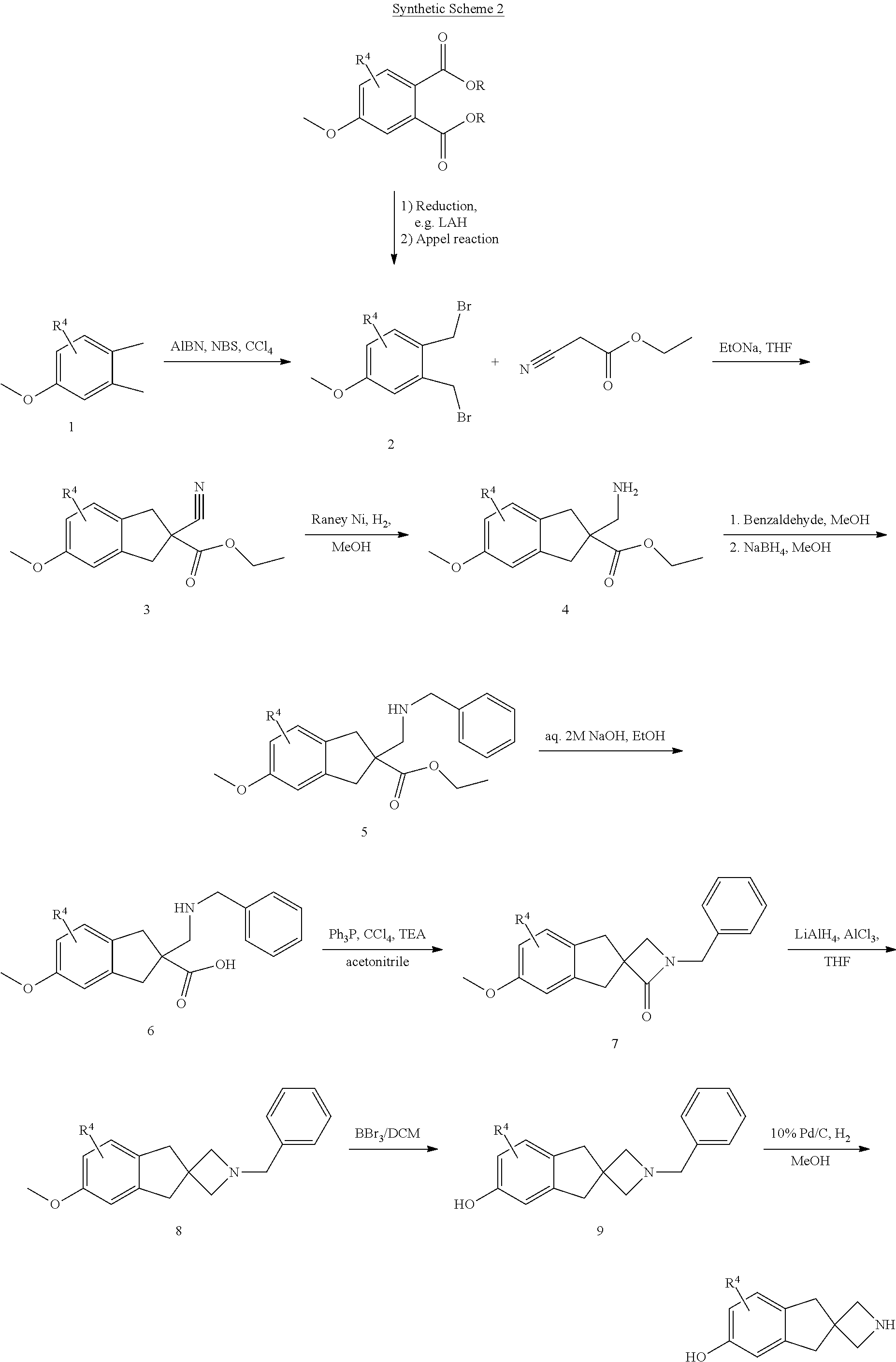

Procedures of Synthetic Scheme 2:

Below the procedure of synthetic scheme 2 is described in detail for compounds wherein R4 is H. Appropriately substituted starting compound 1 can be used to prepare the corresponding substituted 1′,3′-dihydrospiro[azetidine-3,2′-inden]-5′-ol. In brief, R4-substituted 4-methoxy-1,2-dimethylbenzene can be dibrominated in the two benzylic positions to give the corresponding 1,2-bis(bromomethyl)-4-methoxybenzene. Alternatively an R4-substituted 4-methoxyphthalate can be reduced (e.g. lithium aluminium hydride) to the corresponding diol which can be converted under Appel reaction conditions to the equivalent R4-substituted 1,2-bis(bromomethyl)-4-methoxybenzene. Reaction with 2-cyanoacetate under basic conditions yields the corresponding R4-substituted ethyl 2-cyano-5-methoxy-2,3-dihydro-1H-indene-2-carboxylate. Hydrogenation (e.g. Raney Ni) gives the corresponding ethyl 2-(aminomethyl)-5-methoxy-2,3-dihydro-1H-indene-2-carboxylate. Reductive amination with benzyaldehyde gives the corresponding R4-substituted ethyl 2-((benzylamino)methyl)-5-methoxy-2,3-dihydro-1H-indene-2-carboxylate. Saponification (e.g. NaOH) followed by intramolecular amide formation (e.g. Mitsunobu conditions) yields the R4-substituted 1-benzyl-5′-methoxy-1′,3′-dihydrospiro[azetidine-3,2′-inden]-2-one. Reduction of the amide carbonyl (e.g. LAH, AlCl₃) gives the corresponding 1-benzyl-5′-methoxy-1′,3′-dihydrospiro[azetidine-3,2′-indene]. Demethylation (e.g. BBr₃) gives the corresponding 1-benzyl-1′,3′-dihydrospiro[azetidine-3,2′-inden]-5′-ol and hydrogenation (e.g. Pd/C) could yield the R4-substituted 1′,3′-dihydrospiro[azetidine-3,2′-inden]-5′-ol.

### Detailed Procedure of Synthetic Scheme 2 for Compounds Wherein R4 is H

### Synthesis of 1,2-bis(bromomethyl)-4-methoxybenzene (Compound 2)

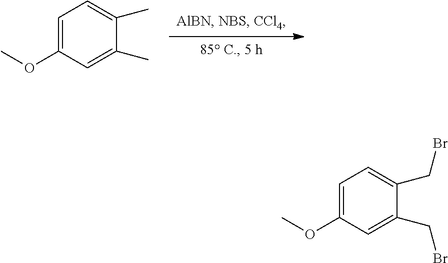

A mixture of 4-methoxy-1,2-dimethylbenzene (50.0 g, 367 mmol), NBS (137 g, 771 mmol), and AIBN (1.21 g, 7.34 mmol) in CCl₄ (600 mL) was refluxed for 5 h under N₂. The resulting succinimide was removed by filtration, and the filtrate was concentrated in vacuo. The residue was chromatographed on silica gel (petroleum ether/dichloromethane=8/1) to give the desired product (35.7 g, 367 mmol, 33.1% yield) as a white solid.

¹H NMR: (400 MHz, CDCL3) δ: 7.31 (d, J=8.4 Hz, 1H), 6.92 (d, J=2.5 Hz, 1H), 6.85 (dd, J=8.4 2.8 Hz, 1H), 4.68 (s, 2H), 4.65 (s, 2H), 3.84 (s, 3H).

### 2 Synthesis of ethyl 2-cyano-5-methoxy-2,3-dihydro-1H-indene-2-carboxylate (Compound 3)

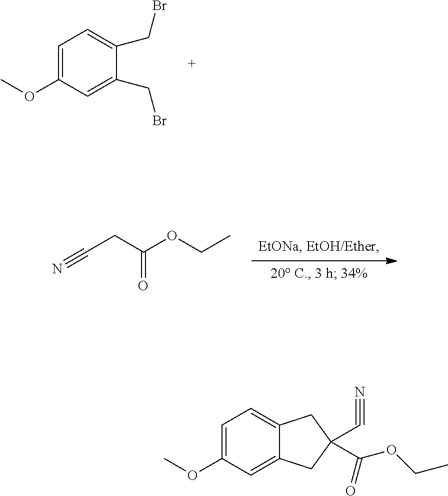

To a mixture of sodium ethoxide (19.83 g, 291 mmol) and THF (200 ml) was added ethyl 2-cyanoacetate (16.48 g, 146 mmol) and a solution of 1,2-bis(bromomethyl)-4-methoxybenzene (35.7 g, 121 mmol) in THF (50 ml) under Nitrogen at 0° C. The mixture was stirred at 25° C. for 3 hr. To the mixture was added H₂O (100 mL) and the mixture was stirred for 5 min. The mixture was extracted with ethyl acetate (300 mL*3). The organic layers were combined, washed with saturated NaCl (100 mL), dried over anhydrous Na₂SO₄, concentrated in vacuo. The residue was purified via column chromatography (petroleum ether/ethyl acetate=10/1) to afford the desired product ethyl 2-cyano-5-methoxy-2,3-dihydro-1H-indene-2-carboxylate (16.6 g, 51.0 mmol, 42.0% yield) as a white solid.

LC-MS: m/z 246.1 (M+H₂O)⁺, RT=1.969 min/3.0 min (Method B);

¹H NMR: (400 MHz, CDCl₃) δ: 7.14 (d, J=8.4 Hz, 1H), 6.82-6.79 (m, 2H), 4.32 (q, J=7.2 Hz, 2H), 3.81 (s, 3H), 3.72-3.62 (m, 2H), 3.56-3.51 (m, 2H), 1.36 (t, J=7.2 Hz, 3H).

### 3 Synthesis of ethyl 2-(aminomethyl)-5-methoxy-2,3-dihydro-1H-indene-2-carboxylate (Compound 4)

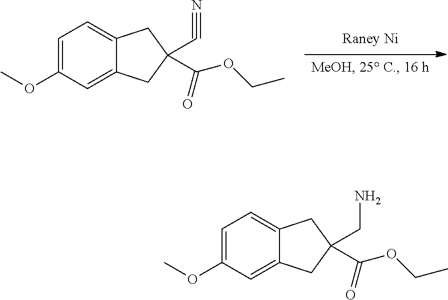

To a solution ethyl 2-cyano-5-methoxy-2,3-dihydro-1H-indene-2-carboxylate (24.66 g, 101 mmol) in Ethanol (500 ml) was added Raney Nickel (35.4 g, 302 mmol). The mixture was stirred under Hydrogen at 25° C. for 16 hr. The mixture was filtered and the filtrate was concentrated in vacuo to afford the desired product ethyl 2-(aminomethyl)-5-methoxy-2,3-dihydro-1H-indene-2-carboxylate (21.5 g, 86 mmol, 86% yield) as a yellow oil.

LC-MS: m/z 250.2 (M+1)+, RT=1.731 min/3.0 min (Method B);

¹H NMR: (400 MHz, DMSO-d6) δ: 7.06 (d, J=8.4 Hz, 1H), 6.76 (d, J=2.0 Hz, 1H), 6.69 (dd, J=8.0 2.0 Hz, 2H), 4.09 (q, J=7.2 Hz, 2H), 3.69 (s, 3H), 3.24-3.13 (m, 2H), 2.96-3.73 (m, 4H), 1.18 (t, J=7.2 Hz, 3H).

### 4 Synthesis of ethyl 2-((benzylamino)methyl)-5-methoxy-2,3-dihydro-1H-indene-2-carboxylate (Compound 5)

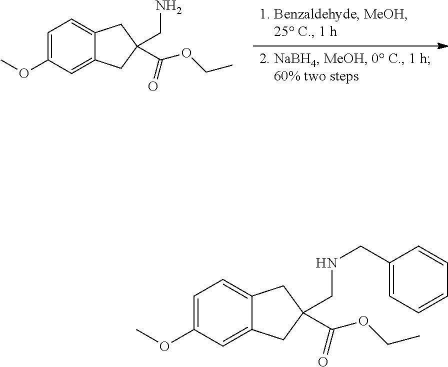

To a solution of ethyl 2-(aminomethyl)-5-methoxy-2,3-dihydro-1H-indene-2-carboxylate (21.5 g, 86 mmol) in Ethanol 150 ml) was added benzaldehyde (8.74 ml, 86 mmol) at 25° C. The mixture was stirred at 25° C. for 1 hr. The mixture was cooled to 0° C. and to it was added sodium borohydride (9.79 g, 259 mmol) portionwise. The mixture was stirred at 0° C. for 1 hr. The mixture was neutralized with 1 N HCl. The mixture was concentrated in vacuo. The residue was extracted with ethyl acetate (200 mL*3). The organic layers were combined, washed with saturated NaCl (100 mL), dried over anhydrous Na₂SO₄, concentrated in vacuo. The residue was purified via flash chromatography (petroleum ether/ethyl acetate=5/1) to afford the desired product ethyl 2-((benzylamino)methyl)-5-methoxy-2,3-dihydro-1H-indene-2-carboxylate (14.0 g, 37.6 mmol, 43.6% yield) as a colorless oil. LC-MS: m/z 340.1 (M+1)⁺, RT=1.55 min/3.0 min (Method B);

¹H NMR: (400 MHz, CDCl₃) δ: 7.40-7.24 (m, 2H), 7.48-7.38 (m, 3H), 7.07 (d, J=8.0 Hz, 1H), 6.74-6.70 (m, 2H), 4.21-4.15 (m, 2H), 3.79 (s, 3H), 3.45-3.35 (m, 2H), 3.05-2.81 (m, 4H), 1.26 (t, J=7.2 Hz, 3H).

### 5 Synthesis of 2-((benzylamino)methyl)-5-methoxy-2,3-dihydro-1H-indene-2-carboxylic acid (Compound 6)

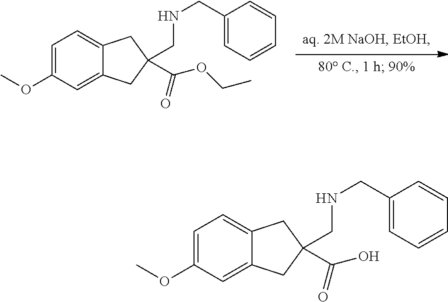 [SMILES: CCOC(=O)C1(CNCc2ccccc2)Cc2ccc(OC)cc2C1 ; "80° C., 1 h; 90%" ; "aq. 2M NaOH, EtOH," ; COc1ccc2c(c1)CC(CNCc1ccccc1)(C(=O)O)C2]

To a solution of ethyl 2-((benzylamino)methyl)-5-methoxy-2,3-dihydro-1H-indene-2-carboxylate (14.0 g, 41.2 mmol) in Ethanol (80 ml) was added 2 M sodium hydroxide (80 ml, 160 mmol) at RT. The mixture was refluxed for 2 hr. The mixture was cooled to RT. To the mixture was added 1 N HCl to adjust PH=7. The organic solvent was evaporated and the resulted mixture was extracted with dichloromethane:methanol (v:v=10:1, 150 mL*3). The organic layers were combined, washed with H₂O (80 mL), saturated NaCl (50 mL), dried over anhydrous Na₂SO₄, concentrated in vacuo to afford the crude product 2-((benzylamino)methyl)-5-methoxy-2,3-dihydro-1H-indene-2-carboxylic acid (9.7 g, 30.0 mmol, 72.7% yield) as a white foam.

LC-MS: m/z 312.1 (M+1)⁺, RT=1.477 min/3.0 min (Method B);

¹H NMR: (400 MHz, DMSO-d6) δ: 9.32 (brs, 1H), 7.35-7.27 (m, 5H), 7.04 (d, J=8.4 Hz, 1H), 6.67 (dd, J=8.0 2.4 Hz, 1H), 3.83 (s, 2H), 3.70 (s, 3H), 3.28-3.16 (m, 2H), 2.88-2.76 (m, 4H), 1.26 (t, J=7.2 Hz, 3H).

### 6 Synthesis of 1-benzyl-5′-methoxy-1′,3′-dihydrospiro[azetidine-3,2′-inden]-2-one (Compound 7)

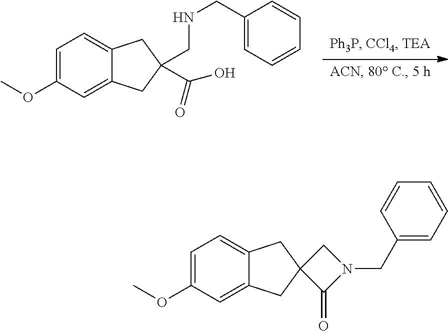 [SMILES: COc1ccc2c(c1)CC(CNCc1ccccc1)(C(=O)O)C2 ; "ACN, 80° C., 5 h" ; "Ph3P, CCl4, TEA" ; COc1ccc2c(c1)CC1(C2)CN(Cc2ccccc2)C1=O]

To a mixture of 2-((benzylamino)methyl)-5-methoxy-2,3-dihydro-1H-indene-2-carboxylic acid (9.85 g, 31.6 mmol), triphenylphosphine (9.96 g, 38.0 mmol) and acetonitrile (20 ml) was added CCl₄ (6.11 mL, 63.3 mmol) and TEA (5.29 ml, 38.0 mmol) under Nitrogen at RT. The mixture was refluxed at 90° C. under Nitrogen for 5 hr. The mixture was concentrated in vacuo. The residue was purified via column chromatography (petroleum ether:ethyl acetate=3:1) to afford the desired product 1-benzyl-5′-methoxy-1′,3′-dihydrospiro[azetidine-3,2′-inden]-2-one (7.7 g, 25.4 mmol, 80% yield) as a yellow oil.

LC-MS: m/z 294.1 (M+1)⁺, RT=1.998 min/3.0 min (Method B);

¹H NMR: (400 MHz, CDCl₃) δ: 7.41-7.27 (m, 5H), 7.10 (d, J=8.4 Hz, 1H), 6.79-6.73 (m, 2H), 4.45 (s, 2H), 3.79 (s, 3H), 3.52-3.43 (m, 2H), 3.19 (s, 2H), 3.10-3.03 (m, 2H).

### 7 Synthesis of 1-benzyl-5′-methoxy-1′,3′-dihydrospiro[azetidine-3,2′-indene] (Compound 8)

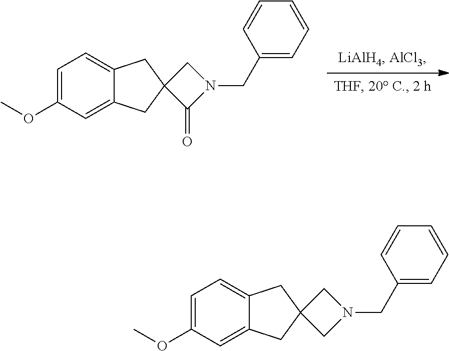 [SMILES: COc1ccc2c(c1)CC1(C2)CN(Cc2ccccc2)C1=O ; "LiAlH4, AlCl3," ; "THF, 20° C., 2 h" ; COc1ccc2c(c1)CC1(C2)CN(Cc2ccccc2)C1]

To a solution of aluminum chloride (7.00 g, 52.5 mmol) in THF (100 mL) was added lithium aluminum hydride (2.99 g, 79 mmol) at 0° C. under Nitrogen. The mixture was stirred at 0° C. for 5 min. To the mixture was added a solution of 1-benzyl-5′-methoxy-1′,3′-dihydrospiro[azetidine-3,2′-inden]-2-one (7.7 g, 26.2 mmol) in THF (10 mL) dropwise at 0° C. under Nitrogen. The mixture was stirred at 25° C. for 16 hr. To the mixture was added H₂O (50 mL), 15% aq NaOH (50 mL), H₂O (150 mL) dropwise at 0° C. and the mixture was stirred at 0° C. for 10 min. To the mixture was added ethyl acetate (100 mL) and the mixture was filtered through celite. The organic layer of the filtrate was separated and the aqueous layer was extracted with ethyl acetate (100 mL*2). The organic layers were combined, washed with H₂O (50 mL), saturated NaCl (50 mL), dried over anhydrous Na₂SO₄, concentrated in vacuo to afford the desired product 1-benzyl-5′-methoxy-1′,3′-dihydrospiro[azetidine-3,2′-indene] (6.5 g, 21.74 mmol, 83% yield) as a colorless oil.

LC-MS: m/z 280.1 (M+1)⁺, RT=1.508 min/3.0 min (Method B);

¹H NMR: (400 MHz, CDCl₃) δ: 7.35-7.24 (m, 5H), 7.09 (d, J=8.0 Hz, 1H), 6.76 (s, 1H), 6.71 (dd, J=8.4 2.8 Hz, 2H), 3.79 (s, 3H), 3.67 (s, 2H), 3.26 (s, 4H), 3.13 (s, 2H), 3.08 (s, 2H).

### 8 Synthesis of 1-benzyl-1′,3′-dihydrospiro[azetidine-3,2′-inden]-5′-ol (Compound 9)

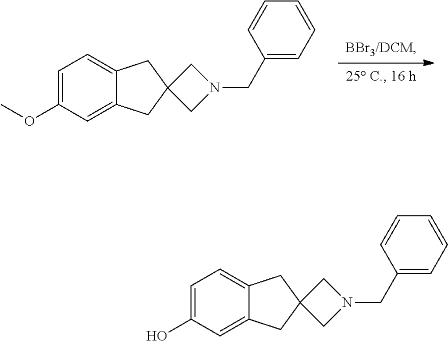 [SMILES: COc1ccc2c(c1)CC1(C2)CN(Cc2ccccc2)C1 ; "25° C., 16 h" ; "BBr3/DCM," ; Oc1ccc2c(c1)CC1(C2)CN(Cc2ccccc2)C1]

To a solution of 1-benzyl-5′-methoxy-1′,3′-dihydrospiro[azetidine-3,2′-indene] (6.50 g, 23.27 mmol) in CH₂Cl₂ (200 ml) was added HCl/Dioxane (20 ml) dropwise at 0° C. The mixture was stirred at 0° C. for 5 min. The mixture was concentrated in vacuo. To the solution of residue in CH₂Cl₂ (200 ml) was added BBr3 (3.30 ml, 34.9 mmol) dropwise at −78° C. under Nitrogen. The mixture was stirred at 25° C. for 16 hr. To the mixture was added sat. NaHCO₃(25 mL) and the mixture was stirred for 15 min. The organic was separated and the aqueous layer was extracted with dichloromethane/methanol (60 mL/6 mL*3). The organic layers were combined and washed with H₂O (20 mL), saturated NaCl (20 mL), dried over anhydrous Na₂SO₄, concentrated in vacuo. The residue was purified via flash chromatography (dichloromethane:methanol=45:1 to 30:1) to afford the desired product 1-benzyl-1′,3′-dihydrospiro[azetidine-3,2′-inden]-5′-ol (4.05 g, 15.26 mmol, 65.6% yield) as a white solid.

LC-MS: m/z 266.1 (M+1)+, RT=1.394 min/3.0 min (Method B);

¹H NMR: (400 MHz, DMSO-d6) δ: 9.15 (s, 1H), 7.48-7.42 (m, 5H), 6.98 (d, J=8.0 Hz, 1H), 6.60 (s, 1H), 6.54 (dd, J=8.0 2.4 Hz, 1H), 4.27 (brs, 1H), 3.92 (brs, 4H), 3.11 (s, 2H), 3.07 (s, 2H).

### 9 Synthesis of Compound 1′,3′-dihydrospiro[azetidine-3,2′-inden]-5′-ol

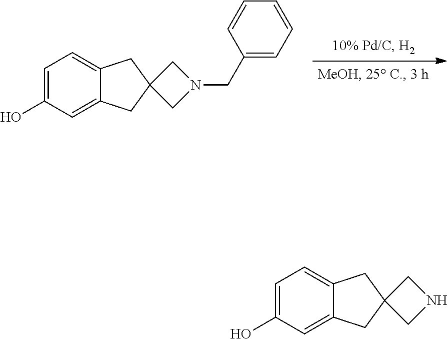 [SMILES: Oc1ccc2c(c1)CC1(C2)CN(Cc2ccccc2)C1 ; "10% Pd/C, H2" ; "MeOH, 25° C., 3 h" ; Oc1ccc2c(c1)CC1(CNC1)C2]

To a solution of 1-benzyl-1′,3′-dihydrospiro[azetidine-3,2′-inden]-5′-ol (8.10 g, 30.5 mmol) in methanol (200 ml) was added 10% Pd/C (24.3 g, 228 mmol) under Nitrogen. The mixture was stirred under hydrogen bag at 25° C. for 3 hr. The mixture was filtered, washed with methanol (100 mL). The filtrate was concentrated in vacuo to afford the desired product 1′,3′-dihydrospiro[azetidine-3,2′-inden]-5′-ol (4.9228 g, 28.1 mmol, 92% yield) as a white solid.

LC-MS: m/z 176 (M-55)⁺, RT=0.802 min/3.0 min (Method A);

¹H NMR: (400 MHz, Methanol-d₆) δ: 7.03 (d, J=8.0 Hz, 1H), 6.67 (s, 1H), 6.62 (dd, J=8.4 2.4 Hz, 1H), 4.07 (s, 4H), 3.20 (s, 2H), 3.17 (s, 2H).

### REFERENCE

J. Org. Chem. 1997, 62, 6598-6602;
Canadian Journal of Research, Section B: Chemical Sciences; vol. 26; (1948); p. 503,509;
Synthetic Communications, 18(3), 247-252 (1988);
WO2013/11285, (2013)

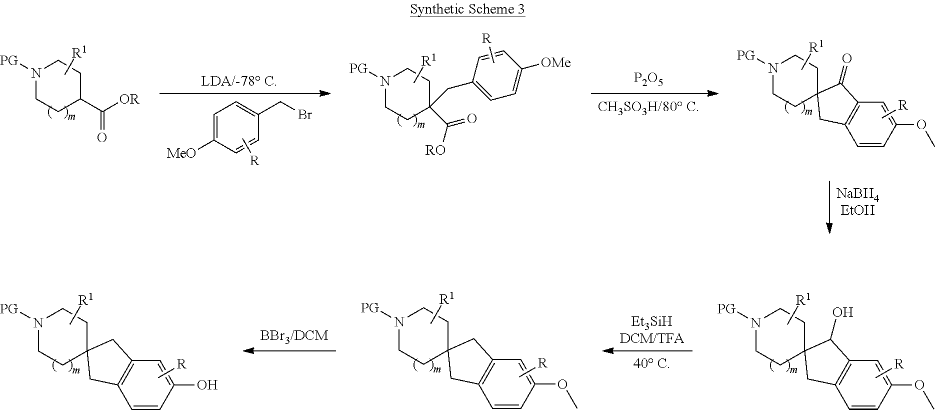

Intermediates leading to compounds of formula I (m=0, 1; X═C) are commercially available or can be prepared in accordance with Synthetic Scheme 3.

Procedures of Synthetic Scheme 3:

N-protected (PG=protection group) pyrollidine 3-carboxylic ester derivatives (m=0) or N-protected piperidine 4-carboxylic ester derivatives (m=1) can be deprotonated with e.g. lithium diisopropyl amine at low temperature (e.g. −78° C.) and then can be alkylated in the alpha-position to the ester moiety with a para-alkoxy benzylhalide derivative (e.g. substituted para-methoxy benzyl bromide). Reaction with a strong dehydrating agent (e.g. phosphorus pentoxide) affords the corresponding spiro-2,3-dihydro-1H-inden-1-one derivative. The carbonyl moiety of the obtained 2,3-dihydro-1H-inden-1-one derivative can be reduced in two steps, e.g. 1) sodium borohydride in ethanol; 2) triethylsilane, trifluoro acetic acid in dichloromethane) to give the corresponding spiro indanyl derivative. Deprotection of the alkoxy indanyl derivative (e.g. with boron tribromide in the case of a methoxy indanyl) yields the corresponding phenol derivative. In case of a benzyl moiety as N-protection group this protection group could be cleaved, e.g. under hydrogenating conditions using hydrogen and Pd/C as catalyst to give the corresponding amino phenol spiroderivative.

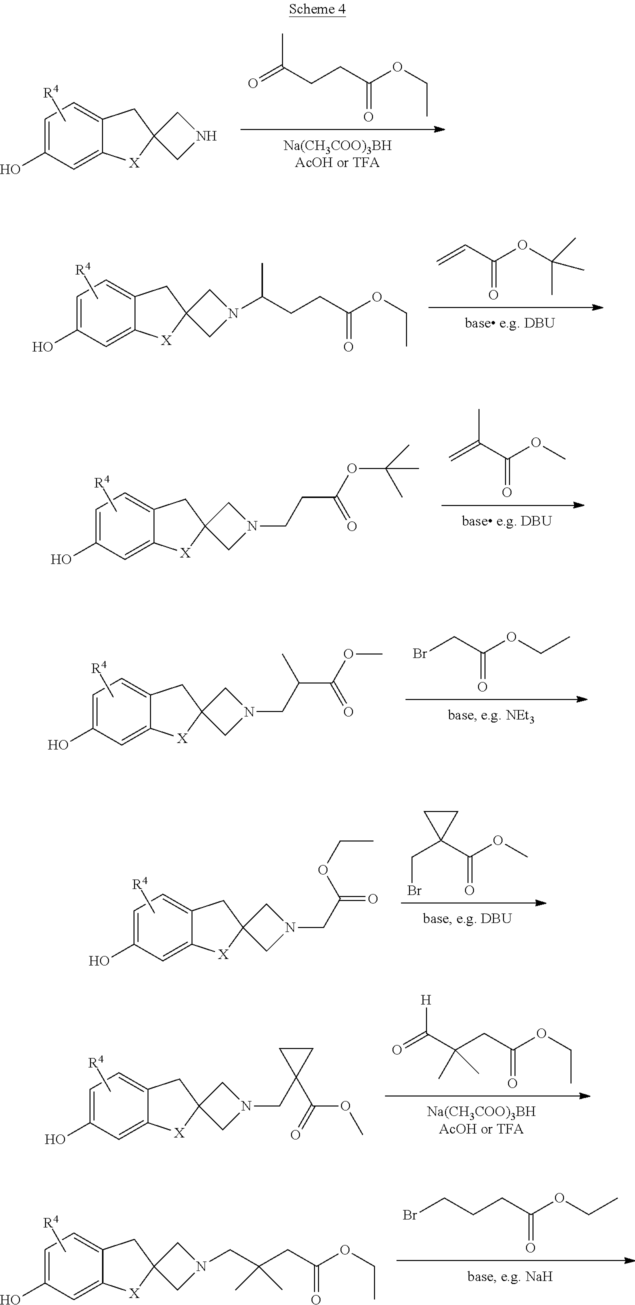

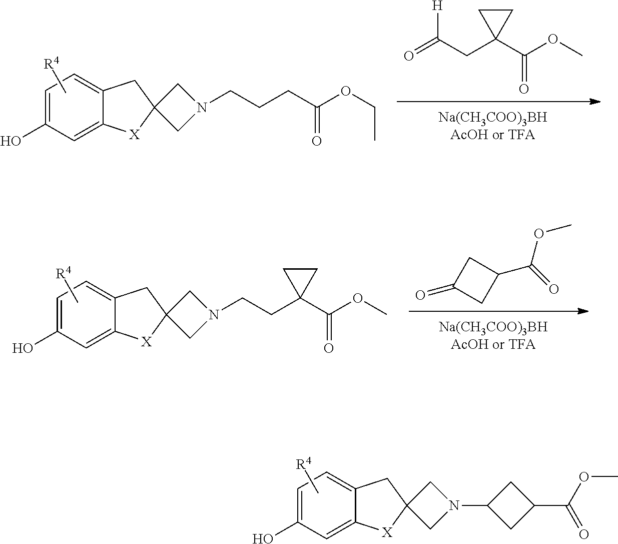

R1-moieties can be introduced starting from the corresponding azetidines, wherein X is O or CH₂ (see synthetic schemes 1 and 2), using alkylating or reductive amination conditions as depicted in scheme 4 above. When the R1-moiety contains an ester the corresponding acid can be obtained by saponification under basic (e.g. NaOH) or acidic (e.g. TFA) conditions. Scheme 4 shows a number of routes starting from R4 substituted 3′H-spiro[azetidine-3,2′-benzofuran]-6′-ol or 1′,3′-dihydrospiro[azetidine-3,2′-inden]-5′-ol. It is clear to a person skilled in the art that these routes are suitable to introduce alternative substituents in these azetindine-containing compounds using the appropriate reagent(s), as well as in the corresponding pyrrolidine- or piperidine-containing compounds.

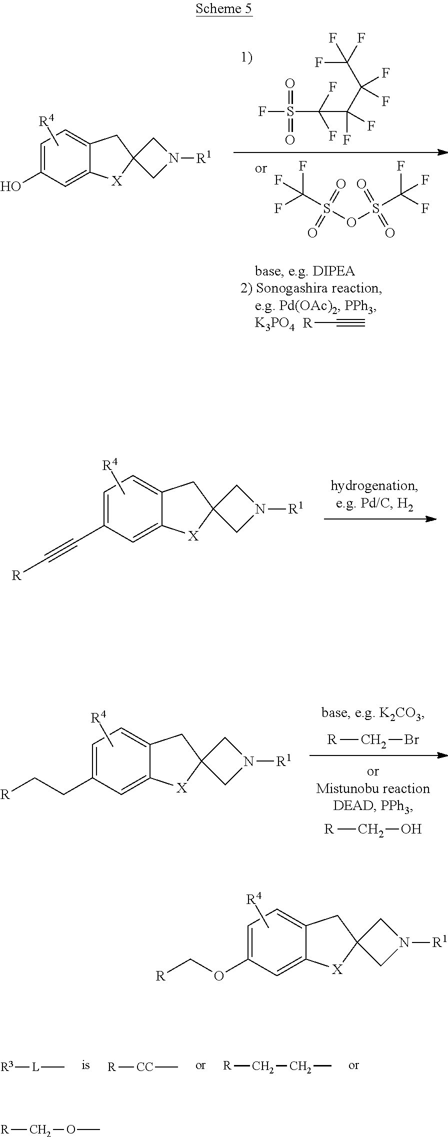

R3-L-moieties can be introduced starting from the corresponding phenols via the corresponding nonaflates or triflates followed by Sonogashira reaction with an substituted alkyne or by the same protocol followed by hydrogenation which can yield the corresponding ethenyl derivative as shown in scheme 5. R3-L-moieties that contain a phenyl ether can be obtained under alkylating conditions using the corresponding alkylhalide precursors or under Mitsunobu conditions using the corresponding alkyl alcohols.

When the R1-moiety contains an ester the corresponding acid can be obtained by saponification under basic (e.g. NaOH) or acidic (e.g. TFA) conditions.

Scheme 5 shows a number of routes starting from R4 substituted 3′H-spiro[azetidine-3,2′-benzofuran]-6′-ol or 1′,3′-dihydrospiro[azetidine-3,2′-inden]-5′-ol. It is clear to a person skilled in the art that these routes are suitable to introduce R3-L-moieties in in the corresponding pyrrolidine- or piperidine-containing compounds as well.

In section 3. Synthesis of compounds according to the invention below, the introduction of alternative linkers L and R3-L-moieties is described. It is clear to a skilled person that the routes described for specific compounds can be used to introduce the same linker L and R3-L-moieties in other compounds according to the invention.

### 3. Synthesis of Compounds According to the Invention

### Example 1. 1-((5′-((4-ethoxyphenyl)ethynyl)-1′,3′-dihydrospiro[azetidine-3,2′-inden]-1-yl)methyl)cyclopropanecarboxylic acid compound with 2,2,2-trifluoroacetic acid (1:1)

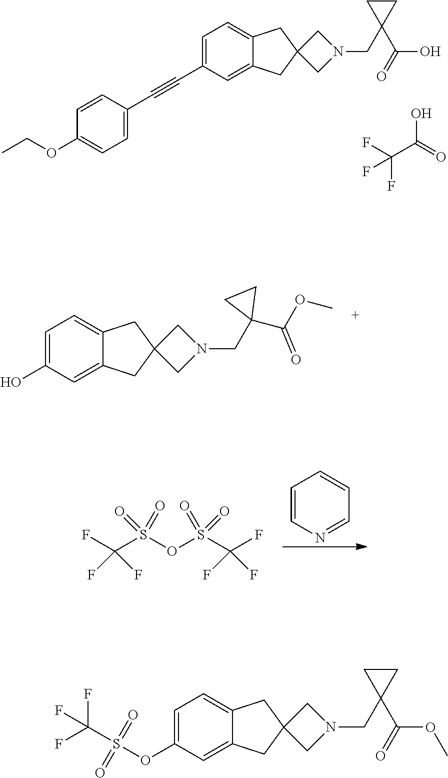

In a 100 mL 3 neck flask was methyl 1-((5′-hydroxy-1′,3′-dihydrospiro[azetidine-3,2′-inden]-1-yl)methyl)cyclopropanecarboxylate (650 mg, 2.262 mmol, prepared as described for example 28) in CH₂Cl₂ (20 mL) to give a light yellow solution. Pyridine (0.45 mL, 5.56 mmol) was added. The mixture was cooled to 0° C. and at this temperature trifluoromethanesulfonic anhydride (2.488 mL, 2.488 mmol) was added dropwise. The color of the solution turned to yellow.

The reaction mixture was diluted with CH₂Cl₂ and washed 2× with sat. NH₄Cl— solution and 1× with saturated NaCl. The organic layer was dried over MgSO₄, filtered and evaporated. The residue was purified using the Isco-Combiflash (12 g, 0-10% MeOH in CH₂Cl₂, 30 mL/min).

Yield: 680 mg (yellow oil)

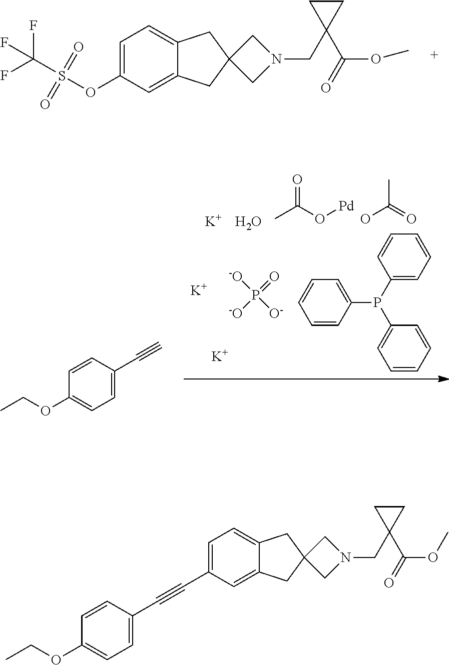

In a Schlenck flask were triphenylphosphine (8 mg, 0,031 mmol), palladium(II) acetate (2 mg, 8.91 μmol) and potassium phosphate tribasic monohydrate (40 mg, 0.174 mmol) dried for 30 min under Argon. In a second flask were 4-ethoxyphenylacetylene (25.1 mg, 0.172 mmol) and methyl 1-((5′-(((trifluoromethyl)sulfonyl)oxy)-1′,3′-dihydrospiro[azetidine-3,2′-inden]-1-yl)methyl)cyclopropanecarboxylate (60 mg, 0.143 mmol) dissolved in DMSO (2 mL) and dried under Argon for 30 min. This solution was put into the Schlenck flask via syringe and heated to 80° C. for 1 h.

The reaction mixture was diluted with CH₂Cl₂ and washed 2× with water and 1× with saturated NaCl. The organic layer was dried over a Chromabond PTS-cartridge and evaporated. The residue was purified using the Isco-Combiflash (12 g, 0-10% MeOH in CH₂Cl₂, 30 mL/min). The crude product contained some DMSO and was used in next step.

Yield: 290 mg (brown oil)

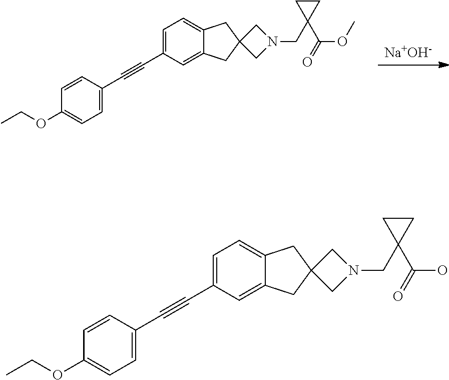

In a 50 mL round-bottomed flask (t=g) was methyl 1-((5′-((4-ethoxyphenyl)ethynyl)-1′,3′-dihydrospiro[azetidine-3,2′-inden]-1-yl)methyl)cyclopropanecarboxylate (290 mg, 0,140 mmol) in MeOH (1 mL) and THF (1 mL) to give a brown solution. Sodium hydroxide (0.349 mL, 2 mol) was added. The reaction mixture was stirred at RT overnight. The reaction mixture was evaporated and the residue was purified by HPLC.

Yield: 6.8 mg (yellow oil)

¹H NMR (600 MHz, Chloroform-d) δ 11.60 (s, OH), 7.43 (d, J=9.5 Hz, 1H), 7.38-7.28 (m, 1H), 7.18 (d, J=7.8 Hz, OH), 6.88-6.83 (m, 1H), 4.53 (d, J=10.3 Hz, 1H), 4.08-3.97 (m, 2H), 3.32 (d, J=19.2 Hz, 2H), 3.13 (d, J=5.9 Hz, 1H), 1.50 (q, J=4.7 Hz, 1H), 1.42 (t, J=7.0 Hz, 2H), 1.03 (q, J=4.7 Hz, 1H).

MS: Calculated mass (C26H27NO3): 401.20, found mass: M+H=402

### Example 2. 1-((5′-((2-methoxy-4-propylbenzyl)oxy)-1′,3′-dihydrospiro[azetidine-3,2′-inden]-1-yl)methyl)cyclopropanecarboxylic acid

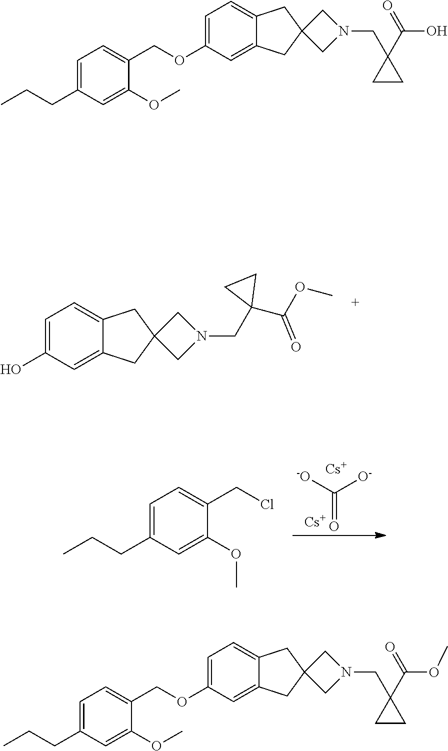

In a 50 mL round-bottomed flask methyl 1-((5′-hydroxy-1′,3′-dihydrospiro[azetidine-3,2′-inden]-1-yl)methyl)cyclopropanecarboxylate (50 mg, 0,174 mmol, prepared as described for example 28) was dissolved in DMF (3 mL) to give a colorless solution. Cesium carbonate (70 mg, 0.215 mmol) and 1-(chloromethyl)-2-methoxy-4-propylbenzene (40 mg, 0.201 mmol) were added. The reaction mixture was stirred at RT overnight.

The reaction mixture was evaporated. The residue was extracted with CH₂Cl₂ and water. After phase separation with a Chromabond PTS-cartridge the organic layer was evaporated. The residue was purified by using the Isco-Combiflash (4 g, 0-10% MeOH in CH₂Cl₂, 18 mL/min)

Yield: 50 mg (colorless oil)

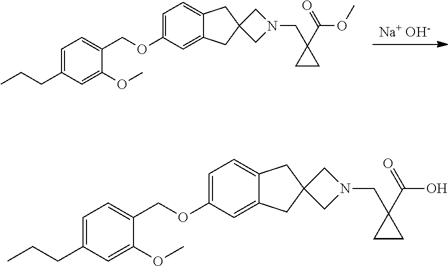

In a 10 mL flask was methyl 1-((5′-((2-methoxy-4-propylbenzyl)oxy)-1′,3′-dihydrospiro[azetidine-3,2′-inden]-1-yl)methyl)cyclopropanecarboxylate (50 mg, 0.111 mmol) dissolved in THF (0.5 mL) and MeOH (0.5 mL) to give a colorless solution. NaOH (0.5 mL, 1.0 mmol) was added. The reaction mixture was stirred at RT overnight.

The reaction mixture was evaporated. The residue was dissolved in water and neutralized with 0.5 mL 2N HCl. The precipitate was filtered, washed with water and dried overnight at 40° C. under vacuum.

Yield: 39 mg (colorless solid)

¹H NMR (600 MHz, DMSO-d6) δ 7.25 (d, J=7.6 Hz, 1H), 7.09 (d, J=8.1 Hz, 1H), 6.86 (dd, J=7.7, 1.9 Hz, 2H), 6.75 (ddd, J=26.5, 7.9, 2.0 Hz, 2H), 4.94 (s, 2H), 3.80 (s, 3H), 3.48 (s, 5H), 3.07 (s, 2H), 3.02 (s, 2H), 2.77 (s, 2H), 2.58-2.51 (m, 6H), 1.64-1.57 (m, 2H), 0.93-0.88 (m, 5H), 0.62 (q, J=3.7 Hz, 2H).

MS: Calculated mass (C27H33NO4): 435.24, found mass: M+H=436

### Example 3. 2-(5′-((4-butyl-2-chlorobenzyl)oxy)-1′,3′-dihydrospiro[azetidine-3,2′-inden]-1-yl)acetic acid

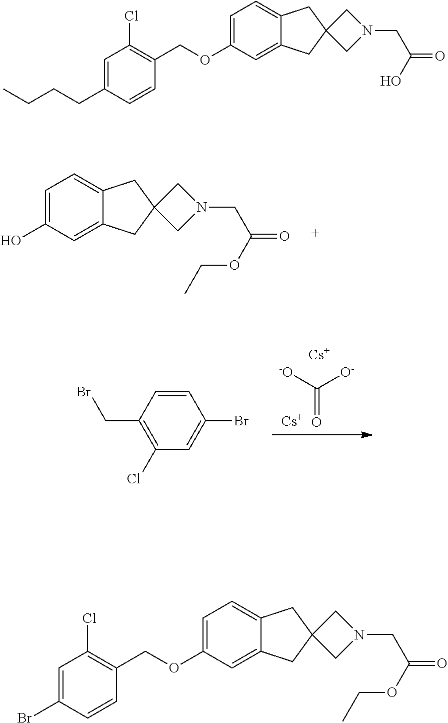

In a 100 mL round-bottom flask ethyl 2-(5′-hydroxy-1′,3′-dihydrospiro[azetidine-3,2′-inden]-1-yl)acetate (150 mg, 0.574 mmol, prepared as described for example 8) was dissolved in DMF (10 mL) to give a colorless solution. Cesium carbonate (230 mg, 0.706 mmol) and 4-bromo-1-(bromomethyl)-2-chlorobenzene (180 mg, 0.633 mmol) were added. The reaction mixture was stirred at RT overnight.

The reaction mixture was evaporated. The residue was dissolved in CH₂Cl₂ and washed 1× with water. After phase separation with a Chromabond PTS-cartridge the organic layer was evaporated. The residue was purified using the Isco-Combiflash (12 g, 0-10% MeOH in CH₂Cl₂)

Yield: 133 mg (yellow oil)

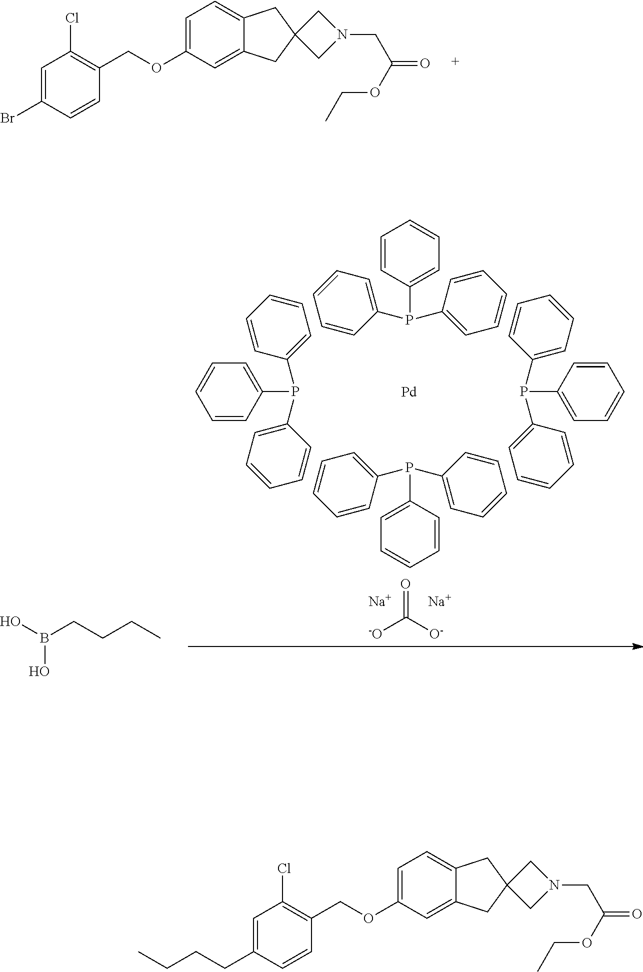

Under an atmosphere of argon ethyl 2-(5′-((4-bromo-2-chlorobenzyl)oxy)-1′,3′-dihydrospiro[azetidine-3,2′-inden]-1-yl)acetate (30 mg, 0.065 mmol) was dissolved in Toluene (PhCH₃) (2 mL) in a microwave flask. n-butylboronic acid (10 mg, 0.098 mmol) and sodium carbonate (0.2 ml, 0.208 mmol) were added to give a yellow suspension. The reaction mixture was stirred for 30 min under argon atmosphere. Tetrakis(triphenylphosphine)palladium(0) (8 mg, 6.92 μmol) was added. The mixture was stirred for 30 min at 100° C. in the Biotage microwave. LC/MS showed conversion, but the main peak was the reactant. The mixture was stirred for further 30 min at 120° C. in the Biotage micowave, LC/MS showed a better conversion rate. The reaction mixture was stirred for further 60 min at 120° C. in the Biotage microwave.

The reaction mixture was evaporated. The residue was dissolved in CH₂Cl₂ and washed 1× with water. After phase separation with a Chromabond PTS Cartridge the organic layer was evaporated. The residue was purified using the Isco-Combiflash (4 g, 0-10% MeOH in CH₂Cl₂).

Yield: 23 mg (yellow oil)

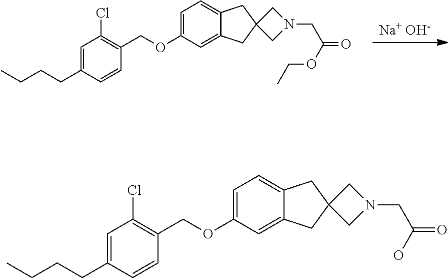 [SMILES: "Na+ OH-" ; CCCCc1ccc(COc2ccc3c(c2)CC2(C3)CN(CC(=O)OCC)C2)c(Cl)c1 ; CCCCc1ccc(COc2ccc3c(c2)CC2(C3)CN(CC(=O)O)C2)c(Cl)c1]

In a 25 mL round-bottomed flask ethyl 2-(5′-((4-butyl-2-chlorobenzyl)oxy)-1′,3′-dihydrospiro[azetidine-3,2′-inden]-1-yl)acetate (23 mg, 0.052 mmol) was dissolved in MeOH (0.5 mL) and THF (0.5 mL) to give a colorless solution. 2M NaOH (100 μL, 0.200 mmol) was added. The reaction mixture was stirred overnight. The reaction mixture was evaporated. The residue was dissolved in water and 100 μl 2N HCl were added. The mixture was stirred for 10 min and CH₂Cl₂ was added. After phase separation with a Chromabond-PTS-cartridge the organic layer was evaporated. The residue was purified using the Isco-Combiflash (4 g, 0-60% MeOH in CH₂Cl₂) and HPLC.

Yield: 2.6 mg (colorless oil)

¹H NMR (500 MHz, DMSO-d6) δ 7.46 (d, J=7.9 Hz, 1H), 7.34 (d, J=1.7 Hz, 1H), 7.20 (dd, J=7.9, 1.7 Hz, 1H), 7.12 (d, J=8.2 Hz, 1H), 6.91 (d, J=2.4 Hz, 1H), 6.79 (dd, J=8.2, 2.6 Hz, 1H), 5.05 (s, 2H), 4.00 (s, 4H), 3.89 (s, 2H), 3.18 (s, 2H), 3.13 (s, 2H), 2.64-2.52 (m, 3H), 2.45 (s, 1H), 1.60-1.50 (m, 2H), 1.34-1.22 (m, 2H), 0.89 (t, J=7.4 Hz, 3H).

MS: Calculated mass (C24H28ClNO3): 413.18, found mass: M+H=414/416

### Example 4. 2-(5′-((2-chloro-4-ethylbenzyl)oxy)-1′,3′-dihydrospiro[azetidine-3,2′-inden]-1-yl)acetic acid

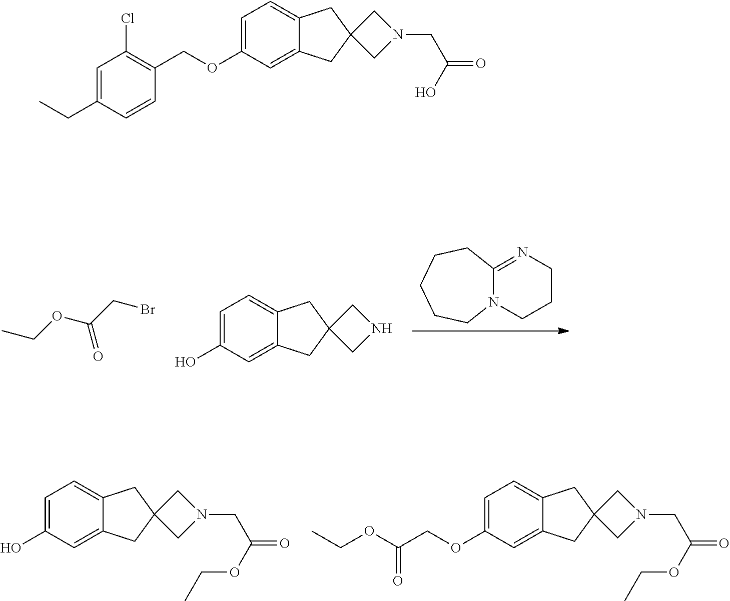

In a 100 mL round-bottomed flask (t=g) was 1′,3′-dihydrospiro[azetidine-3,2′-inden]-5′-ol (1 g, 5.71 mmol) in DMF (40 mL) to give a colorless solution. DBU (1.3 mL, 8.62 mmol) and ethyl bromoacetate (800 μL, 7.21 mmol) were added. The reaction mixture was stirred at RT for 1 hour.

The reaction mixture was evaporated, the residue was dissolved in Ethylacetate and washed 2× with sat. NH₄Cl-solution, 1× with saturated NaCl, the organic layer was dried with MgSO₄, filtered and evaporated. The residue was purified using the Isco-Combiflash (12 g, 0-20% MeOH in CH₂Cl₂, 30 mL/min)

Yield: 633 mg (light brown oil).

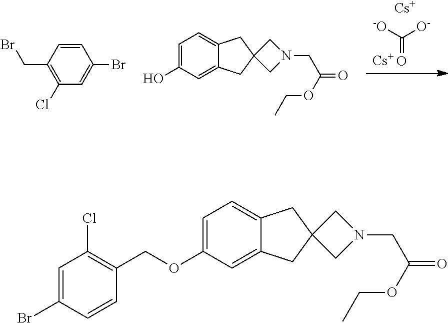

In a 100 mL round-bottom flask was ethyl 2-(5′-hydroxy-1′,3′-dihydrospiro[azetidine-3,2′-inden]-1-yl)acetate (150 mg, 0.574 mmol) in DMF (10 mL) to give a colorless solution. Cesium carbonate (230 mg, 0.706 mmol) and 4-bromo-1-(bromomethyl)-2-chlorobenzene (180 mg, 0.633 mmol) were added. The reaction mixture was stirred at RT overnight.

The reaction mixture was evaporated. The residue was dissolved in CH₂Cl₂ and washed 1× with water. After phase separation with a Chromabond PTS-cartridge the organic layer was evaporated. The residue was purified using the Isco-Combiflash (12 g, 0-10% MeOH in CH₂Cl₂)

Yield=133 mg yellow oil

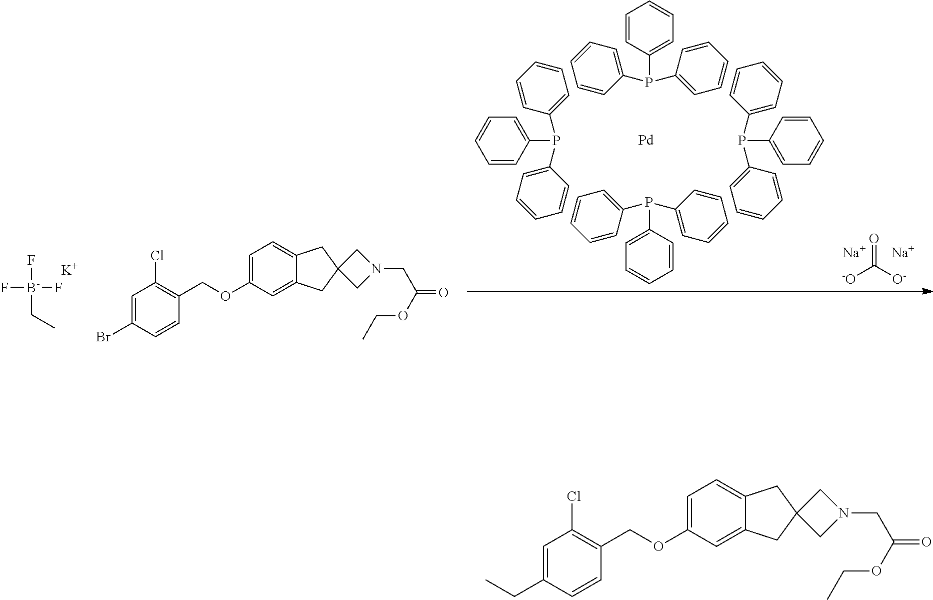

In a microwave flask was ethyl 2-(5′-((4-bromo-2-chlorobenzyl)oxy)-1′,3′-dihydrospiro[azetidine-3,2′-inden]-1-yl)acetate (mixture reactant and product) (50 mg, 0.108 mmol) dissolved in Toluene (PhCH₃) (3 mL). Potassium ethyltrifluoroborate (50 mg, 0.368 mmol) and sodium carbonate (0.3 mL, 0.311 mmol) were added to give a colorless suspension. The reaction mixture was stirred for 30 min under an argon atmosphere. Tetrakis(triphenylphosphine)palladium(0) (20 mg, 0.017 mmol) was added. The reaction was stirred for 2h at 120° C. in the microwave. LC/MS showed conversion to desired product.

The reaction mixture was evaporated. The residue was dissolved in CH₂Cl₂ and washed 1× with water, after phase separation with a Chromabond PTS Cartridge the organic layer was evaporated. The residue was purified using the Isco-Combiflash (4 g, 0-10% MeOH in CH₂Cl₂).

Yield=7 mg yellow oil

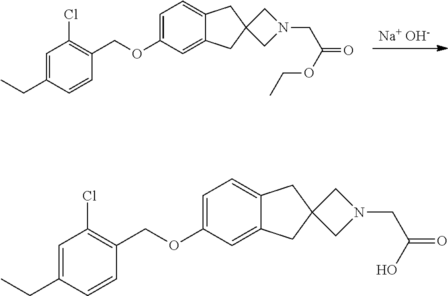 [SMILES: "Na+ OH-" ; CCOC(=O)CN1CC2(Cc3ccc(OCc4ccc(CC)cc4Cl)cc3C2)C1 ; CCc1ccc(COc2ccc3c(c2)CC2(C3)CN(CC(=O)O)C2)c(Cl)c1]

In a 4 mL vial was ethyl 2-(5′-((2-chloro-4-ethylbenzyl)oxy)-1′,3′-dihydrospiro[azetidine-3,2′-inden]-1-yl)acetate (7 mg, 0.017 mmol) dissolved in THF (0.2 mL) and MeOH (0.200 ml) to give a colorless solution. NaOH (0.042 mL, 0.085 mmol) was added. The reaction mixture was stirred overnight.

The reaction mixture was evaporated and the residue was purified by HPLC (Waters XBridge C18 OBD, acetonitrile, water, 0.1% TFA).

Yield: 1.4 mg clear oil (approx 70% purity by HPLC)

¹H NMR (500 MHz, Chloroform-d) δ 7.39 (dd, J=21.0, 7.9 Hz, 1H), 7.19 (s, 1H), 7.08 (dd, J=19.9, 8.0 Hz, 2H), 6.82 (d, J=17.0 Hz, 1H), 6.74 (d, J=7.8 Hz, 1H), 5.00 (s, 2H), 4.31 (s, 2H), 4.04 (s, 1H), 3.20 (d, J=17.4 Hz, 1H), 2.67-2.55 (m, 2H), 1.28-1.16 (m, 6H)

MS: Calculated mass (C22H24ClNO3): 385.14, found mass: M+H=386/388

### Example 5. 3-(5′-((tetrahydro-2H-pyran-3-yl)methoxy)-1′,3′-dihydrospiro[azetidine-3,2′-inden]-1-yl)cyclobutanecarboxylic acid

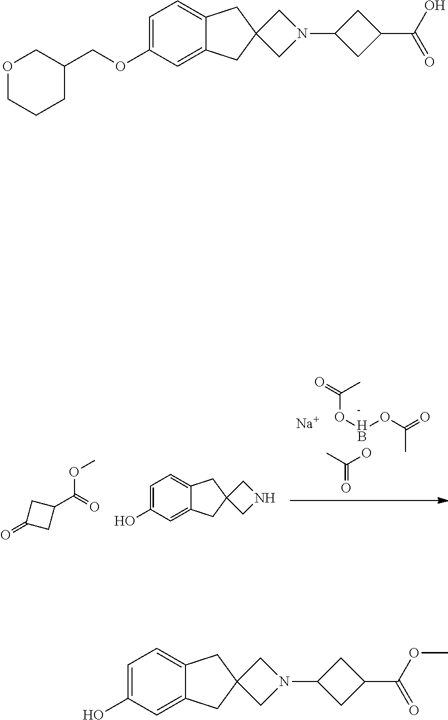

In a 100 mL 3-neck round-bottomed flask 1′,3′-dihydrospiro[azetidine-3,2′-inden]-5′-ol (2 g, 11.41 mmol) was suspended in THF (80 mL). Methyl 3-oxocyclobutanecarboxylate (2.92 g, 22.83 mmol) was added. The mixture was stirred for 60 min at RT. Sodium triacetoxyborohydride (4.84 g, 22.83 mmol) was added. The reaction mixture was stirred overnight at RT.

The reaction mixture was diluted with CH₂Cl₂ and water and stirred for 30 min. CH₂Cl₂ was added, the organic layer was washed 2× with NaHCO₃-solution, 1× with saturated sodium chloride solution, dried over MgSO₄, filtered and evaporated

Crude yield: 3.47 g light red solid

2.5 g of the crude product were absorbed on Celite XTR and purified using the Isco-Combiflash (12 g, 0-20% MeOH in CH₂Cl₂, 35 mL/min)

Yield: 1 g foam

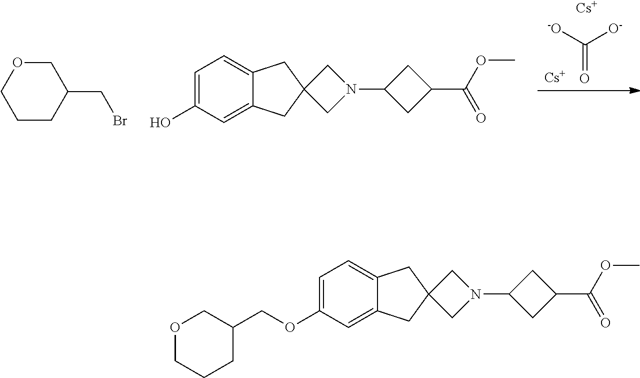

In a 50 mL round-bottom flask was methyl 3-(5′-hydroxy-1′,3′-dihydrospiro[azetidine-3,2′-inden]-1-yl)cyclobutanecarboxylate (50 mg, 0.174 mmol) in DMF (3 mL) to give a colorless solution. Cesium carbonate (85 mg, 0.261 mmol) and 3-(bromomethyl)tetrahydro-2H-pyran (40 mg, 0.223 mmol) were added. The reaction mixture was stirred at RT overnight.

The reaction mixture was evaporated. The residue was dissolved in water and CH₂Cl₂. After phase separation the organic layer was washed 1× with water and 1× with saturated sodium chloride solution. The organic layer was dried over MgSO₄, filtered and evaporated. The residue was purified using the Isco-Combiflash (4 g, 0-10% MeOH in CH₂Cl₂, 18 mL/min)

Yield: 34 mg clear oil

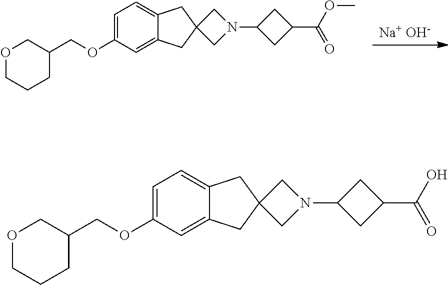 [SMILES: "Na+ OH-" ; COC(=O)C1CC(N2CC3(Cc4ccc(OCC5CCCOC5)cc4C3)C2)C1 ; O=C(O)C1CC(N2CC3(Cc4ccc(OCC5CCCOC5)cc4C3)C2)C1]

In a 50 mL round bottom flask was methyl 3-(5′-((tetrahydro-2H-pyran-3-yl)methoxy)-1′,3′-dihydrospiro[azetidine-3,2′-inden]-1-yl)cyclobutanecarboxylate (34 mg, 0.088 mmol) dissolved in THF (0.5 mL) and MeOH (0.5 mL) to give a yellow solution. NaOH (0.25 mL, 0.500 mmol) was added. The reaction mixture was stirred at RT overnight.

The reaction mixture was evaporated. The residue was dissolved in water and neutralized with 0.25 mL 2n HCl. CH₂Cl₂ was added, the mixture was stirred at RT for 1 h. After phase separation the organic layer was dried over MgSO₄, filtered and evaporated.

Yield: 27.7 mg white foam

¹H NMR (600 MHz, Methanol-d₄) δ 7.10 (d, J=8.2 Hz, 1H), 6.80 (d, J=2.2 Hz, 1H), 6.72 (dd, J=8.2, 2.4 Hz, 1H), 4.04 (s, 4H), 4.04-3.96 (m, 1H), 3.87-3.75 (m, 4H), 3.46 (ddd, J=11.4, 10.0, 3.3 Hz, 1H), 3.35 (dd, J=11.3, 9.2 Hz, 1H), 3.22 (s, 2H), 3.18 (s, 2H), 2.91-2.82 (m, 1H), 2.62-2.50 (m, 2H), 2.19-2.11 (m, 2H), 2.11-2.01 (m, 1H), 1.89 (dq, J=12.9, 4.3 Hz, 1H), 1.72-1.58 (m, 2H), 1.45 (dtd, J=13.1, 10.4, 4.6 Hz, 1H).

MS: Calculated mass (C22H29NO4): 371.21, found mass: M+H=372

### Example 6. 3-(5′-(benzyloxy)-1′,3′-dihydrospiro[azetidine-3,2′-inden]-1-yl)propanoic acid hydrochloride

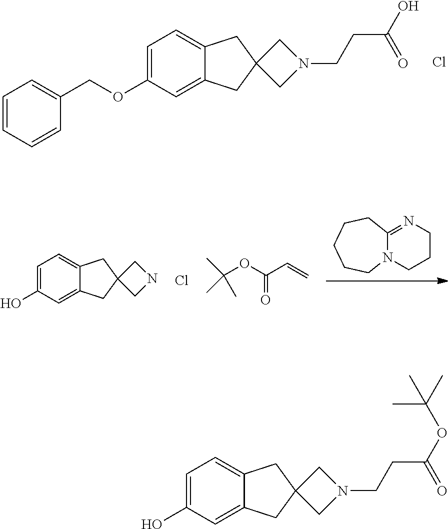

In a 50 mL round-bottomed flask 1′,3′-dihydrospiro[azetidine-3,2′-inden]-5′-ol hydrochloride (100 mg, 0.472 mmol) was dissolved in MeOH (5 mL) to give a colorless solution. DBU (0.5 mL, 3.32 mmol) and tert-butyl acrylate (0.5 mL, 3.41 mmol) were added. The reaction mixture was stirred at RT for 30 min. LC/MS showed complete conversion.

The reaction mixture was evaporated. The residue was dissolved in CH₂Cl₂, the organic layer was washed 2× with sat. NH₄Cl-solution, 1× with saturated sodium chloride solution, dried over MgSO₄, filtered and evaporated.

The crude product (oil) was purified using the Isco-Combiflash (4 g, 0-10% MeOH in CH₂Cl₂)

Yield: 99 mg clear oil

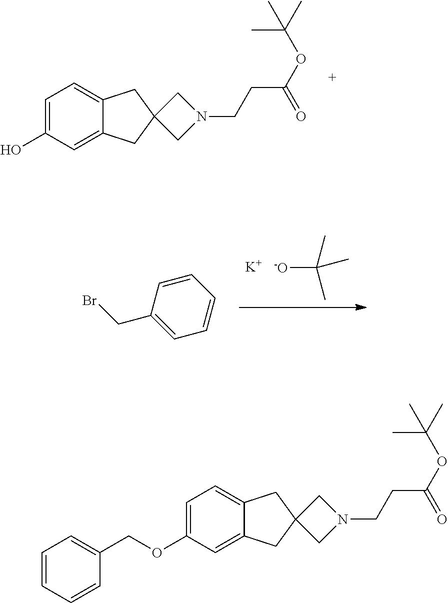

In a Schlenck flask tert-butyl 3-(5′-hydroxy-1′,3′-dihydrospiro[azetidine-3,2′-inden]-1-yl)propanoate (50 mg, 0.165 mmol) was dissolved in DMF (2 mL) to give a colorless solution. The reaction mixture was cooled down to 0° C. and was added. Stirred at 0° C. for 30 min. Benzyl bromide (0.020 mL, 0.165 mmol) was added. After 1 h the reaction was finished.

0.5 mL sat. NH₄Cl-solution was added slowly to the mixture and after that the mixture was diluted with ethyl acetate. The organic layer was washed 1× with water, after phase separation, the organic layer was dried over MgSO₄, filtered and evaporated. The residue was purified using the Isco-Combiflash (4 g, 0-10% MeOH in CH₂Cl₂).

Yield: 41 mg oil

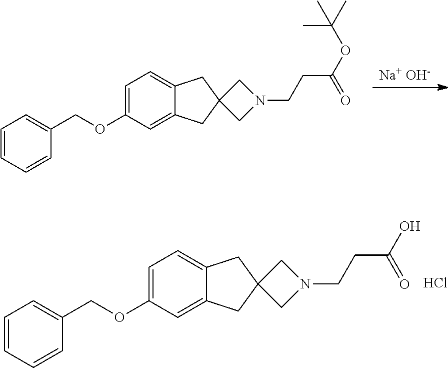 [SMILES: "Na+ OH-" ; CC(C)(C)OC(=O)CCN1CC2(Cc3ccc(OCc4ccccc4)cc3C2)C1 ; O=C(O)CCN1CC2(Cc3ccc(OCc4ccccc4)cc3C2)C1 ; Cl]

In a 25 mL round-bottomed flask tert-butyl 3-(5′-(benzyloxy)-1′,3′-dihydrospiro[azetidine-3,2′-inden]-1-yl)propanoate (41 mg, 0.104 mmol) was dissolved in Methanol (1 mL) and Tetrahydrofuran (1 mL). NaOH (250 μL, 0.500 mmol) was added. The reaction mixture was stirred at RT overnight. LC/MS showed that the conversion was not complete. 300 μL 2n NaOH were added and stirred for further 4h at RT.

The reaction mixture was evaporated. The residue was dissolved in water and acidified with 2n HCl (1 mL, pH-value 1-2). The precipitate was filtered, washed 1× with water, dried overnight under vacuum at 40° C.

Yield: 33 mg white solid

¹H NMR (600 MHz, DMSO-d6) δ 12.74 (s, 1H), 10.21 (s, 1H), 7.46-7.40 (m, 2H), 7.39 (dd, J=8.5, 6.7 Hz, 2H), 7.35-7.29 (m, 1H), 7.12 (d, J=8.3 Hz, 1H), 6.91 (d, J=2.5 Hz, 1H), 6.80 (dd, J=8.3, 2.5 Hz, 1H), 5.05 (s, 2H), 4.05 (s, 4H), 3.14 (d, J=29.5 Hz, 4H), 2.57 (t, J=7.2 Hz, 2H).

MS: Calculated mass (C21H23NO3): 337.17, found mass: M+H=338

Example 7 was prepared analogous to example 6:

### Example 7. 3-(5′-((2-chloro-6-ethylbenzyl)oxy)-1′,3′-dihydrospiro[azetidine-3,2′-inden]-1-yl)propanoic acid hydrochloride

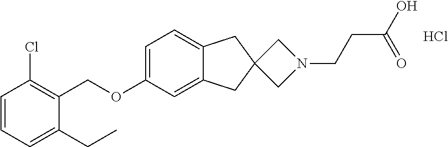 [SMILES: CCc1cccc(Cl)c1COc1ccc2c(c1)CC1(C2)CN(CCC(=O)O)C1 ; Cl]

¹H NMR (600 MHz, DMSO-d6) δ 12.78 (s, OH), 10.16 (s, OH), 7.42-7.33 (m, 2H), 7.28 (dd, J=5.7, 3.3 Hz, 1H), 7.15 (d, J=8.2 Hz, 1H), 6.97 (d, J=2.4 Hz, 1H), 6.83 (dd, J=8.2, 2.5 Hz, 1H), 5.10 (s, 2H), 4.07 (s, 4H), 3.16 (d, J=32.9 Hz, 4H), 2.71 (q, J=7.6 Hz, 2H), 2.57 (t, J=7.2 Hz, 2H), 1.16 (t, J=7.6 Hz, 3H).

MS: Calculated mass (C23H26ClNO3): 399.16, found mass: M+H=400/402

### Example 8. 2-(5′-(benzyloxy)-1′,3′-dihydrospiro[azetidine-3,2′-inden]-1-yl)acetic acid

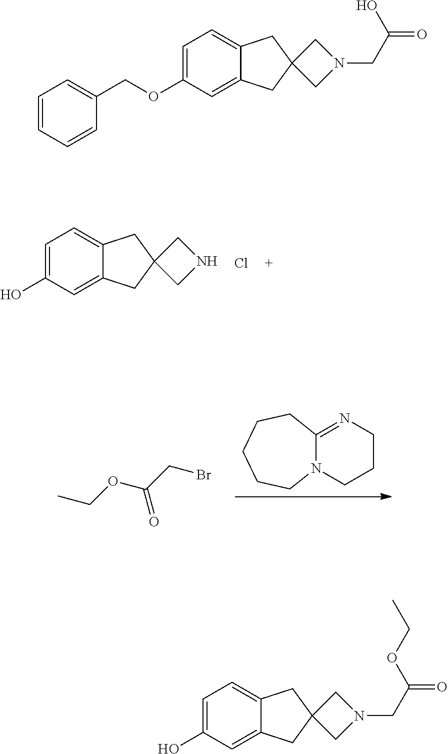

In a 50 mL round-bottomed flask 1′,3′-dihydrospiro[azetidine-3,2′-inden]-5′-0l hydrochloride (100 mg, 0.472 mmol) was dissolved in Acetonitrile (5 mL) to give a colorless solution. DBU (0.2 mL, 1.327 mmol) and ethyl bromoacetate (55 μL, 0.496 mmol) were added. The reaction mixture was stirred for 1 h at RT.

The reaction mixture was evaporated, the residue was dissolved in CH₂Cl₂ and washed 1× with sat. NH₄Cl-solution, the phases were separated with a Chromabond PTS-Cartridge and the organic layer was evaporated. The residue was purified by flash chromatography (silica 4 g, 0-10% MeOH in CH₂Cl₂)

Yield: 81 mg clear oil

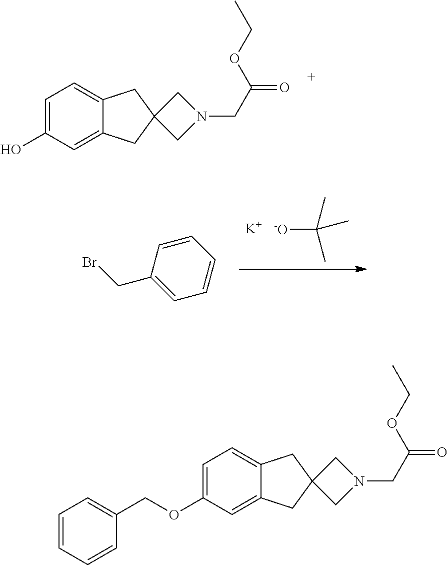

In a Schlenck flask (ethyl 2-(5′-hydroxy-1′,3′-dihydrospiro[azetidine-3,2′-inden]-1-yl)acetate (40 mg, 0.153 mmol) was dissolved in DMF (2 mL) to give a colorless solution. The reaction mixture was cooled to 0° C. and potassium tert-butoxide (20 mg, 0.178 mmol) was added. The reaction mixture was stirred at 0° C. for 30 min. benzyl bromide (20 μL, 0.168 mmol) was added.

(0.5 mL sat. NH₄Cl-solution was added slowly to the mixture and after that the mixture was diluted with ethyl acetate. The organic layer was washed 1× with water, after phase separation, the organic layer was dried over MgSO₄, filtered and evaporated. The residue was absorbed on Celite XTR and purified using the Isco-Combiflash (4 g, 0-10% MeOH in CH₂Cl₂)

Yield: 25 mg clear oil

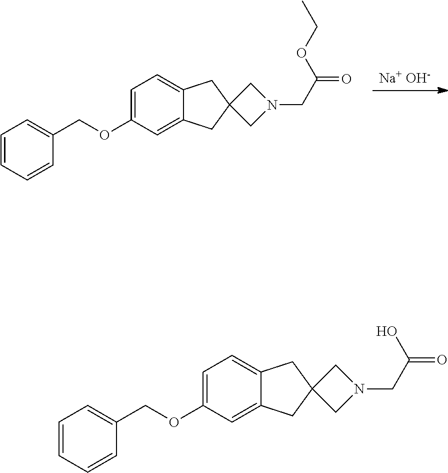 [SMILES: "Na+ OH-" ; CCOC(=O)CN1CC2(Cc3ccc(OCc4ccccc4)cc3C2)C1 ; O=C(O)CN1CC2(Cc3ccc(OCc4ccccc4)cc3C2)C1]

In a 25 mL round-bottomed flask ethyl 2-(5′-(benzyloxy)-1′,3′-dihydrospiro[azetidine-3,2′-inden]-1-yl)acetate (25 mg, 0.071 mmol) was dissolved in MeOH (1 mL) and THF (1 mL) to give a colorless solution. NaOH (0.2 mL, 0.400 mmol) was added. The reaction mixture was stirred at RT overnight.

The reaction mixture was evaporated. The residue was dissolved in water and neutralized with HOAc (to pH value 6). 50 mL CH₂Cl₂ were added. After phase separation with a Chromabond PTS cartridge, the organic layer was evaporated and purified by flash chromatography (silica 4 g, 0-30% MeOH in CH₂Cl₂) The product was dried at 40° C. under vacuum. Yield: 17.7 mg white solid

¹H NMR (600 MHz, DMSO-d6) δ 12.78 (s, OH), 10.16 (s, OH), 7.42-7.33 (m, 2H), 7.28 (dd, J=5.7, 3.3 Hz, 1H), 7.15 (d, J=8.2 Hz, 1H), 6.97 (d, J=2.4 Hz, 1H), 6.83 (dd, J=8.2, 2.5 Hz, 1H), 5.10 (s, 2H), 4.07 (s, 4H), 3.16 (d, J=32.9 Hz, 4H), 2.71 (q, J=7.6 Hz, 2H), 2.57 (t, J=7.2 Hz, 2H), 1.16 (t, J=7.6 Hz, 3H).

MS: Calculated mass (C20H21NO3): 323.15, found mass: M+H+=324

Examples 9-27 were prepared analogous to example 8:

### Example 9. 2,2,2-trifluoroacetic acid compound with 2-(5′-((2-chloro-6-methoxybenzyl)oxy)-1′,3′-dihydrospiro[azetidine-3,2′-inden]-1-yl)acetic acid (1:1)

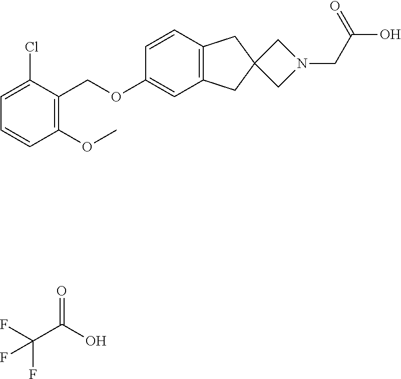 [SMILES: COc1cccc(Cl)c1COc1ccc2c(c1)CC1(C2)CN(CC(=O)O)C1 ; O=C(O)C(F)(F)F]

¹H NMR (600 MHz, DMSO-d6) δ 10.51 (s, 1H), 7.41 (t, J=8.2 Hz, 1H), 7.19-7.05 (m, 3H), 6.93 (d, J=2.3 Hz, 1H), 6.80 (dd, J=8.2, 2.5 Hz, 1H), 5.04 (s, 2H), 4.22 (s, 2H), 4.19-4.10 (m, 4H), 3.21 (s, 3H), 3.16 (s, 2H).

MS: Calculated mass (C21H22ClNO4): 387.12, found mass: M+H=388/390

### Example 10. 2,2,2-trifluoroacetic acid compound with 2-(5′-((2-fluoro-6-methoxybenzyl)oxy)-1′,3′-dihydrospiro[azetidine-3,2′-inden]-1-yl)acetic acid (1:1)

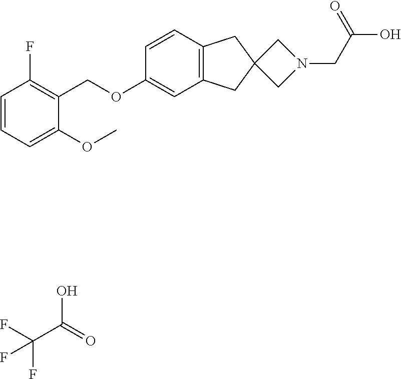 [SMILES: COc1cccc(F)c1COc1ccc2c(c1)CC1(C2)CN(CC(=O)O)C1 ; O=C(O)C(F)(F)F]

¹H NMR (500 MHz, DMSO-d6) δ 7.42 (td, J=8.5, 6.9 Hz, 1H), 7.12 (d, J=8.2 Hz, 1H), 6.98-6.90 (m, 2H), 6.85 (t, J=8.8 Hz, 1H), 6.78 (dd, J=8.2, 2.5 Hz, 1H), 4.97 (d, J=1.5 Hz, 2H), 4.19 (s, 2H), 4.13 (d, J=1.5 Hz, 4H), 3.83 (s, 3H), 3.20 (s, 2H), 3.15 (s, 2H).

MS: Calculated mass (C21H22FNO4): 371.15, found mass: M+H=372

### Example 11. 2-(5′-((4-chlorobenzyl)oxy)-1′,3′-dihydrospiro[azetidine-3,2′-inden]-1-yl)acetic acid

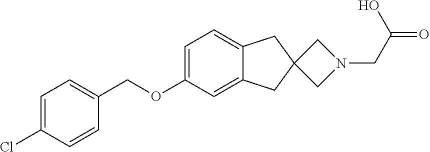 [SMILES: O=C(O)CN1CC2(Cc3ccc(OCc4ccc(Cl)cc4)cc3C2)C1]

¹H NMR (600 MHz, DMSO-d6) δ 7.45 (s, 4H), 7.10 (d, J=8.2 Hz, 1H), 6.88 (d, J=2.3 Hz, 1H), 6.76 (dd, J=8.2, 2.5 Hz, 1H), 5.05 (s, 2H), 3.75 (s, 4H), 3.12 (s, 2H), 3.07 (s, 2H).

MS: Calculated mass (C20H20ClNO3): 357.11, found mass: M+H=358/360

### Example 12. 2,2,2-trifluoroacetic acid compound with 2-(5′-((2-(trifluoromethyl)benzyl)oxy)-1′,3′-dihydrospiro[azetidine-3,2′-inden]-1-yl)acetic acid (1:1)

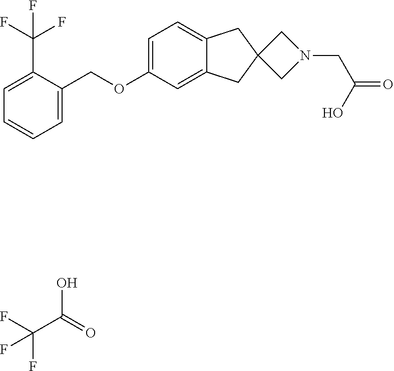 [SMILES: O=C(O)CN1CC2(Cc3ccc(OCc4ccccc4C(F)(F)F)cc3C2)C1 ; O=C(O)C(F)(F)F]

¹H NMR (500 MHz, DMSO-d6) δ 7.80 (d, J=7.8 Hz, 1H), 7.77-7.69 (m, 2H), 7.59 (t, J=7.4 Hz, 1H), 7.14 (d, J=8.2 Hz, 1H), 6.91 (d, J=2.4 Hz, 1H), 6.79 (dd, J=8.3, 2.5 Hz, 1H), 5.18 (s, 2H), 4.16 (s, 1H), 4.12 (s, 4H), 3.20 (s, 2H), 3.15 (s, 2H).

MS: Calculated mass (C21H20F3NO3): 391.14, found mass: M+H=392

### Example 13. 2,2,2-trifluoroacetic acid compound with 2-(5′-((2,6-difluorobenzyl)oxy)-1′,3′-dihydrospiro[azetidine-3,2′-inden]-1-yl)acetic acid (1:1)

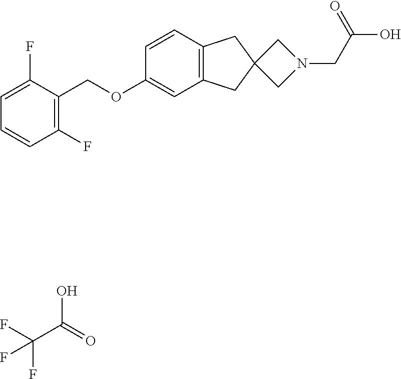 [SMILES: O=C(O)CN1CC2(Cc3ccc(OCc4c(F)cccc4F)cc3C2)C1 ; O=C(O)C(F)(F)F]

¹H NMR (500 MHz, DMSO-d6) δ 7.52 (tt, J=8.5, 6.7 Hz, 1H), 7.17 (t, J=8.0 Hz, 2H), 7.10 (d, J=8.2 Hz, 1H), 6.89 (d, J=2.6 Hz, 1H), 6.76 (dd, J=8.2, 2.4 Hz, 1H), 5.04 (s, 2H), 3.08-2.97 (m, 6H).

MS: Calculated mass (C20H19F2NO3): 359.13, found mass: M+H=360

### Example 14. 2-(5′-((3,5-difluorobenzyl)oxy)-1′,3′-dihydrospiro[azetidine-3,2′-inden]-1-yl)acetic acid

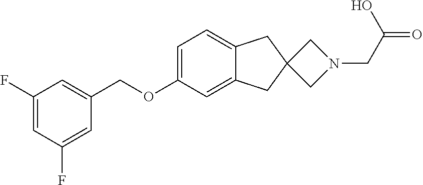 [SMILES: O=C(O)CN1CC2(Cc3ccc(OCc4cc(F)cc(F)c4)cc3C2)C1]

¹H NMR (600 MHz, DMSO-d6) δ 7.22-7.13 (m, 3H), 7.11 (d, J=8.2 Hz, 1H), 6.89 (d, J=2.4 Hz, 1H), 6.78 (dd, J=8.2, 2.5 Hz, 1H), 5.09 (s, 2H), 3.77 (s, 4H), 3.40 (s, 2H), 3.13 (s, 2H), 3.08 (s, 2H).

MS: Calculated mass (C20H19F2NO3): 359.13, found mass: M+H=360

### Example 15. 2-(5′-((2-chloro-6-cyclopropylbenzyl)oxy)-1′,3′-dihydrospiro[azetidine-3,2′-inden]-1-yl)acetic acid

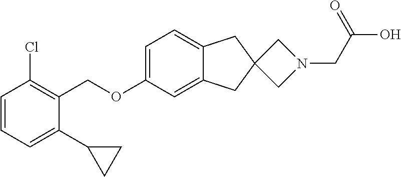 [SMILES: O=C(O)CN1CC2(Cc3ccc(OCc4c(Cl)cccc4C4CC4)cc3C2)C1]

¹H NMR (600 MHz, DMSO-d₆) δ 7.39-7.28 (m, 2H), 7.14 (d, J=8.2 Hz, 1H), 7.04 (d, J=7.3 Hz, 1H), 6.97 (d, J=2.3 Hz, 1H), 6.84 (dd, J=8.2, 2.4 Hz, 1H), 5.26 (s, 2H), 3.92-3.85 (m, 4H), 3.56 (s, 2H), 3.18 (s, 2H), 3.12 (s, 2H), 2.06 (ddd, J=13.7, 8.6, 5.3 Hz, 1H), 0.96-0.88 (m, 2H), 0.70 (q, J=5.3 Hz, 2H).

MS: Calculated mass (C23H24ClNO3): 397.14, found mass: M+H=398/400

### Example 16. 2-(5′-((3-chlorobenzyl)oxy)-1′,3′-dihydrospiro[azetidine-3,2′-inden]-1-yl)acetic acid compound with acetic acid (1:1)

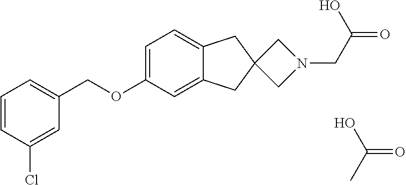

¹H NMR (600 MHz, DMSO-d₆) δ 11.78 (s, 2H), 7.49 (t, J=1.7 Hz, 1H), 7.44-7.37 (m, 3H), 7.10 (d, J=8.2 Hz, 1H), 6.89 (d, J=2.4 Hz, 1H), 6.78 (dd, J=8.2, 2.5 Hz, 1H), 5.07 (s, 2H), 3.78 (s, 4H), 3.41 (s, 2H), 3.13 (s, 2H), 3.08 (s, 2H), 1.91 (s, 6H).

MS: Calculated mass (C20H20ClNO3): 357.11, found mass: M+H=358/360

### Example 17. 2,2,2-trifluoroacetic acid compound with 2-(5′-((2-fluoro-6-methylbenzyl)oxy)-1′,3′-dihydrospiro[azetidine-3,2′-inden]-1-yl)acetic acid (1:1)

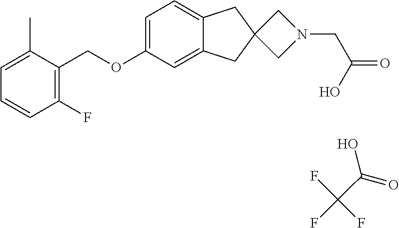 [SMILES: Cc1cccc(F)c1COc1ccc2c(c1)CC1(C2)CN(CC(=O)O)C1 ; O=C(O)C(F)(F)F]

¹H NMR (500 MHz, DMSO-d₆) δ 10.52 (s, 1H), 7.32 (td, J=8.0, 5.9 Hz, 1H), 7.16-7.04 (m, 3H), 6.94 (d, J=2.6 Hz, 1H), 6.82 (dd, J=8.2, 2.5 Hz, 1H), 5.04 (d, J=1.7 Hz, 2H), 4.21 (s, 2H), 4.15 (d, J=1.5 Hz, 4H), 3.21 (s, 2H), 3.16 (s, 2H), 2.36 (s, 4H).

MS: Calculated mass (C21H22FNO3): 355.16, found mass: M+H=356

### Example 18. 2,2,2-trifluoroacetic acid compound with 2-(5′-((2-chloro-6-fluorobenzyl)oxy)-1′,3′-dihydrospiro[azetidine-3,2′-inden]-1-yl)acetic acid (1:1)

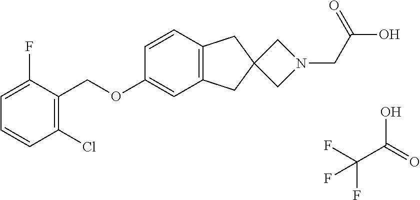 [SMILES: O=C(O)CN1CC2(Cc3ccc(OCc4c(F)cccc4Cl)cc3C2)C1 ; O=C(O)C(F)(F)F]

¹H NMR (500 MHz, DMSO-d₆) δ 7.51 (td, J=8.3, 6.2 Hz, 1H), 7.42 (d, J=8.1 Hz, 1H), 7.31 (t, J=8.8 Hz, 1H), 7.14 (d, J=8.2 Hz, 1H), 6.95 (d, J=2.4 Hz, 1H), 6.82 (dd, J=8.2, 2.4 Hz, 1H), 5.09 (d, J=1.9 Hz, 2H), 4.04 (s, 4H), 3.96 (s, 2H), 3.19 (s, 2H), 3.14 (s, 2H).

MS: Calculated mass (C20H19C1FNO3): 375.10, found mass: M+H=376/378

### Example 19. 2-(5′-((2-fluoro-6-(trifluoromethyl)benzyl)oxy)-1′,3′-dihydrospiro[azetidine-3,2′-inden]-1-yl)acetic acid

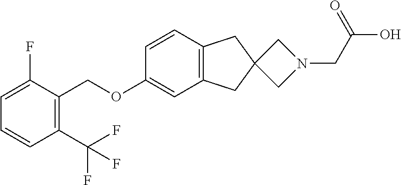 [SMILES: O=C(O)CN1CC2(Cc3ccc(OCc4c(F)cccc4C(F)(F)F)cc3C2)C1]

¹H NMR (500 MHz, DMSO-d₆) δ 7.80 (d, J=7.8 Hz, 1H), 7.77-7.69 (m, 2H), 7.59 (t, J=7.4 Hz, 1H), 7.14 (d, J=8.2 Hz, 1H), 6.91 (d, J=2.4 Hz, 1H), 6.79 (dd, J=8.3, 2.5 Hz, 1H), 5.18 (s, 2H), 4.14 (d, J=20.4 Hz, 6H), 3.20 (s, 2H), 3.15 (s, 2H).

MS: Calculated mass (C21H19F4NO3): 409.13, found mass: M+H=410

### Example 20. 2,2,2-trifluoroacetic acid compound with 2-(5′-((2-chloro-6-(trifluoromethyl)benzyl)oxy)-1′,3′-dihydrospiro[azetidine-3,2′-inden]-1-yl)acetic acid (1:1)

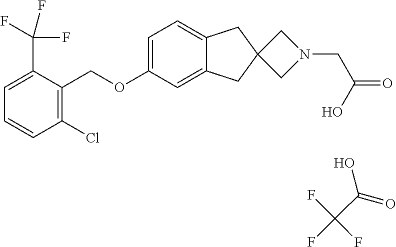 [SMILES: O=C(O)CN1CC2(Cc3ccc(OCc4c(Cl)cccc4C(F)(F)F)cc3C2)C1 ; O=C(O)C(F)(F)F]

¹H NMR (500 MHz, DMSO-d₆) δ 13.89 (s, OH), 10.57 (s, 1H), 7.92 (d, J=8.1 Hz, 1H), 7.84 (d, J=7.9 Hz, 1H), 7.69 (t, J=8.0 Hz, 1H), 7.15 (d, J=8.2 Hz, 1H), 6.97 (d, J=2.3 Hz, 1H), 6.83 (dd, J=8.2, 2.5 Hz, 1H), 5.14 (s, 2H), 4.23 (s, 2H), 4.21-4.12 (m, 4H), 3.23 (s, 2H), 3.17 (s, 2H).

MS: Calculated mass (C21H19C1F3NO3): 425.10, found mass: M+H=426/428

### Example 21. 2-(5′-((2-ethyl-6-fluorobenzyl)oxy)-1′,3′-dihydrospiro[azetidine-3,2′-inden]-1-yl)acetic acid

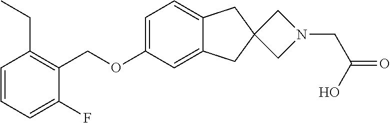 [SMILES: CCc1cccc(F)c1COc1ccc2c(c1)CC1(C2)CN(CC(=O)O)C1]

¹H NMR (600 MHz, DMSO-d₆) δ 7.38 (td, J=8.0, 6.0 Hz, 1H), 7.17-7.11 (m, 2H), 7.08 (ddd, J=9.6, 8.2, 1.1 Hz, 1H), 6.93 (d, J=2.3 Hz, 1H), 6.79 (dd, J=8.2, 2.5 Hz, 1H), 5.02 (d, J=1.7 Hz, 2H), 3.77 (d, J=2.2 Hz, 5H), 3.14 (s, 3H), 3.09 (s, 2H), 2.70 (q, J=7.5 Hz, 2H).

MS: Calculated mass (C22H24FNO3): 369.17, found mass: M+H=370

### Example 22. 2-(5′-((4-bromo-2-chlorobenzyl)oxy)-1′,3′-dihydrospiro[azetidine-3,2′-inden]-1-yl)acetic acid

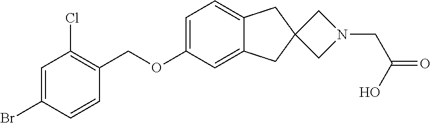 [SMILES: O=C(O)CN1CC2(Cc3ccc(OCc4ccc(Br)cc4Cl)cc3C2)C1]

¹H NMR (600 MHz, DMSO-d₆) δ 7.81 (d, J=2.0 Hz, 1H), 7.61 (dd, J=8.2, 2.0 Hz, 1H), 7.52 (d, J=8.3 Hz, 1H), 7.13 (d, J=8.3 Hz, 1H), 6.91 (d, J=2.4 Hz, 1H), 6.79 (dd, J=8.2, 2.5 Hz, 1H), 5.07 (s, 2H), 3.90 (s, 4H), 3.16 (s, 2H), 3.11 (s, 2H).

MS: Calculated mass (C20H19BrClNO3): 435.02, found mass: M+H=436/438

### Example 23. 2,2,2-trifluoroacetic acid compound with 2-(5′-((2,5-dichlorobenzyl)oxy)-1′,3′-dihydrospiro[azetidine-3,2′-inden]-1-yl)acetic acid (1:1)

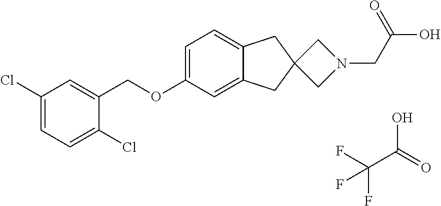 [SMILES: O=C(O)CN1CC2(Cc3ccc(OCc4cc(Cl)ccc4Cl)cc3C2)C1 ; O=C(O)C(F)(F)F]

¹H NMR (500 MHz, DMSO-d₆) δ 7.64 (d, J=2.7 Hz, 1H), 7.56 (d, J=8.5 Hz, 1H), 7.47 (dd, J=8.6, 2.7 Hz, 1H), 7.15 (d, J=8.3 Hz, 1H), 6.95 (d, J=2.4 Hz, 1H), 6.83 (dd, J=8.2, 2.5 Hz, 1H), 5.10 (s, 2H), 4.08 (s, 5H), 3.20 (s, 2H), 3.15 (s, 2H).

MS: Calculated mass (C20H19Cl2NO3): 391.07, found mass: M+H=392/394/396

### Example 24. 2,2,2-trifluoroacetic acid compound with 2-(5′-((2-chloro-6-ethylbenzyl)oxy)-1′,3′-dihydrospiro[azetidine-3,2′-inden]-1-yl)acetic acid (1:1)

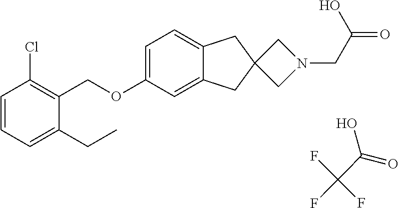 [SMILES: CCc1cccc(Cl)c1COc1ccc2c(c1)CC1(C2)CN(CC(=O)O)C1 ; O=C(O)C(F)(F)F]

¹H NMR (500 MHz, DMSO-d₆) δ 7.37-7.35 (m, 2H), 7.28 (dd, J=5.4, 3.7 Hz, 1H), 7.15 (d, J=8.3 Hz, 1H), 6.96 (d, J=2.4 Hz, 1H), 6.83 (dd, J=8.2, 2.5 Hz, 1H), 5.10 (s, 2H), 4.20-4.10 (m, 6H), 3.22 (s, 2H), 3.16 (s, 2H), 2.71 (q, J=7.5 Hz, 2H), 1.16 (t, J=7.5 Hz, 3H).

MS: Calculated mass (C22H24ClNO3): 385.14, found mass: M+H=386/388

### Example 25. 2,2,2-trifluoroacetic acid compound with 2-(5′-((3,4-dichlorobenzyl)oxy)-1′,3′-dihydrospiro[azetidine-3,2′-inden]-1-yl)acetic acid (1:1)

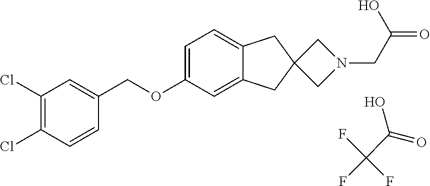

¹H NMR (500 MHz, DMSO-d₆) δ 7.69 (d, J=2.1 Hz, 1H), 7.66 (d, J=8.3 Hz, 1H), 7.42 (dd, J=8.3, 2.1 Hz, 1H), 7.12 (d, J=8.3 Hz, 1H), 6.90 (d, J=2.4 Hz, 1H), 6.79 (dd, J=8.3, 2.5 Hz, 1H), 5.08 (s, 2H), 4.06 (d, J=6.4 Hz, 5H), 3.18 (s, 2H), 3.13 (s, 2H)

MS: Calculated mass (C20H19Cl2NO3): 391.07, found mass: M+H=392/394/396

### Example 26. 2-(5′-((2,6-dimethylbenzyl)oxy)-1′,3′-dihydrospiro[azetidine-3,2′-inden]-1-yl)acetic acid

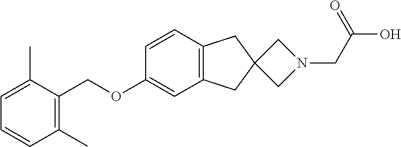 [SMILES: Cc1cccc(C)c1COc1ccc2c(c1)CC1(C2)CN(CC(=O)O)C1]

¹H NMR (600 MHz, DMSO-d₆) δ 7.14 (dt, J=15.7, 7.9 Hz, 2H), 7.06 (d, J=7.6 Hz, 2H), 6.94 (d, J=2.5 Hz, 1H), 6.80 (dd, J=8.2, 2.5 Hz, 1H), 3.78 (d, J=2.0 Hz, 4H), 3.40 (s, 2H), 3.15 (s, 2H), 3.09 (s, 2H), 2.31 (s, 6H).

MS: Calculated mass (C22H25NO3): 351.18, found mass: M+H=352

### Example 27. 2-(5′-((5-fluoro-2-(trifluoromethyl)benzyl)oxy)-1′,3′-dihydrospiro[azetidine-3,2′-inden]-1-yl)acetic acid

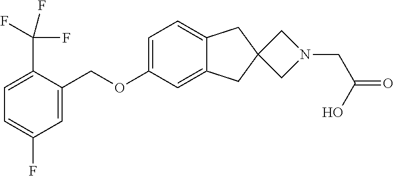 [SMILES: O=C(O)CN1CC2(Cc3ccc(OCc4cc(F)ccc4C(F)(F)F)cc3C2)C1]

¹H NMR (600 MHz, DMSO-d₆) δ 7.88 (dd, J=8.8, 5.4 Hz, 1H), 7.61 (dd, J=9.7, 2.7 Hz, 1H), 7.45-7.39 (m, 1H), 7.09 (d, J=8.2 Hz, 1H), 6.88 (d, J=2.4 Hz, 1H), 6.74 (dd, J=8.2, 2.5 Hz, 1H), 5.18 (s, 2H), 3.05 (s, 4H), 2.98 (s, 2H), 2.93 (s, 2H), 2.66 (s, 2H).

MS: Calculated mass (C21H19F4NO3): 409.13, found mass: M+H=410

### Example 28. 1-((5′-((2-chloro-6-ethylbenzyl)oxy)-1′,3′-dihydrospiro[azetidine-3,2′-inden]-1-yl)methyl)cyclopropanecarboxylic acid

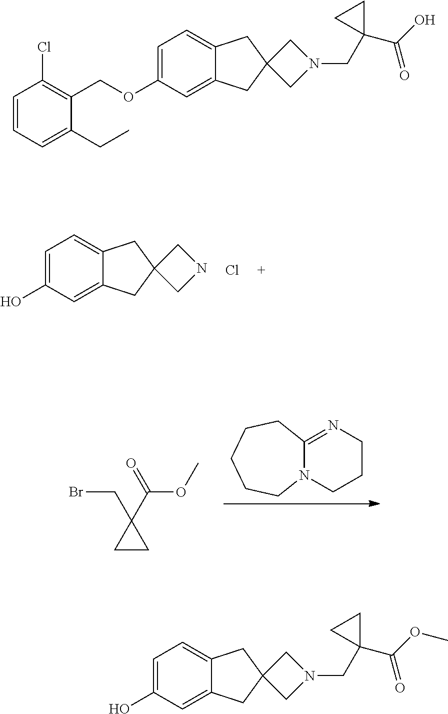

In a 50 mL round-bottomed flask 1′,3′-dihydrospiro[azetidine-3,2′-inden]-5′-ol hydrochloride (100 mg, 0.472 mmol) was dissolved in DMF (3 mL) to give a colorless solution. DBU (0.2 mL, 1.327 mmol) and methyl 1-(bromomethyl)cyclopropanecarboxylate (119 mg, 0.614 mmol) were added. The reaction mixture was stirred at RT for 1 h.

The reaction mixture was evaporated, the residue was dissolved in CH₂Cl₂ and washed 1× with sat. NH₄Cl-solution, the phases were separated with a Chromabond PTS-Cartridge and the organic layer was evaporated. The residue was purified using the Isco-Combiflash (4 g, 0-10% MeOH in CH₂Cl₂)

Yield: 109 mg colorless oil

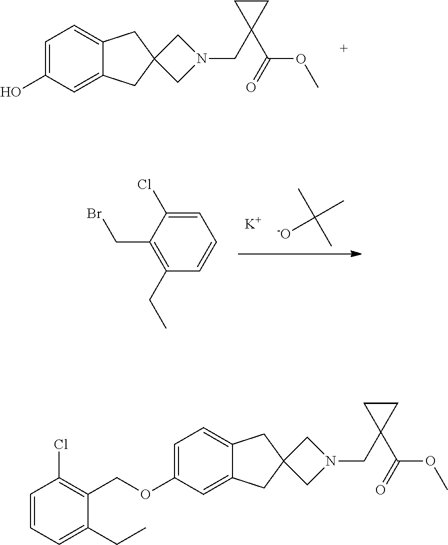

In a 50 mL round-bottomed flask methyl 1-((5′-hydroxy-1′,3′-dihydrospiro[azetidine-3,2′-inden]-1-yl)methyl)cyclopropanecarboxylate (50 mg, 0.174 mmol) was dissolved in DMF (2 mL) to give a colorless solution. The reaction mixture was cooled down to 0° C. and potassium tert-butoxide (20 mg, 0.178 mmol) was added. The reaction mixture was stirred at 0° C. for 30 min. 2-(Bromomethyl)-1-chloro-3-ethylbenzene (41 mg, 0.176 mmol) was added. The reaction mixture was stirred overnight at RT.

1 m L water was added to the mixture and after that the mixture was diluted with CH₂Cl₂. The phases were separated using a Chromabond PTS-cartridge and the organic layer was evaporated. The residue was purified using the Isco-Combiflash (4 g, 0-10% MeOH in CH₂Cl₂).

Yield: 42 mg clear oil

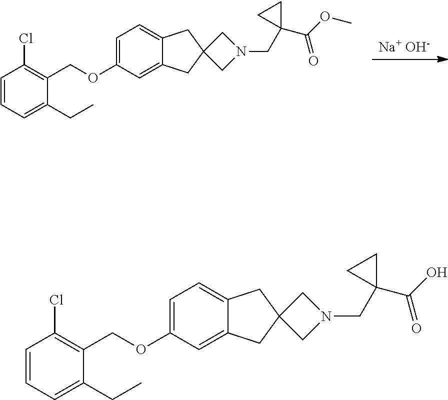 [SMILES: "Na+ OH-" ; CCc1cccc(Cl)c1COc1ccc2c(c1)CC1(C2)CN(CC2(C(=O)OC)CC2)C1 ; CCc1cccc(Cl)c1COc1ccc2c(c1)CC1(C2)CN(CC2(C(=O)O)CC2)C1]

In a 25 mL round-bottomed flask methyl 1-((5′-((2-chloro-6-ethylbenzyl)oxy)-1′,3′-dihydrospiro[azetidine-3,2′-inden]-1-yl)methyl)cyclopropanecarboxylate (42 mg, 0.095 mmol) was dissolved in MeOH (1 mL) and THF (1 mL) to give a colorless solution. NaOH (0.3 mL, 0.600 mmol) was added. The reaction mixture was stirred at RT overnight.

The reaction mixture was evaporated. The residue was dissolved in water and neutralized with HOAc (to pH value 6). 50 mL CH₂Cl₂ were added. After phase separation the organic layer was dried over a GoreTex cartridge, the organic layer was evaporated. The residue was dried under vacuum at 40° C. Yield: 45 mg oil

¹H NMR (600 MHz, DMSO-d₆) δ 7.39-7.34 (m, 2H), 7.28 (dd, J=6.0, 2.9 Hz, 1H), 7.13 (d, J=8.3 Hz, 1H), 6.95 (d, J=2.5 Hz, 1H), 6.81 (dd, J=8.2, 2.5 Hz, 1H), 5.09 (s, 2H), 3.51 (s, 4H), 3.10 (s, 2H), 3.05 (s, 2H), 2.79 (s, 2H), 2.71 (q, J=7.5 Hz, 2H), 1.16 (t, J=7.6 Hz, 3H), 0.92 (q, J=3.7 Hz, 2H), 0.64 (q, J=3.7 Hz, 2H).

MS: Calculated mass (C25H28ClNO3): 425.18, found mass: M+H=426/428

Examples 29-49 were prepared analogous to example 28:

### Example 29. 1-((5′-(benzyloxy)-1′,3′-dihydrospiro[azetidine-3,2′-inden]-1-yl)methyl)cyclopropanecarboxylic acid

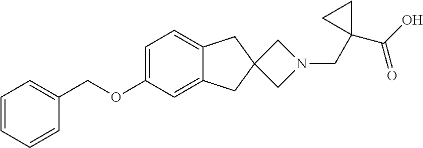 [SMILES: O=C(O)C1(CN2CC3(Cc4ccc(OCc5ccccc5)cc4C3)C2)CC1]

¹H NMR (600 MHz, DMSO-d₆) δ 7.45-7.41 (m, 2H), 7.38 (t, J=7.6 Hz, 2H), 7.34-7.29 (m, 1H), 7.09 (d, J=8.3 Hz, 1H), 6.88 (d, J=2.3 Hz, 1H), 6.76 (dd, J=8.2, 2.5 Hz, 1H), 5.04 (s, 2H), 3.41 (s, 4H), 3.05 (s, 2H), 3.00 (s, 2H), 2.74 (s, 2H), 0.86 (q, J=3.5 Hz, 2H), 0.57 (q, J=3.6 Hz, 2H).

MS: Calculated mass (C23H25NO3): 363.18, found mass: M+H=364

### Example 30. 1-((5′-((2-fluoro-6-methoxybenzyl)oxy)-1′,3′-dihydrospiro[azetidine-3,2′-inden]-1-yl)methyl)cyclopropanecarboxylic acid compound with 2,2,2-trifluoroacetic acid (1:1)

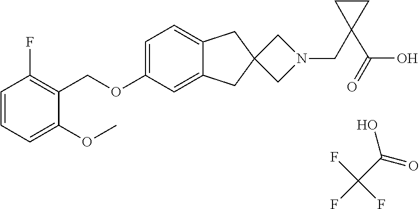

¹H NMR (500 MHz, DMSO-d₆) δ 12.90 (d, J=40.4 Hz, 1H), 9.60 (s, 1H), 7.42 (td, J=8.4, 6.9 Hz, 1H), 7.12 (dd, J=19.6, 8.3 Hz, 1H), 7.01-6.54 (m, 5H), 4.97 (s, 2H), 4.18 (d, J=6.1 Hz, 4H), 3.83 (s, 3H), 1.23-1.05 (m, 4H).

MS: Calculated mass (C24H26FNO4): 411.18, found mass: M+H=412

### Example 31. 1-((5′-((2-chloro-6-methoxybenzyl)oxy)-1′,3′-dihydrospiro[azetidine-3,2′-inden]-1-yl)methyl)cyclopropanecarboxylic acid compound with 2,2,2-trifluoroacetic acid (1:1)

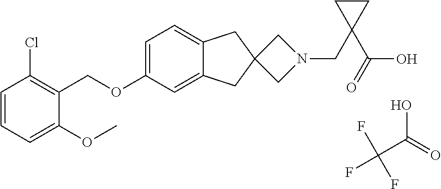

¹H NMR (500 MHz, DMSO-d₆) δ 12.93 (s, 1H), 9.66 (s, 1H), 7.41 (t, J=8.2 Hz, 1H), 7.09 (dd, J=12.4, 8.2 Hz, 3H), 6.93 (d, J=23.2 Hz, 1H), 6.79 (dd, J=8.2, 2.4 Hz, 1H), 5.05 (s, 2H), 4.19 (d, J=6.2 Hz, 4H), 3.82 (s, 2H), 3.23 (d, J=27.7 Hz, 2H), 3.12 (d, J=26.0 Hz, 2H), 1.22 (q, J=4.1, 3.7 Hz, 2H), 1.10 (q, J=4.1 Hz, 2H).

MS: Calculated mass (C24H26ClNO4): 427.16, found mass: M+H=428

### Example 32. 1-((5′-((2-chloro-4-ethoxybenzyl)oxy)-1′,3′-dihydrospiro[azetidine-3,2′-inden]-1-yl)methyl)cyclopropanecarboxylic acid

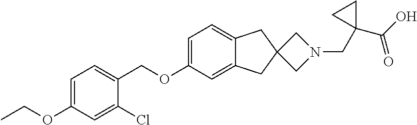 [SMILES: CCOc1ccc(COc2ccc3c(c2)CC2(C3)CN(CC3(C(=O)O)CC3)C2)c(Cl)c1]

¹H NMR (600 MHz, DMSO-d₆) δ 7.47 (d, J=8.5 Hz, 1H), 7.11 (d, J=8.3 Hz, 1H), 7.07 (d, J=2.6 Hz, 1H), 6.94 (dd, J=8.5, 2.6 Hz, 1H), 6.89 (d, J=2.4 Hz, 1H), 6.77 (dd, J=8.2, 2.5 Hz, 1H), 5.00 (s, 2H), 4.06 (q, J=7.0 Hz, 2H), 3.08 (s, 2H), 3.03 (s, 2H), 2.78 (d, J=2.4 Hz, 2H), 1.33 (t, J=7.0 Hz, 3H), 0.89 (q, J=3.5 Hz, 2H), 0.61 (p, J=3.8, 3.2 Hz, 2H).

MS: Calculated mass (C25H28ClNO4): 441.17, found mass: M+H=442/444

### Example 33. 1-((5′-((4-chlorobenzyl)oxy)-1′,3′-dihydrospiro[azetidine-3,2′-inden]-1-yl)methyl)cyclopropanecarboxylic acid

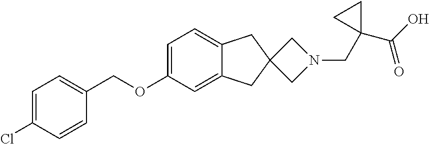 [SMILES: O=C(O)C1(CN2CC3(Cc4ccc(OCc5ccc(Cl)cc5)cc4C3)C2)CC1]

¹H NMR (600 MHz, DMSO-d₆) δ 7.45 (s, 4H), 7.10 (d, J=8.2 Hz, 1H), 6.88 (d, J=2.4 Hz, 1H), 6.76 (dd, J=8.2, 2.5 Hz, 1H), 5.05 (s, 2H), 3.45 (s, 4H), 3.06 (s, 2H), 3.01 (s, 2H), 2.76 (s, 2H), 0.89 (q, J=3.6 Hz, 2H), 0.61 (q, J=3.7 Hz, 2H).

MS: Calculated mass (C23H24ClNO3): 397.14, found mass: M+H=398/400

### Example 34. 1-((5′-((2-chloro-6-fluorobenzyl)oxy)-1′,3′-dihydrospiro[azetidine-3,2′-inden]-1-yl)methyl)cyclopropanecarboxylic acid compound with 2,2,2-trifluoroacetic acid (1:1)

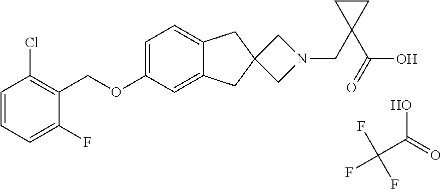

¹H NMR (500 MHz, DMSO-d₆) δ 12.91 (s, 1H), 9.61 (s, 1H), 7.51 (td, J=8.3, 6.2 Hz, 1H), 7.42 (d, J=8.1 Hz, 1H), 7.36-7.27 (m, 1H), 7.20-7.10 (m, 1H), 6.95 (d, J=22.6 Hz, 1H), 6.83 (dd, J=8.5, 2.2 Hz, 1H), 4.19 (d, J=6.2 Hz, 4H), 3.45 (d, J=5.2 Hz, 2H), 3.24-3.08 (m, 4H), 1.27-1.06 (m, 4H).

MS: Calculated mass (C23H23C1FNO3): 415.14, found mass: M+H=416/418

### Example 35. 1-((5′-((4-ethoxybenzyl)oxy)-1′,3′-dihydrospiro[azetidine-3,2′-inden]-1-yl)methyl)cyclopropanecarboxylic acid

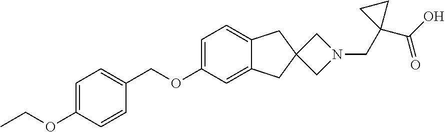 [SMILES: CCOc1ccc(COc2ccc3c(c2)CC2(C3)CN(CC3(C(=O)O)CC3)C2)cc1]

¹H NMR (600 MHz, DMSO-d₆) δ 7.35-7.31 (m, 2H), 7.09 (d, J=8.3 Hz, 1H), 6.94-6.90 (m, 2H), 6.86 (d, J=2.3 Hz, 1H), 6.75 (dd, J=8.2, 2.5 Hz, 1H), 4.95 (s, 2H), 4.01 (q, J=7.0 Hz, 2H), 3.48 (s, 4H), 3.06 (s, 2H), 3.02 (s, 2H), 2.77 (s, 2H), 1.32 (t, J=7.0 Hz, 3H), 0.91 (q, J=3.7 Hz, 2H), 0.63 (q, J=3.7 Hz, 2H).

MS: Calculated mass (C25H29NO4): 407.21, found mass: M+H=408

### Example 36. 1-((5′-((2,5-dichlorobenzyl)oxy)-1′,3′-dihydrospiro[azetidine-3,2′-inden]-1-yl)methyl)cyclopropanecarboxylic acid compound with acetic acid (1:1)

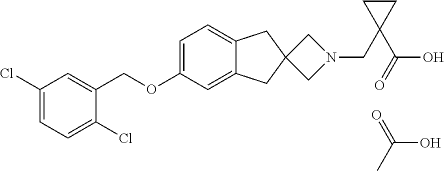

¹H NMR (600 MHz, DMSO-d₆) δ 12.05 (s, 3H), 7.65 (d, J=2.7 Hz, 1H), 7.56 (d, J=8.5 Hz, 1H), 7.48 (dd, J=8.5, 2.6 Hz, 1H), 7.14 (d, J=8.3 Hz, 1H), 6.93 (d, J=2.4 Hz, 1H), 6.81 (dd, J=8.2, 2.5 Hz, 1H), 5.10 (s, 2H), 3.53 (s, 4H), 3.10 (s, 2H), 3.05 (s, 2H), 2.80 (s, 2H), 1.92 (s, 11H), 0.92 (q, J=3.7 Hz, 2H), 0.64 (q, J=3.7 Hz, 2H).

MS: Calculated mass (C23H23Cl2NO3): 431.11, found mass: M+H=432/434/436

### Example 37. 1-((5′-((2,6-difluorobenzyl)oxy)-1′,3′-dihydrospiro[azetidine-3,2′-inden]-1-yl)methyl)cyclopropanecarboxylic acid compound with 2,2,2-trifluoroacetic acid (1:1)

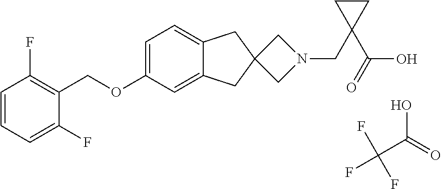

¹H NMR (600 MHz, DMSO-d₆) δ 12.05 (s, 3H), 7.65 (d, J=2.7 Hz, 1H), 7.56 (d, J=8.5 Hz, 1H), 7.48 (dd, J=8.5, 2.6 Hz, 1H), 7.14 (d, J=8.3 Hz, 1H), 6.93 (d, J=2.4 Hz, 1H), 6.81 (dd, J=8.2, 2.5 Hz, 1H), 5.10 (s, 2H), 3.53 (s, 4H), 3.10 (s, 2H), 3.05 (s, 2H), 2.80 (s, 2H), 1.92 (s, 11H), 0.92 (q, J=3.7 Hz, 2H), 0.64 (q, J=3.7 Hz, 2H).

MS: Calculated mass (C23H23F2NO3): 399.16, found mass: M+H=400

### Example 38. 1-((5′-((2,4-dichlorobenzyl)oxy)-1′,3′-dihydrospiro[azetidine-3,2′-inden]-1-yl)methyl)cyclopropanecarboxylic acid

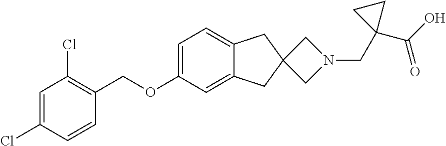 [SMILES: O=C(O)C1(CN2CC3(Cc4ccc(OCc5ccc(Cl)cc5Cl)cc4C3)C2)CC1]

¹H NMR (600 MHz, DMSO-d₆) δ 7.70 (d, J=2.2 Hz, 1H), 7.60 (d, J=8.3 Hz, 1H), 7.48 (dd, J=8.3, 2.2 Hz, 1H), 7.12 (d, J=8.2 Hz, 1H), 6.91 (d, J=2.4 Hz, 1H), 6.79 (dd, J=8.2, 2.5 Hz, 1H), 5.09 (s, 2H), 3.46 (s, 6H), 3.08 (s, 2H), 3.03 (s, 2H), 2.77 (s, 2H), 0.89 (q, J=3.7 Hz, 2H), 0.60 (q, J=3.7 Hz, 2H).

MS: Calculated mass (C23H23Cl2NO3): 431.11, found mass: M+H=432/434/436

### Example 39. 1-((5′-((2,6-dimethylbenzyl)oxy)-1′,3′-dihydrospiro[azetidine-3,2′-inden]-1-yl)methyl)cyclopropanecarboxylic acid compound with 2,2,2-trifluoroacetic acid (1:1)

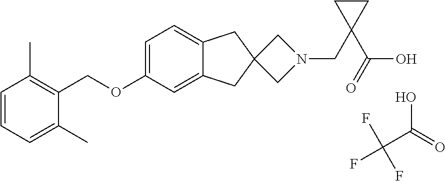

¹H NMR (500 MHz, DMSO-d₆) δ 12.92 (s, 1H), 9.63 (s, 1H), 7.15 (td, J=14.0, 13.5, 7.5 Hz, 2H), 7.07 (t, J=6.1 Hz, 2H), 6.99-6.92 (m, 1H), 6.83 (dd, J=8.1, 2.4 Hz, 1H), 4.98 (s, 2H), 4.19 (d, J=6.2 Hz, 4H), 3.45 (d, J=5.3 Hz, 2H), 3.24 (d, J=28.2 Hz, 2H), 3.13 (d, J=25.5 Hz, 2H), 1.22 (q, J=4.1 Hz, 2H), 1.10 (q, J=4.5, 4.1 Hz, 2H)

MS: Calculated mass (C25H29NO3): 391.21, found mass: M+H=392

### Example 40. 1-((5′-((2-chloro-4-methoxybenzyl)oxy)-1′,3′-dihydrospiro[azetidine-3,2′-inden]-1-yl)methyl)cyclopropanecarboxylic acid

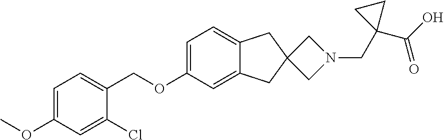 [SMILES: COc1ccc(COc2ccc3c(c2)CC2(C3)CN(CC3(C(=O)O)CC3)C2)c(Cl)c1]

¹H NMR (600 MHz, DMSO-d₆) δ 7.48 (d, J=8.6 Hz, 1H), 7.11 (d, J=8.4 Hz, 1H), 7.09 (d, J=2.6 Hz, 1H), 6.95 (dd, J=8.5, 2.6 Hz, 1H), 6.89 (d, J=2.4 Hz, 1H), 6.77 (dd, J=8.2, 2.5 Hz, 1H), 5.01 (s, 2H), 3.79 (s, 3H), 3.48 (s, 4H), 3.08 (s, 2H), 3.03 (s, 2H), 2.77 (s, 2H), 0.91 (q, J=3.7 Hz, 2H), 0.63 (q, J=3.7 Hz, 2H).

MS: Calculated mass (C24H26ClNO4): 427.16, found mass: M+H=428/430

### Example 41. 1-((5′-((2-(trifluoromethyl)benzyl)oxy)-1′,3′-dihydrospiro[azetidine-3,2′-inden]-1-yl)methyl)cyclopropanecarboxylic acid compound with 2,2,2-trifluoroacetic acid (1:1)

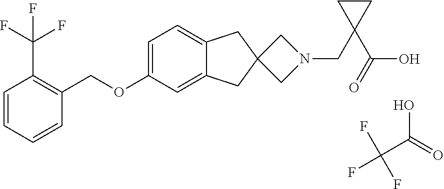

¹H NMR (500 MHz, DMSO-d₆) δ 12.91 (s, 1H), 9.61 (s, 1H), 7.83-7.66 (m, 3H), 7.63-7.53 (m, 1H), 7.14 (s, 1H), 6.91 (d, J=22.5 Hz, 1H), 6.79 (dd, J=8.2, 2.5 Hz, 1H), 5.22-5.14 (m, 2H), 4.26-4.09 (m, 4H), 3.23-3.06 (m, 4H), 1.25-1.07 (m, 4H).

MS: Calculated mass (C24H24F3NO3): 431.17, found mass: M+H=432

### Example 42. 1-((5′-((2-fluoro-6-methylbenzyl)oxy)-1′,3′-dihydrospiro[azetidine-3,2′-inden]-1-yl)methyl)cyclopropanecarboxylic acid compound with 2,2,2-trifluoroacetic acid (1:1)

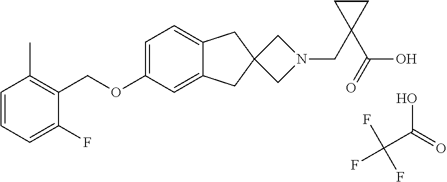

¹H NMR (500 MHz, DMSO-d₆) δ 12.92 (s, 1H), 9.62 (s, 1H), 7.33 (td, J=7.9, 5.9 Hz, 1H), 7.19-7.04 (m, 3H), 6.95 (d, J=23.7 Hz, 1H), 6.82 (dd, J=8.3, 2.4 Hz, 1H), 5.04 (s, 2H), 4.19 (d, J=6.4 Hz, 4H), 3.45 (d, J=5.3 Hz, 2H), 3.23 (d, J=27.6 Hz, 2H), 3.12 (d, J=25.7 Hz, 2H), 2.36 (d, J=2.1 Hz, 3H), 1.22 (q, J=4.1 Hz, 2H), 1.10 (q, J=4.1 Hz, 2H).

MS: Calculated mass (C24H26FNO3): 395.19, found mass: M+H=396

### Example 43. 1-((5′-((5-fluoro-2-(trifluoromethyl)benzyl)oxy)-1′,3′-dihydrospiro[azetidine-3,2′-inden]-1-yl)methyl)cyclopropanecarboxylic acid Compound with 2,2,2-trifluoroacetic acid (1:1)

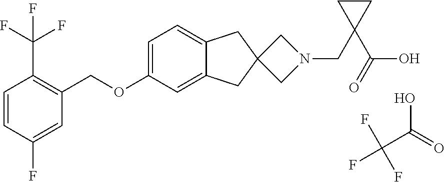

¹H NMR (500 MHz, DMSO-d₆) δ 9.67 (s, 1H), 7.89 (dd, J=8.8, 5.3 Hz, 1H), 7.60 (dd, J=9.9, 2.9 Hz, 1H), 7.44 (td, J=8.5, 2.7 Hz, 1H), 7.17 (d, J=13.2 Hz, 1H), 6.93 (d, J=22.7 Hz, 1H), 6.82 (dd, J=8.2, 2.5 Hz, 1H), 5.20 (s, 2H), 4.19 (d, J=4.5 Hz, 4H), 3.45 (d, J=3.5 Hz, 3H), 3.24 (d, J=28.4 Hz, 2H), 3.13 (d, J=25.1 Hz, 2H), 1.22 (q, J=4.1 Hz, 2H), 1.10 (q, J=4.1 Hz, 2H).

MS: Calculated mass (C24H23F4NO3): 449.16, found mass: M+H=450

### Example 44. 1-((5′-((2-chloro-6-cyclopropylbenzyl)oxy)-1′,3′-dihydrospiro[azetidine-3,2′-inden]-1-yl)methyl)cyclopropanecarboxylic acid compound with acetic acid (1:1)

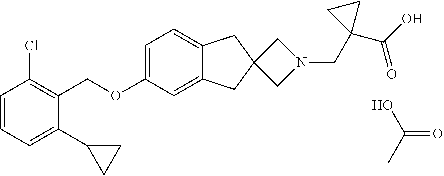

¹H NMR (600 MHz, DMSO-d₆) δ 7.36-7.30 (m, 2H), 7.13 (d, J=8.2 Hz, 1H), 7.04 (dd, J=7.4, 1.6 Hz, 1H), 6.97 (d, J=2.4 Hz, 1H), 6.83 (dd, J=8.2, 2.5 Hz, 1H), 5.26 (s, 2H), 3.56 (s, 4H), 3.11 (s, 2H), 3.06 (s, 2H), 2.06 (tt, J=8.5, 5.3 Hz, 1H), 1.92 (s, 3H), 0.94-0.90 (m, 4H), 0.72-0.68 (m, 2H), 0.65 (q, J=3.7 Hz, 2H).

MS: Calculated mass (C26H28ClNO3): 437.18, found mass: M+H=438/440

### Example 45. 1-((5′-((3-chlorobenzyl)oxy)-1′,3′-dihydrospiro[azetidine-3,2′-inden]-1-yl)methyl)cyclopropanecarboxylic acid

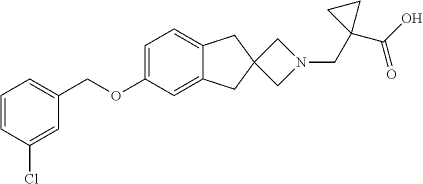 [SMILES: O=C(O)C1(CN2CC3(Cc4ccc(OCc5cccc(Cl)c5)cc4C3)C2)CC1]

¹H NMR (600 MHz, DMSO-d₆) δ 7.49 (t, J=1.8 Hz, 1H), 7.44-7.36 (m, 3H), 7.10 (d, J=8.1 Hz, 1H), 6.89 (d, J=2.3 Hz, 1H), 6.77 (dd, J=8.2, 2.5 Hz, 1H), 5.07 (s, 2H), 3.45 (s, 4H), 3.07 (s, 2H), 3.02 (s, 2H), 2.76 (s, 2H), 0.89 (q, J=3.7 Hz, 2H), 0.61 (q, J=3.7 Hz, 2H).

MS: Calculated mass (C23H24ClNO3): 397.14, found mass: M+H=398/400

### Example 46. 1-((5′-((2,6-dichlorobenzyl)oxy)-1′,3′-dihydrospiro[azetidine-3,2′-inden]-1-yl)methyl)cyclopropanecarboxylic acid

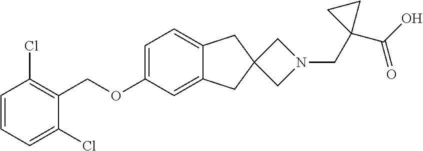 [SMILES: O=C(O)C1(CN2CC3(Cc4ccc(OCc5c(Cl)cccc5Cl)cc4C3)C2)CC1]

¹H NMR (600 MHz, DMSO-d₆) δ 7.57 (d, J=8.1 Hz, 2H), 7.47 (dd, J=8.7, 7.5 Hz, 1H), 7.13 (d, J=8.2 Hz, 1H), 6.94 (d, J=2.4 Hz, 1H), 6.81 (dd, J=8.2, 2.5 Hz, 1H), 5.17 (s, 2H), 3.08 (s, 2H), 3.03 (s, 2H), 2.76 (s, 2H), 0.85 (q, J=3.3 Hz, 2H), 0.56 (q, J=3.3 Hz, 2H).

MS: Calculated mass (C23H23Cl2NO3): 431.11, found mass: M+H=432/434/436

### Example 47. 1-((5′-((2-chloro-6-(trifluoromethyl)benzyl)oxy)-1′,3′-dihydrospiro[azetidine-3,2′-inden]-1-yl)methyl)cyclopropanecarboxylic acid Compound with 2,2,2-trifluoroacetic acid (1:1)

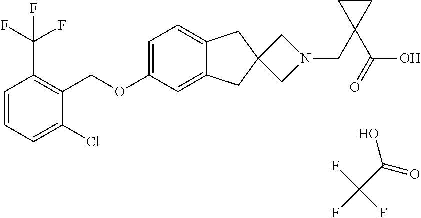 [SMILES: O=C(O)C1(CN2CC3(Cc4ccc(OCc5c(Cl)cccc5C(F)(F)F)cc4C3)C2)CC1 ; O=C(O)C(F)(F)F]

¹H NMR (500 MHz, DMSO-d₆) δ 12.92 (s, 1H), 9.63 (s, 1H), 7.92 (d, J=8.0 Hz, 1H), 7.84 (d, J=7.8 Hz, 1H), 7.69 (t, J=8.0 Hz, 1H), 7.15 (dd, J=19.6, 8.3 Hz, 1H), 6.97 (d, J=23.8 Hz, 1H), 6.86-6.81 (m, 1H), 5.14 (s, 2H), 4.19 (d, J=6.0 Hz, 5H), 3.45 (d, J=5.0 Hz, 2H), 3.25 (d, J=29.1 Hz, 2H), 3.14 (d, J=26.2 Hz, 2H), 1.22 (q, J=4.1, 3.7 Hz, 2H), 1.10 (q, J=4.1 Hz, 2H).

MS: Calculated mass (C24H23C1F3NO3): 465.13, found mass: M+H=466/468

### Example 48. 1-((5′-((2-ethyl-6-fluorobenzyl)oxy)-1′,3′-dihydrospiro[azetidine-3,2′-inden]-1-yl)methyl)cyclopropanecarboxylic acid

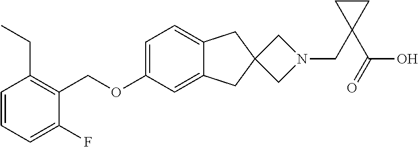 [SMILES: CCc1cccc(F)c1COc1ccc2c(c1)CC1(C2)CN(CC2(C(=O)O)CC2)C1]

¹H NMR (600 MHz, DMSO-d₆) δ 7.38 (td, J=8.0, 6.0 Hz, 1H), 7.16-7.12 (m, 2H), 7.09 (ddd, J=9.5, 8.3, 1.1 Hz, 1H), 6.94 (d, J=2.3 Hz, 1H), 6.81 (dd, J=8.2, 2.5 Hz, 1H), 5.03 (d, J=1.9 Hz, 2H), 3.85 (s, 4H), 3.13 (d, J=30.7 Hz, 4H), 2.70 (q, J=7.6 Hz, 2H), 1.17 (t, J=7.6 Hz, 3H), 1.07 (t, J=3.6 Hz, 2H), 0.88 (s, 2H).

MS: Calculated mass (C25H28FNO3): 409.21, found mass: M+H=410

### Example 49. 1-((5′-((2-fluoro-6-(trifluoromethyl)benzyl)oxy)-1′,3′-dihydrospiro[azetidine-3,2′-inden]-1-yl)methyl)cyclopropanecarboxylic acid

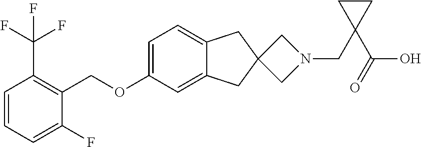 [SMILES: O=C(O)C1(CN2CC3(Cc4ccc(OCc5c(F)cccc5C(F)(F)F)cc4C3)C2)CC1]

¹H NMR (600 MHz, DMSO-d₆) δ 7.75-7.66 (m, 3H), 7.13 (d, J=8.2 Hz, 1H), 6.92 (d, J=2.3 Hz, 1H), 6.79 (dd, J=8.2, 2.5 Hz, 1H), 5.09 (s, 2H), 3.49 (s, 4H), 3.09 (s, 2H), 3.05 (s, 2H), 2.78 (s, 2H), 0.91 (q, J=3.7 Hz, 2H), 0.63 (q, J=3.7 Hz, 2H).

MS: Calculated mass (C24H23F4NO3): 449.16, found mass: M+H=450

### Example 50. 1-((5′-(4-((3-fluorobenzyl)oxy)phenyl)-1′,3′-dihydrospiro[azetidine-3,2′-inden]-1-yl)methyl)cyclopropanecarboxylic acid

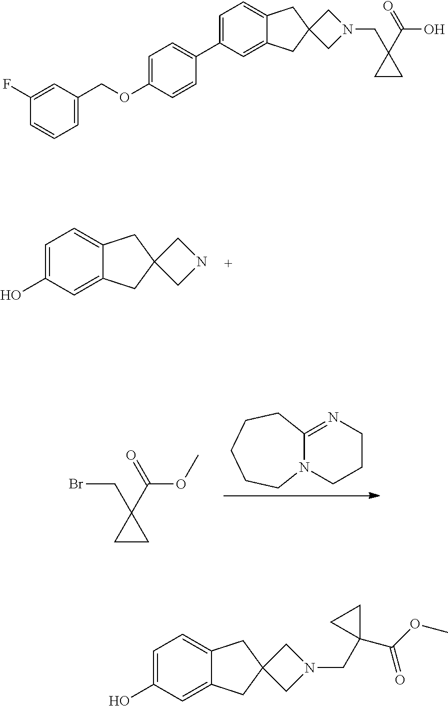

In a 50 mL round-bottomed flask 1′,3′-dihydrospiro[azetidine-3,2′-inden]-5′-ol (700 mg, 3.99 mmol) was dissolved in DMF (10 mL) to give a colorless solution. DBU (660 μL, 4.38 mmol) and methyl 1-(bromomethyl)cyclopropanecarboxylate (1002 mg, 5.19 mmol) were added. The reaction mixture was stirred at RT for 1 h.

The reaction mixture was evaporated, the residue was dissolved in ethyl acetate and washed 2× with sat. NH₄Cl-solution and 1× with saturated sodium chloride solution, the organic layer was dried with MgSO₄, filtered and evaporated. The residue was purified using the Isco-Combiflash (12 g, 0-10% MeOH in CH₂Cl₂).

Yield: 650 mg yellow oil

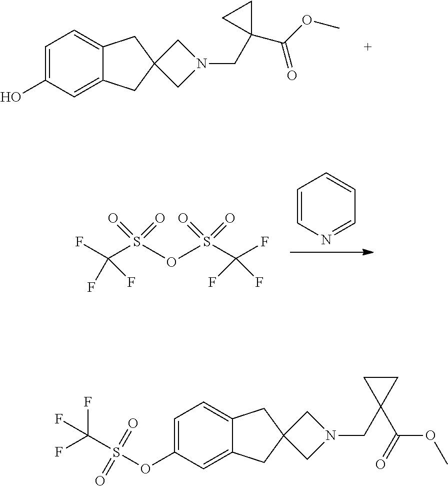

In a 100 mL 3-neck flask methyl 1-((5′-hydroxy-1′,3′-dihydrospiro[azetidine-3,2′-inden]-1-yl)methyl)cyclopropanecarboxylate (650 mg, 2.262 mmol) was dissolved in CH₂Cl₂ (20 mL) to give a light yellow solution. Pyridine (0.45 mL, 5.56 mmol) was added. The mixture was cooled to 0° C. and at this temperature trifluoromethanesulfonic anhydride (2.5 mL, 2.5 mmol) was added dropwise. The color of the solution turned to yellow.

The reaction mixture was diluted with CH₂Cl₂ and washed 2× with sat. NH₄Cl-solution and 1× with saturated sodium chloride solution. The organic layer was dried over MgSO₄, filtered and evaporated. The residue was purified using the Isco-Combiflash (12 g, 0-10% MeOH in CH₂Cl₂, 30 mL/min).

Yield: 680 mg yellow oil

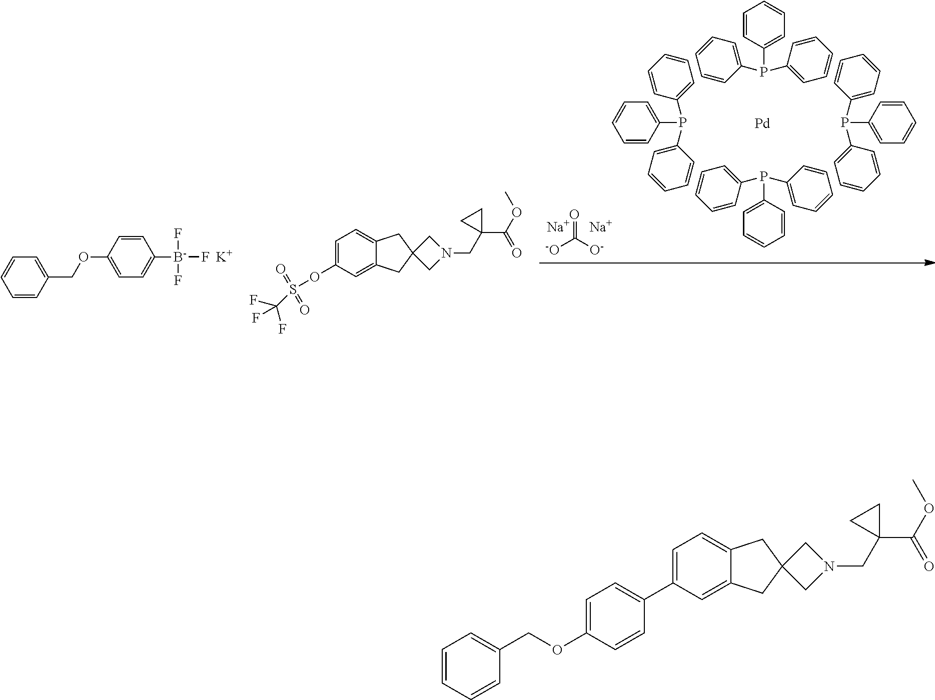

In a CEM microwave flask methyl 1-((5′-(((trifluoromethyl)sulfonyl)oxy)-1′,3′-dihydrospiro[azetidine-3,2′-inden]-1-yl)methyl)cyclopropanecarboxylate (630 mg, 1.502 mmol), potassium (4-benzyloxyphenyl)trifluoroborate (523 mg, 1.803 mmol) were dissolved in N,N-Dimethylformamide (15 mL). Sodium carbonate (3 mL, 3.11 mmol) was added. The reaction mixture was degassed with Argon for 30 min. Tetrakis(triphenylphosphine)palladium(0) (87 mg, 0.075 mmol) was added. The reaction mixture was stirred for 60 min at 120° C. in the CEM microwave. LC/MS showed product mass, the color of the reaction mixture changed during the reaction from yellow to black, reaction was stopped.

The reaction mixture was evaporated. The residue was dissolved in CH₂Cl₂ and washed 2× with water, 1× with saturated sodium chloride solution, dried over MgSO₄, filtered and evaporated. The residue was purified using the Isco-Combiflash (12 g, 0-10% MeOH in CH₂Cl₂, 30 mL/min)

Yield: 280 mg yellow solid (90% pure by HPLC)

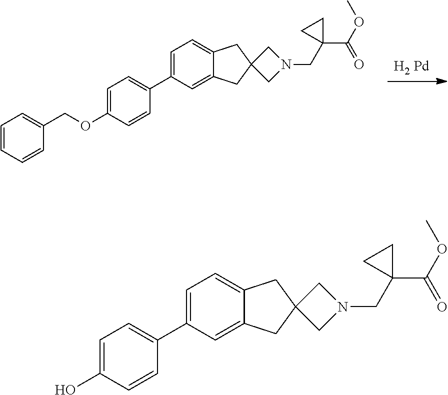 [SMILES: COC(=O)C1(CN2CC3(Cc4ccc(-c5ccc(OCc6ccccc6)cc5)cc4C3)C2)CC1 ; "H2 Pd" ; COC(=O)C1(CN2CC3(Cc4ccc(-c5ccc(O)cc5)cc4C3)C2)CC1]

In a 50 mL 2-neck round-bottomed flask methyl 1-((5′-(4-(benzyloxy)phenyl)-1′,3′-dihydrospiro[azetidine-3,2′-inden]-1-yl)methyl)cyclopropanecarboxylate (250 mg, 0.551 mmol) was dissolved in Tetrahydrofuran (5 mL) and MeOH (5 mL) to give a yellow solution. Pd—C (60 mg, 0.564 mmol) was added under Argon atmosphere. The reaction mixture was stirred at RT under hydrogen atmosphere.

The reaction mixture was filtered and the organic layer was evaporated.

Yield: 200 mg clear oil

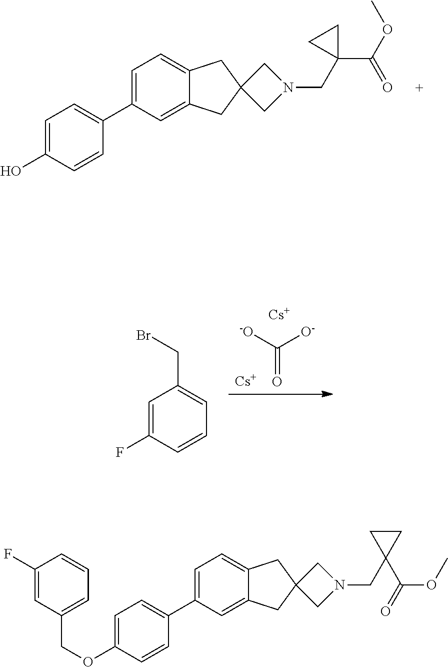

In a 50 mL round-bottomed flask methyl 1-((5′-(4-hydroxyphenyl)-1′,3′-dihydrospiro[azetidine-3,2′-inden]-1-yl)methyl)cyclopropanecarboxylate (65 mg, 0.179 mmol) was dissolved in DMF (3 mL) to give a light yellow solution. Cesium carbonate (87 mg, 0.268 mmol) and 3-fluorobenzyl bromide (0.026 mL, 0.215 mmol) were added. The mixture was stirred overnight at RT.

The reaction mixture was evaporated. The residue was dissolved in CH₂Cl₂/water. After phase separation with a Chromabond PTS-cartridge, the organic layer was evaporated. The residue was purified using the Isco-Combiflash (4 g, 0-10% MeOH in CH₂Cl₂, 18 mL/min)

Yield: 30 mg light brown solid

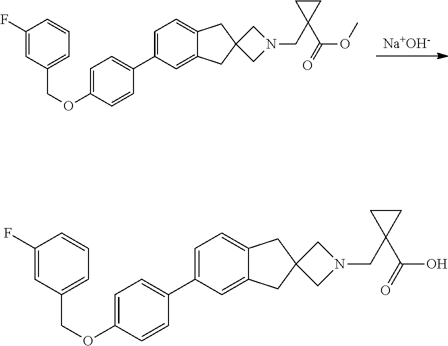 [SMILES: "Na+OH-" ; COC(=O)C1(CN2CC3(Cc4ccc(-c5ccc(OCc6cccc(F)c6)cc5)cc4C3)C2)CC1 ; O=C(O)C1(CN2CC3(Cc4ccc(-c5ccc(OCc6cccc(F)c6)cc5)cc4C3)C2)CC1]

In a 50 mL round-bottomed flask methyl 1-((5′-(4-((3-fluorobenzyl)oxy)phenyl)-1′,3′-dihydrospiro[azetidine-3,2′-inden]-1-yl)methyl)cyclopropanecarboxylate (30 mg, 0.064 mmol) was dissolved in MeOH (1 mL) and THF (1 mL) to give a yellow solution. sodium hydroxide (0.159 mL, 0.318 mmol) was added. The mixture was stirred overnight.

The reaction mixture was neutralized with 318 μL 1 n HCl and concentrated under vacuum. A white precipitate was formed, the solid was filtered, washed with water and dried under vacuum at 50° C. Yield: 22 mg off-white solid

¹H NMR (600 MHz, DMSO-d₆) δ 7.58-7.54 (m, 2H), 7.45 (td, J=8.0, 6.1 Hz, 2H), 7.37 (dd, J=7.8, 1.7 Hz, 1H), 7.33-7.28 (m, 2H), 7.26 (d, J=7.8 Hz, 1H), 7.17 (ddd, J=10.5, 8.1, 2.6 Hz, 1H), 7.10-7.06 (m, 2H), 5.18 (s, 2H), 3.55 (s, 4H), 3.16 (s, 2H), 3.13 (s, 2H), 2.81 (s, 2H), 0.93 (q, J=3.7 Hz, 2H), 0.66 (q, J=3.8 Hz, 2H).

MS: Calculated mass (C29H28FNO3): 457.21, found mass: M+H=458

Examples 51-53 were prepared analogous to example 50:

### Example 51. 1-((5′-(4-(cyclohexylmethoxy)phenyl)-1′,3′-dihydrospiro[azetidine-3,2′-inden]-1-yl)methyl)cyclopropanecarboxylic acid

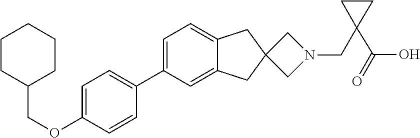 [SMILES: O=C(O)C1(CN2CC3(Cc4ccc(-c5ccc(OCC6CCCCC6)cc5)cc4C3)C2)CC1]

¹H NMR (600 MHz, DMSO-d₆) δ 7.55-7.50 (m, 2H), 7.43 (d, J=1.6 Hz, 1H), 7.36 (dd, J=7.8, 1.8 Hz, 1H), 7.25 (d, J=7.8 Hz, 1H), 6.99-6.96 (m, 2H), 3.80 (d, J=6.4 Hz, 2H), 3.52 (s, 4H), 3.16 (s, 2H), 3.13 (s, 2H), 2.79 (s, 2H), 1.82 (dd, J=12.6, 3.5 Hz, 2H), 1.77-1.62 (m, 4H), 1.31-1.13 (m, 3H), 1.05 (qd, J=12.3, 3.4 Hz, 2H), 0.92 (q, J=3.7 Hz, 2H), 0.65 (q, J=3.8 Hz, 2H).

MS: Calculated mass (C29H35NO3): 445.26, found mass: M+H=446

### Example 52. 1-((5′-(4-((3-chlorobenzyl)oxy)phenyl)-1′,3′-dihydrospiro[azetidine-3,2′-inden]-1-yl)methyl)cyclopropanecarboxylic acid

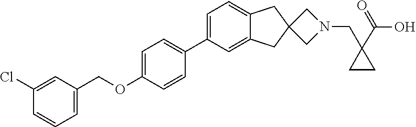 [SMILES: O=C(O)C1(CN2CC3(Cc4ccc(-c5ccc(OCc6cccc(Cl)c6)cc5)cc4C3)C2)CC1]

¹H NMR (600 MHz, DMSO-d₆) δ 7.58-7.53 (m, 3H), 7.44 (dt, J=7.4, 2.9 Hz, 3H), 7.41 (dtd, J=5.7, 3.7, 2.1 Hz, 1H), 7.37 (dd, J=7.8, 1.7 Hz, 1H), 7.26 (d, J=7.8 Hz, 1H), 7.09-7.06 (m, 2H), 5.17 (s, 2H), 3.59 (s, 4H), 3.17 (s, 2H), 3.14 (s, 2H), 2.86 (s, 2H), 0.95 (q, J=3.7 Hz, 2H), 0.69 (q, J=3.8 Hz, 2H).

MS: Calculated mass (C29H28ClNO3): 473.18, found mass: M+H=474/476

### Example 53. 1-((5′-(4-(benzyloxy)phenyl)-1′,3′-dihydrospiro[azetidine-3,2′-inden]-1-yl)methyl)cyclopropanecarboxylic acid

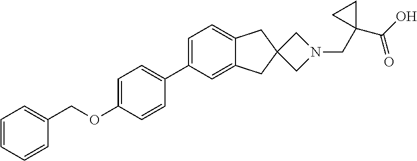 [SMILES: O=C(O)C1(CN2CC3(Cc4ccc(-c5ccc(OCc6ccccc6)cc5)cc4C3)C2)CC1]

¹H NMR (600 MHz, DMSO-d₆) δ 7.57-7.53 (m, 2H), 7.48-7.45 (m, 2H), 7.44 (d, J=1.6 Hz, 1H), 7.43-7.39 (m, 2H), 7.38-7.32 (m, 2H), 7.25 (d, J=7.8 Hz, 1H), 7.09-7.06 (m, 2H), 5.14 (s, 2H), 3.53 (s, 4H), 3.16 (s, 2H), 3.13 (s, 2H), 2.80 (s, 2H), 0.92 (q, J=3.7 Hz, 2H), 0.65 (q, J=3.7 Hz, 2H).

MS: Calculated mass (C29H29NO3): 439.21, found mass: M+H=440

### Example 54. 1-((5′-phenethoxy-1′,3′-dihydrospiro[azetidine-3,2′-inden]-1-yl)methyl)cyclopropanecarboxylic acid

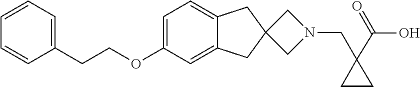 [SMILES: O=C(O)C1(CN2CC3(Cc4ccc(OCCc5ccccc5)cc4C3)C2)CC1]

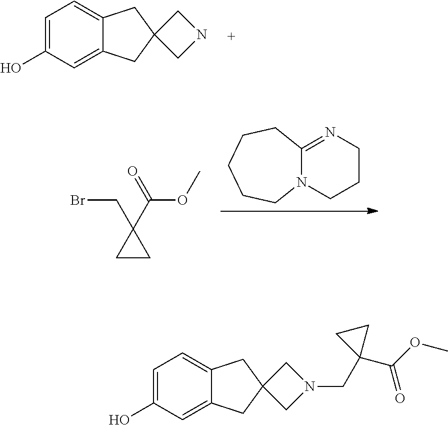

In a 50 mL round-bottomed flask 1′,3′-dihydrospiro[azetidine-3,2′-inden]-5′-ol (1 g, 5.71 mmol) was dissolved in DMF (40 mL) to give a colorless solution. DBU (2.58 mL, 17.12 mmol) and methyl 1-(bromomethyl)cyclopropanecarboxylate (1.432 g, 7.42 mmol) were added. The reaction mixture was stirred at RT for 2 days.

The reaction mixture was evaporated, the residue was dissolved in ethyl acetate and washed 2× with sat. NH₄Cl-solution, the organic layer was dried with MgSO₄, flitrated and evaporated. The residue was purified using the Isco-Combiflash (40 g, 0-15% MeOH in CH₂Cl₂, 40 mL/min).

Yield: 740 mg light yellow oil

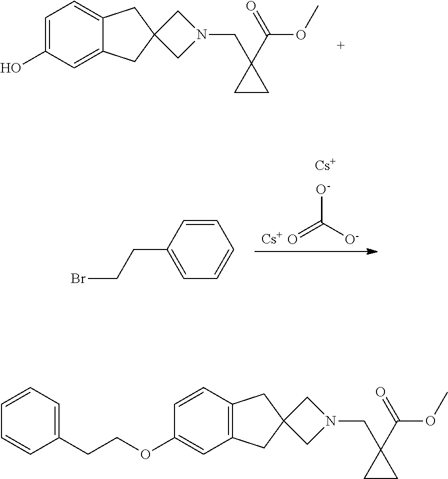

In a 2 mL Microwave Tube methyl 1-((5′-hydroxy-1′,3′-dihydrospiro[azetidine-3,2′-inden]-1-yl)methyl)cyclopropanecarboxylate (50 mg, 0.174 mmol) was dissolved in DMF (1 mL) to give a colorless solution. Cesium carbonate (125 mg, 0.383 mmol) and (2-bromoethyl)benzene (64.4 mg, 0.348 mmol) were added. The reaction mixture was stirred at 100° C. for 20 minutes in a microwave.

The reaction mixture was evaporated. The residue was dissolved in CH₂Cl₂ and water. After phase separation with a Chromabond PTS-cartridge, the organic layer was evaporated. The residue was purified using the Isco-Combiflash (4 g, 0-10% MeOH in CH₂Cl₂, 18 mL/min). Yield: 10.8 mg

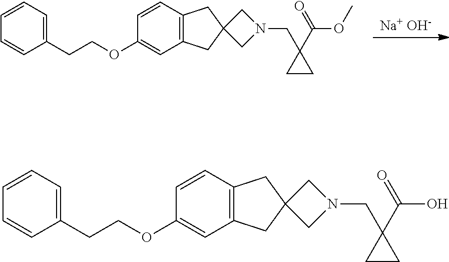 [SMILES: "Na+ OH-" ; COC(=O)C1(CN2CC3(Cc4ccc(OCCc5ccccc5)cc4C3)C2)CC1 ; O=C(O)C1(CN2CC3(Cc4ccc(OCCc5ccccc5)cc4C3)C2)CC1]

In a 2 mL round-bottomed flask methyl 1-((5′-phenethoxy-1′,3′-dihydrospiro[azetidine-3,2′-inden]-1-yl)methyl)cyclopropanecarboxylate (10 mg, 0.026 mmol) was dissolved in MeOH (1 mL) and THF (2 mL) to give a colorless solution. 2M NaOH (64 μL, 0.128 mmol) were added. The mixture was stirred overnight. Additional 2M NaOH (128 μL) was added and stirring was continued overnight.

The reaction mixture was evaporated. The residue was dissolved in water and neutralized with HCl (to pH value 7-8). CH₂Cl₂ was added. The aqueous layer was extracted 2× with CH₂Cl₂. After phase separation the combined organic layers were dried with MgSO₄, filtered and evaporated.

Yield: 8 mg white solid

¹H NMR (600 MHz, DMSO-d₆) δ 7.33-7.28 (m, 4H), 7.22 (tt, J=5.9, 3.0 Hz, 1H), 7.07 (d, J=8.2 Hz, 1H), 6.80 (d, J=2.3 Hz, 1H), 6.69 (dd, J=8.2, 2.5 Hz, 1H), 4.13 (t, J=6.9 Hz, 2H), 3.47 (s, 4H), 3.05 (s, 2H), 3.02-2.99 (m, 4H), 2.77 (s, 2H), 0.90 (q, J=3.7 Hz, 2H), 0.63 (q, J=3.7 Hz, 2H).

MS: Calculated mass (C24H27NO3): 377.20, found mass: M+H=378

Examples 55-59 were prepared analogs to example 54:

### Example 55. 1-((5′-(4-chlorophenethoxy)-1′,3′-dihydrospiro[azetidine-3,2′-inden]-1-yl)methyl)cyclopropanecarboxylic acid

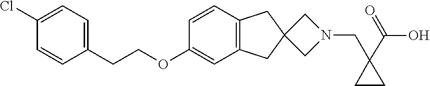 [SMILES: O=C(O)C1(CN2CC3(Cc4ccc(OCCc5ccc(Cl)cc5)cc4C3)C2)CC1]

¹H NMR (600 MHz, DMSO-d₆) δ 12.94 (s, 1H), 7.41-7.29 (m, 4H), 7.09 (d, J=8.3 Hz, 1H), 6.81 (d, J=2.3 Hz, 1H), 6.70 (dd, J=8.2, 2.5 Hz, 1H), 4.21-4.05 (m, 6H), 3.42 (s, 2H), 3.14 (d, J=26.9 Hz, 4H), 3.00 (t, J=6.6 Hz, 2H), 1.20-1.06 (m, 4H).

MS: Calculated mass (C24H26ClNO3: 411.16, found mass: M+H=412

### Example 56. 1-((5′-(2-(trifluoromethyl)phenethoxy)-1′,3′-dihydrospiro[azetidine-3,2′-inden]-1-yl)methyl)cyclopropanecarboxylic acid

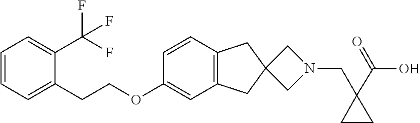 [SMILES: O=C(O)C1(CN2CC3(Cc4ccc(OCCc5ccccc5C(F)(F)F)cc4C3)C2)CC1]

¹H NMR (600 MHz, DMSO-d₆) δ 7.74-7.69 (m, 1H), 7.67-7.60 (m, 2H), 7.48-7.43 (m, 1H), 7.08 (d, J=8.2 Hz, 1H), 6.81 (d, J=2.4 Hz, 1H), 6.70 (dd, J=8.2, 2.5 Hz, 1H), 4.15 (t, J=6.9 Hz, 2H), 3.52 (s, 4H), 3.19 (t, J=7.0 Hz, 2H), 3.06 (s, 2H), 3.02 (s, 2H), 2.81 (s, 2H), 0.92 (q, J=3.7 Hz, 2H), 0.65 (q, J=3.8 Hz, 2H).

MS: Calculated mass (C25H26F3NO3): 445.19, found mass: M+H=446

### Example 57. 1-((5′-(3-chlorophenethoxy)-1′,3′-dihydrospiro[azetidine-3,2′-inden]-1-yl)methyl)cyclopropanecarboxylic acid

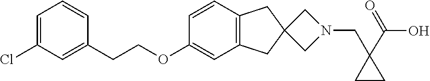 [SMILES: O=C(O)C1(CN2CC3(Cc4ccc(OCCc5cccc(Cl)c5)cc4C3)C2)CC1]

¹H NMR (600 MHz, DMSO-d₆) δ 7.41 (t, J=1.9 Hz, 1H), 7.36-7.32 (m, 1H), 7.29 (dt, J=7.0, 1.9 Hz, 2H), 7.10 (d, J=8.3 Hz, 1H), 6.82 (d, J=2.3 Hz, 1H), 6.71 (dd, J=8.3, 2.5 Hz, 1H), 4.14 (t, J=6.7 Hz, 2H), 4.09 (s, 4H), 3.16 (s, 2H), 3.11 (s, 2H), 3.02 (t, J=6.7 Hz, 2H), 1.18 (q, J=4.0 Hz, 2H), 1.04 (q, J=4.1 Hz, 2H).

MS: Calculated mass (C24H26ClNO3): 411.16, found mass: M+H=412/414

### Example 58. 1-((5′-(2-chlorophenethoxy)-1′,3′-dihydrospiro[azetidine-3,2′-inden]-1-yl)methyl)cyclopropanecarboxylic acid

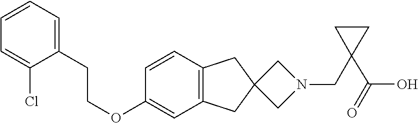 [SMILES: O=C(O)C1(CN2CC3(Cc4ccc(OCCc5ccccc5Cl)cc4C3)C2)CC1]

¹H NMR (600 MHz, DMSO-d₆) δ 7.45 (dd, J=7.6, 1.7 Hz, 2H), 7.33-7.26 (m, 2H), 7.08 (d, J=8.2 Hz, 1H), 6.81 (d, J=2.3 Hz, 1H), 6.70 (dd, J=8.2, 2.5 Hz, 1H), 4.14 (t, J=6.9 Hz, 2H), 3.51-3.43 (m, 4H), 3.14 (t, J=6.9 Hz, 2H), 3.06 (s, 2H), 3.01 (s, 2H), 2.76 (s, 2H), 0.90 (q, J=3.7 Hz, 2H), 0.62 (q, J=3.7 Hz, 2H).

MS: Calculated mass (C24H26ClNO3): 411.16, found mass: M+H=412/414

### Example 59. 1-((5′-(2,6-dichlorophenethoxy)-1′,3′-dihydrospiro[azetidine-3,2′-inden]-1-yl)methyl)cyclopropanecarboxylic acid

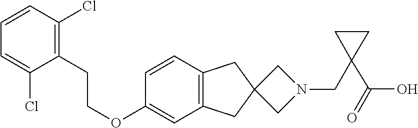 [SMILES: O=C(O)C1(CN2CC3(Cc4ccc(OCCc5c(Cl)cccc5Cl)cc4C3)C2)CC1]

¹H NMR (600 MHz, DMSO-d₆) δ 7.50 (d, J=8.1 Hz, 2H), 7.32 (t, J=8.1 Hz, 1H), 7.09 (d, J=8.3 Hz, 1H), 6.82 (d, J=2.3 Hz, 1H), 6.71 (dd, J=8.1, 2.5 Hz, 1H), 4.09 (t, J=7.4 Hz, 2H), 3.73 (s, 4H), 3.33 (t, J=7.3 Hz, 2H), 3.10 (s, 2H), 3.06 (s, 2H), 3.02 (s, 2H), 1.02 (q, J=3.9 Hz, 2H), 0.80 (d, J=4.2 Hz, 2H).

MS: Calculated mass (C24H25Cl2NO3): 445.12, found mass: M+H=446/448/450

### Example 60. 2-(5′-phenethoxy-1′,3′-dihydrospiro[azetidine-3,2′-inden]-1-yl)acetic acid

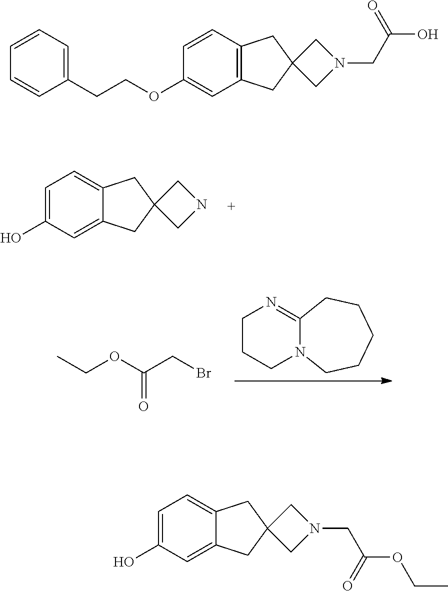

In a 100 mL round-bottomed 1′,3′-dihydrospiro[azetidine-3,2′-inden]-5′-ol (1 g, 5.71 mmol) was dissolved in DMF (40 mL) to give a colorless solution. DBU (2.5 mL, 16.59 mmol) and ethyl bromoacetate (800 μL, 7.21 mmol) were added. The reaction mixture was stirred at RT for 1 hour.

The reaction mixture was evaporated, the residue was dissolved in ethyl acetate and washed 2× with sat. NH₄Cl-solution, the organic layer was dried with MgSO₄, flitrated and evaporated. The residue was purified using the Isco-Combiflash (40 g, 0-15% MeOH in CH₂Cl₂, 30 mL/min).

Yield: 744 mg colorless oil.

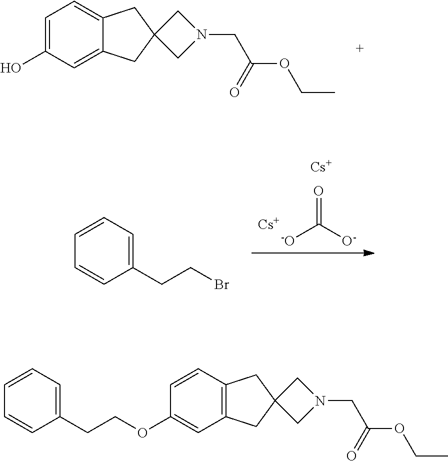

In a 2 mL Microwave Tube ethyl 2-(5′-hydroxy-1′,3′-dihydrospiro[azetidine-3,2′-inden]-1-yl)acetate (50 mg, 0.191 mmol) was dissolved in DMF (1 mL) to give a colorless solution. Cesium carbonate (145 mg, 0.445 mmol) and (2-bromoethyl)benzene (55 μL, 0.404 mmol) were added. The reaction mixture was stirred at 110° C. for 15 minutes in the Biotage microwave.

The reaction mixture was evaporated. The residue was dissolved in CH₂Cl₂ and water. After phase separation with a Chromabond PTS-cartridge, the organic layer was evaporated. The residue was purified using the Isco-Combiflash (4 g, 0-10% MeOH in CH₂Cl₂, 18 mL/min).

Yield: 10 mg clear oil

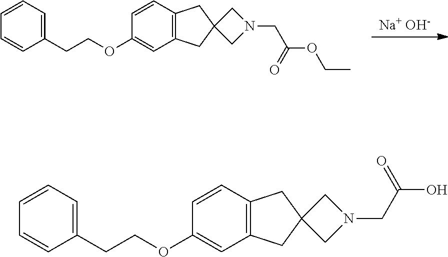 [SMILES: "Na+ OH-" ; CCOC(=O)CN1CC2(Cc3ccc(OCCc4ccccc4)cc3C2)C1 ; O=C(O)CN1CC2(Cc3ccc(OCCc4ccccc4)cc3C2)C1]

In a 20 mL round-bottom-flask ethyl 2-(5′-phenethoxy-1′,3′-dihydrospiro[azetidine-3,2′-inden]-1-yl)acetate (10 mg, 0.027 mmol) was dissolved in MeOH (0.5 mL) and THF (0.5 mL) to give a colorless solution. 2M NaOH (0.068 mL, 0.137 mmol) was added. The reaction mixture was stirred at RT overnight.

The reaction mixture was evaporated. The residue was dissolved in water and neutralized with 2N HCl to pH 7-8. Approx. 100 mL CH₂Cl₂ and a small amount of MeOH were added. After phase separation the organic layer was dried over MgSO₄, filtered and evaporated.

Yield: 7.8 mg white solid

¹H NMR (600 MHz, DMSO-d₆) δ 7.37-7.18 (m, 5H), 7.07 (d, J=8.2 Hz, 1H), 6.80 (d, J=2.3 Hz, 1H), 6.69 (dd, J=8.3, 2.4 Hz, 1H), 4.13 (t, J=6.9 Hz, 2H), 3.77 (s, 4H), 3.09 (d, J=27.1 Hz, 4H), 3.00 (t, J=6.8 Hz, 2H), 1.04 (d, J=6.2 Hz, 3H).

MS: Calculated mass (C21H23NO3): 337.17, found mass: M+H=338

Examples 61-63 were prepared analogous to example 60:

### Example 61. 2-(5′-(2-chlorophenethoxy)-1′,3′-dihydrospiro[azetidine-3,2′-inden]-1-yl)acetic acid

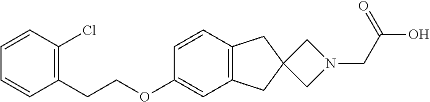 [SMILES: O=C(O)CN1CC2(Cc3ccc(OCCc4ccccc4Cl)cc3C2)C1]

¹H NMR (600 MHz, DMSO-d₆) δ 7.45 (dd, J=7.5, 1.8 Hz, 2H), 7.34-7.25 (m, 2H), 7.08 (d, J=8.2 Hz, 1H), 6.81 (d, J=2.4 Hz, 1H), 6.70 (dd, J=8.2, 2.5 Hz, 1H), 4.14 (t, J=6.9 Hz, 2H), 3.77 (d, J=1.5 Hz, 4H), 3.40 (s, 2H), 3.14 (d, J=6.9 Hz, 2H), 3.11 (s, 2H), 3.06 (s, 2H).

MS: Calculated mass (C21H22ClNO3): 371.13, found mass: M+H=372/374

### Example 62. 2-(5′-(2-chlorophenethoxy)-1′,3′-dihydrospiro[azetidine-3,2′-inden]-1-yl)acetic acid

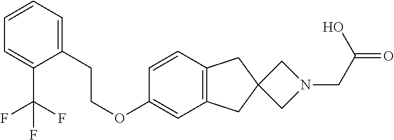 [SMILES: O=C(O)CN1CC2(Cc3ccc(OCCc4ccccc4C(F)(F)F)cc3C2)C1]

¹H NMR (600 MHz, DMSO-d₆) δ 7.73-7.71 (m, 1H), 7.67-7.60 (m, 2H), 7.48-7.44 (m, 1H), 7.08 (d, J=8.2 Hz, 1H), 6.81 (d, J=2.5 Hz, 1H), 6.71 (dd, J=8.3, 2.5 Hz, 1H), 4.15 (t, J=6.9 Hz, 2H), 3.83 (s, 4H), 3.52 (s, 2H), 3.19 (t, J=6.9 Hz, 2H), 3.12 (s, 2H), 3.08 (s, 2H).

MS: Calculated mass (C22H22F3NO3): 405.16, found mass: M+H=406

### Example 63. 2-(5′-(2,6-dichlorophenethoxy)-1′,3′-dihydrospiro[azetidine-3,2′-inden]-1-yl)acetic acid

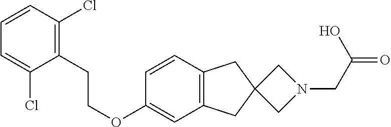 [SMILES: O=C(O)CN1CC2(Cc3ccc(OCCc4c(Cl)cccc4Cl)cc3C2)C1]

¹H NMR (500 MHz, DMSO-d₆) δ 7.49 (d, J=8.0 Hz, 2H), 7.35-7.30 (m, 1H), 7.10 (d, J=8.2 Hz, 1H), 6.83 (d, J=2.6 Hz, 1H), 6.72 (dd, J=8.1, 2.4 Hz, 1H), 4.18 (s, 2H), 4.14-4.07 (m, 6H), 3.18 (s, 2H), 3.13 (s, 2H).

MS: Calculated mass (C21H21Cl2NO3): 405.09, found mass: M+H=406/408/410

### Example 64. 4-(5′-((2-chloro-6-ethylbenzyl)oxy)-1′,3′-dihydrospiro[azetidine-3,2′-inden]-1-yl)-3-methylbutanoic acid

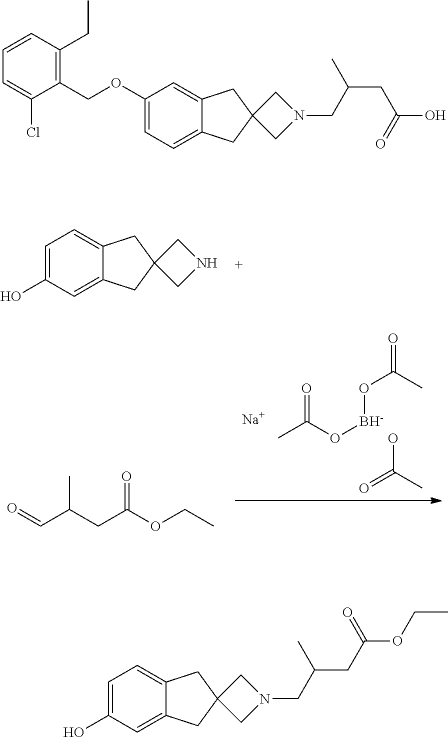

In a 50 mL round-bottomed flask 1′,3′-dihydrospiro[azetidine-3,2′-inden]-5′-ol (100 mg, 0.571 mmol) was suspended in THF (5 mL) and MeOH (1 mL) to give a white suspension. Ethyl 3-methyl-4-oxobutanoate (329 mg, 2.283 mmol) was added (clear solution). After 30 min at RT Sodium triacetoxyborohydride (475 mg, 2.241 mmol) was added. The reaction mixture was stirred at RT overnight. 2 mL water and CH₂Cl₂ were added to the reaction mixture. The mixture was stirred for 10 min at RT. After phase separation with a Chromabond PTS cartridge the organic layer was evaporated. The residue was purified by using the Isco-Combiflash (4 g, 0-20% MeOH in CH₂Cl₂, 18 mL/min)

Yield: 75 mg colorless oil

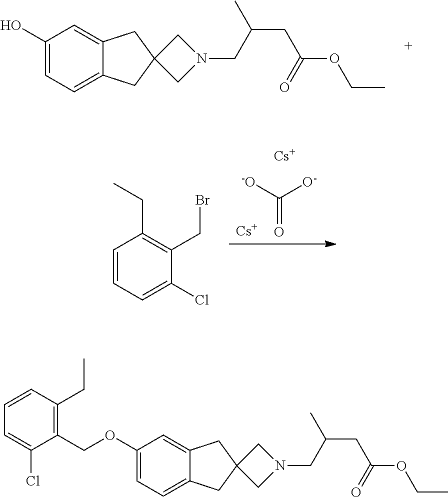

In a 100 mL round-bottomed flask ethyl 4-(5′-hydroxy-1′,3′-dihydrospiro[azetidine-3,2′-inden]-1-yl)-3-methylbutanoate (75 mg, 0.247 mmol) was dissolved in DMF (2 mL) to give a colorless solution. Cesium carbonate (89 mg, 0.272 mmol) and 2-(bromomethyl)-1-chloro-3-ethylbenzene (63.5 mg, 0.272 mmol) were added. The reaction mixture was stirred at RT.

The reaction mixture was evaporated. The residue was dissolved in CH₂Cl₂ and water. After phase separation with a Chromabond PTS-cartridge the organic layer was evaporated. The residue was purified using the Isco-Combiflash (4 g, 0-10% MeOH in CH₂Cl₂, 9 mL/min)

Yield: 58 mg colorless oil

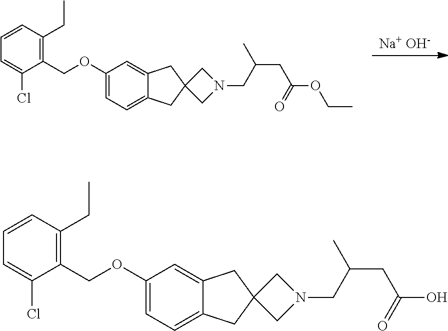 [SMILES: "Na+ OH-" ; CCOC(=O)CC(C)CN1CC2(Cc3ccc(OCc4c(Cl)cccc4CC)cc3C2)C1 ; CCc1cccc(Cl)c1COc1ccc2c(c1)CC1(C2)CN(CC(C)CC(=O)O)C1]

In a 50 mL round-bottomed flask ethyl 4-(5′-((2-chloro-6-ethylbenzyl)oxy)-1′,3′-dihydrospiro[azetidine-3,2′-inden]-1-yl)-3-methylbutanoate (58 mg, 0.127 mmol) was dissolved in MeOH (1 mL) and THF (1 mL) to give a colorless solution. 2M NaOH (0.318 mL, 0.636 mmol) was added. The reaction mixture was stirred overnight at RT.

The reaction mixture was evaporated and the residue was dissolved in water. The mixture was neutralized with 2n HCl (3004) to pH value 6-7. CH₂Cl₂ was added. After phase separation the organic layer was dried over MgSO₄, filtered and evaporated. The residue was dried under vacuum at 40° C.

Yield: 45 mg white foam

¹H NMR (600 MHz, DMSO-d₆) δ 7.38-7.34 (m, 2H), 7.28 (dd, J=6.1, 2.9 Hz, 1H), 7.11 (d, J=8.2 Hz, 1H), 6.93 (d, J=2.4 Hz, 1H), 6.80 (dd, J=8.1, 2.5 Hz, 1H), 5.09 (s, 2H), 3.30-3.17 (m, 4H), 3.04 (s, 2H), 2.99 (s, 2H), 2.71 (q, J=7.6 Hz, 2H), 2.42 (d, J=6.7 Hz, 2H), 2.31 (dd, J=15.6, 6.9 Hz, 1H), 2.04 (dd, J=15.6, 6.4 Hz, 1H), 1.81 (h, J=6.7 Hz, 1H), 1.16 (t, J=7.6 Hz, 3H), 0.86 (d, J=6.8 Hz, 3H).

MS: Calculated mass (C25H30ClNO3): 427.19, found mass: M+H=428/430

Example 65 was prepared analogous to example 64:

### Example 65. 4-(5′-((2-ethyl-6-fluorobenzyl)oxy)-1′,3′-dihydrospiro[azetidine-3,2′-inden]-1-yl)-3-methylbutanoic acid

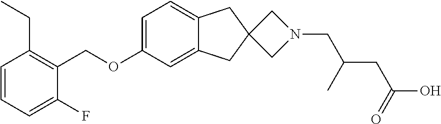 [SMILES: CCc1cccc(F)c1COc1ccc2c(c1)CC1(C2)CN(CC(C)CC(=O)O)C1]

¹H NMR (600 MHz, DMSO-d₆) δ 7.38 (td, J=8.0, 6.0 Hz, 1H), 7.15-7.06 (m, 3H), 6.91 (d, J=2.4 Hz, 1H), 6.78 (dd, J=8.2, 2.5 Hz, 1H), 5.02 (d, J=1.8 Hz, 2H), 3.04 (s, 2H), 2.99 (s, 2H), 2.70 (q, J=7.5 Hz, 2H), 2.31 (dd, J=15.6, 6.9 Hz, 1H), 2.04 (dd, J=15.6, 6.3 Hz, 1H), 1.81 (h, J=6.8 Hz, 1H), 1.17 (t, J=7.5 Hz, 3H), 0.86 (d, J=6.8 Hz, 3H).

MS: Calculated mass (C25H30FNO3): 411.22, found mass: M+H=412

### Example 66. 2-(5′-((2,3-dihydro-1H-inden-1-yl)oxy)-1′,3′-dihydrospiro[azetidine-3,2′-inden]-1-yl)acetic acid

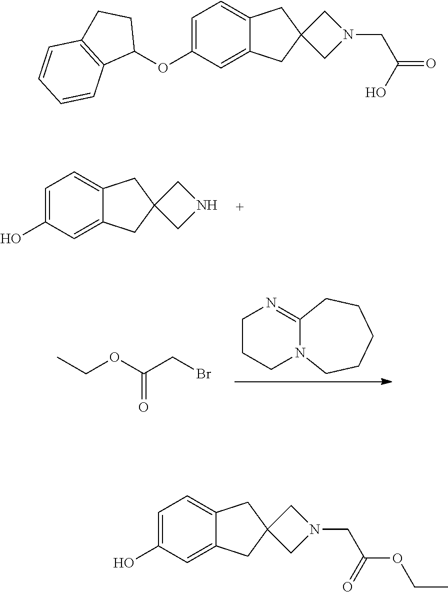

In a 100 mL round-bottomed flask 1′,3′-dihydrospiro[azetidine-3,2′-inden]-5′-ol (1 g, 5.71 mmol) was dissolved in DMF (40 mL) to give a colorless solution. DBU (1.3 mL, 8.62 mmol) and ethyl bromoacetate (800 μL, 7.21 mmol) were added. The reaction mixture was stirred at RT overnight.

The reaction mixture was evaporated, the residue was dissolved in ethyl acetate and washed 2× with water, 1× with saturated sodium chloride solution, the organic layer was dried with MgSO₄, filtered and evaporated. The residue was purified using the Isco-Combiflash (12 g, 0-20% MeOH in CH₂Cl₂, 30 mL/min)

Yield: 990 mg yellow solid

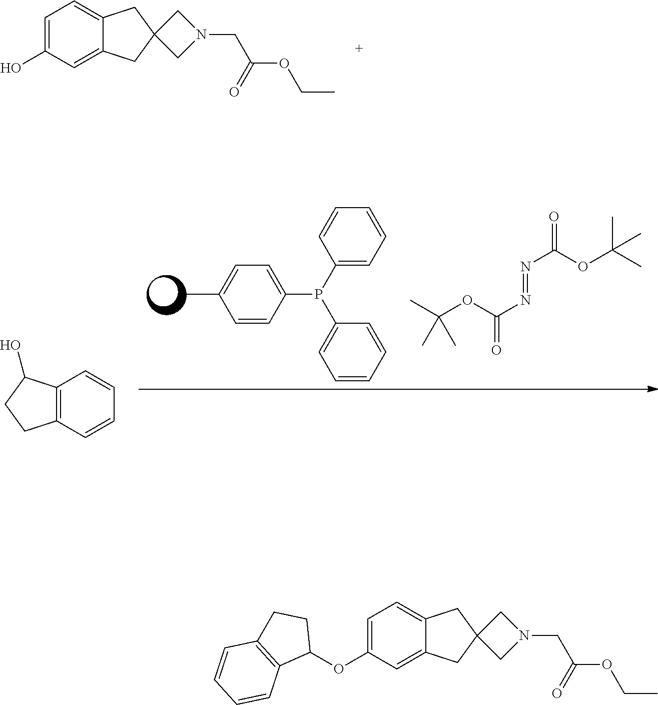

To a 50 mL round bottom flask containing PS-Triphenylphosphin (160 mg, 0.296 mmol) was added to a solution of ethyl 2-(5′-hydroxy-1′,3′-dihydrospiro[azetidine-3,2′-inden]-1-yl)acetate (50 mg, 0.191 mmol) in THF (0.5 ml). The suspension was allowed to stand for 5 min and then a solution of di-tert-butyl azodicarboxylate (66.1 mg, 0.287 mmol) in THF (0.5 mL) was added. A further 0.5 mL THF was added and the solution agitated at RT for 30 min. A solution of 1-INDANOL (26 mg, 0.194 mmol) in THF (0.5 mL) was added and the reaction was stirred overnight.

The reaction mixture was diluted with CH₂Cl₂ and water and stirred for 5 min at RT. After phase separation with a Chromabond PTS-cartridge the organic layer was evaporated. The residue was purified by using the Isco-Combiflash (4 g, 0-10% MeOH in CH₂Cl₂, 18 mL/min)

Yield: 10 mg colorless oil

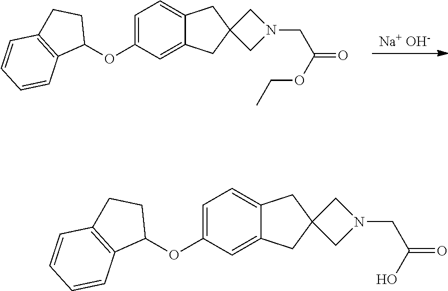 [SMILES: "Na+ OH-" ; CCOC(=O)CN1CC2(Cc3ccc(OC4CCc5ccccc54)cc3C2)C1 ; O=C(O)CN1CC2(Cc3ccc(OC4CCc5ccccc54)cc3C2)C1]

In a 50 mL round bottom flask ethyl 2-(5′-((2,3-dihydro-1H-inden-1-yl)oxy)-1′,3′-dihydrospiro[azetidine-3,2′-inden]-1-yl)acetate (10 mg, 0.026 mmol) was dissolved in THF (0.5 mL) and MeOH (0.5 mL) to give a colorless solution. 2M NaOH (0.1 mL, 0.200 mmol) was added. The reaction was stirred at RT for 2 days.

The reaction mixture was evaporated. The residue was dissolved in water and neutralized with 100 μL 2n HCl. CH₂Cl₂ was added. After phase separation the organic layer was dried over MgSO₄, filtered and evaporated. The residue was dried under vacuum at 40° C.

Yield: 5.6 mg white solid

¹H NMR (600 MHz, DMSO-d₆) δ 7.36 (d, J=7.5 Hz, 1H), 7.33-7.28 (m, 2H), 7.22 (td, J=7.1, 1.9 Hz, 1H), 7.12 (d, J=8.1 Hz, 1H), 6.91 (d, J=2.3 Hz, 1H), 6.80 (dd, J=8.2, 2.5 Hz, 1H), 5.78 (dd, J=6.7, 4.1 Hz, 1H), 3.80 (s, 4H), 3.43 (s, 2H), 3.15 (s, 2H), 3.09 (s, 2H), 3.02 (ddd, J=15.9, 8.6, 5.6 Hz, 1H), 2.86 (ddd, J=16.0, 8.6, 5.4 Hz, 1H), 2.04-1.95 (m, 1H).

MS: Calculated mass (C22H23NO3): 349.17, found mass: M+H=350

Examples 67-69 were prepared analogous to example 66:

### Example 67. 2-(5′-((7-chloro-2,3-dihydro-1H-inden-1-yl)oxy)-1′,3′-dihydrospiro[azetidine-3,2′-inden]-1-yl)acetic acid

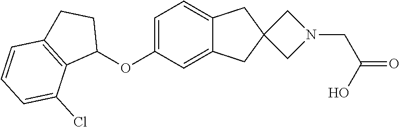 [SMILES: O=C(O)CN1CC2(Cc3ccc(OC4CCc5cccc(Cl)c54)cc3C2)C1]

¹H NMR (600 MHz, DMSO-d₆) δ 7.37 (t, J=7.7 Hz, 1H), 7.31 (dd, J=7.5, 4.1 Hz, 2H), 7.12 (d, J=8.2 Hz, 1H), 6.89 (d, J=2.4 Hz, 1H), 6.77 (dd, J=8.2, 2.5 Hz, 1H), 5.80 (dd, J=6.6, 1.5 Hz, 1H), 3.82-3.74 (m, 4H), 3.15 (s, 2H), 3.09 (s, 2H), 2.94 (ddd, J=16.7, 9.1, 2.5 Hz, 1H), 2.47-2.39 (m, 1H), 2.09 (ddt, J=14.2, 8.0, 2.2 Hz, 1H).

MS: Calculated mass (C22H22ClNO3): 383.13, found mass: M+H=384/386

### Example 68. 2-(5′-((5-bromo-7-fluoro-2,3-dihydro-1H-inden-1-yl)oxy)-1′,3′-dihydrospiro[azetidine-3,2′-inden]-1-yl)acetic acid

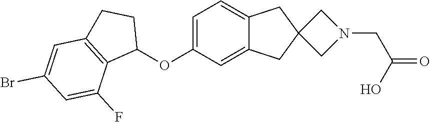 [SMILES: O=C(O)CN1CC2(Cc3ccc(OC4CCc5cc(Br)cc(F)c54)cc3C2)C1]

¹H NMR (600 MHz, DMSO-d₆) δ 7.44 (d, J=1.4 Hz, 1H), 7.39 (dd, J=8.7, 1.5 Hz, 1H), 7.12 (d, J=8.2 Hz, 1H), 6.88 (d, J=2.4 Hz, 1H), 6.76 (dd, J=8.2, 2.5 Hz, 1H), 5.91 (dd, J=6.5, 2.2 Hz, 1H), 3.83-3.73 (m, 4H), 3.17-3.06 (m, 5H), 2.93 (ddd, J=16.9, 9.1, 3.3 Hz, 1H), 2.14-2.04 (m, 1H).

MS: Calculated mass (C22H21BrFNO3): 445.07, found mass: M+H=446/448

### Example 69. 2-(5′-((5,7-dichloro-2,3-dihydro-1H-inden-1-yl)oxy)-1′,3′-dihydrospiro[azetidine-3,2′-inden]-1-yl)acetic acid

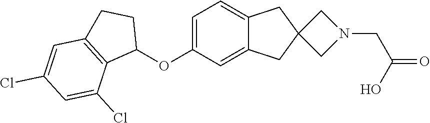 [SMILES: O=C(O)CN1CC2(Cc3ccc(OC4CCc5cc(Cl)cc(Cl)c54)cc3C2)C1]

¹H NMR (600 MHz, DMSO-d₆) δ 7.53-7.38 (m, 2H), 7.12 (d, J=8.2 Hz, 1H), 6.88 (d, J=2.4 Hz, 1H), 6.76 (dd, J=8.2, 2.4 Hz, 1H), 5.77 (dd, J=6.9, 1.8 Hz, 1H), 3.77 (s, 4H), 3.18-3.05 (m, 6H), 2.95 (ddd, J=16.9, 9.1, 2.6 Hz, 2H), 2.10 (ddt, J=14.1, 8.2, 2.3 Hz, 1H), 1.44-1.33 (m, 1H), 1.25 (d, J=21.2 Hz, 1H).

MS: Calculated mass (C22H21Cl2NO3): 417.09, found mass: M+H=418/420/422

### Example 70. 2-(5′-(2-phenoxyethoxy)-1′,3′-dihydrospiro[azetidine-3,2′-inden]-1-yl)acetic acid

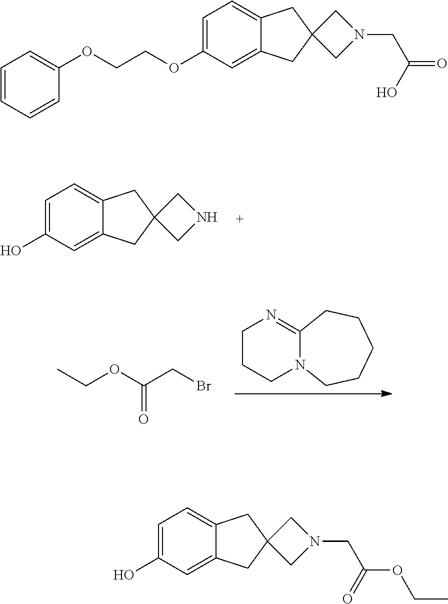

In a 100 mL round-bottomed flask 1′,3′-dihydrospiro[azetidine-3,2′-inden]-5′-ol (1 g, 5.71 mmol) was dissolved in DMF (40 mL) to give a colorless solution. DBU (1.3 mL, 8.62 mmol) and ethyl bromoacetate (800 μL, 7.21 mmol) were added. The reaction mixture was stirred at RT overnight.

The reaction mixture was evaporated, the residue was dissolved in ethyl acetate and washed 2× with water, 1× with saturated sodium chloride solution, the organic layer was dried with MgSO₄, filtered and evaporated. The residue was purified using the Isco-Combiflash (12 g, 0-20% MeOH in CH₂Cl₂, 30 mL/min)

Yield: 990 mg yellow solid

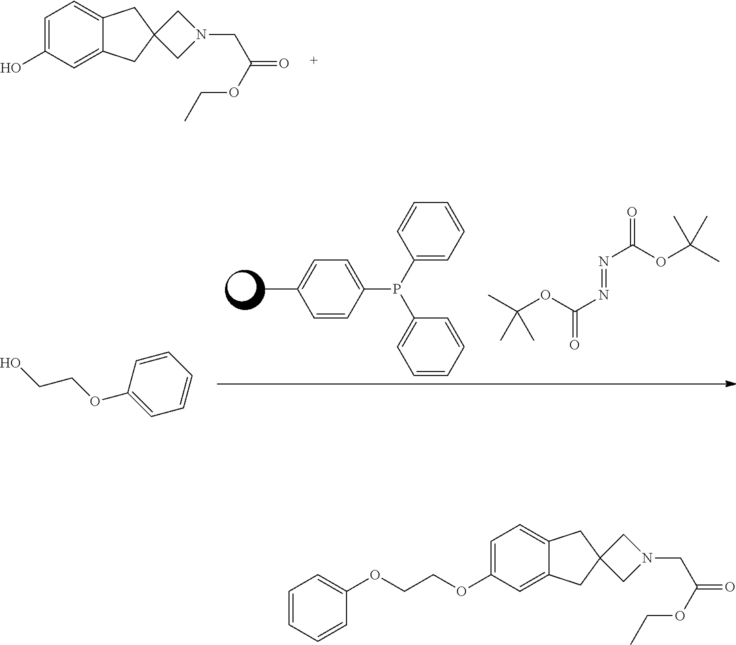

To a 50 mL round bottom flask containing PS-Triphenylphosphin (155 mg, 0.287 mmol) was added a solution of ethyl 2-(5′-hydroxy-1′,3′-dihydrospiro[azetidine-3,2′-inden]-1-yl)acetate (50 mg, 0.191 mmol) in THF (0.5 mL). The suspension was allowed to stand for 5 min and then a solution of di-tert-butyl azodicarboxylate (66.1 mg, 0.287 mmol) in THF (0.5 mL) was added. A further 0.5 mL THF was added and the solution agitated at RT for 30 min. A solution of 2-phenoxyethanol (0.025 mL, 0.201 mmol) in THF (0.5 mL) was added and the reaction was stirred overnight at RT.

The reaction mixture was diluted with CH₂Cl₂ and water and stirred for 5 min at RT. After phase separation with a Chromabond PTS-cartridge the organic layer was evaporated. The residue was purified by using the Isco-Combiflash (4 g, 0-10% MeOH in CH₂Cl₂, 18 mL/min)

Yield: 14 mg colorless oil

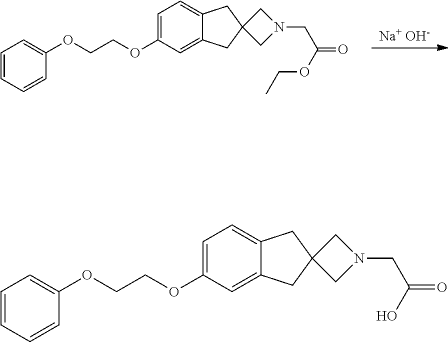 [SMILES: "Na+ OH-" ; CCOC(=O)CN1CC2(Cc3ccc(OCCOc4ccccc4)cc3C2)C1 ; O=C(O)CN1CC2(Cc3ccc(OCCOc4ccccc4)cc3C2)C1]

In a 50 mL round bottom flask ethyl 2-(5′-(2-phenoxyethoxy)-1′,3′-dihydrospiro[azetidine-3,2′-inden]-1-yl)acetate (14 mg, 0.037 mmol) was dissolved in THF (1 mL) and MeOH (1 mL) to give a colorless solution. 2M NaOH (100 μL, 0.2 mmol) was added. The reaction mixture was stirred overnight.

The reaction mixture was evaporated. The residue was dissolved in water and neutralized with 100 μL 2n HCl. The mixture was stirred at RT for 1 h, the precipitate was filtered, washed 1× with 0.5 mL water and dried under vacuum at 40° C. overnight.

Yield: 8.7 mg white solid

¹H NMR (600 MHz, DMSO-d₆) δ 7.32-7.28 (m, 2H), 7.11 (d, J=8.2 Hz, 1H), 6.99-6.93 (m, 3H), 6.86 (d, J=2.4 Hz, 1H), 6.75 (dd, J=8.3, 2.5 Hz, 1H), 4.31-4.22 (m, 4H), 3.76 (s, 4H), 3.13 (s, 2H), 3.08 (s, 2H).

MS: Calculated mass (C21H23NO4): 353.16, found mass: M+H=354

Example 71 was prepared analogous to example 70:

### Example 71. 2-(5′-(2-(4-chlorophenoxy)ethoxy)-1′,3′-dihydrospiro[azetidine-3,2′-inden]-1-yl)acetic acid

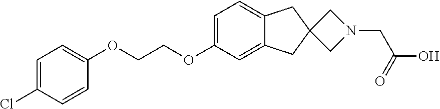 [SMILES: O=C(O)CN1CC2(Cc3ccc(OCCOc4ccc(Cl)cc4)cc3C2)C1]

¹H NMR (600 MHz, DMSO-d₆) δ 7.36-7.31 (m, 2H), 7.11 (d, J=8.3 Hz, 1H), 7.03-6.99 (m, 2H), 6.85 (d, J=2.3 Hz, 1H), 6.74 (dd, J=8.3, 2.5 Hz, 1H), 4.31-4.22 (m, 4H), 3.81 (s, 4H), 3.13 (s, 2H), 3.09 (s, 2H).

MS: Calculated mass (C21H22ClNO4): 387.12, found mass: M+H=388/390

### Example 72. 2-(5′-((2,3-dihydrobenzofuran-7-yl)methoxy)-1′,3′-dihydrospiro[azetidine-3,2′-inden]-1-yl)acetic acid

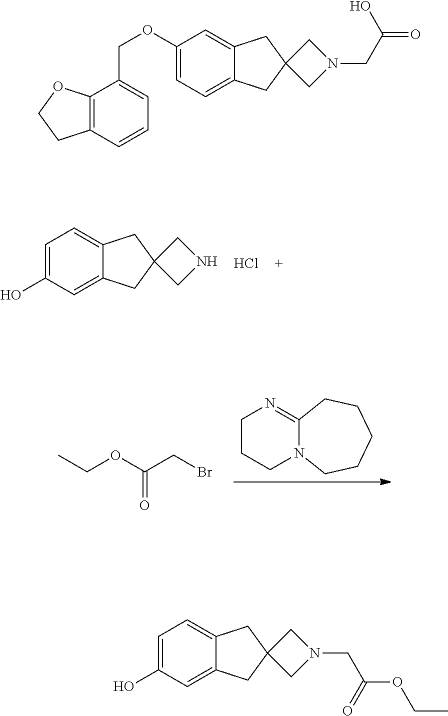

In a 50 mL round-bottomed flask 1′,3′-dihydrospiro[azetidine-3,2′-inden]-5′-ol hydrochloride (100 mg, 0.472 mmol) was dissolved in acetonitrile (5 mL) to give a colorless solution. DBU (0.2 mL, 1.327 mmol) and ethyl bromoacetate (55 μL, 0.496 mmol) were added. The reaction mixture was stirred for 1 h at RT.

The reaction mixture was evaporated, the residue was dissolved in CH₂Cl₂ and washed 1× with sat. NH₄Cl-solution, the phases were separated with a Chromabond PTS-Cartridge and the organic layer was evaporated. The residue was purified by flash chromatography (silica 4 g, 0-10% MeOH in CH₂Cl₂)

Yield: 81 mg colorless oil

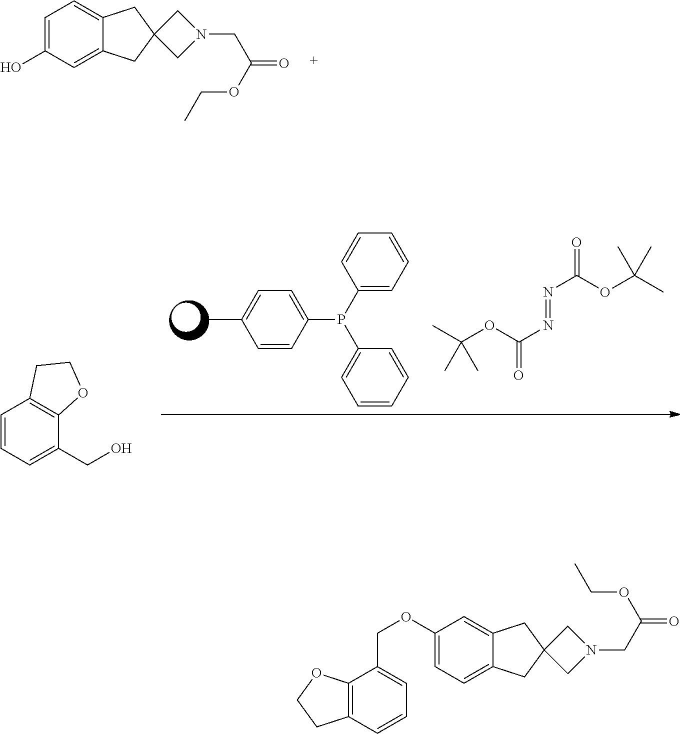

To a 50 mL round bottom flask containing PS-Triphenylphosphine (a resin-bound triphenylphosphine, 183 mg, 0.344 mmol) was added a solution of ethyl 2-(5′-hydroxy-1′,3′-dihydrospiro[azetidine-3,2′-inden]-1-yl)acetate (60 mg, 0.230 mmol) in THF (0.5 mL). The suspension was allowed to stand for 5 min and then a solution of di-tert-butyl azodicarboxylate (79 mg, 0.344 mmol) in THF (0.5 mL) was added. A further 0.5 mL THF was added and the solution agitated at RT for 30 min. A solution of 2,3-dihydrobenzo[b]furan-7-methanol (35 mg, 0.233 mmol) in THF (0.5 mL) was added and the reaction was stirred overnight.

The reaction mixture was diluted with CH₂Cl₂ and water and stirred for 5 min at RT. After phase separation with a Chromabond PTS-cartridge the organic layer was evaporated. The residue was purified by using the Isco-Combiflash (4 g, 0-10% MeOH in CH₂Cl₂, 18 mL/min).

Yield: 15 mg yellow oil

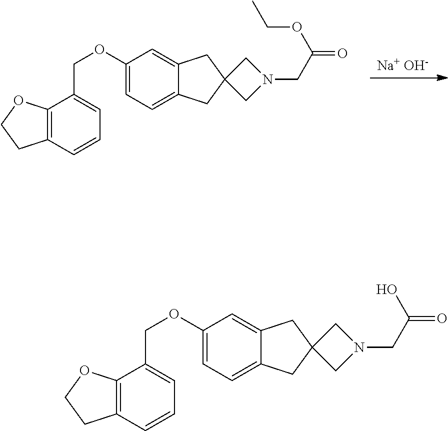 [SMILES: "Na+ OH-" ; CCOC(=O)CN1CC2(Cc3ccc(OCc4cccc5c4OCC5)cc3C2)C1 ; O=C(O)CN1CC2(Cc3ccc(OCc4cccc5c4OCC5)cc3C2)C1]

In a 50 mL round bottom flask ethyl 2-(5′-((2,3-dihydrobenzofuran-7-yl)methoxy)-1′,3′-dihydrospiro[azetidine-3,2′-inden]-1-yl)acetate (15 mg, 0.038 mmol) was dissolved in THF (0.5 mL) and MeOH (0.5 mL) to give a colorless solution. 2M NaOH (150 μL, 0.300 mmol) was added. The reaction mixture was stirred overnight.

The reaction mixture was evaporated. The residue was dissolved in water and neutralized with 150 μL 2n HCl. CH₂Cl₂ was added. After phase separation the organic layer was dried over MgSO₄, filtered and evaporated. The residue was purified using the Isco-Combiflash (4 g, 0-40% MeOH in CH₂Cl₂)

Yield: 10.1 mg white foam

¹H NMR (600 MHz, DMSO-d⁶) δ 7.21 (dd, J=7.3, 1.3 Hz, 1H), 7.15 (dd, J=7.6, 1.2 Hz, 1H), 7.09 (d, J=8.3 Hz, 1H), 6.87 (d, J=2.4 Hz, 1H), 6.83 (t, J=7.5 Hz, 1H), 6.75 (dd, J=8.2, 2.5 Hz, 1H), 4.93 (s, 2H), 4.60-4.45 (m, 2H), 3.76 (d, J=1.6 Hz, 4H), 3.39 (s, 2H), 3.20 (t, J=8.7 Hz, 3H), 3.07 (s, 4H). 23 protons

MS: Calculated mass (C22H23NO4): 365.16, found mass: M+H=366

Examples 73 and 74 were prepared analogous to example 72:

### Example 73. 2-(5′-((2,3-dihydro-1H-inden-2-yl)oxy)-1′,3′-dihydrospiro[azetidine-3,2′-inden]-1-yl)acetic acid

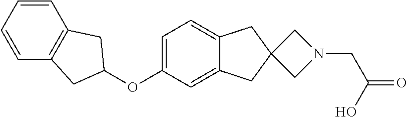 [SMILES: O=C(O)CN1CC2(Cc3ccc(OC4Cc5ccccc5C4)cc3C2)C1]

¹H NMR (600 MHz, DMSO-d⁶) δ 7.21 (ddd, J=52.7, 5.4, 3.2 Hz, 4H), 7.10 (d, J=8.3 Hz, 1H), 6.81 (d, J=2.3 Hz, 1H), 6.70 (dd, J=8.2, 2.5 Hz, 1H), 5.18 (td, J=6.0, 2.9 Hz, 1H), 3.80 (s, 2H), 3.41 (s, 4H), 3.32 (d, J=6.0 Hz, 2H), 3.11 (d, J=29.2 Hz, 4H), 2.99 (dd, J=16.9, 2.5 Hz, 2H).

MS: Calculated mass (C22H23NO3): 349.17, found mass: M+H=350

### Example 74. 2-(5′-((2,3-dihydro-1H-inden-4-yl)methoxy)-1′,3′-dihydrospiro[azetidine-3,2′-inden]-1-yl)acetic acid

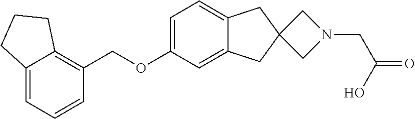 [SMILES: O=C(O)CN1CC2(Cc3ccc(OCc4cccc5c4CCC5)cc3C2)C1]

¹H NMR (600 MHz, DMSO-d⁶) δ 7.19 (t, J=7.6 Hz, 2H), 7.17-7.07 (m, 2H), 6.89 (d, J=2.5 Hz, 1H), 6.77 (dd, J=8.2, 2.5 Hz, 1H), 5.00 (s, 2H), 3.77 (s, 4H), 3.10 (d, J=30.2 Hz, 4H), 2.88 (t, J=7.5 Hz, 5H), 2.02 (p, J=7.5 Hz, 2H).

MS: Calculated mass (C23H25NO3): 363.18, found mass: M+H=364

### Example 75. 2-(5′-((6-isopropoxypyridin-3-yl)ethynyl)-1′,3′-dihydrospiro[azetidine-3,2′-inden]-1-yl)acetic acid

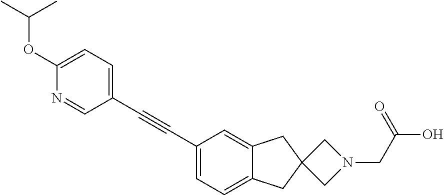 [SMILES: CC(C)Oc1ccc(C#Cc2ccc3c(c2)CC2(C3)CN(CC(=O)O)C2)cn1]

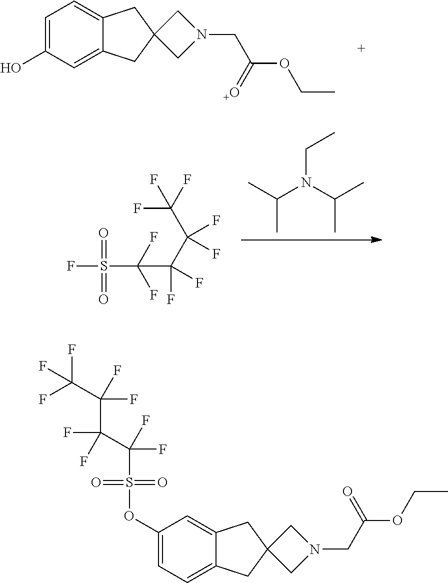

In a 50 mL 3 neck-flask ethyl 2-(5′-hydroxy-1′,3′-dihydrospiro[azetidine-3,2′-inden]-1-yl)acetate (310 mg, 1.186 mmol, prepared as described for example 8) was dissolved in CH₂Cl₂ (10 mL) to give a colorless solution. N,N-diisopropylethylamine (0.810 mL, 4.75 mmol) was added. The reaction mixture was cooled down to 0° C. Nonafluorobutanesulfonyl fluoride (0.533 mL, 2.97 mmol) was added slowly. The reaction mixture was stirred for 1 h at 0° C. LC/MS showed that the reaction was finished.

The reaction mixture was diluted with CH₂Cl₂ and water was added. The mixture was stirred at RT for 10 min. A small amount of sat. NH₄Cl-solution was added. After phase separation the organic layer was washed with saturated NaCl, dried over MgSO₄, filtered and evaporated. The residue was purified using the Isco-Combiflash (12 g, 0-10% MeOH in CH₂Cl₂).

Yield: 302 mg colorless oil

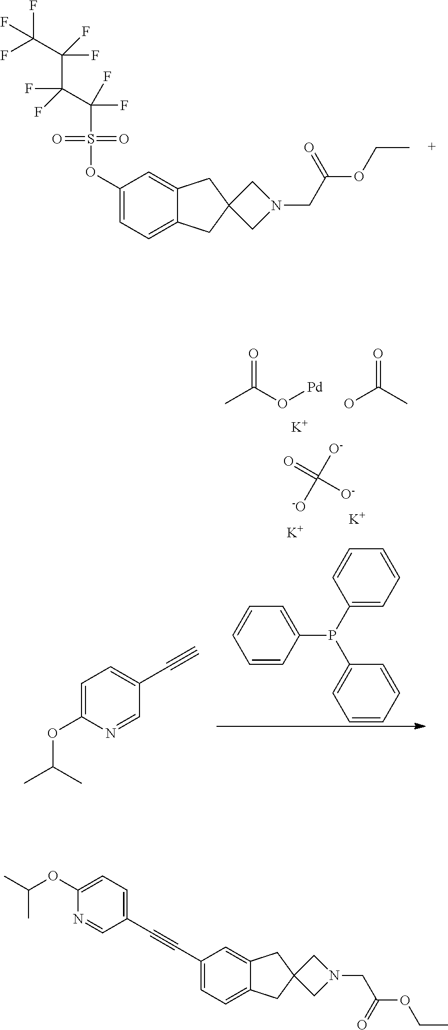

In a 25 mL 3-neck round-bottom flask palladium(II) acetate (2.97 mg, 0.013 mmol), triphenylphosphine (11.58 mg, 0.044 mmol) and potassium phosphate tribasic (28.1 mg, 0.132 mmol) were suspended under stirring in an argon atmosphere for 30 min. In a second flask 5-ethynyl-2-isopropoxypyridine (26.7 mg, 0.166 mmol) and ethyl 2-(5′-(((perfluorobutyl)sulfonyl)oxy)-1′,3′-dihydrospiro[azetidine-3,2′-inden]-1-yl)acetate (60 mg, 0.110 mmol) was dissolved in dimethyl sulfoxide (DMSO) (2 mL) and dried under argon for 30 min. This solution was put into the 3-neck flask via syringe and heated to 80° C. for 1 h.

The reaction mixture was diluted with CH₂Cl₂ and water. The mixture was stirred at RT for 10 min. After phase separation with the organic layer was washed 1× with water and 1× with saturated NaCl, dried over MgSO₄, filtered and evaporated. The residue was purified using the Isco-Combiflash (4 g, 0-10% MeOH in CH₂Cl₂, 18 mL/min)

Yield: 34 mg light brown oil

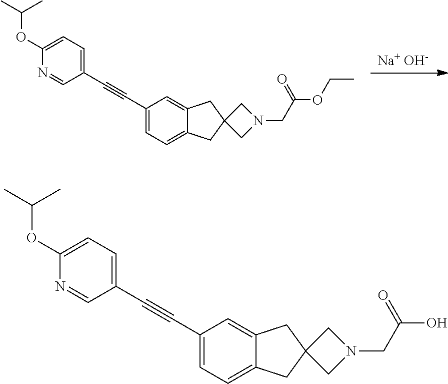

In a 50 mL round bottom flask ethyl 2-(5′-((6-isopropoxypyridin-3-yl)ethynyl)-1′,3′-dihydrospiro[azetidine-3,2′-inden]-1-yl)acetate (34 mg, 0.084 mmol) was dissolved in THF (0.5 mL) and MeOH (0.5 mL) to give a colorless solution. 2M NaOH (250 μL, 0.5 mmol) was added. The reaction mixture was stirred overnight. The reaction mixture was evaporated. The residue was dissolved in water and neutralized with 250 μL 2n HCl. The mixture was stirred at RT for 2 h, the precipitate was filtered, washed 1× with water and dried overnight at 40° C. under vacuum.

Yield: 30 mg brown solid (90% pure by HPLC)

¹H NMR (600 MHz, DMSO-d⁶) δ 8.34 (d, J=2.3 Hz, 1H), 7.81 (dd, J=8.6, 2.4 Hz, 1H), 7.56 (ddd, J=8.0, 7.39 (d, J=1.4 Hz, 1H), 7.32 (dd, J=7.7, 1.5 Hz, 1H), 7.26 (d, J=7.9 Hz, 1H), 6.80 (d, J=8.6 Hz, 1H), 5.26 (hept, J=6.2 Hz, 1H), 3.77 (s, 4H), 3.19 (d, J=5.4 Hz, 4H), 1.30 (d, J=6.2 Hz, 6H).

MS: Calculated mass (C23H24N2O3): 376.18, found mass: M+H=377

Examples 76-82 were prepared analogous to example 75:

### Example 76. 2-(5′-((4-methylcyclohexyl)ethynyl)-1′,3′-dihydrospiro[azetidine-3,2′-inden]-1-yl)acetic acid

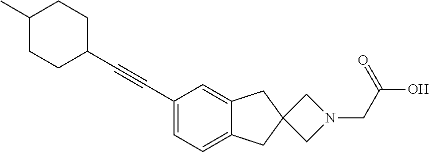 [SMILES: CC1CCC(C#Cc2ccc3c(c2)CC2(C3)CN(CC(=O)O)C2)CC1]

¹H NMR (600 MHz, DMSO-d⁶) δ 7.26-7.09 (m, 3H), 3.75 (d, J=4.3 Hz, 4H), 3.19-3.09 (m, 4H), 2.92 (t, J=4.1 Hz, 1H), 1.98-0.91 (m, 10H), 0.88 (dd, J=17.8, 6.5 Hz, 3H).

MS: Calculated mass (C22H27NO2): 337.20, found mass: M+H=338

### Example 77. 2-(5′-((4-ethoxyphenyl)ethynyl)-1′,3′-dihydrospiro[azetidine-3,2′-inden]-1-yl)acetic acid

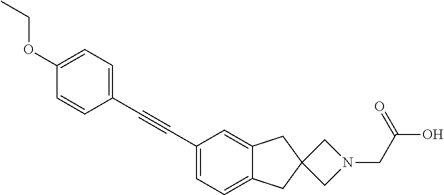 [SMILES: CCOc1ccc(C#Cc2ccc3c(c2)CC2(C3)CN(CC(=O)O)C2)cc1]

¹H NMR (600 MHz, DMSO-d⁶) δ 7.47-7.41 (m, 2H), 7.37 (s, 1H), 7.29 (dd, J=7.7, 1.5 Hz, 1H), 7.24 (d, J=7.8 Hz, 1H), 6.98-6.93 (m, 2H), 4.06 (q, J=7.0 Hz, 2H), 3.77 (s, 4H), 3.18 (d, J=4.7 Hz, 4H), 1.33 (t, J=6.9 Hz, 3H).

MS: Calculated mass (C23H23NO3): 361.17, found mass: M+H=362

### Example 78. 2-(5′-((6-(cyclopentyloxy)pyridin-3-yl)ethynyl)-1′,3′-dihydrospiro[azetidine-3,2′-inden]-1-yl)acetic acid

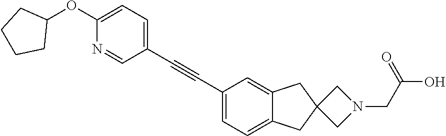 [SMILES: O=C(O)CN1CC2(Cc3ccc(C#Cc4ccc(OC5CCCC5)nc4)cc3C2)C1]

¹H NMR (500 MHz, DMSO-d⁶) δ 8.34 (d, J=2.4 Hz, 1H), 7.80 (dd, J=8.6, 2.4 Hz, 1H), 7.39 (s, 1H), 7.32 (dd, J=7.9, 1.5 Hz, 1H), 7.26 (d, J=7.9 Hz, 1H), 6.80 (d, J=8.6 Hz, 1H), 5.38 (tt, J=6.0, 2.9 Hz, 1H), 3.74 (s, 4H), 3.18 (d, J=4.3 Hz, 4H), 1.99-1.88 (m, 3H), 1.76-1.65 (m, 5H), 1.60 (tdq, J=9.4, 6.6, 3.2, 2.5 Hz, 2H).

MS: Calculated mass (C25H26N2O3): 402.19, found mass: M+H=403

### Example 79. 2-(5′-((6-((1,1,1-trifluoropropan-2-yl)oxy)pyridin-3-yl)ethynyl)-1′,3′-dihydrospiro[azetidine-3,2′-inden]-1-yl)acetic acid

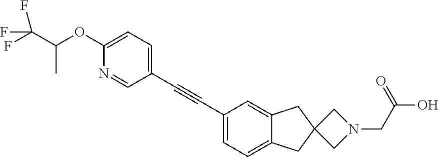 [SMILES: CC(Oc1ccc(C#Cc2ccc3c(c2)CC2(C3)CN(CC(=O)O)C2)cn1)C(F)(F)F]

¹H NMR (500 MHz, DMSO-d⁶) δ 8.40 (d, J=2.4 Hz, 1H), 7.94 (dd, J=8.5, 2.4 Hz, 1H), 7.41 (s, 1H), 7.34 (dd, J=7.9, 1.5 Hz, 1H), 7.27 (d, J=7.9 Hz, 1H), 7.01 (d, J=8.5 Hz, 1H), 5.91 (hept, J=6.7 Hz, 1H), 3.74 (s, 4H), 3.19 (d, J=4.5 Hz, 4H), 1.47 (d, J=6.5 Hz, 3H).

MS: Calculated mass (C23H21F3N2O3): 430.15, found mass: M+H=431

### Example 80. 2-(5′-((2-chlorophenyl)ethynyl)-1′,3′-dihydrospiro[azetidine-3,2′-inden]-1-yl)acetic acid

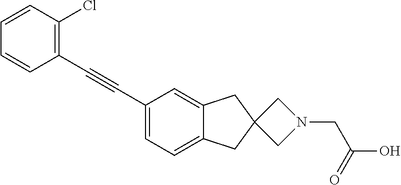 [SMILES: O=C(O)CN1CC2(Cc3ccc(C#Cc4ccccc4Cl)cc3C2)C1]

¹H NMR (600 MHz, DMSO-d⁶) δ 7.65 (td, J=7.0, 6.6, 1.7 Hz, 1H), 7.61-7.57 (m, 1H), 7.46-7.34 (m, 4H), 7.30 (d, J=7.7 Hz, 1H), 3.86 (s, 4H), 3.58 (s, 2H), 3.22 (d, J=4.0 Hz, 4H).

MS: Calculated mass (C21H18ClNO2): 351.10, found mass: M+H=352/354

### Example 81. 2-(5′-((2-fluoro-4-methoxyphenyl)ethynyl)-1′,3′-dihydrospiro[azetidine-3,2′-inden]-1-yl)acetic acid

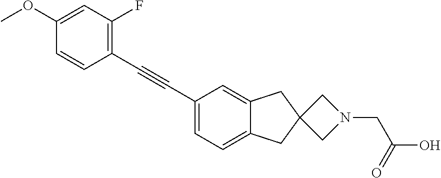 [SMILES: COc1ccc(C#Cc2ccc3c(c2)CC2(C3)CN(CC(=O)O)C2)c(F)c1]

¹H NMR (600 MHz, DMSO-d₆) δ 7.50 (t, J=8.6 Hz, 1H), 7.38 (s, 1H), 7.32-7.24 (m, 2H), 6.98 (dd, J=11.7, 2.5 Hz, 1H), 6.84 (dd, J=8.7, 2.5 Hz, 1H), 3.81 (s, 3H), 3.18 (d, J=4.3 Hz, 4H).

MS: Calculated mass (C22H20FNO3): 365.14, found mass: M+H=366

### Example 82. 2-(5′-(phenylethynyl)-1′,3′-dihydrospiro[azetidine-3,2′-inden]-1-yl)acetic acid

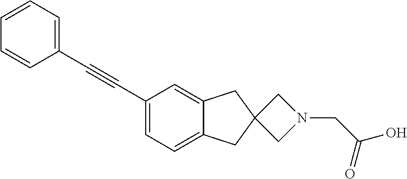 [SMILES: O=C(O)CN1CC2(Cc3ccc(C#Cc4ccccc4)cc3C2)C1]

¹H NMR (600 MHz, Methanol-d⁴) δ 7.51-7.44 (m, 2H), 7.40-7.31 (m, 5H), 7.24 (d, J=7.7 Hz, 1H), 4.17 (s, 4H), 3.81 (s, 2H)

MS: Calculated mass (C21H19NO2): 317.14, found mass: M+H=318

### Example 83. 3-(5′-((2,3-dihydro-1H-inden-4-yl)methoxy)-1′,3′-dihydrospiro[azetidine-3,2′-inden]-1-yl)cyclobutanecarboxylic acid

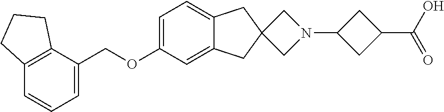 [SMILES: O=C(O)C1CC(N2CC3(Cc4ccc(OCc5cccc6c5CCC6)cc4C3)C2)C1]

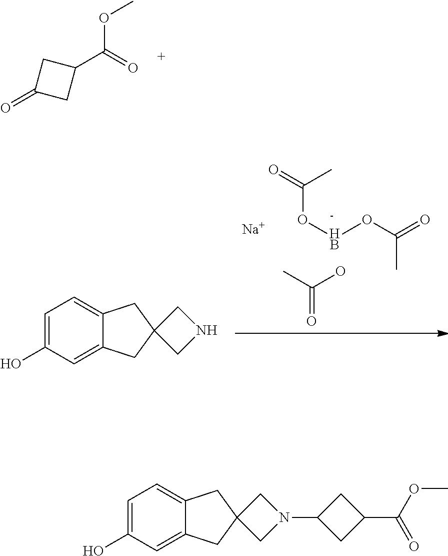

In a 100 mL 3-neck round-bottomed flask 1′,3′-dihydrospiro[azetidine-3,2′-inden]-5′-ol (2 g, 11.41 mmol) was dissolved in THF (80 mL) to give a white suspension. Methyl 3-oxocyclobutanecarboxylate (2.92 g, 22.83 mmol) was added. The mixture was stirred for 60 min at RT. Sodium triacetoxyborohydride (4.84 g, 22.83 mmol) was added. The reaction mixture was stirred overnight at RT.

The reaction mixture was diluted with CH₂Cl₂ and water and stirred for 30 min. CH₂Cl₂ was added, the organic layer was washed 2× with NaHCO₃-solution, 1× with saturated sodium chloride solution, dried over MgSO₄, filtered and evaporated.

Yield: 3.47 g light red solid

The product could either be used without further purification for the next or purified by flash chromatography: 2.5 g were absorbed on Celite XTR and purified using the Isco-Combiflash (12 g, 0-20% MeOH in CH₂Cl₂, 35 mL/min).

Yield: 1 g colorless foam

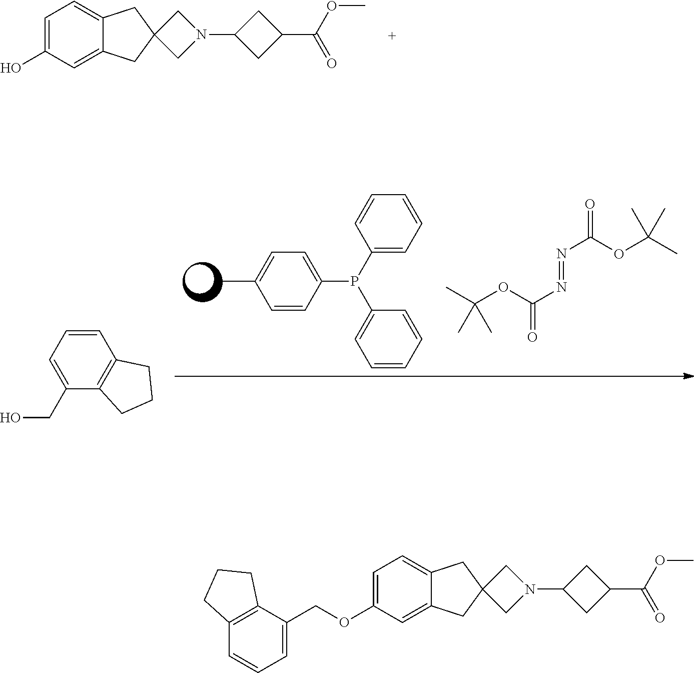

To a 50 mL round bottom flask containing PS-Triphenylphosphin (a resin-bound triphenylphosphine, 357 mg, 0.66 mmol) was added a solution of methyl 3-(5′-hydroxy-1′,3′-dihydrospiro[azetidine-3,2′-inden]-1-yl)cyclobutanecarboxylate (50 mg, 0.174 mmol) in THF (4 mL). The suspension was allowed to stand for 5 min and then a solution of di-tert-butyl azodicarboxylate (60.1 mg, 0.261 mmol) was added. 2,3-DIHYDRO-1H-INDEN-4-YLMETHANOL (30.9 mg, 0.209 mmol) was added and the reaction was stirred for 2 days at RT.

The reaction mixture was diluted with CH₂Cl₂ and water and stirred for 5 min at RT. After phase separation with a Chromabond PTS-cartridge the organic layer was evaporated. The residue was purified by using the Isco-Combiflash (4 g, 0-10% MeOH in CH₂Cl₂)

Yield: 7 mg colorless oil

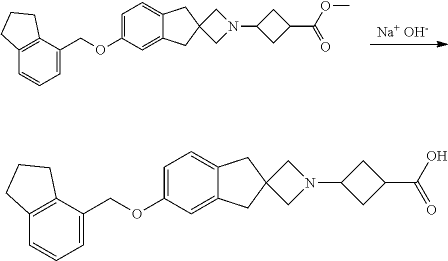 [SMILES: "Na+ OH-" ; COC(=O)C1CC(N2CC3(Cc4ccc(OCc5cccc6c5CCC6)cc4C3)C2)C1 ; O=C(O)C1CC(N2CC3(Cc4ccc(OCc5cccc6c5CCC6)cc4C3)C2)C1]

In a 50 mL round bottom flask methyl 3-(5′-((2,3-dihydro-1H-inden-4-yl)methoxy)-1′,3′-dihydrospiro[azetidine-3,2′-inden]-1-yl)cyclobutanecarboxylate (7 mg, 0.017 mmol) was dissolved in THF (0.5 mL) and MeOH (0.5 mL) to give a yellow solution. 2M NaOH (0.1 mL, 0.200 mmol) was added. The reaction mixture was stirred overnight at RT.

The reaction mixture was evaporated. The residue was dissolved in water and neutralized with 0.1 mL 2n HCl. CH₂Cl₂ was added. After phase separation the organic layer was dried over MgSO₄, filtered and evaporated.

Yield: 3.2 mg colorless oil

¹H NMR (500 MHz, DMSO-d⁶) δ 7.23-7.08 (m, 5H), 6.90 (d, J=2.1 Hz, 1H), 6.79 (dd, J=8.3, 2.4 Hz, 1H), 5.00 (s, 2H), 3.13 (s, 2H), 3.08 (s, 2H), 2.91-2.79 (m, 5H), 2.47 (s, 2H), 2.13 (s, 1H), 2.02 (p, J=7.6 Hz, 2H), 1.37 (d, J=16.2 Hz, 1H), 1.24 (s, 2H), 1.16 (d, J=10.9 Hz, 2H).

MS: Calculated mass (C26H29NO3): 403.21, found mass: M+H=404

Examples 84-86 were prepared analogous to example 83:

### Example 84. 3-(5′-((4-(cyclopropylmethoxy)benzyl)oxy)-1′,3′-dihydrospiro[azetidine-3,2′-inden]-1-yl)cyclobutanecarboxylic acid

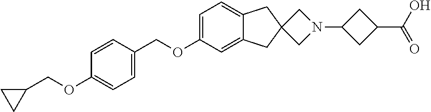 [SMILES: O=C(O)C1CC(N2CC3(Cc4ccc(OCc5ccc(OCC6CC6)cc5)cc4C3)C2)C1]

¹H NMR (600 MHz, DMSO-d⁶) δ 7.34-7.30 (m, 2H), 7.07 (d, J=8.3 Hz, 1H), 6.94-6.89 (m, 2H), 6.85 (d, J=2.4 Hz, 1H), 6.79-6.71 (m, 1H), 4.94 (s, 2H), 3.80 (d, J=7.0 Hz, 2H), 3.47 (s, 1H), 3.19 (d, J=22.2 Hz, 5H), 3.01 (s, 2H), 2.97 (s, 2H), 2.71 (p, J=8.5 Hz, 1H), 2.22-2.14 (m, 2H), 1.99-1.90 (m, 2H), 1.26-1.16 (m, 1H), 0.59-0.53 (m, 2H), 0.34-0.28 (m, 2H).

MS: Calculated mass (C27H31NO4): 433.23, found mass: M+H=434

### Example 85. 3-(5′-((4-ethoxy-2,6-difluorobenzyl)oxy)-1′,3′-dihydrospiro[azetidine-3,2′-inden]-1-yl)cyclobutanecarboxylic acid

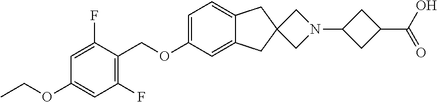 [SMILES: CCOc1cc(F)c(COc2ccc3c(c2)CC2(C3)CN(C3CC(C(=O)O)C3)C2)c(F)c1]

¹H NMR (500 MHz, DMSO-d⁶) δ 7.10 (d, J=8.2 Hz, 1H), 6.89 (d, J=2.2 Hz, 1H), 6.77 (dq, J=8.3, 2.9, 2.3 Hz, 3H), 4.95 (s, 2H), 4.07 (q, J=7.0 Hz, 2H), 3.07 (s, 2H), 3.02 (s, 2H), 2.79-2.71 (m, 1H), 2.45 (d, J=19.3 Hz, 4H), 2.24 (s, 3H), 2.01 (s, 2H), 1.32 (t, J=7.0 Hz, 3H).

MS: Calculated mass (C25H27F2NO4): 443.19, found mass: M+H=444

### Example 86. 3-(5′-((4-ethoxy-2,3-difluorobenzyl)oxy)-1′,3′-dihydrospiro[azetidine-3,2′-inden]-1-yl)cyclobutanecarboxylic acid

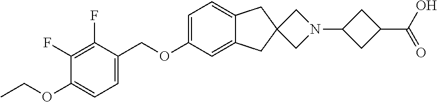 [SMILES: CCOc1ccc(COc2ccc3c(c2)CC2(C3)CN(C3CC(C(=O)O)C3)C2)c(F)c1F]

¹H NMR (600 MHz, Chloroform-d) δ 7.14-7.06 (m, 2H), 6.81 (d, J=2.3 Hz, 1H), 6.80-6.70 (m, 2H), 5.00 (s, 2H), 4.12 (q, J=7.0 Hz, 2H), 3.92 (d, J=56.9 Hz, 3H), 3.60 (p, J=7.0 Hz, 1H), 3.39-3.10 (m, 4H), 2.99 (p, J=8.2 Hz, 1H), 2.67-2.33 (m, 4H), 1.45 (t, J=7.0 Hz, 3H).

MS: Calculated mass (C25H27F2NO4): 443.19, found mass: M+H=444

### Example 87. 3-(5′-((2-cyclopropyl-6-fluorobenzyl)oxy)-1′,3′-dihydrospiro[azetidine-3,2′-inden]-1-yl)cyclobutanecarboxylic acid

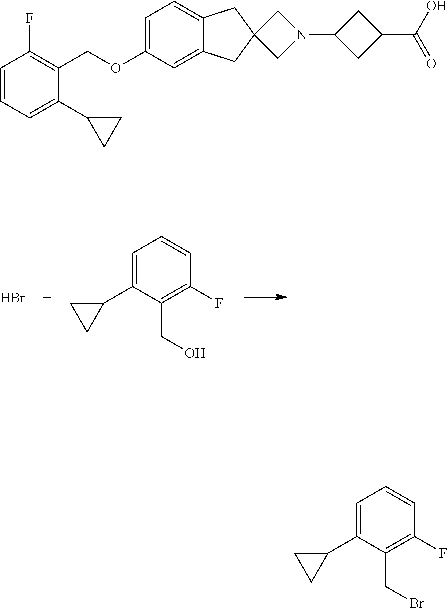 [SMILES: O=C(O)C1CC(N2CC3(Cc4ccc(OCc5c(F)cccc5C5CC5)cc4C3)C2)C1 ; Br ; OCc1c(F)cccc1C1CC1 ; Fc1cccc(C2CC2)c1CBr]

(2-cyclopropyl-6-fluorophenyl)methanol was suspended in 48% aqueous HBr and stirred at RT overnight.

The reaction mixture was dilute with water (20 mL) and extracted with ethyl acetate. The extracts were washed successively with water, saturated NaHCO₃ and water. The organic phase was dried (MgSO₄) and the solvent evaporated in vacuo.

Yield: 550 mg light brown oil

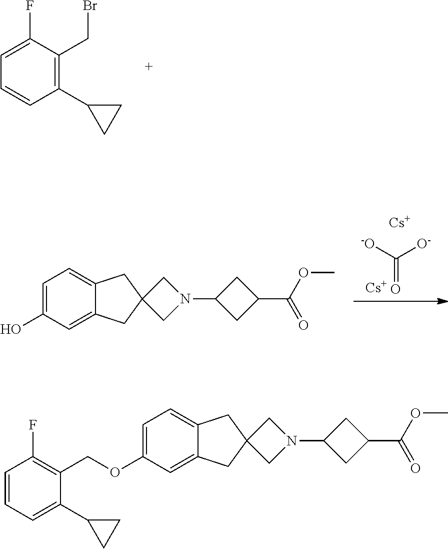

In a 4 mL flask methyl 3-(5′-hydroxy-1′,3′-dihydrospiro[azetidine-3,2′-inden]-1-yl)cyclobutanecarboxylate (50 mg, 0.174 mmol, prepared as described for example 83) was dissolved in DMF (3 mL) to give a colorless solution. Cesium carbonate (85 mg, 0.261 mmol) and 2-(bromomethyl)-1-cyclopropyl-3-fluorobenzene (45 mg, 0.196 mmol) were added. The reaction mixture was stirred at RT overnight.

The reaction mixture was evaporated. The residue was dissolved in water and CH₂Cl₂. After phase separation the organic layer was washed 1× with water and 1× with saturated sodium chloride solution. The organic layer was dried over MgSO₄, filtered and evaporated. The residue was purified using the Isco-Combiflash (4 g, 0-10% MeOH in CH₂Cl₂, 18 mL/min).

Yield: 35 mg colorless oil

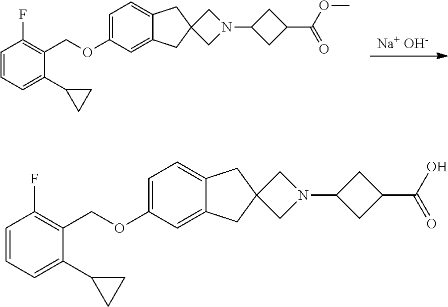 [SMILES: "Na+ OH-" ; COC(=O)C1CC(N2CC3(Cc4ccc(OCc5c(F)cccc5C5CC5)cc4C3)C2)C1 ; O=C(O)C1CC(N2CC3(Cc4ccc(OCc5c(F)cccc5C5CC5)cc4C3)C2)C1]

In a 50 mL round bottom flask methyl 3-(5′-((2-cyclopropyl-6-fluorobenzyl)oxy)-1′,3′-dihydrospiro[azetidine-3,2′-inden]-1-yl)cyclobutanecarboxylate (35 mg, 0.080 mmol) was dissolved in THF (1 mL) and MeOH (1 mL) to give a colorless solution. 2M NaOH (0.4 mL, 0,800 mmol) was added. The reaction mixture was stirred overnight at RT.

The reaction mixture was evaporated. The residue was dissolved in water and neutralized with 0.4 mL 2n HCl. CH₂Cl₂ and a small amount of MeOH were added. After phase separation the organic layer was dried over MgSO₄, filtered and evaporated.

The residue was purified using the Isco-Combiflash (4 g, 0-30% MeOH in CH₂Cl₂, 18 mL/min).

Yield: 10 mg white foam

¹H NMR (600 MHz, DMSO-d⁶) δ 7.33 (td, J=8.0, 6.0 Hz, 1H), 7.10 (dd, J=8.3, 2.5 Hz, 1H), 7.06 (ddd, J=9.5, 8.2, 1.1 Hz, 1H), 6.93 (s, 1H), 6.85 (d, J=7.8 Hz, 1H), 6.80 (dd, J=8.1, 2.4 Hz, 1H), 5.16 (d, J=1.8 Hz, 2H), 3.01 (d, J=30.3 Hz, 4H), 2.70 (q, J=8.5 Hz, 1H), 2.19 (d, J=8.8 Hz, 2H), 2.06 (tt, J=8.5, 5.3 Hz, 1H), 1.94 (d, J=9.9 Hz, 2H), 0.96-0.89 (m, 2H), 0.74-0.66 (m, 2H).

MS: Calculated mass (C26H28FNO3): 421.21, found mass: M+H=422

Examples 88-94 (were prepared analogous to example 87:

### Example 88. 3-(5′-((6-methoxy-2-methylpyridin-3-yl)methoxy)-1′,3′-dihydrospiro[azetidine-3,2′-inden]-1-yl)cyclobutanecarboxylic acid

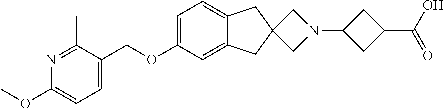 [SMILES: COc1ccc(COc2ccc3c(c2)CC2(C3)CN(C3CC(C(=O)O)C3)C2)c(C)n1]

¹H NMR (600 MHz, DMSO-d⁶) δ 7.68 (d, J=8.3 Hz, 1H), 7.11 (d, J=8.2 Hz, 1H), 6.90 (d, J=2.4 Hz, 1H), 6.79 (dd, J=8.2, 2.4 Hz, 1H), 6.64 (d, J=8.2 Hz, 1H), 4.98 (s, 2H), 3.83 (s, 2H), 3.08 (d, J=6.8 Hz, 2H), 3.03 (d, J=7.1 Hz, 2H), 2.75 (p, J=8.7 Hz, 1H), 2.42 (s, 3H), 2.25 (dqd, J=15.9, 8.5, 8.0, 2.5 Hz, 2H), 2.13 (s, 1H), 2.03 (qd, J=8.9, 2.6 Hz, 2H).

MS: Calculated mass (C24H28N2O4): 408.20, found mass: M+H=409

### Example 89. (1r,3r)-3-(5′-((2,5-dichlorobenzyl)oxy)-1′,3′-dihydrospiro[azetidine-3,2′-inden]-1-yl)cyclobutanecarboxylic acid

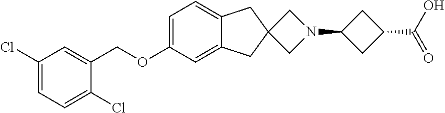 [SMILES: O=C(O)[C@H]1C[C@H](N2CC3(Cc4ccc(OCc5cc(Cl)ccc5Cl)cc4C3)C2)C1]

¹H NMR (500 MHz, Methanol-d⁴) δ 7.54 (d, J=2.5 Hz, 1H), 7.42 (d, J=8.4 Hz, 1H), 7.33 (dd, J=8.5, 2.5 Hz, 1H), 7.14 (d, J=8.3 Hz, 1H), 6.89 (d, J=2.4 Hz, 1H), 6.82 (dd, J=8.2, 2.5 Hz, 1H), 5.11 (s, 2H), 3.92 (s, 6H), 3.22 (s, 2H), 3.18 (s, 2H), 2.98 (tt, J=9.8, 4.8 Hz, 1H), 2.49 (dt, J=13.1, 6.8 Hz, 2H), 2.22 (q, J=10.6, 10.1 Hz, 2H).

MS: Calculated mass (C23H23Cl2NO3): 431.11, found mass: M+H=432/434/436

### Example 90. (1s,3s)-3-(5′-((2,5-dichlorobenzyl)oxy)-1′,3′-dihydrospiro[azetidine-3,2′-inden]-1-yl)cyclobutanecarboxylic acid

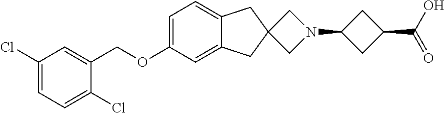 [SMILES: O=C(O)[C@H]1C[C@@H](N2CC3(Cc4ccc(OCc5cc(Cl)ccc5Cl)cc4C3)C2)C1]

¹H NMR (500 MHz, Methanol-d⁴) δ 7.54 (d, J=2.6 Hz, 1H), 7.42 (d, J=8.5 Hz, 1H), 7.32 (dd, J=8.5, 2.6 Hz, 1H), 7.14 (d, J=8.2 Hz, 1H), 6.89 (d, J=2.4 Hz, 1H), 6.82 (dd, J=8.3, 2.4 Hz, 1H), 5.11 (s, 2H), 4.00 (s, 4H), 3.78 (p, J=6.8 Hz, 1H), 3.24 (s, 2H), 3.19 (s, 2H), 2.84 (p, J=7.8 Hz, 1H), 2.60-2.51 (m, 2H), 2.22-2.13 (m, 3H).

MS: Calculated mass (C23H23Cl2NO3): 431.11, found mass: M+H=432/434/436

### Example 91. 3-(5′-((2-chloro-6-cyclopropylbenzyl)oxy)-1′,3′-dihydrospiro[azetidine-3,2′-inden]-1-yl)cyclobutanecarboxylic acid

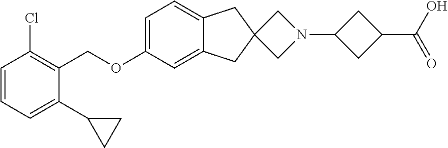 [SMILES: O=C(O)C1CC(N2CC3(Cc4ccc(OCc5c(Cl)cccc5C5CC5)cc4C3)C2)C1]

¹H NMR (600 MHz, DMSO-d⁶) δ 7.37-7.28 (m, 2H), 7.11 (d, J=8.2 Hz, 1H), 7.03 (dd, J=7.4, 1.6 Hz, 1H), 6.94 (d, J=2.5 Hz, 1H), 6.81 (dd, J=8.2, 2.5 Hz, 1H), 5.25 (s, 2H), 3.16 (s, 5H), 2.99 (s, 4H), 2.69 (q, J=8.4 Hz, 1H), 2.17 (d, J=9.4 Hz, 2H), 2.10-2.02 (m, 1H), 1.97-1.89 (m, 2H), 0.95-0.87 (m, 2H), 0.74-0.66 (m, 2H).

MS: Calculated mass (C26H28ClNO3): 437.18, found mass. M+H=438/440

### Example 92. 3-(5′-((2,6-dichlorobenzyl)oxy)-1′,3′-dihydrospiro[azetidine-3,2′-inden]-1-yl)cyclobutanecarboxylic acid

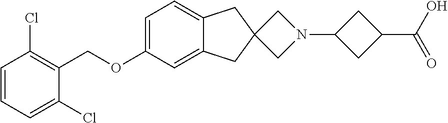 [SMILES: O=C(O)C1CC(N2CC3(Cc4ccc(OCc5c(Cl)cccc5Cl)cc4C3)C2)C1]

¹H NMR (600 MHz, DMSO-d⁶) δ 7.56 (d, J=8.1 Hz, 2H), 7.47 (dd, J=8.7, 7.5 Hz, 1H), 7.11 (d, J=8.2 Hz, 1H), 6.93 (d, J=2.5 Hz, 1H), 6.80 (dd, J=8.2, 2.5 Hz, 1H), 5.16 (s, 2H), 3.20 (s, 4H), 3.17 (t, J=7.4 Hz, 1H), 2.99 (s, 2H), 2.71 (p, J=8.4 Hz, 1H), 2.22-2.10 (m, 2H), 2.03-1.90 (m, 2H).

MS: Calculated mass (C23H23Cl2NO3): 431.11, found mass: M+H=432/434/436

### Example 93. 3-(5′-((2,5-dichlorobenzyl)oxy)-1′,3′-dihydrospiro[azetidine-3,2′-inden]-1-yl)cyclobutanecarboxylic acid

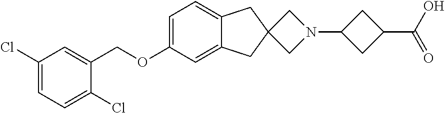 [SMILES: O=C(O)C1CC(N2CC3(Cc4ccc(OCc5cc(Cl)ccc5Cl)cc4C3)C2)C1]

¹H NMR (600 MHz, DMSO-d⁶) δ 7.64 (d, J=2.7 Hz, 1H), 7.55 (dd, J=8.6, 3.2 Hz, 1H), 7.47 (dt, J=8.5, 2.8 Hz, 1H), 7.14-7.06 (m, 1H), 6.93-6.87 (m, 1H), 6.79 (td, J=7.8, 3.8 Hz, 1H), 5.08 (d, J=4.8 Hz, 2H), 3.12 (s, 5H), 3.03 (d, J=2.9 Hz, 2H), 3.00-2.91 (m, 3H), 2.71 (dq, J=16.8, 8.4, 7.8 Hz, 1H), 2.22-2.09 (m, 2H), 2.00-1.88 (m, 2H).

MS: Calculated mass (C23H23Cl2NO3): 431.11, found mass: M+H=432/434/436

### Example 94. 3-(5′-((2-chloro-6-ethylbenzyl)oxy)-1′,3′-dihydrospiro[azetidine-3,2′-inden]-1-yl)cyclobutanecarboxylic acid

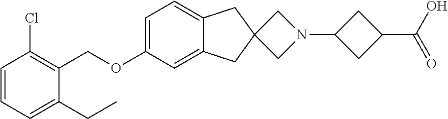 [SMILES: CCc1cccc(Cl)c1COc1ccc2c(c1)CC1(C2)CN(C2CC(C(=O)O)C2)C1]

¹H NMR (600 MHz, DMSO-d⁶) δ 7.40-7.32 (m, 2H), 7.28 (dd, J=6.0, 3.0 Hz, 1H), 7.12 (d, J=8.2 Hz, 1H), 6.93 (d, J=2.5 Hz, 1H), 6.80 (dd, J=8.2, 2.5 Hz, 1H), 5.09 (s, 2H), 3.23 (d, J=21.9 Hz, 6H), 3.06 (s, 2H), 3.00 (s, 2H), 2.71 (q, J=7.6 Hz, 3H), 2.25-2.12 (m, 2H), 1.96 (qd, J=8.5, 2.5 Hz, 2H), 1.16 (t, J=7.5 Hz, 3H).

MS: Calculated mass (C25H28ClNO3): 425.18, found mass: M+H=426/428

### Example 95. 3-(5′-((6-(cyclopentyloxy)pyridin-3-yl)ethynyl)-1′,3′-dihydrospiro[azetidine-3,2′-inden]-1-yl)cyclobutanecarboxylic acid

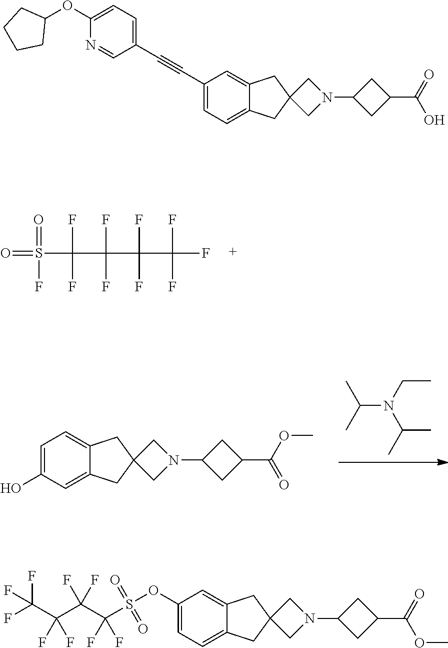

In a 100 mL 3 neck-flask methyl 3-(5′-hydroxy-1′,3′-dihydrospiro[azetidine-3,2′-inden]-1-yl)cyclobutanecarboxylate (1 g, 3.48 mmol, prepared as described for example 83) was dissolved in CH₂Cl₂ (30 mL) to give a colorless solution. N,N-diisopropylethylamine (2.377 mL, 13.92 mmol) was added. The reaction mixture was cooled down to 0° C. Nonafluorobutanesulfonyl fluoride (1.563 mL, 8.70 mmol) was added slowly. The reaction mixture was stirred for 1 h at 0° C.

The reaction mixture was diluted with CH₂Cl₂ and water was added. The mixture was stirred at RT for 10 min. A small amount of sat. NH₄Cl-solution was added. After phase separation the organic layer was washed with saturated NaCl, dried over MgSO₄, filtered and evaporated. The residue was purified using the Isco-Combiflash (12 g, 0-10% MeOH in CH₂Cl₂). The crude product was purified using the Isco-Combiflash (24 g, 0-10% MeOH in CH₂Cl₂, 35 mL/min).

Yield: 980 mg yellow oil

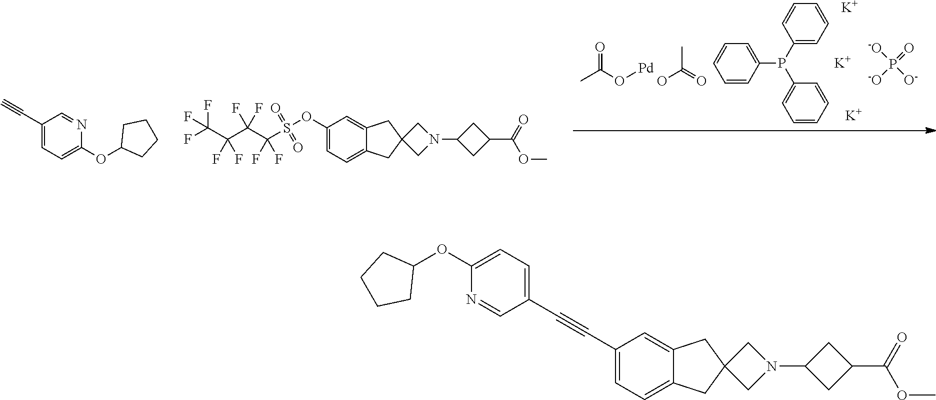

A 25 mL 3-neck round-bottom flask was charged with palladium(II) acetate (3 mg, 0.013 mmol), triphenylphosphine (12 mg, 0.046 mmol) and potassium phosphate tribasic (30 mg, 0.141 mmol) in dimethylsulfoxide (2 mL) under argon atmosphere and the mixture stirred for 30 min. In a second flask 2-(cyclopentyloxy)-5-ethynylpyridine (30 mg, 0.160 mmol) and methyl 3-(5′-(((perfluorobutyl)sulfonyl)oxy)-1′,3′-dihydrospiro[azetidine-3,2′-inden]-1-yl)cyclobutanecarboxylate (60 mg, 0.105 mmol) were dissolved in dimethyl sulfoxide (DMSO) (2 mL) and dried under argon for 30 min. This solution was put into the 3-neck flask via syringe and heated to 80° C. for 60 min.

The reaction mixture was diluted with CH₂Cl₂ and water. The mixture was stirred at RT for 10 min. After phase separation with the organic layer was washed successively with water and saturated sodium chloride solution. The organic phase was dried over MgSO₄, filtered and evaporated. The residue was purified using the Isco-Combiflash (4 g, 0-10% MeOH in CH₂Cl₂, 18 mL/min)

Yield: 26 mg brown oil

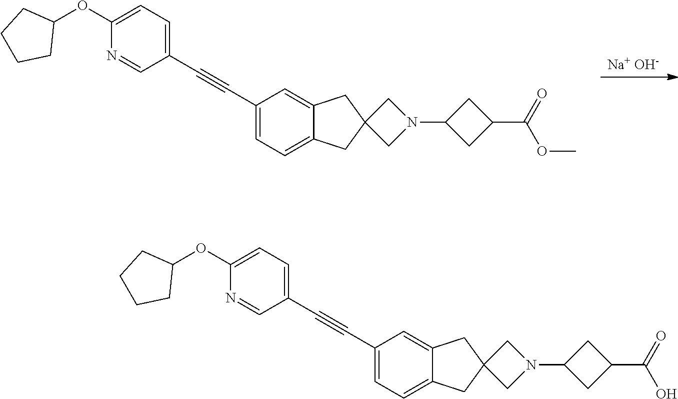 [SMILES: "Na+ OH-" ; COC(=O)C1CC(N2CC3(Cc4ccc(C#Cc5ccc(OC6CCCC6)nc5)cc4C3)C2)C1 ; O=C(O)C1CC(N2CC3(Cc4ccc(C#Cc5ccc(OC6CCCC6)nc5)cc4C3)C2)C1]

In a 50 mL round bottom flask methyl 3-(5′-((6-(cyclopentyloxy)pyridin-3-yl)ethynyl)-1′,3′-dihydrospiro[azetidine-3,2′-inden]-1-yl)cyclobutanecarboxylate (26 mg, 0.057 mmol) was dissolved in THF (0.5 mL) and MeOH (0.5 mL) to give a yellow solution. 2M NaOH (0.2 mL, 0.400 mmol) was added. The reaction mixture was stirred overnight at RT.

The reaction mixture was evaporated. The residue was dissolved in water and neutralized with 0.2 mL 2n HCl. CH₂Cl₂ was added. After phase separation with a Chromabond PTS-cartridge the organic layer was evaporated. The residue was purified using the Isco-Combiflash (4 g, 0-30% MeOH in CH₂Cl₂, 18 mL/min)

Yield: 5 mg yellow foam

¹H NMR (600 MHz, DMSO-d⁶) δ 8.34 (d, J=2.3 Hz, 1H), 7.80 (dd, J=8.6, 2.4 Hz, 1H), 7.37 (s, 1H), 7.31 (dd, J=7.7, 1.5 Hz, 1H), 7.25 (d, J=7.8 Hz, 1H), 6.80 (d, J=8.6 Hz, 1H), 5.38 (tt, J=6.0, 2.8 Hz, 1H), 3.16 (s, 4H), 3.12 (q, J=7.3 Hz, 1H), 2.70 (p, J=8.5 Hz, 1H), 2.16 (dtd, J=9.6, 7.4, 2.5 Hz, 2H), 2.00-1.89 (m, 4H), 1.70 (qdd, J=7.2, 6.0, 5.0, 2.7 Hz, 4H), 1.60 (tdd, J=9.5, 5.1, 2.9 Hz, 2H).

MS: Calculated mass (C28H30N2O3): 442.23, found mass: M+H=443

Examples 96-103 were prepared analogous to example 95:

### Example 96. 3-(5′-((6-isopropoxypyridin-3-yl)ethynyl)-1′,3′-dihydrospiro[azetidine-3,2′-inden]-1-yl)cyclobutanecarboxylic acid

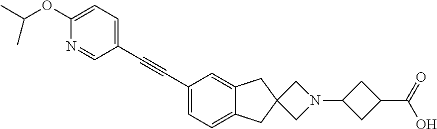 [SMILES: CC(C)Oc1ccc(C#Cc2ccc3c(c2)CC2(C3)CN(C3CC(C(=O)O)C3)C2)cn1]

¹H NMR (600 MHz, DMSO-d⁶) δ 8.34 (d, J=2.3 Hz, 1H), 7.81 (dd, J=8.5, 2.4 Hz, 1H), 7.37 (s, 1H), 7.31 (dd, J=7.7, 1.6 Hz, 1H), 7.25 (d, J=7.8 Hz, 1H), 6.79 (d, J=8.6 Hz, 1H), 5.26 (hept, J=6.2 Hz, 1H), 3.15 (q, J=7.5 Hz, 1H), 3.08 (d, J=4.2 Hz, 4H), 2.70 (p, J=8.5 Hz, 1H), 2.48 (s, 1H), 2.21-2.10 (m, 2H), 1.94 (qd, J=8.6, 2.5 Hz, 2H), 1.30 (d, J=6.2 Hz, 6H), 1.26 (dt, J=15.1, 7.1 Hz, 1H).

MS: Calculated mass (C26H28N2O3): 416.21, found mass: M+H=417

### Example 97. 3-(5′-((4-isopropoxyphenyl)ethynyl)-1′,3′-dihydrospiro[azetidine-3,2′-inden]-1-yl)cyclobutanecarboxylic acid

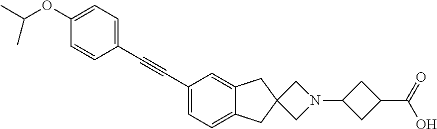 [SMILES: CC(C)Oc1ccc(C#Cc2ccc3c(c2)CC2(C3)CN(C3CC(C(=O)O)C3)C2)cc1]

¹H NMR (600 MHz, DMSO-d⁶) δ 7.43 (d, J=8.6 Hz, 2H), 7.35 (s, 1H), 7.28 (d, J=7.8 Hz, 1H), 7.23 (d, J=7.8 Hz, 1H), 6.94 (d, J=8.4 Hz, 2H), 4.66 (hept, J=6.1 Hz, 1H), 3.21 (s, 4H), 3.16 (d, J=8.8 Hz, 1H), 3.08 (d, J=3.4 Hz, 4H), 2.71 (p, J=8.5 Hz, 1H), 2.22-2.11 (m, 2H), 1.99-1.90 (m, 2H), 1.27 (d, J=6.0 Hz, 6H), 1.23 (s, 1H)

MS: Calculated mass (C27H29NO3): 415.21, found mass: M+H=416

### Example 98. 3-(5′-((4-ethoxyphenyl)ethynyl)-1′,3′-dihydrospiro[azetidine-3,2′-inden]-1-yl)cyclobutanecarboxylic acid

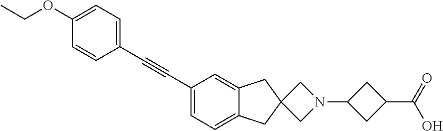 [SMILES: CCOc1ccc(C#Cc2ccc3c(c2)CC2(C3)CN(C3CC(C(=O)O)C3)C2)cc1]

¹H NMR (600 MHz, DMSO-d⁶) δ 7.47-7.42 (m, 2H), 7.35 (s, 1H), 7.28 (dd, J=7.7, 1.5 Hz, 1H), 7.23 (d, J=7.8 Hz, 1H), 6.98-6.93 (m, 2H), 4.06 (q, J=7.0 Hz, 2H), 3.22 (d, J=23.7 Hz, 5H), 3.09 (d, J=3.8 Hz, 4H), 2.71 (p, J=8.6 Hz, 1H), 2.18 (dtd, J=15.0, 12.4, 11.1, 6.6 Hz, 2H), 2.05-1.92 (m, 2H), 1.33 (t, J=7.0 Hz, 3H)

MS: Calculated mass (C26H27NO3): 401.20, found mass: M+H=402

### Example 99. 3-(5′-((6-(cyclopropylmethoxy)pyridin-3-yl)ethynyl)-1′,3′-dihydrospiro[azetidine-3,2′-inden]-1-yl)cyclobutanecarboxylic acid

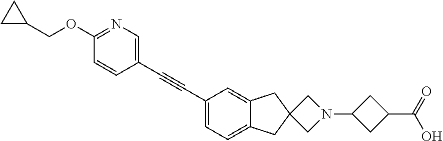 [SMILES: O=C(O)C1CC(N2CC3(Cc4ccc(C#Cc5ccc(OCC6CC6)nc5)cc4C3)C2)C1]

¹H NMR (600 MHz, Methanol-d⁴) δ 8.24 (d, J=2.3 Hz, 1H), 7.74 (dd, J=8.6, 2.4 Hz, 1H), 7.37 (d, J=3.5 Hz, 1H), 7.32 (dt, J=7.9, 2.0 Hz, 1H), 7.23 (d, J=7.9 Hz, 1H), 6.78 (d, J=8.6 Hz, 1H), 4.13 (d, J=7.1 Hz, 2H), 4.06 (s, 4H), 3.82 (p, J=7.1 Hz, 1H), 3.29 (d, J=4.5 Hz, 4H), 2.86 (p, J=8.2 Hz, 1H), 2.56 (dddd, J=19.0, 13.2, 7.7, 3.6 Hz, 2H), 2.34-2.20 (m, 2H), 1.27 (tt, J=7.6, 4.7 Hz, 1H), 0.64-0.55 (m, 2H), 0.34 (dt, J=6.2, 4.4 Hz, 2H).

MS: Calculated mass (C27H28N2O3): 428.21, found mass: M+H=429

### Example 100. 3-(5′-((4-ethoxy-3-fluorophenyl)ethynyl)-1′,3′-dihydrospiro[azetidine-3,2′-inden]-1-yl)cyclobutanecarboxylic acid

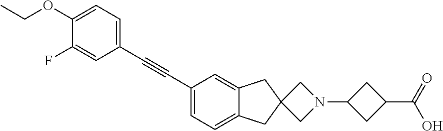 [SMILES: CCOc1ccc(C#Cc2ccc3c(c2)CC2(C3)CN(C3CC(C(=O)O)C3)C2)cc1F]

¹H NMR (600 MHz, Methanol-d⁴) δ 7.36 (d, J=1.4 Hz, 1H), 7.31 (dd, J=7.7, 1.4 Hz, 1H), 7.26-7.18 (m, 3H), 7.06 (t, J=8.6 Hz, 1H), 4.13 (q, J=7.0 Hz, 2H), 4.05 (s, 4H), 3.86-3.78 (m, 1H), 3.29 (d, J=4.5 Hz, 4H), 2.90-2.82 (m, 1H), 2.58 (dddd, J=11.6, 8.6, 5.0, 2.1 Hz, 2H), 2.19-2.11 (m, 2H), 1.42 (t, J=7.0 Hz, 3H).

25 protons

MS: Calculated mass (C26H26FNO3): 419.19, found mass: M+H=420

### Example 101. 3-(5′-((4-methoxy-2-methylphenyl)ethynyl)-1′,3′-dihydrospiro[azetidine-3,2′-inden]-1-yl)cyclobutanecarboxylic acid

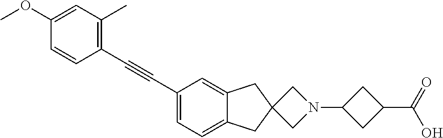 [SMILES: COc1ccc(C#Cc2ccc3c(c2)CC2(C3)CN(C3CC(C(=O)O)C3)C2)c(C)c1]

¹H NMR (500 MHz, DMSO-d⁶) δ 7.42-7.33 (m, 2H), 7.28 (dd, J=7.7, 1.5 Hz, 1H), 7.23 (d, J=7.7 Hz, 1H), 6.90 (d, J=2.7 Hz, 1H), 6.79 (dd, J=8.5, 2.7 Hz, 1H), 3.77 (s, 3H), 3.10 (d, J=32.4 Hz, 8H), 2.69 (p, J=8.4 Hz, 1H), 2.47 (p, J=1.8 Hz, 1H), 2.42 (s, 3H), 2.21-2.11 (m, 2H), 1.92 (qd, J=8.3, 2.5 Hz, 2H).

26 protons

MS: Calculated mass (C26H27NO3): 401.20, found mass: M+H=402

### Example 102. 3-(5′-((4-methylcyclohexyl)ethynyl)-1′,3′-dihydrospiro[azetidine-3,2′-inden]-1-yl)cyclobutanecarboxylic acid

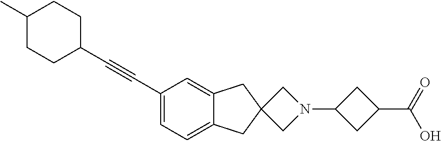 [SMILES: CC1CCC(C#Cc2ccc3c(c2)CC2(C3)CN(C3CC(C(=O)O)C3)C2)CC1]

¹H NMR (600 MHz, DMSO-d⁶) δ 7.40-7.00 (m, 3H), 3.10 (s, 6H), 3.01 (s, 1H), 2.92 (t, J=4.0 Hz, 1H), 2.68 (qd, J=8.4, 1.5 Hz, 1H), 2.15 (dddd, J=11.9, 9.3, 7.1, 2.2 Hz, 2H), 1.91 (dddd, J=17.3, 14.1, 10.2, 3.0 Hz, 3H), 1.73 (dd, J=13.3, 3.7 Hz, 1H), 1.69-1.63 (m, 1H), 1.54 (ddq, J=16.8, 12.5, 3.8 Hz, 3H), 1.36 (dtd, J=24.6, 11.7, 10.5, 3.3 Hz, 3H), 0.95 (td, J=12.5, 11.7, 3.4 Hz, 1H), 0.88 (dd, J=18.1, 6.5 Hz, 3H)

29 protons

MS: Calculated mass (C25H31NO2): 377.24, found mass: M+H=378

### Example 103. 3-(5′-((6-((1,1,1-trifluoropropan-2-yl)oxy)pyridin-3-yl)ethynyl)-1′,3′-dihydrospiro[azetidine-3,2′-inden]-1-yl)cyclobutanecarboxylic acid

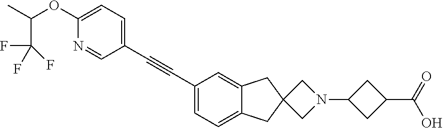 [SMILES: CC(Oc1ccc(C#Cc2ccc3c(c2)CC2(C3)CN(C3CC(C(=O)O)C3)C2)cn1)C(F)(F)F]

¹H NMR (600 MHz, DMSO-d⁶) δ 8.40 (d, J=2.5 Hz, 1H), 7.94 (dd, J=8.6, 2.3 Hz, 1H), 7.40 (s, 1H), 7.33 (dd, J=7.6, 1.6 Hz, 1H), 7.26 (d, J=7.9 Hz, 1H), 7.01 (d, J=8.6 Hz, 1H), 5.91 (h, J=6.7 Hz, 1H), 3.17 (s, 5H), 3.08 (d, J=3.8 Hz, 4H), 2.70 (p, J=8.5 Hz, 1H), 2.56-2.52 (m, 1H), 2.17 (q, J=9.5 Hz, 2H), 1.97-1.89 (m, 2H), 1.47 (d, J=6.5 Hz, 3H).

25 protons

MS: Calculated mass (C26H25F3N2O3): 470.18, found mass: M+H=471

### Example 104. 3-(5′-((2,3-dihydro-1H-inden-1-yl)oxy)-1′,3′-dihydrospiro[azetidine-3,2′-inden]-1-yl)cyclobutanecarboxylic acid

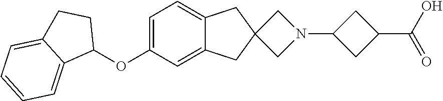 [SMILES: O=C(O)C1CC(N2CC3(Cc4ccc(OC5CCc6ccccc65)cc4C3)C2)C1]

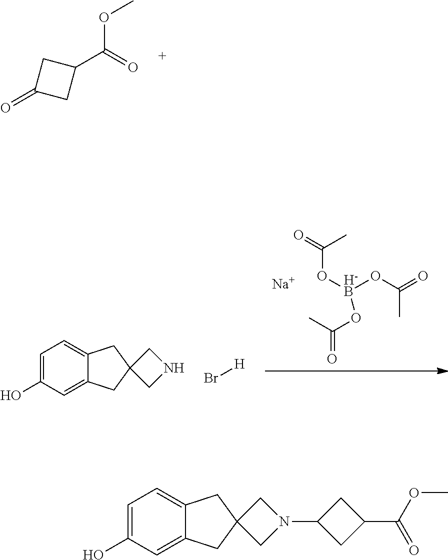

In a 100 mL round-bottomed flask 1′,3′-dihydrospiro[azetidine-3,2′-inden]-5′-ol hydrobromide (1 g, 3.90 mmol) was suspended in THF (40 mL). Methyl 3-oxocyclobutanecarboxylate (1.0 g, 7.81 mmol) was added. The mixture was stirred for 60 min at RT. Sodium triacetoxyborohydride (1.655 g, 7.81 mmol) was added. The reaction mixture was stirred overnight at RT.

The reaction mixture was poured in an Erlenmeyer flask containing 100 mL CH₂Cl₂ and 10 mL water. The mixture was stirred for 30 min. The organic layer was diluted with CH₂Cl₂ and washed twice with NaHCO₃-solution, 1× with saturated NaCl, dried over MgSO₄, filtered and evaporated.

Yield: 1.04 g orange oil

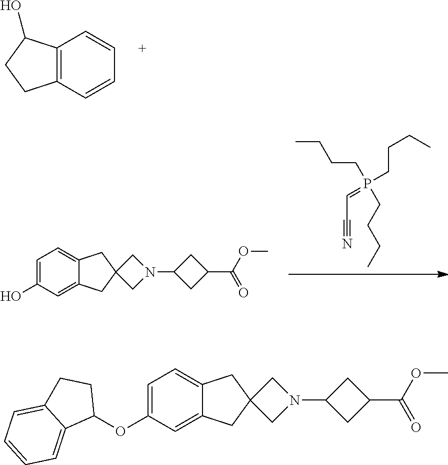 [SMILES: OC1CCc2ccccc21 ; COC(=O)C1CC(N2CC3(Cc4ccc(O)cc4C3)C2)C1 ; CCCCP(=CC#N)(CCCC)CCCC ; COC(=O)C1CC(N2CC3(Cc4ccc(OC5CCc6ccccc65)cc4C3)C2)C1]

In a microwave flask methyl 3-(5′-hydroxy-1′,3′-dihydrospiro[azetidine-3,2′-inden]-1-yl)cyclobutanecarboxylate (50 mg, 0.174 mmol) and 1-INDANOL (60 mg, 0.447 mmol) were dissolved in toluene (PhCH₃) (3 ml). Cyanomethylenetributylphosphorane (0.5 mL, 0.5 mmol) was added. The mixture was stirred for 8 h at 80° C. in the Biotage microwave.

The reaction mixture was evaporated. The residue was dissolved in CH₂Cl₂ and washed once with water. After phase separation with a Chromabond PTS cartridge the organic layer was evaporated. The residue was purified using the Isco-Combiflash (4 g, 0-10% MeOH in CH₂Cl₂, 18 mL/min).

Yield: 73 mg light brown solid

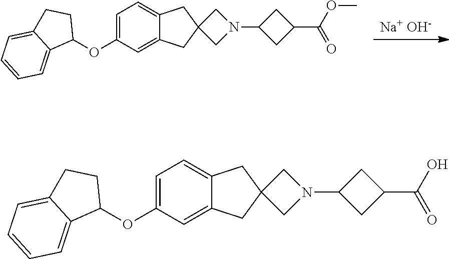 [SMILES: "Na+ OH-" ; COC(=O)C1CC(N2CC3(Cc4ccc(OC5CCc6ccccc65)cc4C3)C2)C1 ; O=C(O)C1CC(N2CC3(Cc4ccc(OC5CCc6ccccc65)cc4C3)C2)C1]

In a 50 mL round bottom flask methyl 3-(5′-((2,3-dihydro-1H-inden-1-yl)oxy)-1′,3′-dihydrospiro[azetidine-3,2′-inden]-1-yl)cyclobutanecarboxylate (73 mg, 0.090 mmol) was dissolved in THF (0.5 mL) and MeOH (0.5 mL) to give a yellow solution. 2M NaOH (0.25 mL, 0.500 mmol) was added. The reaction mixture was stirred at RT overnight.

The reaction mixture was evaporated. The residue was dissolved in water and neutralized with 0.25 mL 2n NaOH. CH₂Cl₂ was added, the mixture was stirred at RT for 1 h. After phase separation with a Chromabond PTS cartridge the organic layer was evaporated. The residue was purified using the Isco-Combiflash (4 g, 0-30% MeOH in CH₂Cl₂, 18 mL/min).

Yield: 10 mg white foam

¹H NMR (600 MHz, Methanol-d⁴) δ 7.36-7.32 (m, 1H), 7.31-7.24 (m, 2H), 7.22-7.16 (m, 1H), 7.14 (d, J=8.2 Hz, 1H), 6.91 (d, J=2.4 Hz, 1H), 6.83 (dd, J=8.2, 2.4 Hz, 1H), 5.76 (dd, J=6.7, 4.2 Hz, 1H), 4.04 (s, 4H), 3.84-3.78 (m, 1H), 3.23 (d, J=24.0 Hz, 4H), 3.08 (ddd, J=15.7, 8.5, 5.6 Hz, 1H), 2.94-2.81 (m, 2H), 2.62-2.49 (m, 3H), 2.17-2.06 (m, 3H). 26 protons MS: Calculated mass (C25H27NO3): 389.20, found mass: M+H=390

Examples 105-107 were prepared analogously to example 104:

### Example 105. 3-(5′-(2-(trifluoromethyl)phenethoxy)-1′,3′-dihydrospiro[azetidine-3,2′-inden]-1-yl)cyclobutanecarboxylic acid

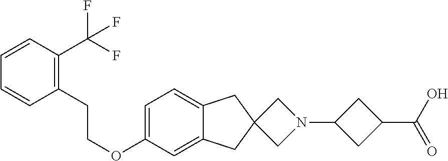 [SMILES: O=C(O)C1CC(N2CC3(Cc4ccc(OCCc5ccccc5C(F)(F)F)cc4C3)C2)C1]

¹H NMR (600 MHz, Methanol-d₄) δ 7.67 (d, J=7.9 Hz, 1H), 7.58-7.53 (m, 2H), 7.40 (td, J=7.0, 6.0, 2.3 Hz, 1H), 7.09 (d, J=8.2 Hz, 1H), 6.79 (d, J=2.4 Hz, 1H), 6.72 (dd, J=8.3, 2.5 Hz, 1H), 4.15 (t, J=6.9 Hz, 2H), 4.01 (s, 4H), 3.79 (p, J=7.0 Hz, 1H), 3.24 (td, J=7.0, 1.3 Hz, 2H), 3.21 (s, 2H), 3.17 (s, 2H), 2.84 (p, J=8.0 Hz, 1H), 2.62-2.49 (m, 2H), 2.25-2.17 (m, 2H).

MS: Calculated mass (C25H26F3NO3): 445.19, found mass: M+H=446

### Example 106. 3-(5′-((7-chloro-2,3-dihydro-1H-inden-1-yl)oxy)-1′,3′-dihydrospiro[azetidine-3,2′-inden]-1-yl)cyclobutanecarboxylic acid

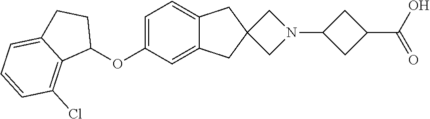 [SMILES: O=C(O)C1CC(N2CC3(Cc4ccc(OC5CCc6cccc(Cl)c65)cc4C3)C2)C1]

¹H NMR (600 MHz, Methanol-d4) δ 7.30 (t, J=7.7 Hz, 1H), 7.27-7.21 (m, 2H), 7.14 (d, J=8.2 Hz, 1H), 6.89 (d, J=2.3 Hz, 1H), 6.81 (dd, J=8.2, 2.4 Hz, 1H), 5.79 (dd, J=6.4, 1.5 Hz, 1H), 4.05 (s, 4H), 3.82 (p, J=7.3 Hz, 1H), 3.25 (s, 2H), 3.21 (s, 2H), 3.20-3.15 (m, 1H), 2.96 (ddd, J=16.4, 9.0, 2.4 Hz, 1H), 2.86 (p, J=8.0 Hz, 1H), 2.62-2.53 (m, 2H), 2.46-2.36 (m, 1H), 2.27-2.18 (m, 3H).

MS: Calculated mass (C25H26ClNO3): 423.16, found mass: M+H=424/426

### Example 107. 3-(5′-((4-ethoxy-3,5-difluorobenzyl)oxy)-1′,3′-dihydrospiro[azetidine-3,2′-inden]-1-yl)cyclobutanecarboxylic acid

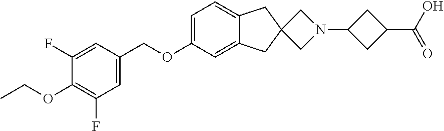 [SMILES: CCOc1c(F)cc(COc2ccc3c(c2)CC2(C3)CN(C3CC(C(=O)O)C3)C2)cc1F]

¹H NMR (600 MHz, Chloroform-d) δ 7.10 (d, J=8.2 Hz, 1H), 7.00-6.93 (m, 2H), 6.79 (d, J=2.4 Hz, 1H), 6.76 (dd, J=8.2, 2.5 Hz, 1H), 4.94 (s, 2H), 4.20 (q, J=7.1 Hz, 2H), 3.29 (ddq, J=6.2, 4.1, 1.9 Hz, 1H), 3.20-3.07 (m, 5H), 2.53 (dddd, J=13.1, 8.4, 5.8, 2.6 Hz, 2H), 2.02 (d, J=12.7 Hz, 2H), 1.39 (t, J=7.0 Hz, 3H).

MS: Calculated mass (C25H27F2NO4): 443.19, found mass: M+H=444

### Example 108. 1-((5′-(4-((4-chlorobenzyl)oxy)phenyl)-1′,3′-dihydrospiro[azetidine-3,2′-inden]-1-yl)methyl)cyclopropanecarboxylic acid

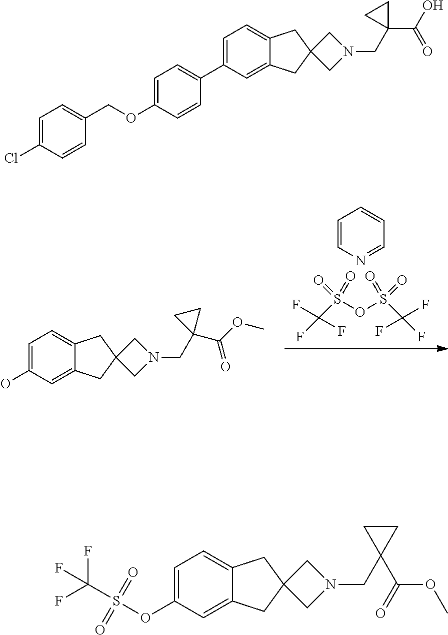

In a Schlenck flask methyl 1-((5′-hydroxy-1′,3′-dihydrospiro[azetidine-3,2′-inden]-1-yl)methyl)cyclopropanecarboxylate (104 mg, 0.362 mmol, prepared as described for example 28) was dissolved in CH₂Cl₂ (3 mL) to give a light yellow solution. Pyridine (0.07 mL, 0.865 mmol) was added. The mixture was cooled to 0° C. and at this temperature trifluoromethanesulfonic anhydride (0.4 mL, 0.400 mmol) was added dropwise. The color of the solution turned to yellow. The reaction mixture was diluted with CH₂Cl₂ and washed twice with sat. NH₄Cl-solution and once with saturated sodium chloride solution. The organic layer was dried over MgSO₄, filtered and evaporated. The residue was purified by flash chromatography (4 g, 0-10% MeOH in CH₂Cl₂).

Yield: 75 mg (49%), yellow oil.

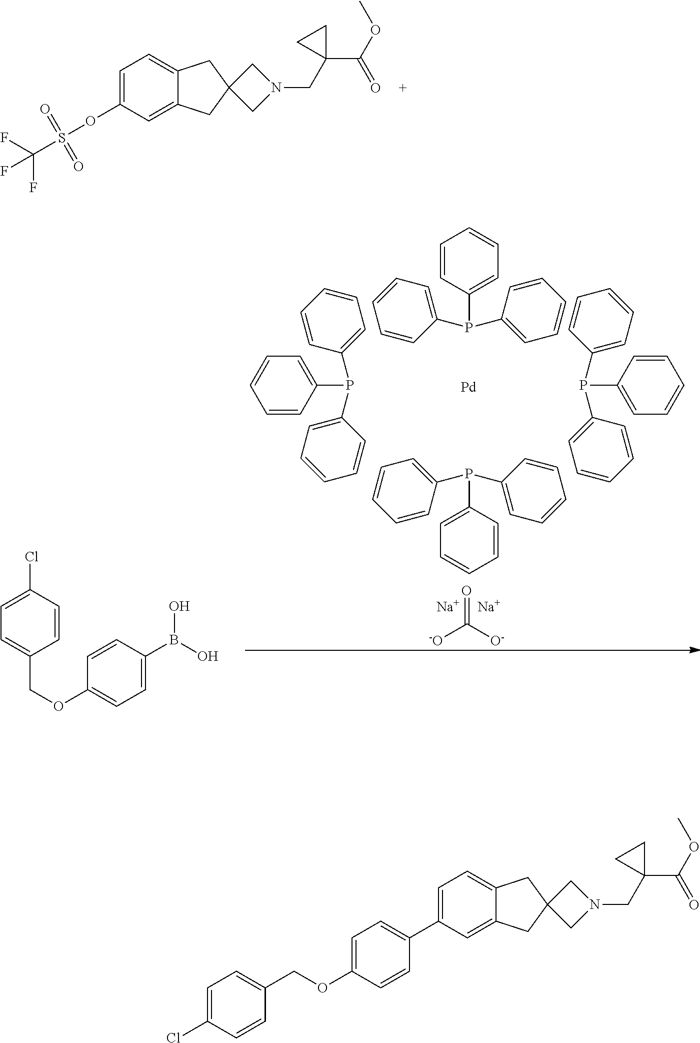

methyl 1-((5′-(((trifluoromethyl)sulfonyl)oxy)-1′,3′-dihydrospiro[azetidine-3,2′-inden]-1-yl)methyl)cyclopropanecarboxylate (90 mg, 0.215 mmol) was dissolved in dry DMF (3 mL) and 4-(4′-chlorobenzyloxy)phenylboronic acid (67.7 mg, 0.258 mmol) and 10% sodium carbonate solution (517 μL, 0.537 mmol) followed by Tetrakis(triphenylphosphine)palladium(0) (9.9 mg, 8.6 μmol) were added. The reaction mixture was heated under stirring in the microwave to 120° C. for 30 min. The organic solvent was removed in vacuo, the residue was treated with CH₂Cl₂ and water. The organic phase was washed with water and dried (MgSO₄). The solvent was evaporated in vacuo and the crude product purified by flash chromatography (silica, n-heptane, ethyl acetate).

Yield: 38.6 mg, yellow oil (36.9%)

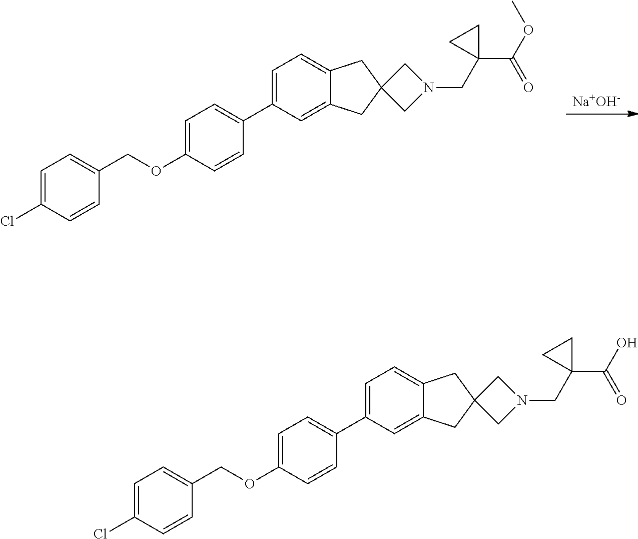 [SMILES: "Na+OH-" ; COC(=O)C1(CN2CC3(Cc4ccc(-c5ccc(OCc6ccc(Cl)cc6)cc5)cc4C3)C2)CC1 ; O=C(O)C1(CN2CC3(Cc4ccc(-c5ccc(OCc6ccc(Cl)cc6)cc5)cc4C3)C2)CC1]

methyl 1-((5′-(4-((4-chlorobenzyl)oxy)phenyl)-1′,3′-dihydrospiro[azetidine-3,2′-inden]-1-yl)methyl)cyclopropanecarboxylate (38 mg, 0.078 mmol) was dissolved in THF (1 mL) and MeOH (1 mL). 1N NaOH (800 μL, 0.8 mmol) was added under stirring at RT. Stirring was continued at RT for 20 h. 1N HCL (800 μL, 0.8 mmol) was added followed by water (1 mL). The organic phase was evaporated in vacuo. The product precipitated as colorless solid. 1-((5′-(4-((4-chlorobenzyl)oxy)phenyl)-1′,3′-dihydrospiro[azetidine-3,2′-inden]-1-yl)methyl)cyclopropanecarboxylic acid was obtained by filtration and dried over phosphorus pentoxide in vacuo at 50° C. The product was purified by flash chromatography (silica, CH₂Cl₂/MeOH 95/5).

Yield: 8.5 mg, colorless solid (23%).

1H NMR (600 MHz, DMSO-d₆) δ 7.58-7.53 (m, 2H), 7.52-7.43 (m, 6H), 7.39 (dd, J=7.9, 1.7 Hz, 1H), 7.27 (d, J=8.0 Hz, 1H), 7.10-7.04 (m, 2H), 5.15 (s, 2H), 4.02 (s, 5H), 3.23 (d, J=17.1 Hz, 5H), 1.14 (t, J=4.0 Hz, 2H), 0.98 (s, 2H).

28 Protons

Calculated mass (C29H28ClNO3): 473.991, found mass: M+H⁺=474/476

### Example 109. 2-(5′-((2,6-dichlorobenzyl)oxy)-1′,3′-dihydrospiro[azetidine-3,2′-inden]-1-yl)acetic acid

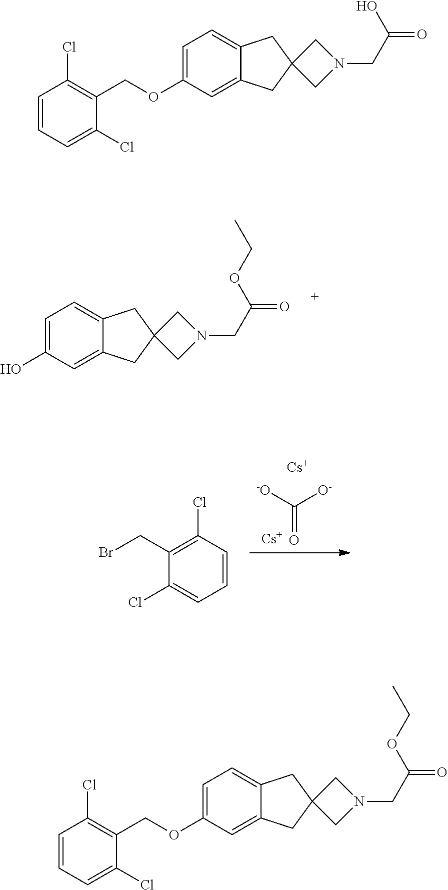

Ethyl 2-(5′-hydroxy-1′,3′-dihydrospiro[azetidine-3,2′-inden]-1-yl)acetate (52 mg, 0.199 mmol, prepared as described for example 8) was dissolved in dry DMF (3 mL). Cs₂CO₃ (78 mg, 0.239 mmol) and 2,6-dichlorobenzyl bromide (52.5 mg, 0.219 mmol) were added under stirring. The reaction mixture was stirred at RT for 2 h. The organic solvent was removed in vacuo and the residue treated with CH₂Cl₂ and water. The organic phase was washed with water and dried (MgSO₄). The crude product was purified by flash chromatography (silica, CH₂Cl₂, MeOH).

Yield: 44 mg (52.6%), pale yellow oil.

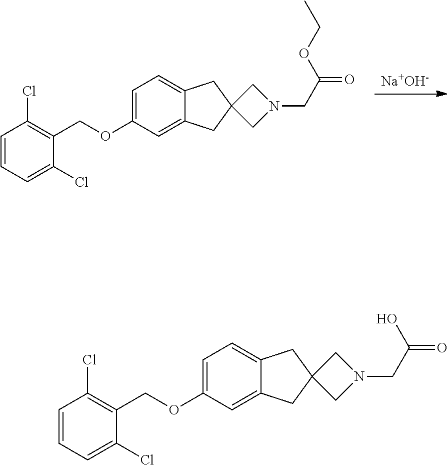 [SMILES: "Na+OH-" ; CCOC(=O)CN1CC2(Cc3ccc(OCc4c(Cl)cccc4Cl)cc3C2)C1 ; O=C(O)CN1CC2(Cc3ccc(OCc4c(Cl)cccc4Cl)cc3C2)C1]

ethyl 2-(5′-((2,6-dichlorobenzyl)oxy)-1′,3′-dihydrospiro[azetidine-3,2′-inden]-1-yl)acetate (44 mg, 0.105 mmol) was dissolved in THF (2 mL) and MeOH (2 mL). 1M NaOH (1 mL, 1.0 mmol) was added under stirring. The reaction mixtures was stirred under RT for 20 h. 1N HCl (1 mL) was added. The organic solvent was evaporated in vacuo. The crude product was extracted with CH₂Cl₂, dried (MgSO₄) and concentrated in vacuo.

Yield: 34 mg, colorless solid (83%).

¹H NMR (600 MHz, DMSO-d₆) δ 7.56 (d, J=8.1 Hz, 2H), 7.47 (dd, J=8.7, 7.5 Hz, 1H), 7.13 (d, J=8.2 Hz, 1H), 6.95 (d, J=2.3 Hz, 1H), 6.81 (dd, J=8.2, 2.5 Hz, 1H), 5.17 (s, 2H), 3.81-3.76 (m, 4H), 3.42 (s, 2H), 3.13 (d, J=31.3 Hz, 4H).

18 Protons

Calculated mass (C20H19Cl2NO3): 392.276, found mass: M+H±=392

Examples 110 and 111 were prepared analogous to example 109:

### Example 110. 2-(5′-((2-cyclopropyl-6-fluorobenzyl)oxy)-1′,3′-dihydrospiro[azetidine-3,2′-inden]-1-yl)acetic acid

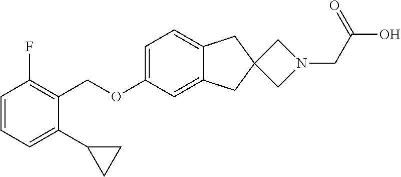 [SMILES: O=C(O)CN1CC2(Cc3ccc(OCc4c(F)cccc4C4CC4)cc3C2)C1]

¹H NMR (600 MHz, DMSO-d₆) δ 7.33 (td, J=8.1, 6.1 Hz, 1H), 7.12 (d, J=8.3 Hz, 1H), 7.06 (ddd, J=9.6, 8.2, 1.1 Hz, 1H), 6.95 (d, J=2.4 Hz, 1H), 6.90-6.79 (m, 2H), 5.16 (d, J=1.8 Hz, 2H), 3.85-3.78 (m, 4H), 3.46 (s, 2H), 3.12 (d, J=31.9 Hz, 4H), 2.05 (tt, J=8.4, 5.3 Hz, 1H), 0.99-0.88 (m, 2H), 0.74-0.66 (m, 2H).

23 Protons

Calculated mass (C23H24FNO3): 381.440, found mass: M+H⁺=382

### Example 111. 2-(5′-((2-cyclopropylbenzyl)oxy)-1′,3′-dihydrospiro[azetidine-3,2′-inden]-1-yl)acetic acid

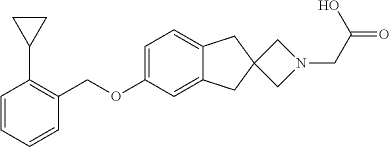 [SMILES: O=C(O)CN1CC2(Cc3ccc(OCc4ccccc4C4CC4)cc3C2)C1]

¹H NMR (600 MHz, DMSO-d₆) δ 7.38 (dd, J=7.5, 1.4 Hz, 1H), 7.25 (td, J=7.5, 1.4 Hz, 1H), 7.18 (td, J=7.4, 1.3 Hz, 1H), 7.11 (d, J=8.3 Hz, 1H), 7.01 (dd, J=7.7, 1.2 Hz, 1H), 6.93 (d, J=2.3 Hz, 1H), 6.80 (dd, J=8.2, 2.5 Hz, 1H), 5.18 (s, 2H), 3.78 (s, 4H), 3.41 (s, 2H), 3.14 (s, 2H), 3.08 (s, 2H), 2.02 (tt, J=8.6, 5.4 Hz, 2H), 0.95-0.86 (m, 2H), 0.70-0.61 (m, 2H).

25 Protons

Calculated mass (C23H25NO3): 363.450, found mass: M+H±=364

### Example 112. 1-((5′-((4-ethoxyphenyl)ethynyl)-1′,3′-dihydrospiro[azetidine-3,2′-inden]-1-yl)methyl)cyclopropanecarboxylic acid

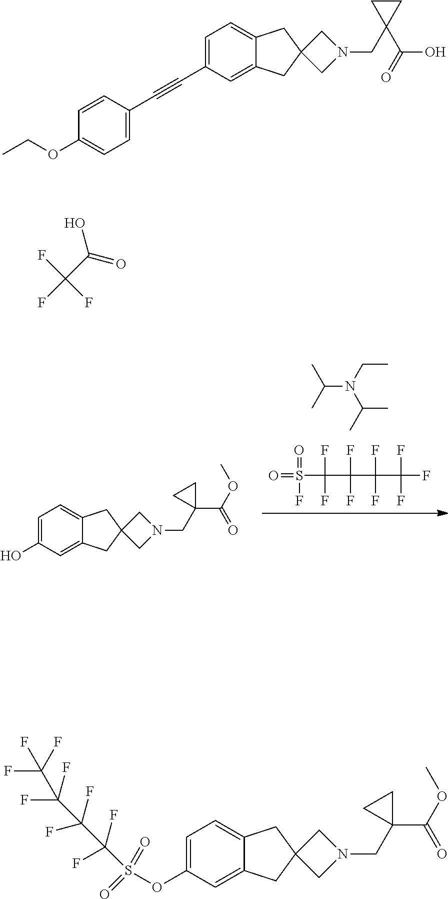

Methyl 1-((5′-hydroxy-1′,3′-dihydrospiro[azetidine-3,2′-inden]-1-yl)methyl)cyclopropanecarboxylate (1.64 g, 5.71 mmol, prepared as described for example 28) was dissolved in CH₂Cl₂ (40 mL) under an atmosphere of argon. N,N-diisopropylethylamine (3.9 mL, 22.83 mmol) was added and three reaction mixture cooled to 0° C. At that temperature Nonafluorobutanesulfonyl fluoride (2.56 mL, 14.27 mmol) was added under stirring. After stirring at 0° C. for 2 h the reaction mixture was allowed to warm to RT. Stirring was continued for 3 h. The reaction mixture was diluted with CH₂Cl₂ (50 mL) and washed with water (twice), dried (MgSO₄) and concentrated in vacuo. The crude product was purified by flash chromatography (silica, CH₂Cl₂, MeOH).

Yield: 3.1 g (95%), yellow oil.

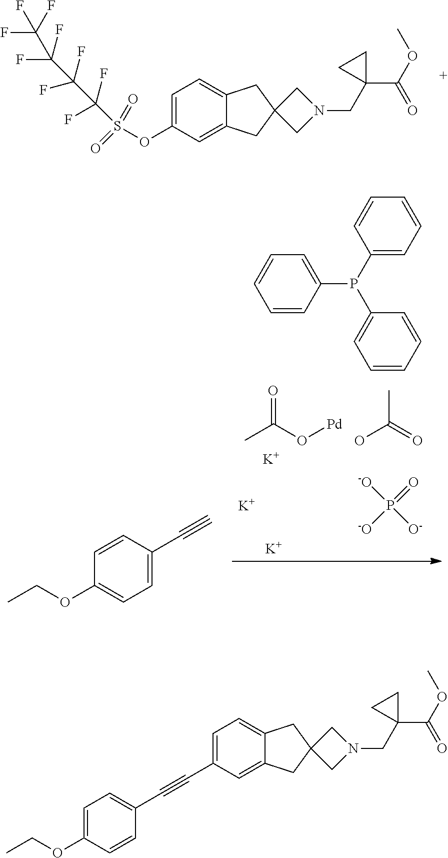

Methyl 1-((5′-(((perfluorobutyl)sulfonyl)oxy)-1′,3′-dihydrospiro[azetidine-3,2′-inden]-1-yl)methyl)cyclopropanecarboxylate (100 mg, 0.176 mmol) was dissolved in dry DMSO (2 mL) and 4-ethoxyphenylacetylene (38.5 mg, 0.263 mmol) was added under an atmosphere of argon. The reaction mixture was degased with argon for 5 min. Potassium phosphate tribasic (44.7 mg, 0.211 mmol), palladium(II) acetate (4.73 mg, 0.021 mmol) and triphenylphosphine (18.4 mg, 0.07 mmol) were added. The reaction mixture was heated under stirring and an argon atmosphere to 80° C. for 1 h. The reaction mixture was diluted with CH₂Cl₂, successively washed with water (twice) and saturated sodium bicarbonate solution. The organic phase was dried (MgSO₄) and concentrated in vacuo. The cured product was purified by flash chromatography (silica, CH₂Cl₂, MeOH).

Yield: 65 mg (89%), yellow oil.

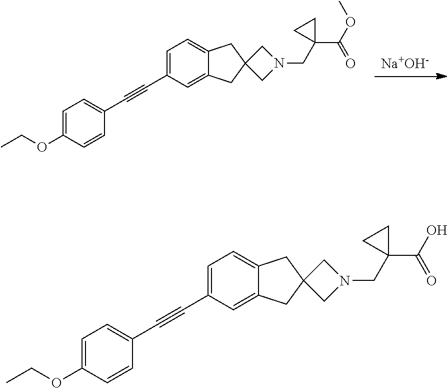 [SMILES: "Na+OH-" ; CCOc1ccc(C#Cc2ccc3c(c2)CC2(C3)CN(CC3(C(=O)OC)CC3)C2)cc1 ; CCOc1ccc(C#Cc2ccc3c(c2)CC2(C3)CN(CC3(C(=O)O)CC3)C2)cc1]

methyl 1-((5′-((4-ethoxyphenyl)ethynyl)-1′,3′-dihydrospiro[azetidine-3,2′-inden]-1-yl)methyl)cyclopropanecarboxylate (65 mg, 0.156 mmol) was dissolved in THF (3 mL) and MeOH (3 mL). 1 M NaOH (1.6 mL, 1.6 mmol) was added and the reaction mixture heated to 50° under stirring for 2 h. Stirring was continued at RT overnight. 1N HCl (1.6 ml, 1.6 mmol) was added and the reaction mixture diluted with water (5 mL). The organic solvents were removed in vacuo. The product precipitate and was obtained by filtration. 1-((5′-((4-ethoxyphenyl)ethynyl)-1′,3′-dihydrospiro[azetidine-3,2′-inden]-1-yl)methyl)cyclopropanecarboxylic acid was dried in vacuo over phosphorus pentoxide at 40° C.

Yield: 59 mg (94%), pale yellow solid.

¹H NMR (600 MHz, DMSO-d₆) δ 7.48-7.42 (m, 2H), 7.37 (d, J=1.4 Hz, 1H), 7.29 (dd, J=7.7, 1.5 Hz, 1H), 7.24 (d, J=7.8 Hz, 1H), 6.99-6.93 (m, 2H), 4.06 (q, J=7.0 Hz, 2H), 3.48 (s, 4H), 3.13 (d, J=1.8 Hz, 4H), 2.77 (s, 2H), 1.33 (t, J=7.0 Hz, 3H), 0.92 (q, J=3.7 Hz, 2H), 0.64 (q, J=3.8 Hz, 2H).

26 Protons

Calculated mass (C26H27NO3): 401.497, found mass: M+H⁺=402

Examples 113-118 were prepared analogous to example 112:

### Example 113. 1-((5′-((4-isopropoxyphenyl)ethynyl)-1′,3′-dihydrospiro[azetidine-3,2′-inden]-1-yl)methyl)cyclopropanecarboxylic acid

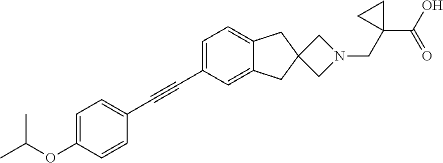 [SMILES: CC(C)Oc1ccc(C#Cc2ccc3c(c2)CC2(C3)CN(CC3(C(=O)O)CC3)C2)cc1]

¹H NMR (500 MHz, DMSO-d₆) δ 7.46-7.40 (m, 2H), 7.37 (s, 1H), 7.33-7.27 (m, 1H), 7.25 (d, J=7.7 Hz, 1H), 6.97-6.91 (m, 2H), 4.66 (p, J=6.0 Hz, 1H), 3.78 (s, 4H), 3.18 (s, 4H), 3.05 (s, 2H), 1.27 (d, J=6.0 Hz, 6H), 1.05 (q, J=3.9 Hz, 2H), 0.84 (q, J=4.1 Hz, 2H).

28 Protons

Calculated mass (C27H29NO3): 415.524, found mass: M+H⁺=416

### Example 114. 1-((5′-((6-isopropoxypyridin-3-yl)ethynyl)-1′,3′-dihydrospiro[azetidine-3,2′-inden]-1-yl)methyl)cyclopropanecarboxylic acid

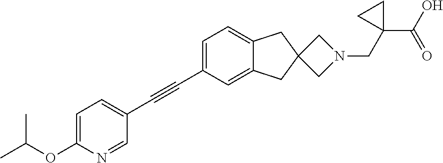 [SMILES: CC(C)Oc1ccc(C#Cc2ccc3c(c2)CC2(C3)CN(CC3(C(=O)O)CC3)C2)cn1]

¹H NMR (500 MHz, DMSO-d₆) δ 8.34 (d, J=2.4 Hz, 1H), 7.81 (dd, J=8.6, 2.4 Hz, 1H), 7.39 (d, J=1.5 Hz, 1H), 7.32 (dd, J=7.7, 1.5 Hz, 1H), 7.26 (d, J=7.7 Hz, 1H), 6.79 (d, J=8.5 Hz, 1H), 5.27 (dq, J=12.4, 6.1 Hz, 1H), 3.50 (s, 4H), 3.14 (s, 2H), 2.79 (s, 2H), 1.30 (d, J=6.2 Hz, 6H), 0.94 (q, J=3.8 Hz, 2H), 0.67 (q, J=3.8 Hz, 2H).

25 Protons

Calculated mass (C26H28N2O3): 416.512, found mass: M+H⁺=417

### Example 115. 1-((5′-((4-methylcyclohexyl)ethynyl)-1′,3′-dihydrospiro[azetidine-3,2′-inden]-1-yl)methyl)cyclopropanecarboxylic acid

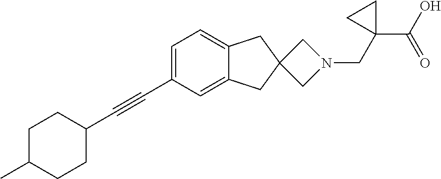 [SMILES: CC1CCC(C#Cc2ccc3c(c2)CC2(C3)CN(CC3(C(=O)O)CC3)C2)CC1]

¹H NMR (600 MHz, DMSO-d₆) δ 7.32-7.07 (m, 3H), 3.82 (s, 4H), 3.15 (t, J=5.3 Hz, 4H), 3.10 (s, 2H), 2.92 (t, J=4.0 Hz, 1H), 1.93 (dt, J=14.0, 4.5 Hz, 1H), 1.77-1.63 (m, 2H), 1.54 (ddq, J=20.6, 10.4, 3.8 Hz, 3H), 1.44-1.27 (m, 3H), 1.06 (q, J=3.3, 2.8 Hz, 2H), 0.95 (qd, J=13.1, 3.5 Hz, 1H), 0.88 (dd, J=17.1, 6.4 Hz, 5H).

31 Protons

Calculated mass (C25H31NO2): 377.519, found mass: M+H⁺=378 cis/trans mixture

### Example 116. 1-((5′-((6-(cyclopentyloxy)pyridin-3-yl)ethynyl)-1′,3′-dihydrospiro[azetidine-3,2′-inden]-1-yl)methyl)cyclopropanecarboxylic acid

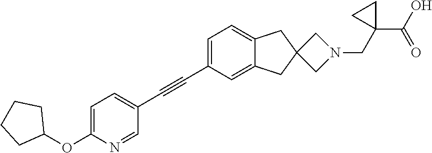 [SMILES: O=C(O)C1(CN2CC3(Cc4ccc(C#Cc5ccc(OC6CCCC6)nc5)cc4C3)C2)CC1]

¹H NMR (600 MHz, DMSO-d₆) δ 8.35 (d, J=2.4 Hz, 1H), 7.81 (dd, J=8.6, 2.4 Hz, 1H), 7.39 (d, J=1.5 Hz, 1H), 7.32 (dd, J=7.6, 1.5 Hz, 1H), 7.26 (d, J=7.8 Hz, 1H), 6.81 (d, J=8.5 Hz, 1H), 5.38 (td, J=6.0, 3.0 Hz, 1H), 3.49 (s, 5H), 3.14 (t, J=1.6 Hz, 5H), 2.77 (s, 2H), 1.99-1.89 (m, 2H), 1.75-1.66 (m, 4H), 1.60 (dddd, J=11.7, 9.5, 5.1, 2.1 Hz, 2H), 0.92 (q, J=3.7 Hz, 2H), 0.65 (q, J=3.8 Hz, 2H).

31 Protons

Calculated mass (C28H30N2O3): 442.549, found mass: M+H⁺=443

### Example 117. 1-((5′-((6-((1,1,1-trifluoropropan-2-yl)oxy)pyridin-3-yl)ethynyl)-1′,3′-dihydrospiro[azetidine-3,2′-inden]-1-yl)methyl)cyclopropanecarboxylic acid

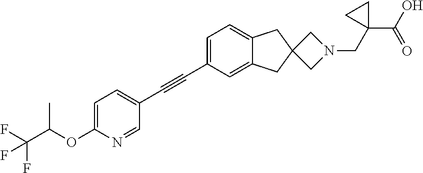 [SMILES: CC(Oc1ccc(C#Cc2ccc3c(c2)CC2(C3)CN(CC3(C(=O)O)CC3)C2)cn1)C(F)(F)F]

¹H NMR (600 MHz, DMSO-d₆) δ 8.40 (d, J=2.3 Hz, 1H), 7.94 (dd, J=8.6, 2.4 Hz, 1H), 7.41 (d, J=1.4 Hz, 1H), 7.34 (dd, J=7.7, 1.4 Hz, 1H), 7.28 (d, J=7.8 Hz, 1H), 7.01 (d, J=8.5 Hz, 1H), 5.91 (p, J=6.7 Hz, 1H), 3.49 (s, 5H), 3.14 (d, J=2.3 Hz, 4H), 2.77 (s, 2H), 1.47 (d, J=6.5 Hz, 4H), 0.92 (q, J=3.7 Hz, 2H), 0.64 (q, J=3.8 Hz, 2H).

26 Protons

Calculated mass (C26H25F3N2O3): 470.484, found mass: M+H⁺=471

### Example 118. 1-((5′-((4-methoxy-2-methylphenyl)ethynyl)-1′,3′-dihydrospiro[azetidine-3,2′-inden]-1-yl)methyl)cyclopropanecarboxylic acid

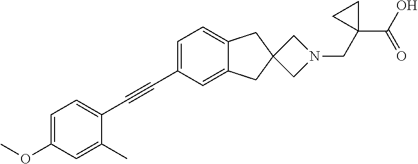 [SMILES: COc1ccc(C#Cc2ccc3c(c2)CC2(C3)CN(CC3(C(=O)O)CC3)C2)c(C)c1]

¹H NMR (600 MHz, Methanol-d4) δ 7.37 (d, J=8.6 Hz, 2H), 7.32 (dd, J=7.8, 1.4 Hz, 1H), 7.22 (d, J=7.8 Hz, 1H), 6.80 (d, J=2.6 Hz, 1H), 6.73 (dd, J=8.5, 2.6 Hz, 1H), 4.20-4.16 (m, 4H), 3.81 (s, 3H), 3.30 (s, 4H), 3.27 (s, 2H), 2.47 (s, 3H), 1.20 (q, J=4.1 Hz, 2H), 0.75 (q, J=4.1 Hz, 2H).

26 Protons

Calculated mass (C26H27NO3): 401.497, found mass: M+H⁺=402

### Example 119. 1-((5′-(2-(6-isopropoxypyridin-3-yl)ethyl)-1′,3′-dihydrospiro[azetidine-3,2′-inden]-1-yl)methyl)cyclopropanecarboxylic acid

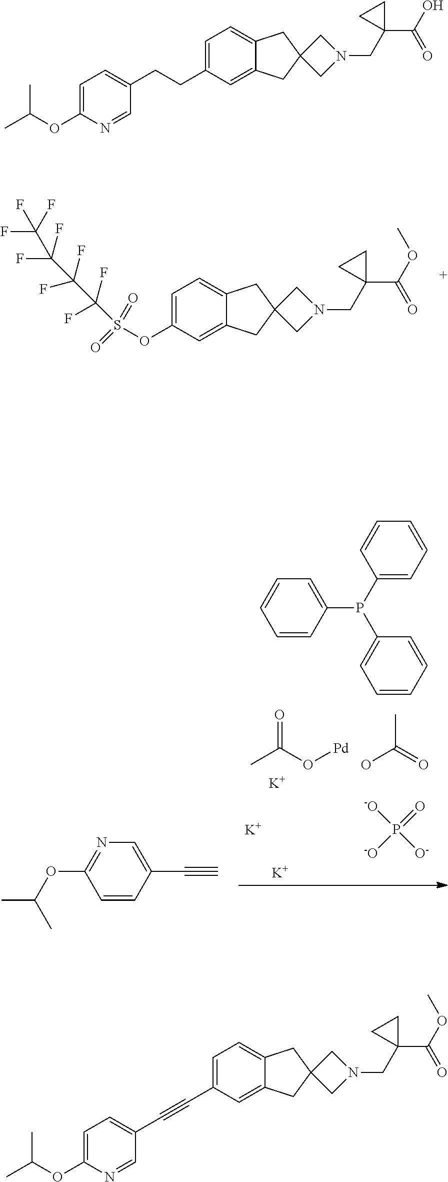

Methyl 1-((5′-(((perfluorobutyl)sulfonyl)oxy)-1′,3′-dihydrospiro[azetidine-3,2′-inden]-1-yl)methyl)cyclopropanecarboxylate (100 mg, 0.176 mmol, prepared as described for example 112) was dissolved in dry DMSO (3 mL) under an atmosphere of argon. 5-ethynyl-2-(propan-2-yloxy)pyridine (42.5 mg, 0.263 mmol) was added. The reaction mixture was degased with argon for 5 min. Potassium phosphate (44.7 mg, 0.211 mmol), palladium acetate (4.7 mg, 0.021 mmol) and triphenylphosphine (18.4 mg, 0.07 mmol) were added. The reaction mixture was stirred at 80° C. for 1 h. The reaction mixture was cooled to RT, diluted with CH₂Cl₂, successively washed with water (twice, 20 mL), saturated sodium bicarbonate (20 mL), water (20 mL) and dried (MgSO₄). The organic solvent was removed in vacuo and the crude product was purified by flash chromatography (silica, CH₂Cl₂/MeOH 95/5).

Yield: 62 mg (82%, pale yellow oil).

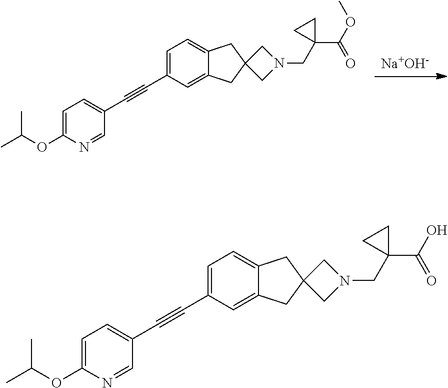 [SMILES: "Na+OH-" ; COC(=O)C1(CN2CC3(Cc4ccc(C#Cc5ccc(OC(C)C)nc5)cc4C3)C2)CC1 ; CC(C)Oc1ccc(C#Cc2ccc3c(c2)CC2(C3)CN(CC3(C(=O)O)CC3)C2)cn1]

Methyl 1-((5′-((6-isopropoxypyridin-3-yl)ethynyl)-1′,3′-dihydrospiro[azetidine-3,2′-inden]-1-yl)methyl)cyclopropanecarboxylate (62 mg, 0.044 mmol) was dissolved in THF (3 mL) and MeOH (3 mL). 1M NaOH (1.5 mL, 1.5 mmol) was added and the reaction mixture stirred at 50° C. for 3 h. The reaction m mixture was cooled to RT and 1N HCl (1.5 mL, 1.5 mmol) was added. The reaction mixture was diluted with water (5 mL) and the organic solvents were removed in vacuo. The aqueous phase was extracted with CH₂Cl₂ and the combined extracts were dried (MgSO₄). The crude product was used for the next step without purification.

Yield: 60 mg (quantitative, light brown foam).

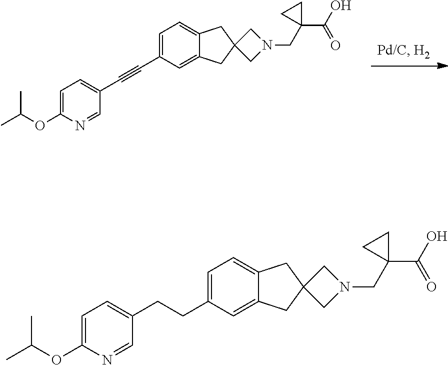 [SMILES: CC(C)Oc1ccc(C#Cc2ccc3c(c2)CC2(C3)CN(CC3(C(=O)O)CC3)C2)cn1 ; "Pd/C, H2" ; CC(C)Oc1ccc(CCc2ccc3c(c2)CC2(C3)CN(CC3(C(=O)O)CC3)C2)cn1]

1-((5′-((6-isopropoxypyridin-3-yl)ethynyl)-1′,3′-dihydrospiro[azetidine-3,2′-inden]-1-yl)methyl)cyclopropanecarboxylic acid (35 mg, 0.084 mmol) was dissolved in Ethanol (4 mL), degassed with argon and 10% Pd—C (20 mg, 0.188 mmol) was added. The reaction mixture was stirred under an atmosphere of hydrogen for 2 h. The catalyst was removed by filtration and the solvent was evaporated in vacuo. The crude product was purified by flash chromatography (silica, CH₂Cl₂, MeOH).

Yield: 20 mg (56.6%, pale yellow solid).

¹H NMR (600 MHz, DMSO-d₆) δ 7.94 (d, J=2.4 Hz, 1H), 7.54 (dd, J=8.5, 2.5 Hz, 1H), 7.13-7.06 (m, 2H), 6.98 (dd, J=7.6, 1.6 Hz, 1H), 6.67-6.62 (m, 1H), 5.18 (p, J=6.2 Hz, 1H), 3.51 (s, 4H), 3.07 (d, J=4.8 Hz, 4H), 2.82-2.72 (m, 6H), 1.25 (d, J=6.2 Hz, 6H), 0.92 (q, J=3.7 Hz, 2H), 0.65 (q, J=3.8 Hz, 2H).

31 Protons

Calculated mass (C26H32N2O3): 420.544, found mass: M+H⁺=421

### Example 120. 1-((5′-((4-ethoxy-3,5-difluorobenzyl)oxy)-1′,3′-dihydrospiro[azetidine-3,2′-inden]-1-yl)methyl)cyclopropanecarboxylic acid

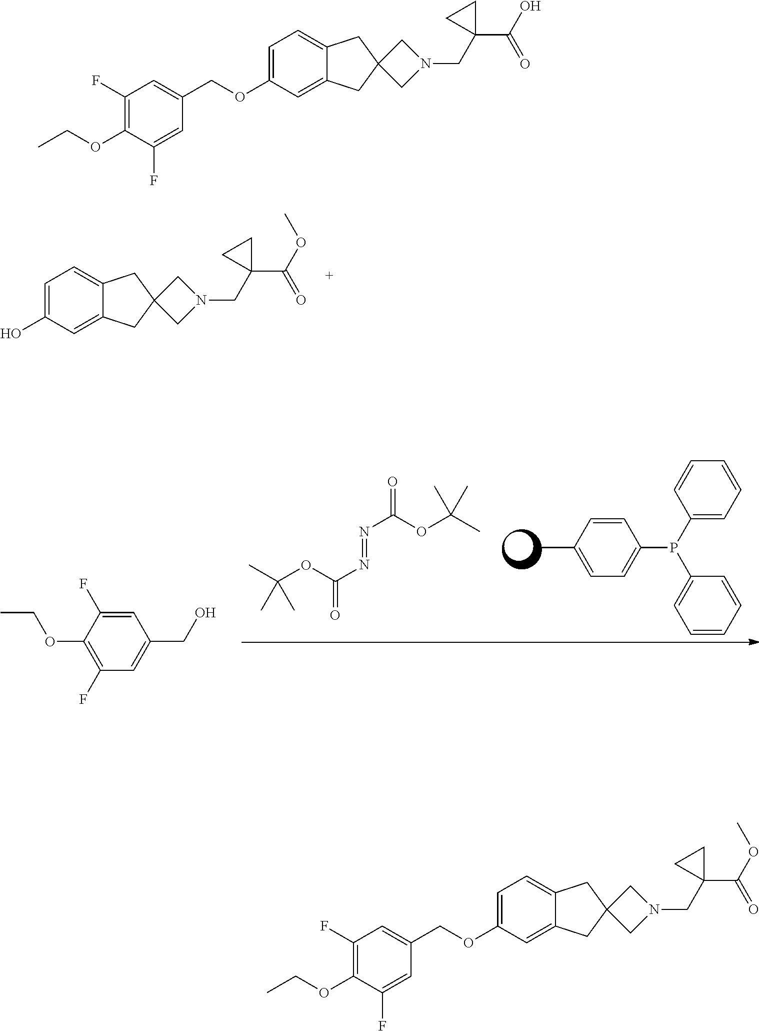 [SMILES: CCOc1c(F)cc(COc2ccc3c(c2)CC2(C3)CN(CC3(C(=O)O)CC3)C2)cc1F ; COC(=O)C1(CN2CC3(Cc4ccc(O)cc4C3)C2)CC1 ; CCOc1c(F)cc(CO)cc1F ; CC(C)(C)OC(=O)/N=N/C(=O)OC(C)(C)C ; Cc1ccc(P(c2ccccc2)c2ccccc2)cc1 ; CCOc1c(F)cc(COc2ccc3c(c2)CC2(C3)CN(CC3(C(=O)OC)CC3)C2)cc1F]

Methyl 1-((5′-hydroxy-1′,3′-dihydrospiro[azetidine-3,2′-inden]-1-yl)methyl)cyclopropanecarboxylate (60 mg, 0.209 mmol, prepared as described for example 28) was dissolved in dry THF (2 mL) and polystryrene bound triphenylphosphine (167 mg, 1.88 mmol/g, 0.313 mmol) was added. After 5 min 4-ethoxy-3,5-difluorobenzyl alcohol (47.1 mg, 0.251 mmol) and di-tert-butyl azodicarboxylate (72.1 mg, 0.313 mmol) were added. The reaction mixture was stirred at RT for 20 h. The polymer was removed by filtration and the filtrate concentrated in vacuo. The crude product was purified by flash chromatography (silica, CH₂Cl₂/MeOH 95/5).

Yield: 66 mg (69.1%, colorless oil).

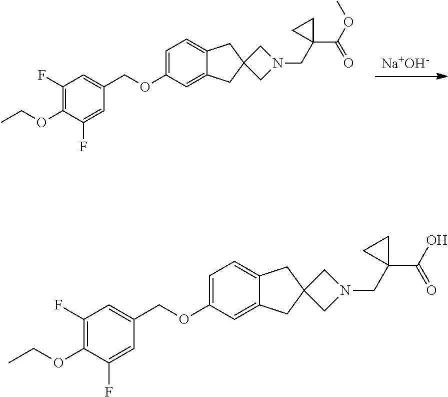 [SMILES: "Na+OH-" ; CCOc1c(F)cc(COc2ccc3c(c2)CC2(C3)CN(CC3(C(=O)OC)CC3)C2)cc1F ; CCOc1c(F)cc(COc2ccc3c(c2)CC2(C3)CN(CC3(C(=O)O)CC3)C2)cc1F]

Methyl 1-((5′-((4-ethoxy-3,5-difluorobenzyl)oxy)-1′,3′-dihydrospiro[azetidine-3,2′-inden]-1-yl)methyl)cyclopropanecarboxylate (65 mg, 0.142 mmol) was dissolved in THF (3 mL) and MeOH (3 mL) and 1M NaOH (1.5 mL, 1.5 mmol) was added. The reaction mixture was stirred at 50° C. for 2 h and then overnight at RT. 1N HCl (1.5 mL, 1.5 mmol) was added and the reaction mixture diluted with water (3 mL). The organic solvents were removed in vacuo. The aqueous phase was extracted with CH₂Cl₂ (three times, 20 mL) and the combined extracts were dried (MgSO₄). The solvent was evaporated in vacuo and the crude product was purified by flash chromatography (silica, CH₂Cl₂, MeOH) and then crystallized from n-pentane.

Yield: 45 mg (71.4%, colorless solid).

¹H NMR (600 MHz, DMSO-d₆) δ 7.25-7.17 (m, 2H), 7.11 (d, J=8.3 Hz, 1H), 6.88 (d, J=2.4 Hz, 1H), 6.77 (dd, J=8.2, 2.4 Hz, 1H), 5.00 (s, 2H), 4.14 (q, J=7.0 Hz, 2H), 3.49 (s, 4H), 3.05 (d, J=29.2 Hz, 4H), 2.78 (s, 2H), 1.29 (t, J=7.0 Hz, 3H), 0.91 (q, J=3.7 Hz, 2H), 0.63 (q, J=3.7 Hz, 2H).

26 Protons

Calculated mass (C25H27F2NO4): 443.483, found mass: M+H⁺=444

Examples 121-130 were prepared analogous to example 120:

### Example 121. 1-((5′-((6-methoxy-2-methylpyridin-3-yl)methoxy)-1′,3′-dihydrospiro[azetidine-3,2′-inden]-1-yl)methyl)cyclopropanecarboxylic acid

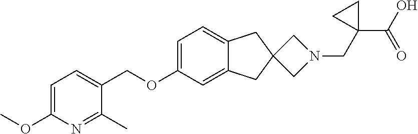 [SMILES: COc1ccc(COc2ccc3c(c2)CC2(C3)CN(CC3(C(=O)O)CC3)C2)c(C)n1]

¹H NMR (600 MHz, DMSO-d₆) δ 7.68 (d, J=8.3 Hz, 1H), 7.11 (d, J=8.3 Hz, 1H), 6.90 (d, J=2.4 Hz, 1H), 6.79 (dd, J=8.2, 2.5 Hz, 1H), 6.64 (d, J=8.3 Hz, 1H), 4.98 (s, 2H), 3.83 (s, 3H), 3.50 (s, 4H), 3.06 (d, J=28.6 Hz, 4H), 2.79 (s, 2H), 2.56-2.51 (m, 1H), 2.42 (s, 3H), 0.91 (q, J=3.6 Hz, 2H), 0.64 (q, J=3.7 Hz, 2H).

28 Protons

Calculated mass (C24H28N2O4): 408.490, found mass: M+H⁺=409

### Example 122. 1-((5′-((4-ethoxy-2,6-difluorobenzyl)oxy)-1′,3′-dihydrospiro[azetidine-3,2′-inden]-1-yl)methyl)cyclopropanecarboxylic acid

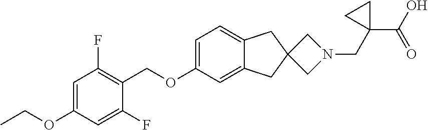 [SMILES: CCOc1cc(F)c(COc2ccc3c(c2)CC2(C3)CN(CC3(C(=O)O)CC3)C2)c(F)c1]

¹H NMR (600 MHz, DMSO-d₆) δ 7.11 (d, J=8.2 Hz, 1H), 6.89 (d, J=2.2 Hz, 1H), 6.82-6.74 (m, 3H), 4.94 (s, 2H), 4.07 (q, J=7.0 Hz, 2H), 3.49 (s, 4H), 3.05 (d, J=27.7 Hz, 4H), 2.78 (s, 2H), 1.32 (t, J=7.0 Hz, 3H), 0.91 (q, J=3.7 Hz, 2H), 0.63 (q, J=3.7 Hz, 2H).

26 Protons

Calculated mass (C25H27F2NO4): 443.483, found mass: M+H⁺=444

### Example 123. 1-((5′-((4-ethoxy-2,3-difluorobenzyl)oxy)-1′,3′-dihydrospiro[azetidine-3,2′-inden]-1-yl)methyl)cyclopropanecarboxylic acid

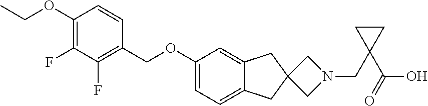 [SMILES: CCOc1ccc(COc2ccc3c(c2)CC2(C3)CN(CC3(C(=O)O)CC3)C2)c(F)c1F]

¹H NMR (600 MHz, DMSO-d₆) δ 7.27 (td, J=8.4, 2.1 Hz, 1H), 7.11 (d, J=8.2 Hz, 1H), 7.03 (td, J=8.3, 7.7, 1.7 Hz, 1H), 6.90 (d, J=2.3 Hz, 1H), 6.77 (dd, J=8.2, 2.5 Hz, 1H), 5.03 (s, 2H), 4.15 (q, J=7.0 Hz, 2H), 3.48 (s, 4H), 3.05 (d, J=28.3 Hz, 4H), 2.77 (s, 2H), 1.36 (t, J=7.0 Hz, 3H), 0.91 (q, J=3.7 Hz, 2H), 0.63 (q, J=3.8 Hz, 2H).

26 Protons

Calculated mass (C25H27F2NO4): 443.483, found mass: M+H⁺=444

### Example 124. 1-((5′-((2,5-dichloropyridin-3-yl)methoxy)-1′,3′-dihydrospiro[azetidine-3,2′-inden]-1-yl)methyl)cyclopropanecarboxylic acid

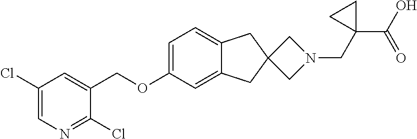 [SMILES: O=C(O)C1(CN2CC3(Cc4ccc(OCc5cc(Cl)cnc5Cl)cc4C3)C2)CC1]

¹H NMR (600 MHz, DMSO-d₆) δ 8.52 (d, J=2.7 Hz, 1H), 8.15 (d, J=2.6 Hz, 1H), 7.14 (d, J=8.3 Hz, 1H), 6.96 (d, J=2.3 Hz, 1H), 6.84 (dd, J=8.2, 2.5 Hz, 1H), 5.10 (s, 2H), 3.52 (s, 4H), 3.08 (d, J=30.4 Hz, 7H), 2.80 (s, 2H), 0.92 (q, J=3.7 Hz, 2H), 0.65 (q, J=3.7 Hz, 2H).

24 Protons

Calculated mass (C22H22C12N2O3): 433.328, found mass: M+H⁺=433/435

### Example 125. 1-((5′-((2,3-dihydro-1H-inden-4-yl)methoxy)-1′,3′-dihydrospiro[azetidine-3,2′-inden]-1-yl)methyl)cyclopropanecarboxylic acid

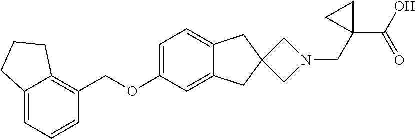 [SMILES: O=C(O)C1(CN2CC3(Cc4ccc(OCc5cccc6c5CCC6)cc4C3)C2)CC1]

¹H NMR (600 MHz, DMSO-d₆) δ 7.19 (t, J=7.0 Hz, 2H), 7.16-7.07 (m, 2H), 6.89 (d, J=2.4 Hz, 1H), 6.77 (dd, J=8.2, 2.5 Hz, 1H), 5.00 (s, 2H), 3.49 (s, 4H), 3.05 (d, J=28.1 Hz, 4H), 2.88 (t, J=7.4 Hz, 4H), 2.78 (s, 2H), 2.02 (p, J=7.5 Hz, 2H), 0.91 (q, J=3.7 Hz, 2H), 0.63 (q, J=3.7 Hz, 2H).

28 Protons

Calculated mass (C26H29NO3): 403.513, found mass: M+H⁺=404

### Example 126. 1-((5′-(cyclohexylmethoxy)-1′,3′-dihydrospiro[azetidine-3,2′-inden]-1-yl)methyl)cyclopropanecarboxylic acid

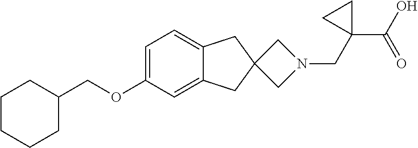 [SMILES: O=C(O)C1(CN2CC3(Cc4ccc(OCC5CCCCC5)cc4C3)C2)CC1]

¹H NMR (600 MHz, DMSO-d₆) δ 7.08 (d, J=8.2 Hz, 1H), 6.79 (d, J=2.3 Hz, 1H), 6.68 (dd, J=8.3, 2.4 Hz, 1H), 3.71 (d, J=6.3 Hz, 2H), 3.64 (s, 4H), 3.07 (d, J=27.8 Hz, 5H), 2.93 (s, 2H), 1.79 (dd, J=12.9, 3.7 Hz, 2H), 1.68 (ddt, J=33.1, 12.3, 3.9 Hz, 5H), 1.30-1.09 (m, 4H), 1.07-0.92 (m, 4H), 0.73 (q, J=4.0 Hz, 2H).

33 Protons

Calculated mass (C23H31NO3): 369.497, found mass: M+H⁺=370

### Example 127. 1-((5′-((2,3-dihydro-1H-inden-2-yl)oxy)-1′,3′-dihydrospiro[azetidine-3,2′-inden]-1-yl)methyl)cyclopropanecarboxylic acid

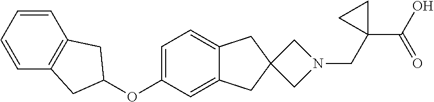 [SMILES: O=C(O)C1(CN2CC3(Cc4ccc(OC5Cc6ccccc6C5)cc4C3)C2)CC1]

¹H NMR (600 MHz, DMSO-d₆) δ 7.25 (dd, J=5.4, 3.3 Hz, 2H), 7.21-7.12 (m, 2H), 7.10 (d, J=8.2 Hz, 1H), 6.81 (d, J=2.3 Hz, 1H), 6.69 (dd, J=8.2, 2.4 Hz, 1H), 5.17 (td, J=5.9, 2.9 Hz, 1H), 3.49 (s, 4H), 3.33 (dd, J=16.8, 6.1 Hz, 3H), 3.10-2.95 (m, 5H), 2.78 (s, 2H), 0.91 (q, J=3.7 Hz, 2H), 0.63 (q, J=3.7 Hz, 2H).

26 Protons

Calculated mass (C25H27NO3): 389.487, found mass: M+H⁺=390

### Example 128. 1-((5′-((4-(cyclopropylmethoxy)benzyl)oxy)-1′,3′-dihydrospiro[azetidine-3,2′-inden]-1-yl)methyl)cyclopropanecarboxylic acid

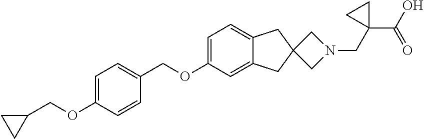 [SMILES: O=C(O)C1(CN2CC3(Cc4ccc(OCc5ccc(OCC6CC6)cc5)cc4C3)C2)CC1]

¹H NMR (600 MHz, DMSO-d₆) δ 7.35-7.29 (m, 2H), 7.08 (d, J=8.2 Hz, 1H), 6.94-6.88 (m, 2H), 6.86 (d, J=2.3 Hz, 1H), 6.75 (dd, J=8.2, 2.5 Hz, 1H), 4.94 (s, 2H), 3.80 (d, J=7.0 Hz, 2H), 3.49 (s, 4H), 3.04 (d, J=27.2 Hz, 4H), 2.78 (s, 2H), 1.21 (ddt, J=9.3, 7.5, 3.9 Hz, 1H), 0.90 (q, J=3.7 Hz, 2H), 0.62 (q, J=3.7 Hz, 2H), 0.59-0.51 (m, 2H), 0.36-0.26 (m, 2H).

30 Protons

Calculated mass (C27H31NO4): 433.539, found mass: M+H⁺=434

### Example 129. 1-((5′-((7-chloro-2,3-dihydro-1H-inden-1-yl)oxy)-1′,3′-dihydrospiro[azetidine-3,2′-inden]-1-yl)methyl)cyclopropanecarboxylic acid

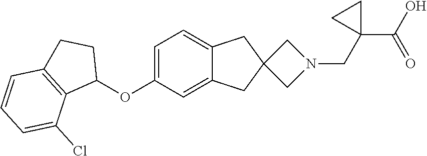 [SMILES: O=C(O)C1(CN2CC3(Cc4ccc(OC5CCc6cccc(Cl)c65)cc4C3)C2)CC1]

¹H NMR (600 MHz, DMSO-d₆) δ 7.41-7.28 (m, 3H), 7.12 (d, J=8.3 Hz, 1H), 6.89 (d, J=2.3 Hz, 1H), 6.77 (dd, J=8.2, 2.4 Hz, 1H), 5.79 (dd, J=6.5, 1.5 Hz, 1H), 3.54-3.52 (m, 4H), 3.07 (d, J=29.7 Hz, 7H), 2.94 (ddt, J=19.3, 10.3, 5.2 Hz, 1H), 2.80 (s, 2H), 2.47-2.40 (m, 1H), 2.10 (ddt, J=14.1, 8.2, 2.1 Hz, 1H), 0.91 (q, J=3.7 Hz, 2H), 0.64 (q, J=3.7 Hz, 2H). 27 Protons Calculated mass (C25H26ClNO3): 423.932, found mass: M+H±=424/426

### Example 130. 1-((5′-((2,3-dihydro-1H-inden-1-yl)oxy)-1′,3′-dihydrospiro[azetidine-3,2′-inden]-1-yl)methyl)cyclopropanecarboxylic acid

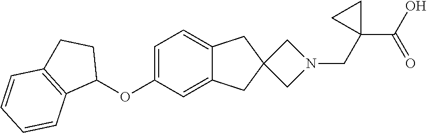 [SMILES: O=C(O)C1(CN2CC3(Cc4ccc(OC5CCc6ccccc65)cc4C3)C2)CC1]

¹H NMR (600 MHz, DMSO-d₆) δ 7.36 (d, J=7.5 Hz, 1H), 7.35-7.27 (m, 2H), 7.22 (td, J=7.2, 1.8 Hz, 1H), 7.12 (d, J=8.3 Hz, 1H), 6.91 (d, J=2.5 Hz, 1H), 6.80 (dd, J=8.2, 2.5 Hz, 1H), 5.79 (s, 1H), 3.49 (d, J=1.6 Hz, 4H), 3.09-2.99 (m, 5H), 2.86 (ddd, J=16.0, 8.6, 5.4 Hz, 1H), 2.78 (s, 2H), 2.57-2.50 (m, 1H), 1.99 (dddd, J=13.9, 8.6, 5.4, 4.1 Hz, 1H), 0.91 (q, J=3.7 Hz, 2H), 0.63 (q, J=3.7 Hz, 2H).

26 Protons

Calculated mass (C25H27NO3): 389.487, found mass: M+H⁺=390

### Example 131. 1-((5′-((2-cyclopropyl-6-fluorobenzyl)oxy)-1′,3′-dihydrospiro[azetidine-3,2′-inden]-1-yl)methyl)cyclopropanecarboxylic acid

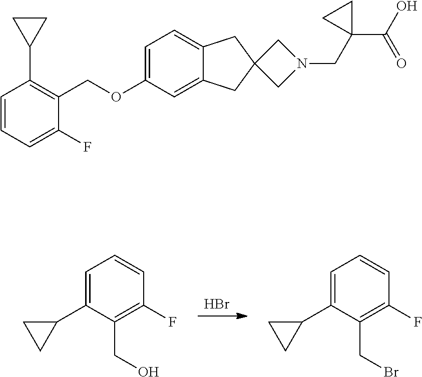 [SMILES: O=C(O)C1(CN2CC3(Cc4ccc(OCc5c(F)cccc5C5CC5)cc4C3)C2)CC1 ; OCc1c(F)cccc1C1CC1 ; Br ; Fc1cccc(C2CC2)c1CBr]

(2-cyclopropyl-6-fluorophenyl)methanol (400 mg, 2.407 mmol) was suspended in 48% hydrogen bromide (5 mL) and the reaction mixture was stirred at RT overnight. The reaction mixture was diluted with water (20 mL) and extracted with ethyl acetate (20 mL). The organic phase was successively washed with saturated sodium bicarbonate (twice) and water and dried (MgSO₄). The solvent was removed in vacuo. The crude 2-(bromomethyl)-1-cyclopropyl-3-fluorobenzene was used without further purification for the next step.

Yield: 550 mg (quantitative, light brown oil).

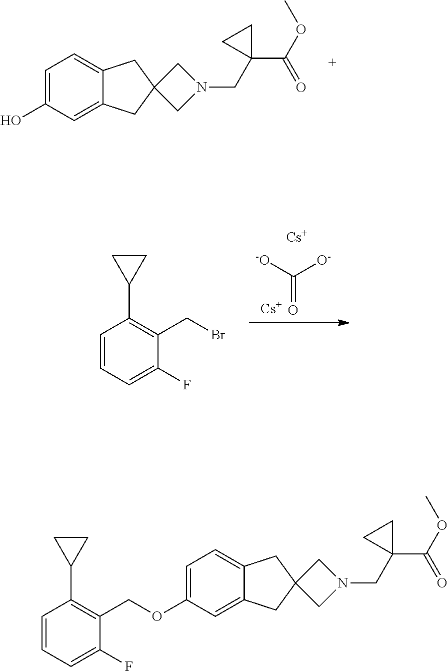

Methyl 1-((5′-hydroxy-1′,3′-dihydrospiro[azetidine-3,2′-inden]-1-yl)methyl)cyclopropanecarboxylate (100 mg, 0.348 mmol, prepared as described for example 28) was dissolved in dry DMF (4 mL). Cesium carbonate (227 mg, 0.696 mmol) and 2-(bromomethyl)-1-cyclopropyl-3-fluorobenzene (120 mg, 0.522 mmol) were added and the reaction mixture stirred at RT for 2 h. The DMF was removed in vacuo, the residue was treated with CH₂Cl₂ and water. The organic phase was washed with water (twice) and dried (MgSO₄). The crude product was purified by flash chromatography (silica, CH₂Cl₂/MeOH 95/5).

Yield: 47 mg (31%, pale yellow oil).

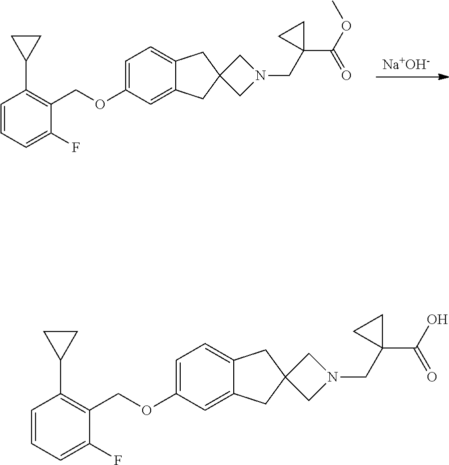 [SMILES: "Na+OH-" ; COC(=O)C1(CN2CC3(Cc4ccc(OCc5c(F)cccc5C5CC5)cc4C3)C2)CC1 ; O=C(O)C1(CN2CC3(Cc4ccc(OCc5c(F)cccc5C5CC5)cc4C3)C2)CC1]

Methyl 1-((5′-((2-cyclopropyl-6-fluorobenzyl)oxy)-1′,3′-dihydrospiro[azetidine-3,2′-inden]-1-yl)methyl)cyclopropanecarboxylate (47 mg, 0.108 mmol) was dissolved in THF (2 mL) and MeOH (2 mL). 1M NaOH (1.1 mL, 1.1 mmol) was added and the reaction mixture was stirred at 50° C. for 2 h. The reaction mixture was cooled to RT and 1N HCl (1.1 mL, 1.1 mmol) and water (5 mL) were added. The organic solvents were removed in vacuo and the aqueous phase was extracted with CH₂Cl₂ (twice). The solvent was removed in vacuo and the crude product was purified by flash chromatography (silica, CH₂Cl₂, MeOH) and crystallized from n-pentane.

Yield: 31 mg (68.2%, colorless solid).

¹H NMR (600 MHz, DMSO-d₆) δ 7.33 (td, J=8.1, 6.0 Hz, 1H), 7.12 (d, J=8.2 Hz, 1H), 7.06 (ddd, J=9.5, 8.2, 1.0 Hz, 1H), 6.95 (d, J=2.3 Hz, 1H), 6.85 (d, J=7.7 Hz, 1H), 6.82 (dd, J=8.2, 2.5 Hz, 1H), 5.16 (d, J=1.7 Hz, 2H), 3.52 (s, 4H), 3.07 (d, J=29.8 Hz, 4H), 2.80 (s, 2H), 2.05 (tt, J=8.4, 5.2 Hz, 1H), 0.96-0.89 (m, 4H), 0.73-0.67 (m, 2H), 0.64 (q, J=3.7 Hz, 2H).

27 Protons

Calculated mass (C26H28FNO3): 421.504, found mass: M+H⁺=422

### Example 132. 1-((5′-(2-cyclohexylethoxy)-1′,3′-dihydrospiro[azetidine-3,2′-inden]-1-yl)methyl)cyclopropanecarboxylic acid

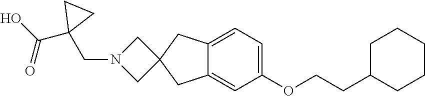 [SMILES: O=C(O)C1(CN2CC3(Cc4ccc(OCCC5CCCCC5)cc4C3)C2)CC1]

A 4 mL scintillation vial was charged with a stir bar, 5004 of a solution of methyl 1-((5′-hydroxy-1′,3′-dihydrospiro[azetidine-3,2′-inden]-1-yl)methyl)cyclopropanecarboxylate (prepared as described for example 28) in toluene anhydrous (30.0 mg, 0.104 mmol), 3504 of a 0.6 mmol preweighed vial containing a solution of cyclopentylmethanol dissolved in toluene anhydrous (20.9 mg, 0.21 mmol, 2 eq), and Cyanomethylenetributylphospharane (CMBP)(63.0 mg, 0.26 mmol, 2.5 eq). The vial was capped and placed to heat at 80° C. for 16 hours. Upon completion the solvent is removed under a N₂ blower and the crude material is redissolved in 500 μL of THF. To this 1500 μL of a 1M aqueous solution of LiOH in 75% MeOH is added, the vial capped once more and placed to heat at 60° C. for 1 hour. The crude material is then passed through a cartridge containing 300 mg of celite and washed with Acetonitrile 2×1000 μL. The recovered crude solution is then dried under N2 and dissolved once again with 1500 μL of DMSO/MeOH (1:1 v/v), and sent to APS for reverse phase HPLC purification using the ammonium acetate method, to recover 1-((5′-(2-cyclohexylethoxy)-1′,3′-dihydrospiro[azetidine-3,2′-inden]-1-yl)methyl)cyclopropanecarboxylic acid. (8.4 mg, 28.7%).

¹H NMR (400 MHz, DMSO-d₆) δ 7.07 (d, J=8.2 Hz, 1H), 6.77 (d, J=2.4 Hz, 1H), 6.68 (dd, J=8.1, 2.4 Hz, 1H), 3.95 (t, J=6.6 Hz, 2H), 3.66 (s, 4H), 3.11 (s, 2H), 3.06 (s, 2H), 2.90 (s, 2H), 1.74-1.64 (m, 3H), 1.59 (q, J=6.6 Hz, 3H), 1.46 (dtt, J=14.0, 6.9, 3.6 Hz, 1H), 1.28-1.12 (m, 3H), 1.00 (dd, J=12.4, 3.0 Hz, 1H), 0.96 (q, J=3.7 Hz, 3H), 0.94-0.85 (m, 1H), 0.62 (q, J=3.7 Hz, 2H). MS (APCI+) m/z 384.3 (M+H⁺).

Examples 133-148 were prepared analogous to example 132:

### Example 133. 1-((5′-((4-methylbenzyl)oxy)-1′,3′-dihydrospiro[azetidine-3,2′-inden]-1-yl)methyl)cyclopropanecarboxylic acid

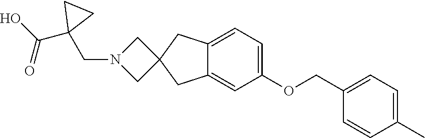 [SMILES: Cc1ccc(COc2ccc3c(c2)CC2(C3)CN(CC3(C(=O)O)CC3)C2)cc1]

(3.2 mg, 32.7%). ¹H NMR (400 MHz, DMSO-d₆) δ 7.30 (d, J=7.8 Hz, 2H), 7.18 (d, J=7.8 Hz, 2H), 7.08 (d, J=8.2 Hz, 1H), 6.86 (d, J=2.3 Hz, 1H), 6.75 (dd, J=8.3, 2.4 Hz, 1H), 4.99 (s, 2H), 3.46 (s, 4H), 3.06 (s, 2H), 3.01 (s, 2H), 2.76 (s, 2H), 2.30 (s, 3H), 0.90 (q, J=3.7 Hz, 2H), 0.61 (q, J=3.8 Hz, 2H). MS (APCI+) m/z 378.2 (M+H⁺).

### Example 134. 1-((5′-(2-methylphenethoxy)-1′,3′-dihydrospiro[azetidine-3,2′-inden]-1-yl)methyl)cyclopropanecarboxylic acid

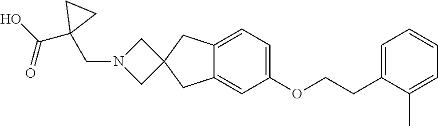 [SMILES: Cc1ccccc1CCOc1ccc2c(c1)CC1(C2)CN(CC2(C(=O)O)CC2)C1]

(21.1 mg, 72.2%). ¹H NMR (400 MHz, DMSO-d₆) δ 7.23-7.20 (m, 1H), 7.18-7.05 (m, 4H), 6.77 (d, J=2.4 Hz, 1H), 6.69 (dd, J=8.2, 2.5 Hz, 1H), 4.14 (t, J=6.8 Hz, 2H), 3.65 (s, 4H), 3.10 (s, 2H), 3.06 (s, 2H), 3.00 (t, J=6.8 Hz, 2H), 2.89 (s, 2H), 2.31 (s, 3H), 0.96 (q, J=3.7 Hz, 2H), 0.62 (q, J=3.7 Hz, 2H). MS (APCI+) m/z 392.3 (M+H⁺).

### Example 135. 1-((5′-(4-methoxyphenethoxy)-1′,3′-dihydrospiro[azetidine-3,2′-inden]-1-yl)methyl)cyclopropanecarboxylic acid

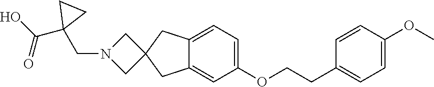 [SMILES: COc1ccc(CCOc2ccc3c(c2)CC2(C3)CN(CC3(C(=O)O)CC3)C2)cc1]

(19.0 mg, 64.9%). ¹H NMR (400 MHz, DMSO-d₆) δ 7.22-7.17 (m, 2H), 7.07 (d, J=8.2 Hz, 1H), 6.88-6.83 (m, 2H), 6.77 (d, J=2.3 Hz, 1H), 6.68 (dd, J=8.2, 2.5 Hz, 1H), 4.12 (t, J=6.7 Hz, 2H), 3.73 (s, 2H), 3.63 (s, 4H), 3.10 (s, 2H), 3.06 (s, 2H), 2.93 (t, J=6.7 Hz, 2H), 2.88 (s, 2H), 0.95 (q, J=3.7 Hz, 2H), 0.61 (q, J=3.7 Hz, 2H). MS (APCI+) m/z 408.5 (M+H⁺).

### Example 136. 1-((5′-(2-fluorophenethoxy)-1′,3′-dihydrospiro[azetidine-3,2′-inden]-1-yl)methyl)cyclopropanecarboxylic acid

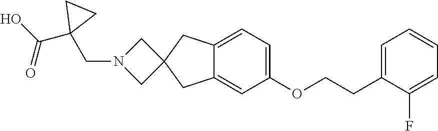 [SMILES: O=C(O)C1(CN2CC3(Cc4ccc(OCCc5ccccc5F)cc4C3)C2)CC1]

(21.8 mg, 74.5%). ¹H NMR (400 MHz, DMSO-d₆) δ 7.36 (t, J=7.6 Hz, 1H), 7.30-7.24 (m, 1H), 7.16-7.05 (m, 3H), 6.78-6.73 (m, 1H), 6.67 (dd, J=8.0, 2.2 Hz, 1H), 4.17 (t, J=6.7 Hz, 2H), 3.57 (s, 4H), 3.08 (s, 2H), 3.06-3.02 (m, 4H), 2.86 (s, 2H), 0.93 (q, J=3.7 Hz, 2H), 0.58 (q, J=3.5 Hz, 2H). MS (APCI+) m/z 396.2 (M+H⁺).

### Example 137. 1-((5′-(4-fluorophenethoxy)-1′,3′-dihydrospiro[azetidine-3,2′-inden]-1-yl)methyl)cyclopropanecarboxylic acid

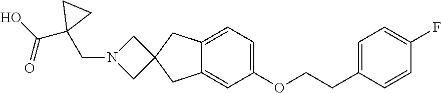 [SMILES: O=C(O)C1(CN2CC3(Cc4ccc(OCCc5ccc(F)cc5)cc4C3)C2)CC1]

(23.7 mg, 81%) ¹H NMR (400 MHz, DMSO-d₆) δ 7.31 (dd, J=8.4, 5.6 Hz, 2H), 7.10-7.03 (m, 3H), 6.77 (s, 1H), 6.71-6.66 (m, 1H), 4.15 (t, J=6.5 Hz, 2H), 3.84-3.37 (m, 4H), 3.08 (d, J=17.1 Hz, 4H), 2.99 (t, J=6.5 Hz, 3H), 0.96 (s, 2H), 0.63 (s, 2H).

MS (APCI+) m/z 396.3 (M+H⁺).

### Example 138. 1-((5′-((2-methylbenzyl)oxy)-1′,3′-dihydrospiro[azetidine-3,2′-inden]-1-yl)methyl)cyclopropanecarboxylic acid

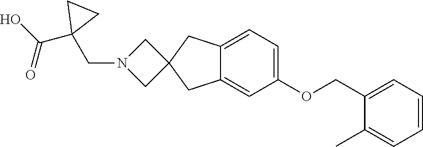 [SMILES: Cc1ccccc1COc1ccc2c(c1)CC1(C2)CN(CC2(C(=O)O)CC2)C1]

(12.4 mg, 42.4%). ¹H NMR (400 MHz, DMSO-d₆) δ 7.38 (t, J=8.8 Hz, 1H), 7.28-7.11 (m, 4H), 7.13-7.04 (m, 1H), 6.89 (d, J=9.2 Hz, 1H), 6.81 (d, J=8.3 Hz, 1H), 5.03 (s, 2H), 3.68 (s, 5H), 3.11 (d, J=8.5 Hz, 3H), 2.90 (s, 2H), 2.32 (s, 2H), 0.97-0.91 (m, 2H), 0.64-0.56 (m, 2H). MS (APCI+) m/z 378.2 (M+H⁺).

### Example 139. 1-((5′-((4-methoxybenzyl)oxy)-1′,3′-dihydrospiro[azetidine-3,2′-inden]-1-yl)methyl)cyclopropanecarboxylic acid

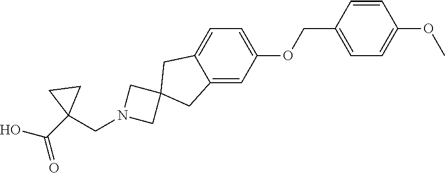 [SMILES: COc1ccc(COc2ccc3c(c2)CC2(C3)CN(CC3(C(=O)O)CC3)C2)cc1]

(22.6 mg, 77.2%). ¹H NMR (400 MHz, DMSO-d₆) δ 7.35-7.31 (m, 2H), 7.08 (d, J=8.2 Hz, 1H), 6.95-6.90 (m, 2H), 6.85 (d, J=2.4 Hz, 1H), 6.76 (dd, J=8.2, 2.5 Hz, 1H), 4.96 (s, 2H), 3.76 (s, 3H), 3.66 (s, 4H), 3.11 (s, 2H), 3.07 (s, 2H), 2.90 (s, 2H), 0.96 (q, J=3.7 Hz, 2H), 0.62 (q, J=3.7 Hz, 2H). MS (APCI+) m/z 394.1 (M+H⁺).

### Example 140. 1-((5′-((2-fluorobenzyl)oxy)-1′,3′-dihydrospiro[azetidine-3,2′-inden]-1-yl)methyl)cyclopropanecarboxylic acid

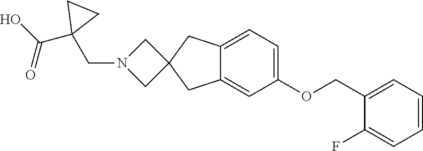 [SMILES: O=C(O)C1(CN2CC3(Cc4ccc(OCc5ccccc5F)cc4C3)C2)CC1]

(20.4 mg, 69.8%). ¹H NMR (400 MHz, DMSO-d₆) δ 7.50 (td, J=7.6, 1.7 Hz, 1H), 7.39 (tdd, J=7.6, 5.3, 1.7 Hz, 1H), 7.24-7.15 (m, 2H), 7.11 (d, J=8.2 Hz, 1H), 6.88 (d, J=2.3 Hz, 1H), 6.79 (dd, J=8.2, 2.4 Hz, 1H), 5.08 (s, 2H), 3.67 (s, 4H), 3.13 (s, 2H), 3.08 (s, 2H), 2.90 (s, 2H), 0.97 (q, J=3.8 Hz, 2H), 0.63 (q, J=3.8 Hz, 2H). MS (APCI+) m/z 382.1 (M+H⁺).

### Example 141. 1-((5′-((2-chlorobenzyl)oxy)-1′,3′-dihydrospiro[azetidine-3,2′-inden]-1-yl)methyl)cyclopropanecarboxylic acid

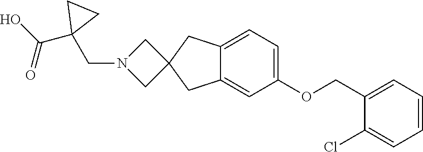 [SMILES: O=C(O)C1(CN2CC3(Cc4ccc(OCc5ccccc5Cl)cc4C3)C2)CC1]

(22.3 mg, 76.2%). ¹H NMR (400 MHz, DMSO-d₆) δ 7.55 (dd, J=5.6, 3.8 Hz, 1H), 7.49-7.44 (m, 1H), 7.39-7.33 (m, 2H), 7.11 (d, J=8.2 Hz, 1H), 6.89 (d, J=2.3 Hz, 1H), 6.79 (dd, J=8.2, 2.4 Hz, 1H), 5.11 (s, 2H), 3.66 (s, 4H), 3.13 (s, 2H), 3.08 (s, 2H), 2.90 (s, 2H), 0.97 (q, J=3.7 Hz, 2H), 0.63 (q, J=3.7 Hz, 2H). MS (APCI+) m/z 398.2 (M+H⁺).

### Example 142. 1-((5′-((4-cyanobenzyl)oxy)-1′,3′-dihydrospiro[azetidine-3,2′-inden]-1-yl)methyl)cyclopropanecarboxylic acid

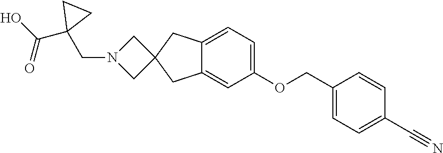 [SMILES: N#Cc1ccc(COc2ccc3c(c2)CC2(C3)CN(CC3(C(=O)O)CC3)C2)cc1]

(9.1 mg, 31.1%). ¹H NMR (400 MHz, DMSO-d₆) δ 7.78 (d, J=8.3 Hz, 2H), 7.61 (d, J=8.1 Hz, 2H), 7.09 (d, J=8.2 Hz, 1H), 6.88-6.86 (m, 1H), 6.79-6.76 (m, 1H), 5.14 (s, 2H), 3.52 (s, 4H), 3.08 (s, 2H), 3.04 (s, 2H), 2.84 (s, 2H), 0.94-0.87 (m, 2H), 0.59-0.52 (m, 2H). MS (APCI+) m/z 389.2 (M+H⁺).

### Example 143. 1-((5′-(benzo[d][1,3]dioxol-5-ylmethoxy)-1′,3′-dihydrospiro[azetidine-3,2′-inden]-1-yl)methyl)cyclopropanecarboxylic acid

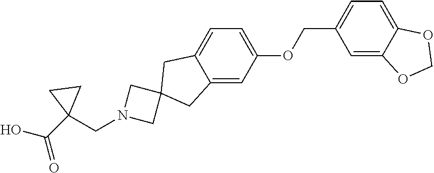 [SMILES: O=C(O)C1(CN2CC3(Cc4ccc(OCc5ccc6c(c5)OCO6)cc4C3)C2)CC1]

(18.1 mg, 61.8%). ¹H NMR (400 MHz, DMSO-d₆) δ 7.08 (d, J=8.2 Hz, 1H), 6.94-6.82 (m, 4H), 6.76 (dd, J=8.2, 2.3 Hz, 1H), 5.96 (d, J=1.1 Hz, 2H), 4.94 (s, 2H), 3.66 (s, 4H), 3.11 (s, 2H), 3.07 (s, 2H), 2.90 (s, 2H), 0.96 (q, J=3.7 Hz, 2H), 0.62 (q, J=3.8 Hz, 2H). MS (APCI+) m/z 408.3 (M+H⁺).

### Example 144. 1-((5′-(2-(1H-indol-3-yl)ethoxy)-1′,3′-dihydrospiro[azetidine-3,2′-inden]-1-yl)methyl)cyclopropanecarboxylic acid

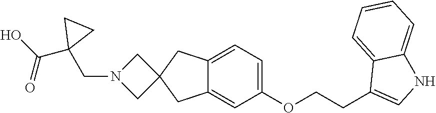 [SMILES: O=C(O)C1(CN2CC3(Cc4ccc(OCCc5c[nH]c6ccccc56)cc4C3)C2)CC1]

(3.1 mg, 10.8%) ¹H NMR (400 MHz, DMSO-d₆) δ 7.56 (dt, J=7.8, 1.1 Hz, 1H), 7.36 (dt, J=8.1, 1.0 Hz, 1H), 7.17 (s, 1H), 7.10-7.05 (m, 2H), 6.99 (ddd, J=8.0, 7.0, 1.1 Hz, 1H), 6.79 (d, J=2.4 Hz, 1H), 6.71 (dd, J=8.2, 2.5 Hz, 1H), 4.20 (t, J=6.8 Hz, 2H), 3.63 (s, 4H), 3.13 (dd, J=6.8, 0.9 Hz, 1H), 3.10 (s, 2H), 3.06 (s, 2H), 2.88 (s, 2H), 0.95 (q, J=3.7 Hz, 2H), 0.60 (q, J=3.7 Hz, 2H). MS (APCI+) m/z 417.2 (M+H⁺).

### Example 145. 1-((5′-(2-(2-chlorophenoxy)ethoxy)-1′,3′-dihydrospiro[azetidine-3,2′-inden]-1-yl)methyl)cyclopropanecarboxylic acid

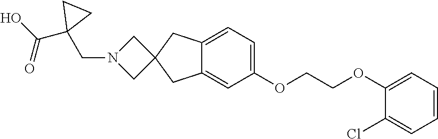 [SMILES: O=C(O)C1(CN2CC3(Cc4ccc(OCCOc5ccccc5Cl)cc4C3)C2)CC1]

(20.2 mg, 68.8%). ¹H NMR (400 MHz, DMSO-d₆) δ 7.39 (dd, J=7.9, 1.6 Hz, 1H), 7.29 (ddd, J=8.9, 7.4, 1.6 Hz, 1H), 7.18 (dd, J=8.2, 1.4 Hz, 1H), 7.10 (d, J=8.2 Hz, 1H), 6.97 (td, J=7.6, 1.4 Hz, 1H), 6.85 (s, 1H), 6.75 (dd, J=8.2, 2.5 Hz, 1H), 4.37 (dd, J=5.5, 3.1 Hz, 2H), 4.30 (dd, J=5.9, 3.0 Hz, 2H), 3.64 (s, 4H), 3.11 (s, 2H), 3.07 (s, 2H), 2.89 (s, 2H), 0.96 (q, J=3.7 Hz, 2H), 0.61 (q, J=3.7 Hz, 2H). MS (APCI+) m/z 428.4 (M+H⁺).

### Example 146. 1-((5′-(3-(6-methylpyridin-2-yl)propoxy)-1′,3′-dihydrospiro[azetidine-3,2′-inden]-1-yl)methyl)cyclopropanecarboxylic acid

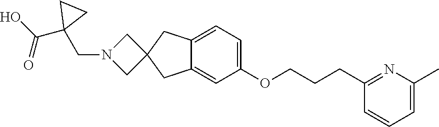 [SMILES: Cc1cccc(CCCOc2ccc3c(c2)CC2(C3)CN(CC3(C(=O)O)CC3)C2)n1]

(1.2 mg, 4.1%). ¹H NMR (400 MHz, DMSO-d₆) δ 7.55 (t, J=7.7 Hz, 1H), 7.09-7.02 (m, 3H), 6.76 (s, 1H), 6.68 (d, J=8.0 Hz, 1H), 3.97 (t, J=6.5 Hz, 3H), 2.86-2.79 (m, 2H), 2.43 (s, 3H), 2.12-2.04 (m, 2H), 1.52-1.28 (m, 2H), 0.99-0.93 (m, 1H), 0.89 (t, J=7.2 Hz, 2H), 0.67-0.57 (m, 2H). MS (APCI+) m/z 407.2 (M+H⁺).

### Example 147. 1-((5′-((3-cyanobenzyl)oxy)-1′,3′-dihydrospiro[azetidine-3,2′-inden]-1-yl)methyl)cyclopropanecarboxylic acid

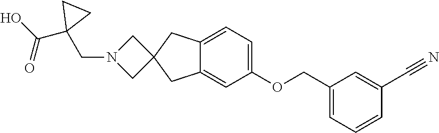 [SMILES: N#Cc1cccc(COc2ccc3c(c2)CC2(C3)CN(CC3(C(=O)O)CC3)C2)c1]

(18.3 mg, 62.6%). ¹H NMR (400 MHz, DMSO-d₆) δ 7.81 (s, 1H), 7.78-7.71 (m, 2H), 7.59 (t, J=7.8 Hz, 1H), 7.11 (d, J=8.3 Hz, 1H), 6.89 (d, J=2.3 Hz, 1H), 6.80 (dd, J=8.3, 2.5 Hz, 1H), 5.12 (s, 2H), 3.66 (s, 4H), 3.12 (s, 2H), 3.08 (s, 2H), 2.90 (s, 2H), 0.97 (q, J=3.8 Hz, 2H), 0.63 (q, J=3.8 Hz, 2H). MS (APCI+) m/z 390.1 (M+H⁺).

### Example 148. 1-((5′-(3-fluorophenethoxy)-1′,3′-dihydrospiro[azetidine-3,2′-inden]-1-yl)methyl)cyclopropanecarboxylic acid

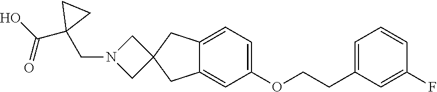 [SMILES: O=C(O)C1(CN2CC3(Cc4ccc(OCCc5cccc(F)c5)cc4C3)C2)CC1]

(17.8 mg, 60.8%)¹H NMR (400 MHz, DMSO-d₆) δ 7.32 (td, J=7.9, 6.2 Hz, 1H), 7.14-7.06 (m, 3H), 6.99 (td, J=8.7, 2.6 Hz, 1H), 6.77 (d, J=2.3 Hz, 1H), 6.69 (dd, J=8.2, 2.4 Hz, 1H), 4.18 (t, J=6.5 Hz, 2H), 3.64 (s, 4H), 3.10 (s, 2H), 3.06 (s, 2H), 3.02 (t, J=6.5 Hz, 2H), 2.88 (s, 2H), 0.95 (q, J=3.7 Hz, 2H), 0.61 (q, J=3.7 Hz, 2H).

MS (APCI+) m/z 396.6 (M+H⁺).

### Example 149. 2-(6′-((4-chlorophenyl)ethynyl)-3′H-spiro[azetidine-3,2′-benzofuran]-1-yl)acetic acid

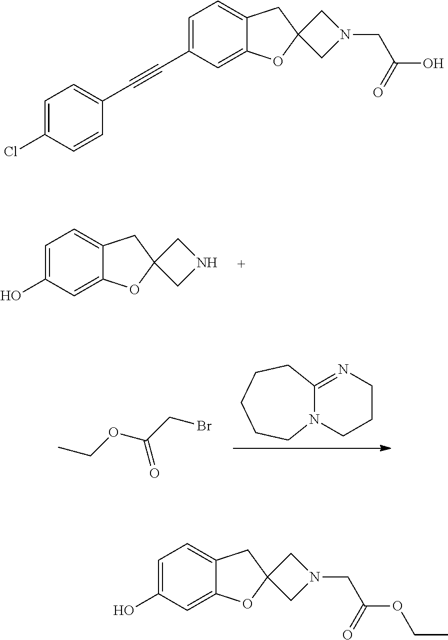

3′H-spiro[azetidine-3,2′-benzofuran]-6′-ol (242 mg, 1.366 mmol) was dissolved in dry DMF (20 mL) and 1,8-diazabicyclo[5.4.0]undec-7-ene (0.244 mL, 2.049 mmol) and ethyl bromoacetate (0.197 mL, 1.775 mmol) were added at RT. The reaction mixture was stirred at RT for 2 h. The solvent was removed in vacuo and the residue treated with CH₂Cl₂ (40 mL) and water (20 mL). The organic phase was washed with saturated sodium chloride solution and dried (MgSO₄). The crude product was purified by flash chromatography (silica n-heptane, ethyl acetate).

Yield: 158 mg (43.9%), pale yellow solid.

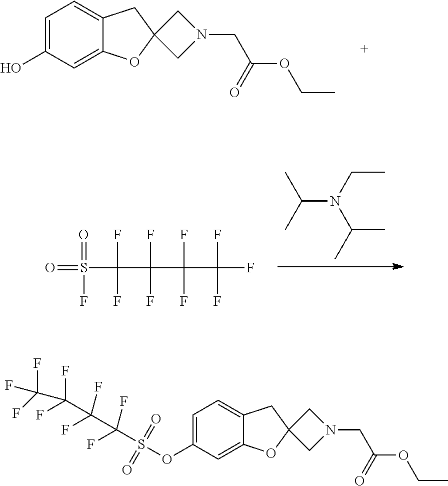

Ethyl 2-(6′-hydroxy-3′H-spiro[azetidine-3,2′-benzofuran]-1-yl)acetate (247 mg, 0.938 mmol) was dissolved in CH₂Cl₂ (5 mL) and N,N-diisopropylethylamine (0.64 mL, 3.75 mmol) were added. The reaction mixture was cooled to 0° C. and Nonafluorobutanesulfonyl fluoride (0.42 mL, 2.35 mmol) were added. After 2 h stirring at 0° C. the reaction mixture was allowed to come to RT and stirring was continued for additional 2 h. The reaction mixture was diluted with CH₂Cl₂ (20 mL) and washed with water (twice). The organic solution was dried (MgSO₄) and concentrated in vacuo. The crude product was purified by flash chromatography (silica, n-heptane, ethyl acetate).

Yield: 400 mg (78%), colorless oil.

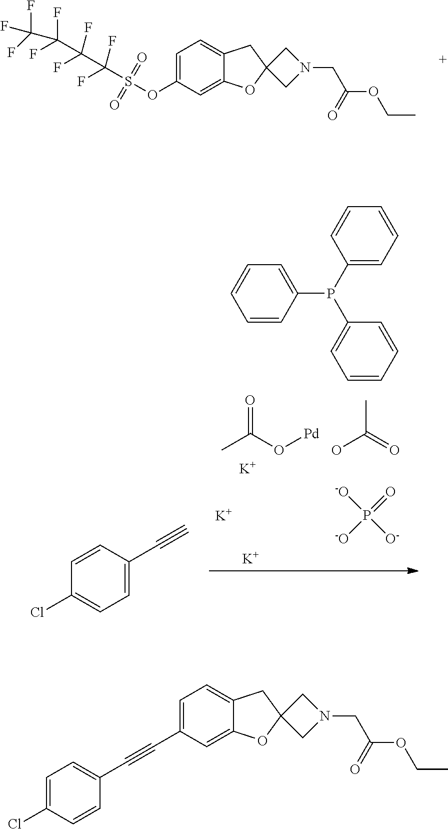

Ethyl 2-(6′-(((perfluorobutyl)sulfonyl)oxy)-3′H-spiro[azetidine-3,2′-benzofuran]-1-yl)acetate (100 mg, 0.183 mmol) was dissolved in dry DMSO (2 mL) under an argon atmosphere. 1-chloro-4-ethynylbenzene (37.6 mg, 0.275 mmol) was added. The reaction mixture was degased with argon for 5 min. Potassium phosphate (46.7 mg, 0.220 mmol), palladium acetate (4.94 mg, 0.022 mmol) and triphenyl phosphine (19.24 mg, 0.073 mmol) were added. The reaction mixture was heated to 80° C. under stirring for 1 h. The reaction mixture was diluted with CH₂Cl₂ and successively washed with water (twice), saturated sodium bicarbonate solution and water. The organic phase was dried (MgSO₄) and concentrated in vacuo. The crude product was purified by flash chromatography (silica, CH₂Cl₂, MeOH).

Yield: 63 mg (90%), pale yellow solid.

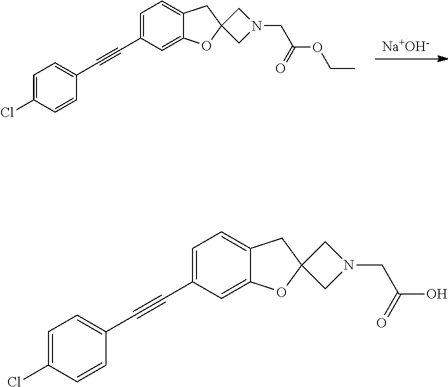 [SMILES: "Na+OH-" ; CCOC(=O)CN1CC2(Cc3ccc(C#Cc4ccc(Cl)cc4)cc3O2)C1 ; O=C(O)CN1CC2(Cc3ccc(C#Cc4ccc(Cl)cc4)cc3O2)C1]

Ethyl 2-(6′-((4-chlorophenyl)ethynyl)-3′H-spiro[azetidine-3,2′-benzofuran]-1-yl)acetate (61 mg, 0.160 mmol) was dissolved in THF (3 mL) and MeOH (3 mL). 1M NaOH (1 mL, 1.0 mmol) was added and the reaction mixture was stirred at RT for 1 h. 1N HCl (1.0 mL) was added. The organic solvents were removed in vacuo. 2-(6′-((4-chlorophenyl)ethynyl)-3′H-spiro[azetidine-3,2′-benzofuran]-1-yl)acetic acid precipitated and was obtained by filtration and washed with water. The product was dried in vacuo at 40° C.

Yield: 50 mg (88%), pale yellow solid.

¹H NMR (600 MHz, DMSO-d₆) δ 7.58-7.55 (m, 2H), 7.51-7.47 (m, 2H), 7.28 (d, J=7.6 Hz, 1H), 7.08 (dd, J=7.6, 1.4 Hz, 1H), 6.97 (d, J=1.3 Hz, 1H), 3.72-3.68 (m, 2H), 3.62-3.58 (m, 2H), 3.49 (s, 2H), 3.33 (s, 2H).

MS: Calculated for (C20H16ClNO3): 353.08, found mass: M+H⁺=354

Examples 150-155 were prepared analogously to example 149:

### Example 150. 2-(6′-(cyclohexylethynyl)-3′H-spiro[azetidine-3,2′-benzofuran]-1-yl)acetic acid

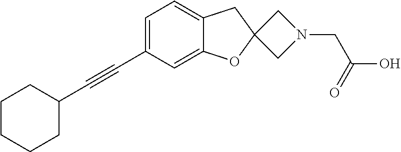 [SMILES: O=C(O)CN1CC2(Cc3ccc(C#CC4CCCCC4)cc3O2)C1]

¹H NMR (600 MHz, DMSO-d₆) δ 7.17 (d, J=7.7 Hz, 1H), 6.87 (dd, J=7.6, 1.4 Hz, 1H), 6.75 (s, 1H), 3.72-3.66 (m, 2H), 3.59 (d, J=9.2 Hz, 2H), 1.84-1.78 (m, 2H), 1.67 (dq, J=9.1, 2.7 Hz, 2H), 1.55-1.39 (m, 3H), 1.38-1.25 (m, 3H).

MS: Calculated for (C20H23NO3): 325.17, found mass: M+H⁺=326

### Example 151. 2-(6′-(phenylethynyl)-3′H-spiro[azetidine-3,2′-benzofuran]-1-yl)acetic acid

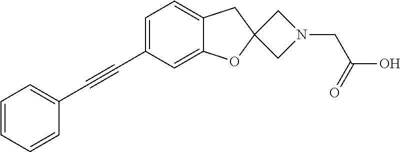 [SMILES: O=C(O)CN1CC2(Cc3ccc(C#Cc4ccccc4)cc3O2)C1]

¹H NMR (600 MHz, DMSO-d₆) δ 7.53 (td, J=5.5, 4.9, 3.0 Hz, 2H), 7.42 (dd, J=5.0, 2.0 Hz, 3H), 7.26 (d, J=7.6 Hz, 1H), 7.07 (dd, J=7.6, 1.4 Hz, 1H), 6.95 (d, J=1.3 Hz, 1H), 3.68 (d, J=8.7 Hz, 2H), 3.58 (d, J=8.7 Hz, 2H), 3.49 (s, 2H), 3.32 (s, 2H).

MS: Calculated for (C20H17NO3): 319.12, found mass: M+H⁺=320

### Example 152. 2-(6′-((3-chlorophenyl)ethynyl)-3′H-spiro[azetidine-3,2′-benzofuran]-1-yl)acetic acid

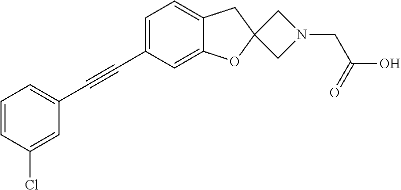 [SMILES: O=C(O)CN1CC2(Cc3ccc(C#Cc4cccc(Cl)c4)cc3O2)C1]

¹H NMR (600 MHz, DMSO-d₆) δ 7.62 (t, J=1.9 Hz, 1H), 7.53-7.48 (m, 2H), 7.47-7.43 (m, 1H), 7.28 (d, J=7.6 Hz, 1H), 7.09 (dd, J=7.6, 1.4 Hz, 1H), 6.97 (d, J=1.3 Hz, 1H), 3.70-3.65 (m, 2H), 3.60-3.56 (m, 2H), 3.49 (s, 2H), 3.32 (s, 2H).

MS: Calculated for (C20H16ClNO3): 353.08, found mass: M+H⁺=354

### Example 153. 2-(6′-((2-chlorophenyl)ethynyl)-3′H-spiro[azetidine-3,2′-benzofuran]-1-yl)acetic acid

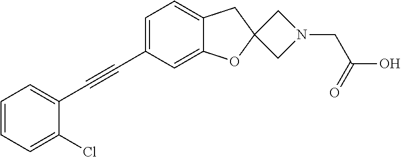 [SMILES: O=C(O)CN1CC2(Cc3ccc(C#Cc4ccccc4Cl)cc3O2)C1]

¹H NMR (600 MHz, DMSO-d₆) δ 7.66 (ddd, J=6.7, 4.8, 1.8 Hz, 1H), 7.59 (dt, J=8.0, 1.7 Hz, 1H), 7.41 (dtt, J=24.8, 7.5, 2.2 Hz, 2H), 7.31 (dd, J=24.2, 7.6 Hz, 1H), 7.09 (d, J=7.6 Hz, 1H), 6.95 (s, 1H), 4.25 (s, 1H), 4.12 (q, J=5.3 Hz, 1H), 3.17 (d, J=3.4 Hz, 4H).

MS: Calculated for (C20H16ClNO3): 353.08, found mass: M+H+=354

### Example 154. 2-(6′-((4-ethoxyphenyl)ethynyl)-3′H-spiro[azetidine-3,2′-benzofuran]-1-yl)acetic acid

10684304-0991

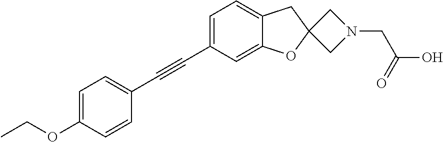 [SMILES: CCOc1ccc(C#Cc2ccc3c(c2)OC2(C3)CN(CC(=O)O)C2)cc1]

¹H NMR (600 MHz, DMSO-d₆) δ 7.47-7.43 (m, 2H), 7.24 (d, J=7.6 Hz, 1H), 7.02 (dd, J=7.5, 1.4 Hz, 1H), 6.97-6.93 (m, 2H), 6.91 (d, J=1.3 Hz, 1H), 4.06 (q, J=7.0 Hz, 2H), 3.69-3.63 (m, 2H), 3.59-3.54 (m, 2H), 3.47 (s, 2H), 3.31 (s, 2H), 1.33 (t, J=7.0 Hz, 3H).

MS: Calculated for (C22H21NO4): 363.15, found mass: M+H⁺=364

### Example 155. 2-(6′-((4-methylcyclohexyl)ethynyl)-3′H-spiro[azetidine-3,2′-benzofuran]-1-yl)acetic acid

10684304-0992

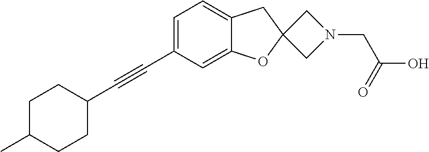 [SMILES: CC1CCC(C#Cc2ccc3c(c2)OC2(C3)CN(CC(=O)O)C2)CC1]

¹H NMR (600 MHz, DMSO-d₆) δ 7.16 (dd, J=9.4, 7.7 Hz, 1H), 6.87 (ddd, J=19.4, 7.5, 1.4 Hz, 1H), 6.74 (dd, J=16.2, 1.3 Hz, 1H), 3.65 (dd, J=9.5, 3.5 Hz, 2H), 3.58-3.52 (m, 2H), 3.43 (d, J=3.6 Hz, 2H), 3.30 (d, J=2.7 Hz, 2H), 1.96-1.89 (m, 1H), 1.77-1.69 (m, 1H), 1.66 (dd, J=13.8, 3.5 Hz, 1H), 1.53 (ddd, J=12.7, 10.1, 6.2 Hz, 2H), 1.42-1.27 (m, 3H), 0.94 (dd, J=11.8, 3.3 Hz, 1H), 0.87 (dd, J=19.6, 6.5 Hz, 3H).

MS: Calculated for (C21H25NO3): 339.18, found mass: M+H⁺=340

### Example 156. 1-((6′-((2-chloro-6-ethylbenzyl)oxy)-3′H-spiro[azetidine-3,2′-benzofuran]-1-yl)methyl)cyclopropanecarboxylic acid

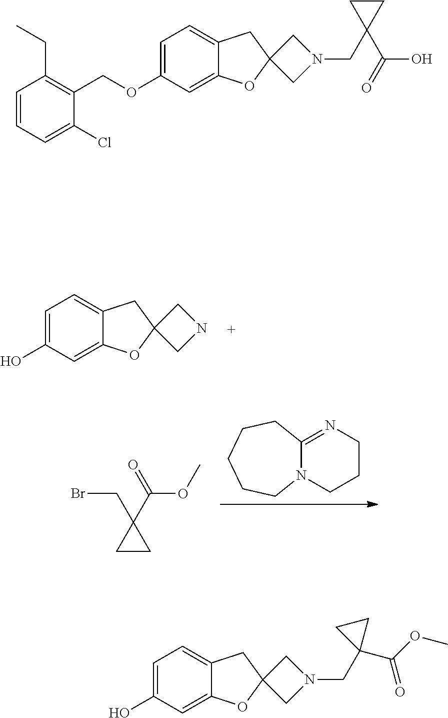

In a 50 mL round-bottomed flask 3′H-spiro[azetidine-3,2′-benzofuran]-6′-ol (50 mg, 0.282 mmol) was dissolved in DMF (2 mL) to give a colorless solution. DBU (0.064 mL, 0.423 mmol) and methyl 1-(bromomethyl)cyclopropane-carboxylate (70.8 mg, 0.367 mmol) were added. Stirred at RT overnight.

The residue was evaportated. The mixture was extracted with ethyl acetate and water. The organic layer was washed with sodium chloride solution. The residue dried over MgSO₄ and evaporated. The residue was purified by flash chromatography (silica 4 g, 0-10% MeOH in CH₂Cl₂, 18 mL/min)

Yield: 25.4 mg (0.088 mmol, 31%, colorless oil).

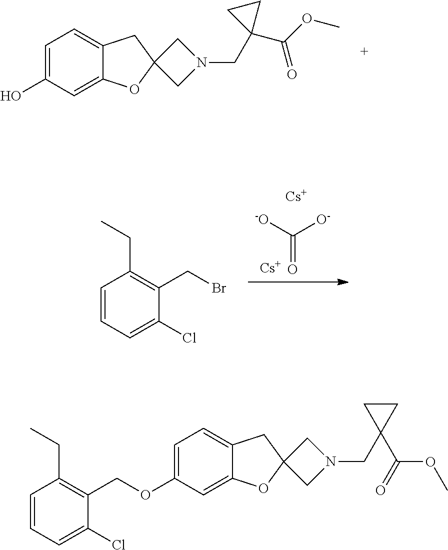

In a 50 mL round-bottomed flask methyl 1-((6′-hydroxy-3′H-spiro[azetidine-3,2′-benzofuran]-1-yl)methyl)cyclopropanecarboxylate (25.4 mg, 0.088 mmol) was dissolved in DMF (2 mL) to give a colorless solution. Cesium carbonate (40 mg, 0.123 mmol) and 2-(bromomethyl)-1-chloro-3-ethylbenzene (25 mg, 0.107 mmol) were added. Stirred at RT for 2 days. The residue was evaportated. The mixture was extracted with CH₂Cl₂ and water. After phase separation with a Chromabond PTS-cartridge the organic layer was evaporated. The residue was purified by flash chromatography (silica 4 g, 0-10% MeOH in CH₂Cl₂, 18 mL/min)

Yield: 25 mg (0,057 mmol, 64%, colorless oil).

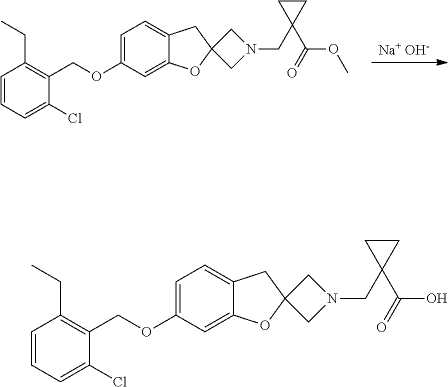 [SMILES: "Na+ OH-" ; CCc1cccc(Cl)c1COc1ccc2c(c1)OC1(C2)CN(CC2(C(=O)OC)CC2)C1 ; CCc1cccc(Cl)c1COc1ccc2c(c1)OC1(C2)CN(CC2(C(=O)O)CC2)C1]

In a 50 mL round-bottomed flask methyl 1-((6′-((2-chloro-6-ethylbenzyl)oxy)-3′H-spiro[azetidine-3,2′-benzofuran]-1-yl)methyl)cyclopropanecarboxylate (25 mg, 0.057 mmol) was dissolved in THF (0.5 mL) and MeOH (0.5 mL) to give a colorless solution. 1N NaOH (250 μL, 0.500 mmol) were added. Stirred at RT. The reaction mixture was evaporated, the residue was dissolved in water and neutralized with 250 μL. CH₂Cl₂ was added. After phase separation the organic layer was dried with MgSO₄, filtered and evaporated. Crude product: 22 mg white foam. The foam was purified by flash chromatography (silica 4 g, 0-50% MeOH in CH₂Cl₂, 18 mL/min)

Yield: 19 mg (0.044 mmol, 78%, white foam).

¹H NMR (600 MHz, DMSO-d₆) δ 7.38-7.34 (m, 2H), 7.27 (dd, J=6.0, 3.0 Hz, 1H), 7.10 (dd, J=8.0, 1.3 Hz, 1H), 6.56-6.52 (m, 2H), 5.08 (s, 2H), 3.63-3.55 (m, 2H), 3.53-3.46 (m, 2H), 2.73 (s, 2H), 2.70 (q, J=7.6 Hz, 2H), 1.15 (t, J=7.5 Hz, 3H), 0.97 (q, J=3.8 Hz, 2H), 0.74 (q, J=3.9 Hz, 2H).

MS: Calculated mass (C24H26ClNO4): 427.16, found mass: M+H=438/430

### Example 157. 4-(6′-((2-chloro-6-ethylbenzyl)oxy)-3′H-spiro[azetidine-3,2′-benzofuran]-1-yl)-3-methylbutanoic acid

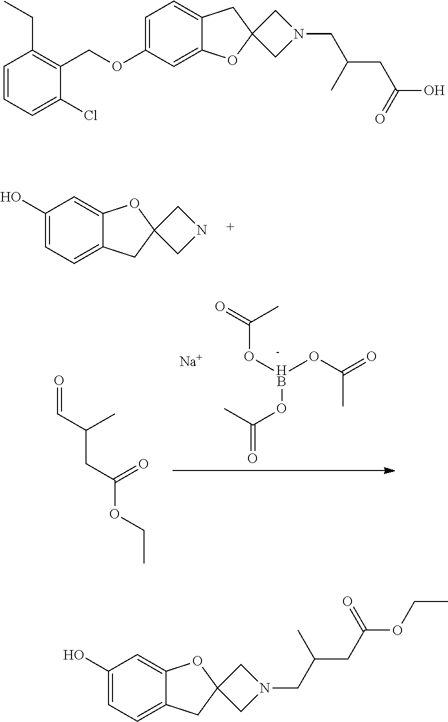

In a 250 mL round-bottomed flask 3′H-spiro[azetidine-3,2′-benzofuran]-6′-ol (50 mg, 0.282 mmol) was dissolved in THF (5 mL) with Ethyl 3-methyl-4-oxobutanoate (50 mg, 0.347 mmol) to give a yellow solution. Stirred for 30 min at RT. Sodium triacetoxyborohydride (90 mg, 0.423 mmol) was added. Stirred for 30 min at RT. The mixture was extracted with 10 mL CH₂Cl₂ and 2 mL water. Stirred for 10 min at RT. After phase separation with a Chromabond PTS-cartridge the organic layer was evaporated. The residue was purified by flash chromatography (silica 4 g, 0-10% MeOH in CH₂Cl₂, 12 mL/min)

Yield: 59 mg (0.193 mmol, 68.5%, clear oil).

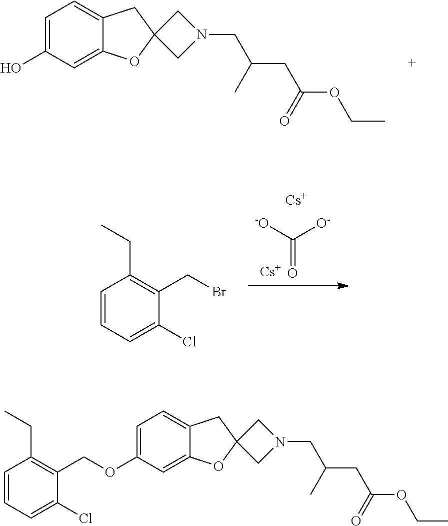

In a 50 mL round-bottomed flask ethyl 4-(6′-hydroxy-3′H-spiro[azetidine-3,2′-benzofuran]-1-yl)-3-methylbutanoate (59 mg, 0.193 mmol) was dissolved in DMF (4 mL) to give a colorless solution. Cesium carbonate (70 mg, 0.215 mmol) and 2-(bromomethyl)-1-chloro-3-ethylbenzene (50 mg, 0.214 mmol) were added. Stirred at RT over the night. The residue was evaportated. The mixture was extracted with CH₂Cl₂ and water. After phase separation with a Chromabond PTS-cartridge the organic layer was evaporated. The residue was purified by flash chromatography (silica 4 g, 0-10% MeOH in CH₂Cl₂, 18 mL/min)

Yield: 48 mg (0.105 mmol, 54%, colorless oil).

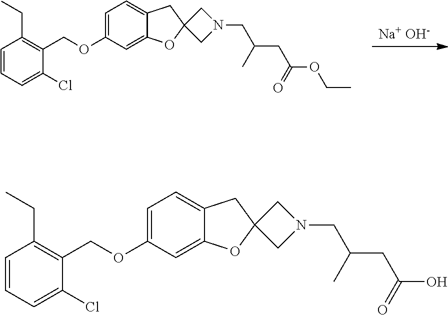 [SMILES: "Na+ OH-" ; CCOC(=O)CC(C)CN1CC2(Cc3ccc(OCc4c(Cl)cccc4CC)cc3O2)C1 ; CCc1cccc(Cl)c1COc1ccc2c(c1)OC1(C2)CN(CC(C)CC(=O)O)C1]

In a 50 mL round-bottom flask ethyl 4-(6′-((2-chloro-6-ethylbenzyl)oxy)-3′H-spiro[azetidine-3,2′-benzofuran]-1-yl)-3-methylbutanoate (48 mg, 0.105 mmol) was dissolved in THF (0.5 mL) and MeOH (0.5 mL) to give a colorless solution. 1M NaOH (0.5 ml, 1.000 mmol) was added. The reaction mixture was evaporated. The residue was dissolved in water and neutralized to pH 7 with ˜0.5 mL 2N HCl. CH₂Cl₂ was added. After phase separation the organic layer was dried over MgSO₄, filtered and evaporated.

Yield: 44 mg (0.102 mmol, 98%, colorless foam).

¹H NMR (600 MHz, DMSO-d₆) δ 7.38-7.35 (m, 2H), 7.27 (dd, J=5.9, 3.1 Hz, 1H), 7.11 (d, J=7.9 Hz, 1H), 6.54 (d, J=7.9 Hz, 2H), 5.09 (s, 2H), 2.70 (q, J=7.5 Hz, 2H), 2.35 (dd, J=15.6, 5.7 Hz, 1H), 2.01 (dd, J=15.6, 7.9 Hz, 1H), 1.86 (s, 1H), 1.15 (t, J=7.6 Hz, 3H), 0.90 (d, J=6.7 Hz, 3H).

MS: Calculated mass (C24H28ClNO4): 429.17, found mass: M+H=430/432

### Example 158. 2-(6′-((2-chloro-6-ethylbenzyl)oxy)-3′H-spiro[azetidine-3,2′-benzofuran]-1-yl)acetic acid

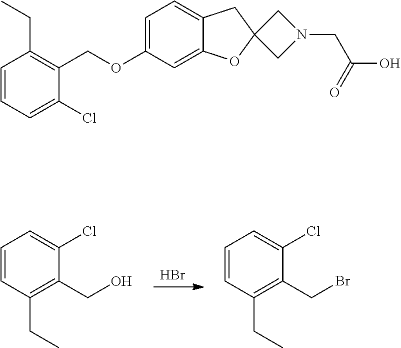 [SMILES: CCc1cccc(Cl)c1COc1ccc2c(c1)OC1(C2)CN(CC(=O)O)C1 ; CCc1cccc(Cl)c1CO ; Br ; CCc1cccc(Cl)c1CBr]

A solution of (2-chloro-6-ethylphenyl)methanol (6 g, 35.2 mmol) in aqueous 40% HBr (20 mL) was stirred for 12 hr at 50° C. Ethyl acetate was added, the organic layer was separated and washed with saturated sodium bicarbonate solution, dried with anhydrous Na₂SO₄. The solvent was removed to give 2-(bromomethyl)-1-chloro-3-ethylbenzene.

Yield: 7.215 g (27.2 mmol, 77% yield, oil).

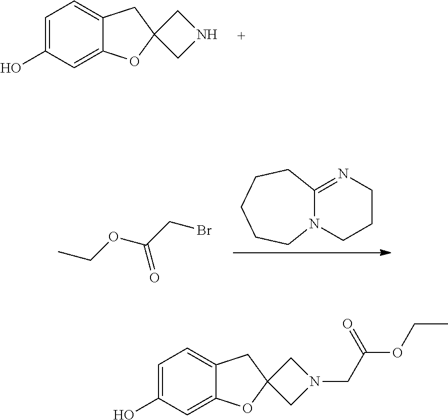

In a 50 mL round-bottomed flask 3′H-spiro[azetidine-3,2′-benzofuran]-6′-ol (50 mg, 0.282 mmol) was dissolved in DMF (2 mL) to give a colorless solution. DBU (64 μL, 0.425 mmol) and ethyl bromoacetate (42 μL, 0.379 mmol) were added. Stirred at RT overnight. The reaction mixture was evaporated. The residue was extracted with CH₂Cl₂ and water. After phase separation with a Chromabond PTS cartridge the organic layer was evaporated. The residue was purified by flash chromatography (silica 4 g, 0-10% MeOH in CH₂Cl₂, 18 mL/min).

Yield: 55 mg (colorless oil).

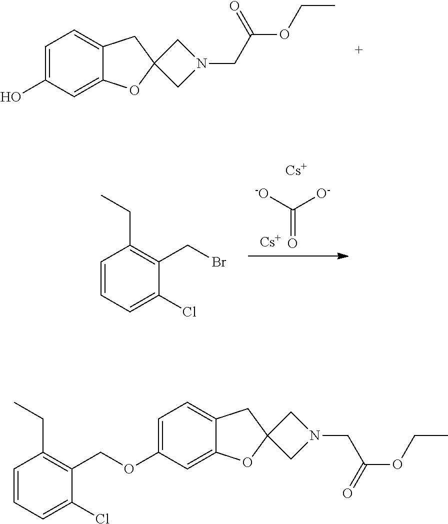

In a 50 mL round-bottomed flask ethyl 2-(6′-hydroxy-3′H-spiro[azetidine-3,2′-benzofuran]-1-yl)acetate (55 mg, 0.209 mmol) was dissolved in DMF (2 mL) to give a colorless solution. Cesium carbonate (90 mg, 0.276 mmol) and 2-(bromomethyl)-1-chloro-3-ethylbenzene (60 mg, 0.257 mmol) were added. Stirred at RT for 30 min. The reaction mixture was evaporated. The residue was extracted with CH₂Cl₂ and water. After phase separation with a Chromabond PTS-cartridge the organic layer was evaporated. The residue was purified by flash chromatography (silica 4 g, 0-10% MeOH in CH₂Cl₂, 18 mL/min).

Yield: 21 mg (yellow oil).

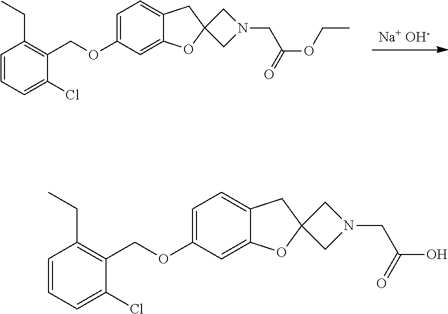 [SMILES: "Na+ OH-" ; CCOC(=O)CN1CC2(Cc3ccc(OCc4c(Cl)cccc4CC)cc3O2)C1 ; CCc1cccc(Cl)c1COc1ccc2c(c1)OC1(C2)CN(CC(=O)O)C1]

In a 10 mL flask ethyl 2-(6′-((2-chloro-6-ethylbenzyl)oxy)-3′H-spiro[azetidine-3,2′-benzofuran]-1-yl)acetate (21 mg, 0.050 mmol) was dissolved in THF (0.5 mL) and MeOH (0.5 mL) to give a colorless solution. NaOH (0.2 mL, 0.400 mmol) was added. Stirred at RT overnight. The reaction mixture was evaporated. The residue was dissolved in water and neutralized with 2N HCl (0.2 mL). CH₂Cl₂ was added. After phase separation the organic layer was dried over MgSO₄, filtered and evaporated.

Yield: 17 mg (87%, 0.044 mmol, colorless solid).

¹H NMR (600 MHz, DMSO-d₆) δ 7.38-7.34 (m, 2H), 7.27 (dd, J=5.9, 3.1 Hz, 1H), 7.11 (d, J=8.0 Hz, 1H), 6.57-6.52 (m, 2H), 5.09 (s, 2H), 3.67 (d, J=8.7 Hz, 2H), 3.57 (d, J=8.7 Hz, 2H), 2.70 (q, J=7.5 Hz, 2H), 1.15 (t, J=7.6 Hz, 3H).

MS: Calculated mass (C21H22ClNO4): 387.12, found mass: M+H=388/390

### Example 159. 2-(5-((2-ethyl-6-fluorobenzyl)oxy)-1,3-dihydrospiro[indene-2,4′-piperidin]-1′-yl)acetic acid hydrochloride

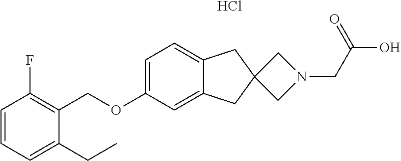 [SMILES: Cl ; CCc1cccc(F)c1COc1ccc2c(c1)CC1(C2)CN(CC(=O)O)C1]

### 1,3-dihydrospiro[indene-2,4′-piperidin]-5-ol

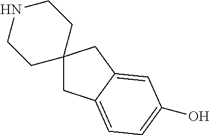 [SMILES: Oc1ccc2c(c1)CC1(CCNCC1)C2]

To a solution of commercially available 1′-benzyl-1,3-dihydrospiro[indene-2,4′-piperidin]-5-ol (1.3 g, 4.43 mmol) in 10 ml methanol) was added Pd/C (10%, 1.415 g) at 20° C. After stirring at 20° C. for 2h under hydrogen the catalyst was filtered off. The filtrate was concentrated to give 1,3-dihydrospiro[indene-2,4′-piperidin]-5-ol (710.1 mg) as a white solid.

### Ethyl 2-(5-hydroxy-1,3-dihydrospiro[indene-2,4′-piperidin]-1′-yl)acetate

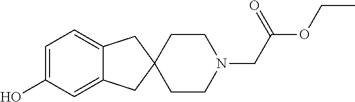 [SMILES: CCOC(=O)CN1CCC2(CC1)Cc1ccc(O)cc1C2]

To a solution of 1,3-dihydrospiro[indene-2,4′-piperidin]-5-ol (268 mg, 1.3 mmol) in 12 ml DMF was added DBU (301 mg; 2 mmol) and ethyl 2-bromoacetate (242 mg, 1.4 mmol). After stirring at RT for 75 minutes sat NH₄Cl solution was added. The reaction mixture was extracted with ethyl acetate and the combined organic layers discarded. The aqueous layer was reduced to dryness and the residue was stirred consecutively first with 500 ml ethyl acetate and then with 500 ml DCM. The organic layers were reduced to dryness affording 418 mg and 147 mg of product still containing inorganic impurities. This material was subsequently used without further purification.

### Ethyl 2-(5-((2-ethyl-6-fluorobenzyl)oxy)-1,3-dihydrospiro[indene-2,4′-piperidin]-1′-yl)acetate

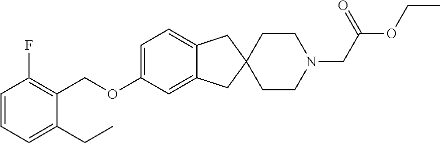 [SMILES: CCOC(=O)CN1CCC2(CC1)Cc1ccc(OCc3c(F)cccc3CC)cc1C2]

To a solution of ethyl 2-(5-hydroxy-1,3-dihydrospiro[indene-2,4′-piperidin]-1′-yl)acetate (78 mg, 0.27 mmol) in DMF was added potassium carbonate (56 mg, 0.4 mmol) and 2-(bromomethyl)-1-ethyl-3-fluorobenzene (65 mg, 0.3 mmol). After stirring at RT overnight water was added and the reaction mixture was extracted 3× with ethyl acetate. Combined organic layers were washed with sat NH₄C1 solution, dried over Na₂SO₄ and the solved evaporated. The residue was purified by chromatography affording 36 mg of desired product.

### 2-(5-((2-ethyl-6-fluorobenzyl)oxy)-1,3-dihydrospiro[indene-2,4′-piperidin]-1′-yl)acetic acid hydrochloride

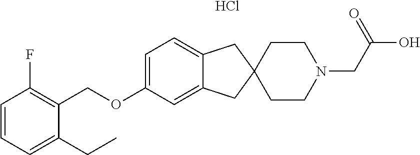 [SMILES: Cl ; CCc1cccc(F)c1COc1ccc2c(c1)CC1(CCN(CC(=O)O)CC1)C2]

To a solution of ethyl 2-(5-((2-ethyl-6-fluorobenzyl)oxy)-1,3-dihydrospiro[indene-2,4′-piperidin]-1′-yl)acetate (36 mg, 0.08 mmol) in THF was added sodium hydroxide (2M in water, 0.25 ml, 0.5 mmol). After stirring at RT overnight the solvent was evaporated, then water was added and the mixture adjusted to pH1 with 2N HCl. The precipitate was filtered of and dried affording 29 mg of desired product as a white solid.

¹H-NMR: (600 MHz, DMSO-d₆) δ 14.05 (s, 1H), 9.83 (s, 1H), 7.38 (td, J=8.0, 6.0 Hz, 1H), 7.17-7.05 (m, 4H), 6.90 (d, J=2.4 Hz, 1H), 6.81 (dd, J=8.2, 2.5 Hz, 1H), 5.03 (d, J=1.7 Hz, 2H), 4.14 (s, 2H), 2.81-2.76 (m, 2H), 2.70 (q, J=7.6 Hz, 2H), 1.82 (s, 4H), 1.17 (t, J=7.6 Hz, 3H).

MS: 398 (M+H⁺)

Examples 160 and 161 were prepared analogous to example 159

### Example 160. 2-(5-((2,6-dichlorobenzyl)oxy)-1,3-dihydrospiro[indene-2,4′-piperidin]-1′-yl)acetic acid

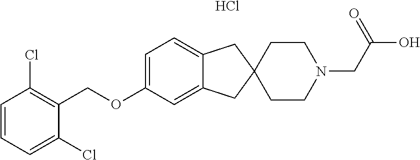 [SMILES: Cl ; O=C(O)CN1CCC2(CC1)Cc1ccc(OCc3c(Cl)cccc3Cl)cc1C2]

Example 160 was prepared as described for example 159 using 2-(bromomethyl)-1,3-dichlorobenzene instead of 2-(bromomethyl)-1-ethyl-3-fluorobenzene

MS: 420 (M+H+), isotope pattern for two chlorines

1H NMR (600 MHz, DMSO-d6; some signals may be covered by solvent) δ 7.56 (d, J=8.1 Hz, 2H), 7.50-7.44 (m, 1H), 7.11 (d, J=8.2 Hz, 1H), 6.90 (d, J=2.3 Hz, 1H), 6.80 (dd, J=8.2, 2.5 Hz, 1H), 5.17 (s, 2H), 3.03 (br, s, 4H), 2.78 (m, 2H), 2.73 (m, 2H), 1.73 (t, J=5.6 Hz, 4H).

### Example 161. 2-(5-((2-chloro-6-ethylbenzyl)oxy)-1,3-dihydrospiro[indene-2,4′-piperidin]-1′-yl)acetic acid hydrochloride

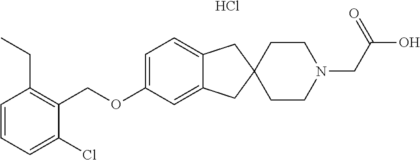 [SMILES: Cl ; CCc1cccc(Cl)c1COc1ccc2c(c1)CC1(CCN(CC(=O)O)CC1)C2]

### Example 161 was prepared as described for example 159 using 2-(bromomethyl)-1-chloro-3-ethylbenzene instead of 2-(bromomethyl)-1-ethyl-3-fluorobenzene

MS: 414 (M+H+), isotope pattern for one chlorine

1H NMR (600 MHz, DMSO-d6; some signals may be covered by solvent) δ 14.06 (br, s, 1H), 9.8 (br, s, 1H), 7.39-7.33 (m, 2H), 7.28 (dd, J=5.8, 3.1 Hz, 1H), 7.12 (d, J=8.2 Hz, 1H), 6.91 (d, J=2.3 Hz, 1H), 6.82 (dd, J=8.2, 2.5 Hz, 1H), 5.10 (s, 2H), 4.12 (s, 2H), 2.83-2.80 (m, 2H), 2.78-2.75 (m, 2H), 2.72 (q, J=7.5 Hz, 2H), 1.82 (m, 4H), 1.17 (t, J=7.5 Hz, 3H).

### 4. Assessment of Agonist Potency

Agonistic activity was measured as described below. The results are shown in table 1.

Genetically Engineered Cells

Cell clone CHO-A21-Edg1#17 carries the transgenes human EDG1 (S1PR1) receptor (Accession number NP_001391.2), mitochondrially targeted Aequorin (active part corresponds to accession number 1SL8_A) and chimaeric Gaqi5=Gaq modified with the 5 C-terminal amino-acids replaced with those of the Gai protein (DCGLF). CHO-A2-S1P3 Mix is a cell pool ectopically expressing human EDG3 (S1PR3) receptor (Accession number NP_005217.2) mitochondrially targeted Aequorin (active part has a sequence similar to accession number AY601106.1) and GNA16 (Accession number NP_002059.3). Cell clone CHO-A21-EDG8#12 carries the transgenes human EDG8 (S1PR5) receptor (Acession number NP_110387.1), mitochondrially expressed Aequorin (active part corresponds to accession number 1SL8_A) and chimaeric Gaqi5 (Gaq modified to present the 5 last amino-acids of the Gai protein “DCGLF”; see above). Cells are grown to mid-log phase in culture medium (HAM's F12, 10% FBS, 100 IU/mL penicillin, 100 μg/mL streptomycin, 250 μg/mL Zeocin, 400 μg/mL G418). 18 hours prior to frozen cells preparation, the medium is changed to remove the antibiotics.

Aequorin Assay

18 hours prior to the test, vials of frozen cells are quickly thawed in a 37° C. water bath, cells are recovered by centrifugation and resuspended in assay buffer (DMEM/HAM's F12 with HEPES, without phenol red+0.1% fatty acid-free BSA). Cells are gently agitated in suspension overnight at RT in presence of 5 μM of Coelenterazine h (Molecular Probes). On the day of the test, cells are diluted to their final working concentrations in assay buffer and agitated in suspension for 1 h at RT. Cells are then placed in the luminescence reader (Hamamatsu Functional Drug Screening System 6000, FDSS6000). During cells incubation, compounds are prepared in 100% DMSO, and subsequently diluted in assay buffer. Compounds are then dispensed in the assay plate (black, clear-bottom, 384-well plate). After binding of agonists to the human S1P receptor the intracellular calcium concentration increases and binding of calcium to the Aequorin/Coelenterazine complex leads to an oxidation reaction of coelenterazine, which results in the production of Aequorin, coelenteramide, CO2 and light (Dmax 469 nm). The luminescent response is dependent on the agonist concentration. For agonist testing, 30 μL of cell suspension are injected on 30 μL of test compound or reference agonist in the assay plate. The resulting emission of light is recorded for 90 seconds using the FDSS6000. Dose response curves with the reference compounds are performed before testing the compounds. S1P is the reference agonist and JTE-013 the reference antagonist for S1P2.

Following an incubation of 3 min after the first injection, 30 μL of the reference agonist for a final concentration corresponding to its EC80 is injected on the 60 μL of cell suspension and test compound mixture, for antagonist testing. The resulting emission of light is recorded for 90 seconds using the FDSS6000.

Luminescence data are integrated over the reading interval for agonist and antagonist modes. To standardize the emission of recorded light (determination of the “100% signal”) across plates and across different experiments, some of the wells contain 100 μM digitonin or a saturating concentration of ATP (20 μM). Plates also contain the reference agonist at a concentration equivalent to the EC80 obtained during the test validation and the EC100. Dose-response data from test compounds were analyzed with XLfit (IDBS) software using nonlinear regression applied to a sigmoidal dose-response model and the following equation:

### XL Fit fit Model 203: 4 Parameter Logistic Model

### A: Bottom

### B: TOP

### C: Log EC50

### D: Hill

fit=(A+((B−A)/(1+(((10\hat{ }C)/x)\hat{ }D))))

inv=((10\hat{ }C)/((((B−A)/(y−A))−1)\hat{ }(1/D)))

res=(y−fit)

TABLE 1

| | | S1P5 | | | |
|---|---|---|---|---|---|
| | | EC50 | | | |
| | | range | S1P1 | S1P3 | S1P4 |
| Ex. | Name | # | EC50 | EC50 | EC50 |
| 1 | 1((5′-((4-ethoxyphenyl)ethynyl)-1′,3′- | D | >1 μM | | >1 μM |
| | dihydrospiro[azetidine-3,2′-inden]-1- | | | | |
| | yl)methyl)cyclopropanecarboxylic acid | | | | |
| 2 | 1-((5′-((2-methoxy-4-propylbenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 3 | 2-(5′-((4-butyl-2-chlorobenzyl)oxy)-1′,3′- | D | >1 μM | | <1 μM |
| | dihydrospiro[azetidine-3,2′-inden]-1-yl)acetic acid | | | | |
| 4 | 2-(5′-((2-chloro-4-ethylbenzyl)oxy)-1′,3′- | C | >1 μM | | <1 μM |
| | dihydrospiro[azetidine-3,2′-inden]-1-yl)acetic acid | | | | |
| 5 | 3-(5′-((tetrahydro-2H-pyran-3-yl)methoxy)-1′,3′- | D | >1 μM | | >1 μM |
| | dihydrospiro[azetidine-3,2′-inden]-1- | | | | |
| | yl)cyclobutanecarboxylic acid | | | | |
| 6 | 3-(5′-(benzyloxy)-1′,3′-dihydrospiro[azetidine-3,2′- | D | >1 μM | >1 μM | <1 μM |
| | inden]-1-yl)propanoic acid | | | | |
| 7 | 3-(5′-((2-chloro-6-ethylbenzyl)oxy)-1′,3′- | A | >1 μM | >1 μM | <1 μM |
| | dihydrospiro[azetidine-3,2′-inden]-1-yl)propanoic | | | | |
| | acid | | | | |
| 8 | 2-(5′-(benzyloxy)-1′,3′-dihydrospiro[azetidine-3,2′- | C | >1 μM | >1 μM | <1 μM |
| | inden]-1-yl)acetic acid | | | | |
| 9 | 2-(5′-((2-chloro-6-methoxybenzyl)oxy)-1′,3′- | C | >1 μM | | >1 μM |
| | dihydrospiro[azetidine-3,2′-inden]-1-yl)acetic acid | | | | |
| 10 | 2-(5′-((2-fluoro-6-methoxybenzyl)oxy)-1′,3′- | C | >1 μM | | <1 μM |
| | dihydrospiro[azetidine-3,2′-inden]-1-yl)acetic acid | | | | |
| 11 | -(5′-((4-chlorobenzyl)oxy)-1′,3′- | D | >1 μM | >1 μM | <1 μM |
| | dihydrospiro[azetidine-3,2′-inden]-1-yl)acetic acid | | | | |
| 12 | 2-(5′-((2-(trifluoromethyl)benzyl)oxy)-1′,3′- | B | >1 μM | | <1 μM |
| | dihydrospiro[azetidine-3,2′-inden]-1-yl)acetic acid | | | | |
| 13 | 2-(5′-((2,6-difluorobenzyl)oxy)-1′,3′- | B | >1 μM | | <1 μM |
| | dihydrospiro[azetidine-3,2′-inden]-1-yl)acetic acid | | | | |
| 14 | 2-(5′-((3,5-difluorobenzyl)oxy)-1′,3′- | B | >1 μM | >1 μM | <1 μM |
| | dihydrospiro[azetidine-3,2′-inden]-1-yl)acetic acid | | | | |
| 15 | 2-(5′-((2-chloro-6-cyclopropylbenzyl)oxy)-1′,3′- | A | >1 μM | >1 μM | <1 μM |
| | dihydrospiro[azetidine-3,2′-inden]-1-yl)acetic acid | | | | |
| 16 | 2-(5′-((3-chlorobenzyl)oxy)-1′,3′- | A | >1 μM | >1 μM | <1 μM |
| | dihydrospiro[azetidine-3,2′-inden]-1-yl)acetic acid | | | | |
| | compound with acetic acid | | | | |
| 17 | 2-(5′-((2-fluoro-6-methylbenzyl)oxy)-1′,3′- | A | >1 μM | | <1 μM |
| | dihydrospiro[azetidine-3,2′-inden]-1-yl)acetic acid | | | | |
| 18 | 2-(5′-((2-chloro-6-fluorobenzyl)oxy)-1′,3′- | A | >1 μM | | <1 μM |
| | dihydrospiro[azetidine-3,2′-inden]-1-yl)acetic acid | | | | |
| 19 | 2-(5′-((2-fluoro-6-(trifluoromethyl)benzyl)oxy)- | A | >1 μM | | <1 μM |
| | 1′,3′-dihydrospiro[azetidine-3,2′-inden]-1-yl)acetic | | | | |
| | acid | | | | |
| 20 | 2-(5′-((2-chloro-6-(trifluoromethyl)benzyl)oxy)- | A | >1 μM | | <1 μM |
| | 1′,3′-dihydrospiro[azetidine-3,2′-inden]-1-yl)acetic | | | | |
| | acid | | | | |
| 21 | 2-(5′-((2-ethyl-6-fluorobenzyl)oxy)-1′,3′- | A | >1 μM | | <1 μM |
| | dihydrospiro[azetidine-3,2′-inden]-1-yl)acetic acid | | | | |
| 22 | 2-(5′-((4-bromo-2-chlorobenzyl)oxy)-1′,3′- | B | >1 μM | | <1 μM |
| | dihydrospiro[azetidine-3,2′-inden]-1-yl)acetic acid | | | | |
| 23 | 2-(5′-((2,5-dichlorobenzyl)oxy)-1′,3′- | A | >1 μM | >1 μM | <1 μM |
| | dihydrospiro[azetidine-3,2′-inden]-1-yl)acetic acid | | | | |
| 24 | 2-(5′-((2-chloro-6-ethylbenzyl)oxy)-1′,3′- | A | >1 μM | >1 μM | <1 μM |
| | dihydrospiro[azetidine-3,2′-inden]-1-yl)acetic acid | | | | |
| 25 | 2-(5′-((3,4-dichlorobenzyl)oxy)-1′,3′- | B | >1 μM | >1 μM | <1 μM |
| | dihydrospiro[azetidine-3,2′-inden]-1-yl)acetic acid | | | | |
| 26 | 2-(5′-((2,6-dimethylbenzyl)oxy)-1′,3′- | | | | |
| | dihydrospiro[azetidine-3,2′-inden]-1-yl)acetic acid | | | | |
| 27 | 2-(5′-((5-fluoro-2-(trifluoromethyl)benzyl)oxy)- | A | >1 μM | | <1 μM |
| | 1′,3′-dihydrospiro[azetidine-3,2′-inden]-1-yl)acetic | | | | |
| | acid | | | | |
| 28 | 1-((5′-((2-chloro-6-ethylbenzyl)oxy)-1′,3′- | A | >1 μM | >1 μM | <1 μM |
| | dihydrospiro[azetidine-3,2′-inden]-1- | | | | |
| | yl)methyl)cyclopropanecarboxylic acid | | | | |
| 29 | 1-((5′-(benzyloxy)-1′,3′-dihydrospiro[azetidine- | D | >1 μM | >1 μM | >1 μM |
| | 3,2′-inden]-1-yl)methyl)cyclopropanecarboxylic | | | | |
| | acid | | | | |
| 30 | 1-((5′-((2-fluoro-6-methoxybenzyl)oxy)-1′,3′- | D | >1 μM | | >1 μM |
| | dihydrospiro[azetidine-3,2′-inden]-1- | | | | |
| | yl)methyl)cyclopropanecarboxylic acid | | | | |
| 31 | 1-((5′-((2-chloro-6-methoxybenzyl)oxy)-1′,3′- | C | >1 μM | | <1 μM |
| | dihydrospiro[azetidine-3,2′-inden]-1- | | | | |
| | yl)methyl)cyclopropanecarboxylic acid | | | | |
| 32 | 1-((5′-((2-chloro-4-ethoxybenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 33 | 1-((5′-((4-chlorobenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 34 | 1-((5′-((2-chloro-6-fluorobenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 35 | 1-((5′-((4-ethoxybenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 36 | 1-((5′-((2,5-dichlorobenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 37 | 1-((5′-((2,6-difluorobenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 38 | 1-((5′-((2,4-dichlorobenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 39 | 1-((5′-((2,6-dimethylbenzyl)oxy)-1′,3′- | A | >1 μM | >1 μM | <1 μM |
| | dihydrospiro[azetidine-3,2′-inden]-1- | | | | |
| | yl)methyl)cyclopropanecarboxylic acid | | | | |
| 40 | 1-((5′-((2-chloro-4-methoxybenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 41 | 1-((5′-((2-(trifluoromethyl)benzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 42 | 1-((5′-((2-fluoro-6-methylbenzyl)oxy)-1′,3′- | C | >1 μM | | <1 μM |
| | dihydrospiro[azetidine-3,2′-inden]-1- | | | | |
| | yl)methyl)cyclopropanecarboxylic acid | | | | |
| 43 | 1-((5′-((5-fluoro-2-(trifluoromethyl)benzyl)oxy)- | D | >1 μM | | <1 μM |
| | 1′,3′-dihydrospiro[azetidine-3,2′-inden]-1- | | | | |
| | yl)methyl)cyclopropanecarboxylic acid | | | | |
| 44 | 1-((5′-((2-chloro-6-cyclopropylbenzyl)oxy)-1′,3′- | A | >1 μM | >1 μM | <1 μM |
| | dihydrospiro[azetidine-3,2′-inden]-1- | | | | |
| | yl)methyl)cyclopropanecarboxylic acid | | | | |
| 45 | 1-((5′-((3-chlorobenzyl)oxy)-1′,3′- | B | >1 μM | >1 μM | <1 μM |
| | dihydrospiro[azetidine-3,2′-inden]-1- | | | | |
| | yl)methyl)cyclopropanecarboxylic acid | | | | |
| 46 | 1-((5′-((2,6-dichlorobenzyl)oxy)-1′,3′- | A | >1 μM | >1 μM | <1 μM |
| | dihydrospiro[azetidine-3,2′-inden]-1- | | | | |
| | yl)methyl)cyclopropanecarboxylic acid | | | | |
| 47 | 1-((5′-((2-chloro-6-(trifluoromethyl)benzyl)oxy)- | A | >1 μM | | <1 μM |
| | 1′,3′-dihydrospiro[azetidine-3,2′-inden]-1- | | | | |
| | yl)methyl)cyclopropanecarboxylic acid | | | | |
| 48 | 1-((5′-((2-ethyl-6-fluorobenzyl)oxy)-1′,3′- | A | >1 μM | | <1 μM |
| | dihydrospiro[azetidine-3,2′-inden]-1- | | | | |
| | yl)methyl)cyclopropanecarboxylic acid | | | | |
| 49 | 1-((5′-((2-fluoro-6-(trifluoromethyl)benzyl)oxy)- | A | >1 μM | | <1 μM |
| | 1′,3′-dihydrospiro[azetidine-3,2′-inden]-1- | | | | |
| | yl)methyl)cyclopropanecarboxylic acid | | | | |
| 50 | 1-((5′-(4-((3-fluorobenzyl)oxy)phenyl)-1′,3′- | A | >1 μM | >1 μM | <1 μM |
| | dihydrospiro[azetidine-3,2′-inden]-1- | | | | |
| | yl)methyl)cyclopropanecarboxylic acid | | | | |
| 51 | 1-((5′-(4-(cyclohexylmethoxy)phenyl)-1′,3′- | B | >1 μM | >1 μM | >1 μM |
| | dihydrospiro[azetidine-3,2′-inden]-1- | | | | |
| | yl)methyl)cyclopropanecarboxylic acid | | | | |
| 52 | 1-((5′-(4-((3-chlorobenzyl)oxy)phenyl)-1′,3′- | B | >1 μM | >1 μM | <1 μM |
| | dihydrospiro[azetidine-3,2′-inden]-1- | | | | |
| | yl)methyl)cyclopropanecarboxylic acid | | | | |
| 53 | 1-((5′-(4-(benzyloxy)phenyl)-1′,3′- | C | >1 μM | | <1 μM |
| | dihydrospiro[azetidine-3,2′-inden]-1- | | | | |
| | yl)methyl)cyclopropanecarboxylic acid | | | | |
| 54 | 1-((5′-phenethoxy-1′,3′-dihydrospiro[azetidine- | D | >1 μM | | <1 μM |
| | 3,2′-inden]-1-yl)methyl)cyclopropanecarboxylic | | | | |
| | acid | | | | |
| 55 | 1-((5′-(4-chlorophenethoxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 56 | 1-((5′-(2-(trifluoromethyl)phenethoxy)-1′,3′- | B | >1 μM | | <1 μM |
| | dihydrospiro[azetidine-3,2′-inden]-1- | | | | |
| | yl)methyl)cyclopropanecarboxylic acid | | | | |
| 57 | 1-((5′-(3-chlorophenethoxy)-1′,3′- | C | <1 μM | | <1 μM |
| | dihydrospiro[azetidine-3,2′-inden]-1- | | | | |
| | yl)methyl)cyclopropanecarboxylic acid | | | | |
| 58 | 1-((5′-(2-chlorophenethoxy)-1′,3′- | C | >1 μM | | <1 μM |
| | dihydrospiro[azetidine-3,2′-inden]-1- | | | | |
| | yl)methyl)cyclopropanecarboxylic acid | | | | |
| 59 | 1-((5′-(2,6-dichlorophenethoxy)-1′,3′- | A | >1 μM | >1 μM | <1 μM |
| | dihydrospiro[azetidine-3,2′-inden]-1- | | | | |
| | yl)methyl)cyclopropanecarboxylic acid | | | | |
| 60 | 2-(5′-phenethoxy-1′,3′-dihydrospiro[azetidine-3,2′- | D | >1 μM | | >1 μM |
| | inden]-1-yl)acetic acid | | | | |
| 61 | 2-(5′-(2-chlorophenethoxy)-1′,3′- | B | >1 μM | | <1 μM |
| | dihydrospiro[azetidine-3,2′-inden]-1-yl)acetic acid | | | | |
| 62 | 2-(5′-(2-chlorophenethoxy)-1′,3′- | A | >1 μM | >1 μM | <1 μM |
| | dihydrospiro[azetidine-3,2′-inden]-1-yl)acetic acid | | | | |
| 63 | 2-(5′-(2,6-dichlorophenethoxy)-1′,3′- | A | >1 μM | >1 μM | <1 μM |
| | dihydrospiro[azetidine-3,2′-inden]-1-yl)acetic acid | | | | |
| 64 | 4-(5′-((2-chloro-6-ethylbenzyl)oxy)-1′,3′- | A | | >1 μM | <1 μM |
| | dihydrospiro[azetidine-3,2′-inden]-1-yl)-3- | | | | |
| | methylbutanoic acid | | | | |
| 65 | 4-(5′-((2-ethyl-6-fluorobenzyl)oxy)-1′,3′- | B | >1 μM | | <1 μM |
| | dihydrospiro[azetidine-3,2′-inden]-1-yl)-3- | | | | |
| | methylbutanoic acid | | | | |
| 66 | 2-(5′-((2,3-dihydro-1H-inden-1-yl)oxy)-1′,3′- | A | >1 μM | | <1 μM |
| | dihydrospiro[azetidine-3,2′-inden]-1-yl)acetic acid | | | | |
| 67 | 2-(5′-((7-chloro-2,3-dihydro-1H-inden-1-yl)oxy)- | A | >1 μM | | <1 μM |
| | 1′,3′-dihydrospiro[azetidine-3,2′-inden]-1-yl)acetic | | | | |
| | acid | | | | |
| 68 | 2-(5′-((5-bromo-7-fluoro-2,3-dihydro-1H-inden-1- | A | >1 μM | | <1 μM |
| | yl)oxy)-1′,3′-dihydrospiro[azetidine-3,2′-inden]-1- | | | | |
| | yl)acetic acid | | | | |
| 69 | 2-(5′-((5,7-dichloro-2,3-dihydro-1H-inden-1- | A | >1 μM | | <1 μM |
| | yl)oxy)-1′,3′-dihydrospiro[azetidine-3,2′-inden]-1- | | | | |
| | yl)acetic acid | | | | |
| 70 | 2-(5′-(2-phenoxyethoxy)-1′,3′- | C | >1 μM | | <1 μM |
| | dihydrospiro[azetidine-3,2′-inden]-1-yl)acetic acid | | | | |
| 71 | 2-(5′-(2-(4-chlorophenoxy)ethoxy)-1′,3′- | D | >1 μM | | >1 μM |
| | dihydrospiro[azetidine-3,2′-inden]-1-yl)acetic acid | | | | |
| 72 | 2-(5′-((2,3-dihydrobenzofuran-7-yl)methoxy)-1′,3′- | C | >1 μM | | <1 μM |
| | dihydrospiro[azetidine-3,2′-inden]-1-yl)acetic acid | | | | |
| 73 | 2-(5′-((2,3-dihydro-1H-inden-2-yl)oxy)-1′,3′- | C | >1 μM | | <1 μM |
| | dihydrospiro[azetidine-3,2′-inden]-1-yl)acetic acid | | | | |
| 74 | 2-(5′-((2,3-dihydro-1H-inden-4-yl)methoxy)-1′,3′- | A | >1 μM | | <1 μM |
| | dihydrospiro[azetidine-3,2′-inden]-1-yl)acetic acid | | | | |
| 75 | 2-(5′-((6-isopropoxypyridin-3-yl)ethynyl)-1′,3′- | A | <1 μM | | >1 μM |
| | dihydrospiro[azetidine-3,2′-inden]-1-yl)acetic acid | | | | |
| 76 | 2-(5′-((4-methylcyclohexyl)ethynyl)-1′,3′- | A | >1 μM | | <1 μM |
| | dihydrospiro[azetidine-3,2′-inden]-1-yl)acetic acid | | | | |
| 77 | 2-(5′-((4-ethoxyphenyl)ethynyl)-1′,3′- | A | <1 μM | | <1 μM |
| | dihydrospiro[azetidine-3,2′-inden]-1-yl)acetic acid | | | | |
| 78 | 2-(5′-((6-(cyclopentyloxy)pyridin-3-yl)ethynyl)- | A | <1 μM | | <1 μM |
| | 1′,3′-dihydrospiro[azetidine-3,2′-inden]-1-yl)acetic | | | | |
| | acid | | | | |
| 79 | 2-(5′-((6-((1,1,1-trifluoropropan-2-yl)oxy)pyridin- | A | <1 μM | | <1 μM |
| | 3-yl)ethynyl)-1′,3′-dihydrospiro[azetidine-3,2′- | | | | |
| | inden]-1-yl)acetic acid | | | | |
| 80 | 2-(5′((2-chlorophenyl)ethynyl)-1′,3′- | A | >1 μM | | <1 μM |
| | dihydrospiro[azetidine-3,2′-inden]-1-yl)acetic acid | | | | |
| 81 | 2-(5′-((2-fluoro-4-methoxyphenyl)ethynyl)-1′,3′- | | | | |
| | dihydrospiro[azetidine-3,2′-inden]-1-yl)acetic acid | | | | |
| 82 | 2-(5′-(phenylethynyl)-1′,3′-dihydrospiro[azetidine- | B | >1 μM | | <1 μM |
| | 3,2′-inden]-1-yl)acetic acid | | | | |
| 83 | 3-(5′-((2,3-dihydro-1H-inden-4-yl)methoxy)-1′,3′- | B | >1 μM | | <1 μM |
| | dihydrospiro[azetidine-3,2′-inden]-1- | | | | |
| | yl)cyclobutanecarboxylic acid | | | | |
| 84 | 3-(5′-((4-(cyclopropylmethoxy)benzyl)oxy)-1′,3′- | D | >1 μM | | >1 μM |
| | dihydrospiro[azetidine-3,2′-inden]-1- | | | | |
| | yl)cyclobutanecarboxylic acid | | | | |
| 85 | 3-(5′-((4-ethoxy-2,6-difluorobenzyl)oxy)-1′,3′- | D | >1 μM | | <1 μM |
| | dihydrospiro[azetidine-3,2′-inden]-1- | | | | |
| | yl)cyclobutanecarboxylic acid | | | | |
| 86 | 3-(5′-((4-ethoxy-2,3-difluorobenzyl)oxy)-1′,3′- | D | >1 μM | | <1 μM |
| | dihydrospiro[azetidine-3,2′-inden]-1- | | | | |
| | yl)cyclobutanecarboxylic acid | | | | |
| 87 | 3-(5′((2-cyclopropyl-6-fluorobenzyl)oxy)-1′,3′- | A | >1 μM | | <1 μM |
| | dihydrospiro[azetidine-3,2′-inden]-1- | | | | |
| | yl)cyclobutanecarboxylic acid | | | | |
| 88 | 3-(5′-((6-methoxy-2-methylpyridin-3-yl)methoxy)- | D | >1 μM | | <1 μM |
| | 1′,3′-dihydrospiro[azetidine-3,2′-inden]-1- | | | | |
| | yl)cyclobutanecarboxylic acid | | | | |
| 89 | (1r,3r)-3-(5′-((2,5-dichlorobenzyl)oxy)-1′,3′- | B | >1 μM | | <1 μM |
| | dihydrospiro[azetidine-3,2′-inden]-1- | | | | |
| | yl)cyclobutanecarboxylic acid | | | | |
| 90 | (1s,3s)-3-(5′-((2,5-dichlorobenzyl)oxy)-1′,3′- | C | >1 μM | | <1 μM |
| | dihydrospiro[azetidine-3,2′-inden]-1- | | | | |
| | yl)cyclobutanecarboxylic acid | | | | |
| 91 | 3-(5′-((2-chloro-6-cyclopropylbenzyl)oxy)-1′,3′- | A | <1 μM | | <1 μM |
| | dihydrospiro[azetidine-3,2′-inden]-1- | | | | |
| | yl)cyclobutanecarboxylic acid | | | | |
| 92 | 3-(5′-((2,6-dichlorobenzyl)oxy)-1′,3′- | A | >1 μM | | <1 μM |
| | dihydrospiro[azetidine-3,2′-inden]-1- | | | | |
| | yl)cyclobutanecarboxylic acid | | | | |
| 93 | 3-(5′-((2,5-dichlorobenzyl)oxy)-1′,3′- | B | >1 μM | | <1 μM |
| | dihydrospiro[azetidine-3,2′-inden]-1- | | | | |
| | yl)cyclobutanecarboxylic acid | | | | |
| 94 | 3-(5′-((2-chloro-6-ethylbenzyl)oxy)-1′,3′- | A | <1 μM | | <1 μM |
| | dihydrospiro[azetidine-3,2′-inden]-1- | | | | |
| | yl)cyclobutanecarboxylic acid | | | | |
| 95 | 3-(5′-((6-(cyclopentyloxy)pyridin-3-yl)ethynyl)- | A | <1 μM | | <1 μM |
| | 1′,3′-dihydrospiro[azetidine-3,2′-inden]-1- | | | | |
| | yl)cyclobutanecarboxylic acid | | | | |
| 96 | 3-(5′((6-isopropoxypyridin-3-yl)ethynyl)-1′,3′- | B | <1 μM | | >1 μM |
| | dihydrospiro[azetidine-3,2′-inden]-1- | | | | |
| | yl)cyclobutanecarboxylic acid | | | | |
| 97 | 3-(5′-((4-isopropoxyphenyl)ethynyl)-1′,3′- | A | <1 μM | | >1 μM |
| | dihydrospiro[azetidine-3,2′-inden]-1- | | | | |
| | yl)cyclobutanecarboxylic acid | | | | |
| 98 | 3-(5′-((4-ethoxyphenyl)ethynyl)-1′,3′- | B | <1 μM | | >1 μM |
| | dihydrospiro[azetidine-3,2′-inden]-1- | | | | |
| | yl)cyclobutanecarboxylic acid | | | | |
| 99 | 3-(5′-((6-(cyclopropylmethoxy)pyridin-3- | C | <1 μM | | >1 μM |
| | yl)ethynyl)-1′,3′-dihydrospiro[azetidine-3,2′- | | | | |
| | inden]-1-yl)cyclobutanecarboxylic acid | | | | |
| 100 | 3-(5′-((4-ethoxy-3-fluorophenyl)ethynyl)-1′,3′- | A | <1 μM | | >1 μM |
| | dihydrospiro[azetidine-3,2′-inden]-1- | | | | |
| | yl)cyclobutanecarboxylic acid | | | | |
| 101 | 3-(5′-((4-methoxy-2-methylphenyl)ethynyl)-1′,3′- | D | >1 μM | | <1 μM |
| | dihydrospiro[azetidine-3,2′-inden]-1- | | | | |
| | yl)cyclobutanecarboxylic acid | | | | |
| 102 | 3-(5′-((4-methylcyclohexyl)ethynyl)-1′,3′- | A | >1 μM | | <1 μM |
| | dihydrospiro[azetidine-3,2′-inden]-1- | | | | |
| | yl)cyclobutanecarboxylic acid | | | | |
| 103 | 3-(5′-((6-((1,1,1-trifluoropropan-2-yl)oxy)pyridin- | A | <1 μM | | <1 μM |
| | 3-yl)ethynyl)-1′,3′-dihydrospiro[azetidine-3,2′- | | | | |
| | inden]-1-yl)cyclobutanecarboxylic acid | | | | |
| 104 | 3-(5′-((2,3-dihydro-1H-inden-1-yl)oxy)-1′,3′- | A | >1 μM | | <1 μM |
| | dihydrospiro[azetidine-3,2′-inden]-1- | | | | |
| | yl)cyclobutanecarboxylic acid | | | | |
| 105 | 3-(5′-(2-(trifluoromethyl)phenethoxy)-1′,3′- | D | >1 μM | | <1 μM |
| | dihydrospiro[azetidine-3,2′-inden]-1- | | | | |
| | yl)cyclobutanecarboxylic acid | | | | |
| 106 | 3-(5′-((7-chloro-2,3-dihydro-1H-inden-1-yl)oxy)- | A | <1 μM | | <1 μM |
| | 1′,3′-dihydrospiro[azetidine-3,2′-inden]-1- | | | | |
| | yl)cyclobutanecarboxylic acid | | | | |
| 107 | 3-(5′-((4-ethoxy-3,5-difluorobenzyl)oxy)-1′,3′- | | | | |
| | dihydrospiro[azetidine-3,2′-inden]-1- | | | | |
| | yl)cyclobutanecarboxylic acid | | | | |
| 108 | 1-((5′-(4-((4-chlorobenzyl)oxy)phenyl)-1′,3′- | B | >1 μM | >1 μM | >1 μM |
| | dihydrospiro[azetidine-3,2′-inden]-1- | | | | |
| | yl)methyl)cyclopropanecarboxylic acid | | | | |
| 109 | 2-(5′-((2,6-dichlorobenzyl)oxy)-1′,3′- | A | >1 μM | >1 μM | <1 μM |
| | dihydrospiro[azetidine-3,2′-inden]-1-yl)acetic acid | | | | |
| 110 | 2-(5′-((2-cyclopropyl-6-fluorobenzyl)oxy)-1′,3′- | A | >1 μM | >1 μM | <1 μM |
| | dihydrospiro[azetidine-3,2′-inden]-1-yl)acetic acid | | | | |
| 111 | 2-(5′-((2-cyclopropylbenzyl)oxy)-1′,3′- | A | >1 μM | >1 μM | <1 μM |
| | dihydrospiro[azetidine-3,2′-inden]-1-yl)acetic acid | | | | |
| 112 | 1-((5′-((4-ethoxyphenyl)ethynyl)-1′,3′- | C | >1 μM | | >1 μM |
| | dihydrospiro[azetidine-3,2′-inden]-1- | | | | |
| | yl)methyl)cyclopropanecarboxylic acid | | | | |
| 113 | 1-((5′-((4-isopropoxyphenyl)ethynyl)-1′,3′- | A | <1 μM | | >1 μM |
| | dihydrospiro[azetidine-3,2′-inden]-1- | | | | |
| | yl)methyl)cyclopropanecarboxylic acid | | | | |
| 114 | 1-((5′-((6-isopropoxypyridin-3-yl)ethynyl)-1′,3′- | C | >1 μM | | >1 μM |
| | dihydrospiro[azetidine-3,2′-inden]-1- | | | | |
| | yl)methyl)cyclopropanecarboxylic acid | | | | |
| 115 | 1-((5′-((4-methylcyclohexyl)ethynyl)-1′,3′- | A | >1 μM | | <1 μM |
| | dihydrospiro[azetidine-3,2′-inden]-1- | | | | |
| | yl)methyl)cyclopropanecarboxylic acid | | | | |
| 116 | 1-((5′-((6-(cyclopentyloxy)pyridin-3-yl)ethynyl)- | B | >1 μM | | >1 μM |
| | 1′,3′-dihydrospiro[azetidine-3,2′-inden]-1- | | | | |
| | yl)methyl)cyclopropanecarboxylic acid | | | | |
| 117 | 1-((5′-((6-((1,1,1-trifluoropropan-2-yl)oxy)pyridin- | A | <1 μM | | >1 μM |
| | 3-yl)ethynyl)-1′,3′-dihydrospiro[azetidine-3,2′- | | | | |
| | inden]-1-yl)methyl)cyclopropanecarboxylic acid | | | | |
| 118 | 1-((5′-((4-methoxy-2-methylphenyl)ethynyl)-1′,3′- | D | >1 μM | | <1 μM |
| | dihydrospiro[azetidine-3,2′-inden]-1- | | | | |
| | yl)methyl)cyclopropanecarboxylic acid | | | | |
| 119 | 1-((5′-(2-(6-isopropoxypyridin-3-yl)ethyl)-1′,3′- | D | >1 μM | | >1 μM |
| | dihydrospiro[azetidine-3,2′-inden]-1- | | | | |
| | yl)methyl)cyclopropanecarboxylic acid | | | | |
| 120 | 1-((5′-((4-ethoxy-3,5-difluorobenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 121 | 1-((5′-((6-methoxy-2-methylpyridin-3- | D | >1 μM | | >1 μM |
| | yl)methoxy)-1′,3′-dihydrospiro[azetidine-3,2′- | | | | |
| | inden]-1-yl)methyl)cyclopropanecarboxylic acid | | | | |
| 122 | 1-((5′-((4-ethoxy-2,6-difluorobenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 123 | 1-((5′-((4-ethoxy-2,3-difluorobenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 124 | 1-((5′-((2,5-dichloropyridin-3-yl)methoxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 125 | 1-((5′-((2,3-dihydro-1H-inden-4-yl)methoxy)-1′,3′- | B | >1 μM | | <1 μM |
| | dihydrospiro[azetidine-3,2′-inden]-1- | | | | |
| | yl)methyl)cyclopropanecarboxylic acid | | | | |
| 126 | 1-((5′-(cyclohexylmethoxy)-1′,3′- | C | >1 μM | | <1 μM |
| | dihydrospiro[azetidine-3,2′-inden]-1- | | | | |
| | yl)methyl)cyclopropanecarboxylic acid | | | | |
| 127 | 1-((5′-((2,3-dihydro-1H-inden-2-yl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 128 | 1-((5′-((4-(cyclopropylmethoxy)benzyl)oxy)-1′,3′- | D | >1 μM | | >1 μM |
| | dihydrospiro[azetidine-3,2′-inden]-1- | | | | |
| | yl)methyl)cyclopropanecarboxylic acid | | | | |
| 129 | 1-((5′-((7-chloro-2,3-dihydro-1H-inden-1-yl)oxy)- | A | >1 μM | | <1 μM |
| | 1′,3′-dihydrospiro[azetidine-3,2′-inden]-1- | | | | |
| | yl)methyl)cyclopropanecarboxylic acid | | | | |
| 130 | 1-((5′-((2,3-dihydro-1H-inden-1-yl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 131 | 1-((5′-((2-cyclopropyl-6-fluorobenzyl)oxy)-1′,3′- | A | >1 μM | | <1 μM |
| | dihydrospiro[azetidine-3,2′-inden]-1- | | | | |
| | yl)methyl)cyclopropanecarboxylic acid | | | | |
| 132 | 1-((5′-(2-cyclohexylethoxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 133 | 1-((5′-((4-methylbenzyl)oxy)-1′,3′- | | | | |
| | dihydrospiro[azetidine-3,2′-inden]-1- | | | | |
| | yl)methyl)cyclopropanecarboxylic acid | | | | |
| 134 | 1-((5′-(2-methylphenethoxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 135 | 1-((5′-(4-methoxyphenethoxy)-1′,3′- | D | >1 μM | | >1 μM |
| | dihydrospiro[azetidine-3,2′-inden]-1- | | | | |
| | yl)methyl)cyclopropanecarboxylic acid | | | | |
| 136 | 1-((5′-(2-fluorophenethoxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 137 | 1-((5′-(4-fluorophenethoxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 138 | 1-((5′-((2-methylbenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 139 | 1-((5′-((4-methoxybenzyl)oxy)-1′,3′- | D | >1 μM | | >1 μM |
| | dihydrospiro[azetidine-3,2′-inden]-1- | | | | |
| | yl)methyl)cyclopropanecarboxylic acid | | | | |
| 140 | 1*((5′-((2-fluorobenzyl)oxy)-1′,3′- | D | >1 μM | | >1 μM |
| | dihydrospiro[azetidine-3,2′-inden]-1- | | | | |
| | yl)methyl)cyclopropanecarboxylic acid | | | | |
| 141 | 1-((5′-((2-chlorobenzyl)oxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 142 | 1-((5′-((4-cyanobenzyl)oxy)-1′,3′- | D | >1 μM | | >1 μM |
| | dihydrospiro[azetidine-3,2′-inden]-1- | | | | |
| | yl)methyl)cyclopropanecarboxylic acid | | | | |
| 143 | 1-((5′-(benzo[d][1,3]dioxo1-5-ylmethoxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 144 | 1-((5′-(2-(1H-indol-3-yl)ethoxy)-1′,3′- | D | >1 μM | | >1 μM |
| | dihydrospiro[azetidine-3,2′-inden]-1- | | | | |
| | yl)methyl)cyclopropanecarboxylic acid | | | | |
| 145 | 1-((5′-(2-(2-chlorophenoxy)ethoxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 146 | 1-((5′-(3-(6-methylpyridin-2-yl)propoxy)-1′,3′- | D | >1 μM | | >1 μM |
| | dihydrospiro[azetidine-3,2′-inden]-1- | | | | |
| | yl)methyl)cyclopropanecarboxylic acid | | | | |
| 147 | 1-((5′-((3-cyanobenzyl)oxy)-1′,3′- | C | >1 μM | | <1 μM |
| | dihydrospiro[azetidine-3,2′-inden]-1- | | | | |
| | yl)methyl)cyclopropanecarboxylic acid | | | | |
| 148 | 1-((5′-(3-fluorophenethoxy)-1′,3′- | D | >1 μM | | <1 μM |
| | dihydrospiro[azetidine-3,2′-inden]-1- | | | | |
| | yl)methyl)cyclopropanecarboxylic acid | | | | |
| 149 | 2-(6′-((4-chlorophenyl)ethynyl)-3′H- | D | >1 μM | | >1 μM |
| | spiro[azetidine-3,2′-benzofuran]-1-yl)acetic acid | | | | |
| 150 | 2-(6′-(cyclohexylethynyl)-3′H-spiro[azetidine-3,2′- | A | >1 μM | | <1 μM |
| | benzofuran]-1-yl)acetic acid | | | | |
| 151 | 2-(6′-(phenylethynyl)-3′H-spiro[azetidine-3,2′- | A | >1 μM | | <1 μM |
| | benzofuran]-1-yl)acetic acid | | | | |
| 152 | 2-(6′-((3-chlorophenyl)ethynyl)-3′H- | A | >1 μM | | <1 μM |
| | spiro[azetidine-3,2′-benzofuran]-1-yl)acetic acid | | | | |
| 153 | 2-(6′-((2-chlorophenyl)ethynyl)-3′H- | A | >1 μM | | <1 μM |
| | spiro[azetidine-3,2′-benzofuran]-1-yl)acetic acid | | | | |
| 154 | 2-(6′-((4-ethoxyphenyl)ethynyl)-3′H- | B | <1 μM | | >1 μM |
| | spiro[azetidine-3,2′-benzofuran]-1-yl)acetic acid | | | | |
| 155 | 2-(6′-((4-methylcyclohexyl)ethynyl)-3′H- | C | >1 μM | | <1 μM |
| | spiro[azetidine-3,2′-benzofuran]-1-yl)acetic acid | | | | |
| 156 | 1-((6′-((2-chloro-6-ethylbenzyl)oxy)-3′H- | A | >1 μM | | <1 μM |
| | spiro[azetidine-3,2′-benzofuran]-1- | | | | |
| | yl)methyl)cyclopropanecarboxylic acid | | | | |
| 157 | 4-(6′-((2-chloro-6-ethylbenzyl)oxy)-3′H- | A | >1 μM | | <1 μM |
| | spiro[azetidine-3,2′-benzofuran]-1-yl)-3- | | | | |
| | methylbutanoic acid | | | | |
| 158 | 2-(6′-((2-chloro-6-ethylbenzyl)oxy)-3′H- | A | <1 μM | | <1 μM |
| | spiro[azetidine-3,2′-benzofuran]-1-yl)acetic acid | | | | |
| 159 | 2-(5((2-ethyl-6-fluorobenzyl)oxy)-1,3- | A | >1 μM | >1 μM | <1 μM |
| | dihydrospiro[indene-2,4′-piperidin]-1′-yl)acetic | | | | |
| | acid | | | | |
| 160 | 2-(5-((2,6-dichlorobenzyl)oxy)-1,3- | A | >1 μM | >1 μM | <1 μM |
| | dihydrospiro[indene-2,4′-piperidin]-1′-yl)acetic | | | | |
| | acid | | | | |
| 161 | 2-(5-((2-chloro-6-ethylbenzyl)oxy)-1,3- | A | >1 μM | >1 μM | <1 μM |
| | dihydrospiro[indene-2,4′-piperidin]-1′-yl)acetic | | | | |
| | acid | | | | |

# A: EC50 ≤ 10 nM
B: 10 nM < EC50 ≤ 100 nM
C: 100 nM < EC50 ≤ 1 μM
D: 1 μM EC50 < EC50 ≤ 10 μM

1. A compound of formula (I): 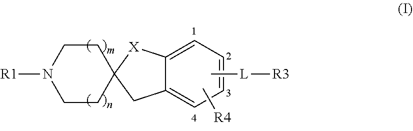
or a pharmaceutically acceptable salt thereof, wherein
X is CH₂ or O;
m and n are independently 0 or 1;
R1 is selected from the group consisting of —(C1-4)alkylene-R2, —(C3-6)cycloalkylene-R2, —(C1-3)alkylene-(C3-6)cycloalkylene-R2 and —(C3-6)cycloalkylene-(C1-3)alkylene-R2, wherein the (C1-4)alkylene is optionally substituted with up to 3 carbon atoms, with (CH₂)₂ to form a cyclopropyl moiety or with (CH₂)₃ to form a cyclobutyl moiety and wherein R2 is selected from the group consisting of —COOH, —OH, —OPO₃H₂, —PO₃H₂, —COO(C1-4)alkyl and tetrazol-5-yl;
L is attached to atom 1, 2, 3 or 4 and is a group —W—(CH₂)p-T- wherein:
W is attached to the phenylene moiety and selected from the group consisting of —O—, —CO—, —S—, —SO—, —SO₂—, —NH—, —CH₂—CH₂—, —CF₂—CH₂—, —CH₂—CF₂—, —CH═CH—, —C(CF₃)═CH—, —CH═C(CF₃)—, —C≡C—, phenyl, —(C3-7)cycloalkyl-, -pyridyl-, -thienyl- and -thiazolyl-, wherein the phenyl, (C3-7)cycloalkyl, pyridyl, thienyl or thiazolyl is optionally substituted with one or more substituents independently selected from the group consisting of a halogen atom, hydroxy, (C1-4)alkyl optionally substituted with one or more halogen atoms, and (C1-4)alkoxy optionally substituted with one or more halogen atoms;
p is 0 or an integer from 1 to 4; and
T is absent or attached to R3 and selected from the group consisting of —O—, —O—(C1-4)alkyl-, —S—, —SO—, —SO₂—, —NH—, —CO—, —CH═CH—, —C≡C— and cyclopropylene;
R3 is selected from the group consisting of (C3-6)cycloalkyl, (C4-6)cycloalkenyl, phenyl, biphenyl, naphthyl, a monocyclic heterocycle and a 8-10 membered fused bicyclic group, each optionally substituted with one or more substituents independently selected from the group consisting of:
halogen,
cyano,
(C1-6)alkyl optionally substituted with one or more fluoro atoms,
(C1-4)alkoxy optionally substituted with one or more fluoro atoms or with (C3-6)cycloalkyl,
(C3-6)cycloalkoxy optionally substituted with one or more fluoro atoms;
—S—(C1-4)-alkyl,
—SF₅,
phenoxy, benzyl or benzyloxy, and
(C3-8)cycloalkyl optionally substituted with phenyl, (C1-4)alkyl, (C1-4)alkoxy or a halogen atom;
R4 is absent or selected from the group consisting of a halogen atom, (C1-4)alkyl optionally substituted with one or more halogen atoms, and (C1-4)alkoxy optionally substituted with one or more halogen atoms.

2. A compound according to claim 1 wherein m and n are both 0 or both 1.

3. A compound according to claim 2 wherein the compound has the formula (Ia)

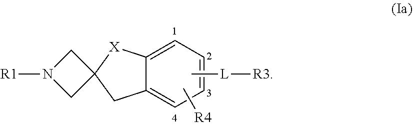

4. A compound according to claim 3 wherein R1 is selected from the group consisting of -1,3-cyclobutylene-R2 and —(C1-3)alkylene-R2, wherein the (C1)alkyl is unsubstituted, and the (C2)alkyl and the (C3)alkyl are substituted with up to two CH₃ groups, with (CH₂)₂ to form a cyclopropyl moiety or with (CH₂)₃ to form a cyclobutyl moiety.

5. A compound according to claim 4 wherein X is CH₂.

6. A compound according to claim 3 wherein R3 is selected from the group consisting of:
(C3-6)cycloalkyl, preferably cyclohexyl, optionally substituted with (C1-4) alkyl;
monocyclic heterocycle, preferably oxanyl or pyridyl, optionally substituted with 1 or 2 substituents independently selected from the group consisting of:
halogen,
cyano,
(C1-4)alkyl optionally substituted with one or more fluoro atoms,
(C1-4)alkoxy optionally substituted with one or more fluoro atoms or with (C3-6)cycloalkyl,
(C3-5)cycloalkoxy optionally substituted with one or more fluoro atoms; and
(C3-6)cycloalkyl optionally substituted with (C1-4)alkyl, (C1-4)alkoxy or a halogen atom,
indanyl, optionally substituted with one or two halogen atoms
8-10 membered fused bicyclic group, preferably preferably 2,3-dihydrobenzofuranyl, indanyl, indoly or 1,3-dioxaindanyl, optionally substituted with one or two halogen atoms;
phenyl optionally substituted with 1 to 3 substituents independently selected from the group consisting of:
halogen,
cyano,
(C1-4)alkyl optionally substituted with one or more fluoro atoms,
(C1-4)alkoxy optionally substituted with one or more fluoro atoms or with (C3-6)cycloalkyl,
(C3-5)cycloalkoxy optionally substituted with one or more fluoro atoms; and
(C3-6)cycloalkyl optionally substituted with (C1-4)alkyl, (C1-4)alkoxy or a halogen atom.

7. A compound according to claim 6 wherein if R3 is selected from the group consisting of phenyl, and pyridyl, optionally substituted with one or more substituents independently selected from the group consisting of:
a halogen atom,
(C1-4)alkyl optionally substituted with one or more fluoro atoms,
(C1-4)alkoxy optionally substituted with one or more fluoro atoms or with (C3-6)cycloalkyl,
(C3-6)cycloalkoxy optionally substituted with one or more fluoro atoms and
(C3-6)cycloalkyl.

8. A compound according to claim 7 wherein said phenyl, indanyl, oxanyl or pyridyl is substituted with two substituents independently selected from the group consisting of a halogen atom, (C1-4)alkyl optionally substituted with one or more fluoro atoms, (C1-4)alkoxy optionally substituted with one or more fluoro atoms, and (C3-6)cycloalkyl.

9. A compound according to claim 6 wherein R3 is phenyl, indanyl, oxanyl or pyridyl and wherein said one or more substituents are at one or more of the ortho and meta positions of said phenyl with respect to L.

10. A compound according to claim 1 wherein W is selected from the group consisting of —O—, —CO—, —S—, —SO—, —SO₂—, —CH═CH—, —C(CF₃)═CH—, —CH═C(CF₃)—, —C≡C—, phenylene, and (C3-6)cycloalkylene, p is 0, 1 or 2 and T is absent or selected from the group consisting of —O— and —O—(C1-4)alkyl-.

11. A compound according to claim 3 wherein R4 is absent.

12. A compound selected from the group consisting of:
1-((5′-((4-ethoxyphenyl)ethynyl)-1′,3′-dihydrospiro[azetidine-3,2′-inden]-1-yl)methyl)cyclopropanecarboxylic acid;
1-((5′-((2-methoxy-4-propylbenzyl)oxy)-1′,3′-dihydrospiro[azetidine-3,2′-inden]-1-yl)methyl)cyclopropanecarboxylic acid;
2-(5′-((4-butyl-2-chlorobenzyl)oxy)-1′,3′-dihydrospiro[azetidine-3,2′-inden]-1-yl)acetic acid;
2-(5′-((2-chloro-4-ethylbenzyl)oxy)-1′,3′-dihydrospiro[azetidine-3,2′-inden]-1-yl)acetic acid;
3-(5′-((tetrahydro-2H-pyran-3-yl)methoxy)-1′,3′-dihydrospiro[azetidine-3,2′-inden]-1-yl)cyclobutanecarboxylic acid;
3-(5′-(benzyloxy)-1′,3′-dihydrospiro[azetidine-3,2′-inden]-1-yl)propanoic acid;
3-(5′-((2-chloro-6-ethylbenzyl)oxy)-1′,3′-dihydrospiro[azetidine-3,2′-inden]-1-yl)propanoic acid;
2-(5′-(benzyloxy)-1′,3′-dihydrospiro[azetidine-3,2′-inden]-1-yl)acetic acid;
2-(5′-((2-chloro-6-methoxybenzyl)oxy)-1′,3′-dihydrospiro[azetidine-3,2′-inden]-1-yl)acetic acid;
2-(5′-((2-fluoro-6-methoxybenzyl)oxy)-1′,3′-dihydrospiro[azetidine-3,2′-inden]-1-yl)acetic acid;
-(5′-((4-chlorobenzyl)oxy)-1′,3′-dihydrospiro[azetidine-3,2′-inden]-1-yl)acetic acid;
2-(5′-((2-(trifluoromethyl)benzyl)oxy)-1′,3′-dihydrospiro[azetidine-3,2′-inden]-1-yl)acetic acid;
2-(5′-((2,6-difluorobenzyl)oxy)-1′,3′-dihydrospiro[azetidine-3,2′-inden]-1-yl)acetic acid;
2-(5′-((3,5-difluorobenzyl)oxy)-1′,3′-dihydrospiro[azetidine-3,2′-inden]-1-yl)acetic acid;
2-(5′-((2-chloro-6-cyclopropylbenzyl)oxy)-1′,3′-dihydrospiro[azetidine-3,2′-inden]-1-yl)acetic acid;
2-(5′-((3-chlorobenzyl)oxy)-1′,3′-dihydrospiro[azetidine-3,2′-inden]-1-yl)acetic acid;
2-(5′-((2-fluoro-6-methylbenzyl)oxy)-1′,3′-dihydrospiro[azetidine-3,2′-inden]-1-yl)acetic acid;
2-(5′-((2-chloro-6-fluorobenzyl)oxy)-1′,3′-dihydrospiro[azetidine-3,2′-inden]-1-yl)acetic acid;
2-(5′-((2-fluoro-6-(trifluoromethyl)benzyl)oxy)-1′,3′-dihydrospiro[azetidine-3,2′-inden]-1-yl)acetic acid;
2-(5′-((2-chloro-6-(trifluoromethyl)benzyl)oxy)-1′,3′-dihydrospiro[azetidine-3,2′-inden]-1-yl)acetic acid;
2-(5′-((2-ethyl-6-fluorobenzyl)oxy)-1′,3′-dihydrospiro[azetidine-3,2′-inden]-1-yl)acetic acid;
2-(5′-((4-bromo-2-chlorobenzyl)oxy)-1′,3′-dihydrospiro[azetidine-3,2′-inden]-1-yl)acetic acid;
2-(5′-((2,5-dichlorobenzyl)oxy)-1′,3′-dihydrospiro[azetidine-3,2′-inden]-1-yl)acetic acid;
2-(5′-((2-chloro-6-ethylbenzyl)oxy)-1′,3′-dihydrospiro[azetidine-3,2′-inden]-1-yl)acetic acid;
2-(5′-((3,4-dichlorobenzyl)oxy)-1′,3′-dihydrospiro[azetidine-3,2′-inden]-1-yl)acetic acid;
2-(5′-((2,6-dimethylbenzyl)oxy)-1′,3′-dihydrospiro[azetidine-3,2′-inden]-1-yl)acetic acid;
2-(5′-((5-fluoro-2-(trifluoromethyl)benzyl)oxy)-1′,3′-dihydrospiro[azetidine-3,2′-inden]-1-yl)acetic acid;
1-((5′-((2-chloro-6-ethylbenzyl)oxy)-1′,3′-dihydrospiro[azetidine-3,2′-inden]-1-yl)methyl)cyclopropanecarboxylic acid;
1-((5′-(benzyloxy)-1′,3′-dihydrospiro[azetidine-3,2′-inden]-1-yl)methyl)cyclopropanecarboxylic acid;
1-((5′-((2-fluoro-6-methoxybenzyl)oxy)-1′,3′-dihydrospiro[azetidine-3,2′-inden]-1-yl)methyl)cyclopropanecarboxylic acid;
1-((5′-((2-chloro-6-methoxybenzyl)oxy)-1′,3′-dihydrospiro[azetidine-3,2′-inden]-1-yl)methyl)cyclopropanecarboxylic acid;
1-((5′-((2-chloro-4-ethoxybenzyl)oxy)-1′,3′-dihydrospiro[azetidine-3,2′-inden]-1-yl)methyl)cyclopropanecarboxylic acid;
1-((5′-((4-chlorobenzyl)oxy)-1′,3′-dihydrospiro[azetidine-3,2′-inden]-1-yl)methyl)cyclopropanecarboxylic acid;
1-((5′-((2-chloro-6-fluorobenzyl)oxy)-1′,3′-dihydrospiro[azetidine-3,2′-inden]-1-yl)methyl)cyclopropanecarboxylic acid;
1-((5′-((4-ethoxybenzyl)oxy)-1′,3′-dihydrospiro[azetidine-3,2′-inden]-1-yl)methyl)cyclopropanecarboxylic acid;
1-((5′-((2,5-dichlorobenzyl)oxy)-1′,3′-dihydrospiro[azetidine-3,2′-inden]-1-yl)methyl)cyclopropanecarboxylic acid;
1-((5′-((2,6-difluorobenzyl)oxy)-1′,3′-dihydrospiro[azetidine-3,2′-inden]-1-yl)methyl)cyclopropanecarboxylic acid;
1-((5′-((2,4-dichlorobenzyl)oxy)-1′,3′-dihydrospiro[azetidine-3,2′-inden]-1-yl)methyl)cyclopropanecarboxylic acid;
1-((5′-((2,6-dimethylbenzyl)oxy)-1′,3′-dihydrospiro[azetidine-3,2′-inden]-1-yl)methyl)cyclopropanecarboxylic acid;
1-((5′-((2-chloro-4-methoxybenzyl)oxy)-1′,3′-dihydrospiro[azetidine-3,2′-inden]-1-yl)methyl)cyclopropanecarboxylic acid;
1-((5′-((2-(trifluoromethyl)benzyl)oxy)-1′,3′-dihydrospiro[azetidine-3,2′-inden]-1-yl)methyl)cyclopropanecarboxylic acid;
1-((5′-((2-fluoro-6-methylbenzyl)oxy)-1′,3′-dihydrospiro[azetidine-3,2′-inden]-1-yl)methyl)cyclopropanecarboxylic acid;
1-((5′-((5-fluoro-2-(trifluoromethyl)benzyl)oxy)-1′,3′-dihydrospiro[azetidine-3,2′-inden]-1-yl)methyl)cyclopropanecarboxylic acid;
1-((5′-((2-chloro-6-cyclopropylbenzyl)oxy)-1′,3′-dihydrospiro[azetidine-3,2′-inden]-1-yl)methyl)cyclopropanecarboxylic acid;
1-((5′-((3-chlorobenzyl)oxy)-1′,3′-dihydrospiro[azetidine-3,2′-inden]-1-yl)methyl)cyclopropanecarboxylic acid;
1-((5′-((2,6-dichlorobenzyl)oxy)-1′,3′-dihydrospiro[azetidine-3,2′-inden]-1-yl)methyl)cyclopropanecarboxylic acid;
1-((5′-((2-chloro-6-(trifluoromethyl)benzyl)oxy)-1′,3′-dihydrospiro[azetidine-3,2′-inden]-1-yl)methyl)cyclopropanecarboxylic acid;
1-((5′-((2-ethyl-6-fluorobenzyl)oxy)-1′,3′-dihydrospiro[azetidine-3,2′-inden]-1-yl)methyl)cyclopropanecarboxylic acid;
1-((5′-((2-fluoro-6-(trifluoromethyl)benzyl)oxy)-1′,3′-dihydrospiro[azetidine-3,2′-inden]-1-yl)methyl)cyclopropanecarboxylic acid;
1-((5′-(4-((3-fluorobenzyl)oxy)phenyl)-1′,3′-dihydrospiro[azetidine-3,2′-inden]-1-yl)methyl)cyclopropanecarboxylic acid;
1-((5′-(4-(cyclohexylmethoxy)phenyl)-1′,3′-dihydrospiro[azetidine-3,2′-inden]-1-yl)methyl)cyclopropanecarboxylic acid;
1-((5′-(4-((3-chlorobenzyl)oxy)phenyl)-1′,3′-dihydrospiro[azetidine-3,2′-inden]-1-yl)methyl)cyclopropanecarboxylic acid;
1-((5′-(4-(benzyloxy)phenyl)-1′,3′-dihydrospiro[azetidine-3,2′-inden]-1-yl)methyl)cyclopropanecarboxylic acid;
1-((5′-phenethoxy-1′,3′-dihydrospiro[azetidine-3,2′-inden]-1-yl)methyl)cyclopropanecarboxylic acid;
1-((5′-(4-chlorophenethoxy)-1′,3′-dihydrospiro[azetidine-3,2′-inden]-1-yl)methyl)cyclopropanecarboxylic acid;
1-((5′-(2-(trifluoromethyl)phenethoxy)-1′,3′-dihydrospiro[azetidine-3,2′-inden]-1-yl)methyl)cyclopropanecarboxylic acid;
1-((5′-(3-chlorophenethoxy)-1′,3′-dihydrospiro[azetidine-3,2′-inden]-1-yl)methyl)cyclopropanecarboxylic acid;
1-((5′-(2-chlorophenethoxy)-1′,3′-dihydrospiro[azetidine-3,2′-inden]-1-yl)methyl)cyclopropanecarboxylic acid;
1-((5′-(2,6-dichlorophenethoxy)-1′,3′-dihydrospiro[azetidine-3,2′-inden]-1-yl)methyl)cyclopropanecarboxylic acid;
2-(5′-phenethoxy-1′,3′-dihydrospiro[azetidine-3,2′-inden]-1-yl)acetic acid;
2-(5′-(2-chlorophenethoxy)-1′,3′-dihydrospiro[azetidine-3,2′-inden]-1-yl)acetic acid;
2-(5′-(2-chlorophenethoxy)-1′,3′-dihydrospiro[azetidine-3,2′-inden]-1-yl)acetic acid;
2-(5′-(2,6-dichlorophenethoxy)-1′,3′-dihydrospiro[azetidine-3,2′-inden]-1-yl)acetic acid;
4-(5′-((2-chloro-6-ethylbenzyl)oxy)-1′,3′-dihydrospiro[azetidine-3,2′-inden]-1-yl)-3-methylbutanoic acid;
4-(5′-((2-ethyl-6-fluorobenzyl)oxy)-1′,3′-dihydrospiro[azetidine-3,2′-inden]-1-yl)-3-methylbutanoic acid;
2-(5′-((2,3-dihydro-1H-inden-1-yl)oxy)-1′,3′-dihydrospiro[azetidine-3,2′-inden]-1-yl)acetic acid;
2-(5′-((7-chloro-2,3-dihydro-1H-inden-1-yl)oxy)-1′,3′-dihydrospiro[azetidine-3,2′-inden]-1-yl)acetic acid;
2-(5′-((5-bromo-7-fluoro-2,3-dihydro-1H-inden-1-yl)oxy)-1′,3′-dihydrospiro[azetidine-3,2′-inden]-1-yl)acetic acid;
2-(5′-((5,7-dichloro-2,3-dihydro-1H-inden-1-yl)oxy)-1′,3′-dihydrospiro[azetidine-3,2′-inden]-1-yl)acetic acid;
2-(5′-(2-phenoxyethoxy)-1′,3′-dihydrospiro[azetidine-3,2′-inden]-1-yl)acetic acid;
2-(5′-(2-(4-chlorophenoxy)ethoxy)-1′,3′-dihydrospiro[azetidine-3,2′-inden]-1-yl)acetic acid;
2-(5′-((2,3-dihydrobenzofuran-7-yl)methoxy)-1′,3′-dihydrospiro[azetidine-3,2′-inden]-1-yl)acetic acid;
2-(5′-((2,3-dihydro-1H-inden-2-yl)oxy)-1′,3′-dihydrospiro[azetidine-3,2′-inden]-1-yl)acetic acid;
2-(5′-((2,3-dihydro-1H-inden-4-yl)methoxy)-1′,3′-dihydrospiro[azetidine-3,2′-inden]-1-yl)acetic acid;
2-(5′-((6-isopropoxypyridin-3-yl)ethynyl)-1′,3′-dihydrospiro[azetidine-3,2′-inden]-1-yl)acetic acid;
2-(5′-((4-methylcyclohexyl)ethynyl)-1′,3′-dihydrospiro[azetidine-3,2′-inden]-1-yl)acetic acid;
2-(5′-((4-ethoxyphenyl)ethynyl)-1′,3′-dihydrospiro[azetidine-3,2′-inden]-1-yl)acetic acid;
2-(5′-((6-(cyclopentyloxy)pyridin-3-yl)ethynyl)-1′,3′-dihydrospiro[azetidine-3,2′-inden]-1-yl)acetic acid;
2-(5′-((6-((1,1,1-trifluoropropan-2-yl)oxy)pyridin-3-yl)ethynyl)-1′,3′-dihydrospiro[azetidine-3,2′-inden]-1-yl)acetic acid;
2-(5′-((2-chlorophenyl)ethynyl)-1′,3′-dihydrospiro[azetidine-3,2′-inden]-1-yl)acetic acid;
2-(5′-((2-fluoro-4-methoxyphenyl)ethynyl)-1′,3′-dihydrospiro[azetidine-3,2′-inden]-1-yl)acetic acid;
2-(5′-(phenylethynyl)-1′,3′-dihydrospiro[azetidine-3,2′-inden]-1-yl)acetic acid;
3-(5′-((2,3-dihydro-1H-inden-4-yl)methoxy)-1′,3′-dihydrospiro[azetidine-3,2′-inden]-1-yl)cyclobutanecarboxylic acid;
3-(5′-((4-(cyclopropylmethoxy)benzyl)oxy)-1′,3′-dihydrospiro[azetidine-3,2′-inden]-1-yl)cyclobutanecarboxylic acid;
3-(5′-((4-ethoxy-2,6-difluorobenzyl)oxy)-1′,3′-dihydrospiro[azetidine-3,2′-inden]-1-yl)cyclobutanecarboxylic acid;
3-(5′-((4-ethoxy-2,3-difluorobenzyl)oxy)-1′,3′-dihydrospiro[azetidine-3,2′-inden]-1-yl)cyclobutanecarboxylic acid;
3-(5′-((2-cyclopropyl-6-fluorobenzyl)oxy)-1′,3′-dihydrospiro[azetidine-3,2′-inden]-1-yl)cyclobutanecarboxylic acid;
3-(5′-((6-methoxy-2-methylpyridin-3-yl)methoxy)-1′,3′-dihydrospiro[azetidine-3,2′-inden]-1-yl)cyclobutanecarboxylic acid;
(1r,3r)-3-(5′-((2,5-dichlorobenzyl)oxy)-1′,3′-dihydrospiro[azetidine-3,2′-inden]-1-yl)cyclobutanecarboxylic acid;
(1s,3s)-3-(5′-((2,5-dichlorobenzyl)oxy)-1′,3′-dihydrospiro[azetidine-3,2′-inden]-1-yl)cyclobutanecarboxylic acid;
3-(5′-((2-chloro-6-cyclopropylbenzyl)oxy)-1′,3′-dihydrospiro[azetidine-3,2′-inden]-1-yl)cyclobutanecarboxylic acid;
3-(5′-((2,6-dichlorobenzyl)oxy)-1′,3′-dihydrospiro[azetidine-3,2′-inden]-1-yl)cyclobutanecarboxylic acid;
3-(5′-((2,5-dichlorobenzyl)oxy)-1′,3′-dihydrospiro[azetidine-3,2′-inden]-1-yl)cyclobutanecarboxylic acid;
3-(5′-((2-chloro-6-ethylbenzyl)oxy)-1′,3′-dihydrospiro[azetidine-3,2′-inden]-1-yl)cyclobutanecarboxylic acid;
3-(5′-((6-(cyclopentyloxy)pyridin-3-yl)ethynyl)-1′,3′-dihydrospiro[azetidine-3,2′-inden]-1-yl)cyclobutanecarboxylic acid;
3-(5′-((6-isopropoxypyridin-3-yl)ethynyl)-1′,3′-dihydrospiro[azetidine-3,2′-inden]-1-yl)cyclobutanecarboxylic acid;
3-(5′-((4-isopropoxyphenyl)ethynyl)-1′,3′-dihydrospiro[azetidine-3,2′-inden]-1-yl)cyclobutanecarboxylic acid;
3-(5′-((4-ethoxyphenyl)ethynyl)-1′,3′-dihydrospiro[azetidine-3,2′-inden]-1-yl)cyclobutanecarboxylic acid;
3-(5′-((6-(cyclopropylmethoxy)pyridin-3-yl)ethynyl)-1′,3′-dihydrospiro[azetidine-3,2′-inden]-1-yl)cyclobutanecarboxylic acid;
3-(5′-((4-ethoxy-3-fluorophenyl)ethynyl)-1′,3′-dihydrospiro[azetidine-3,2′-inden]-1-yl)cyclobutanecarboxylic acid;
3-(5′-((4-methoxy-2-methylphenyl)ethynyl)-1′,3′-dihydrospiro[azetidine-3,2′-inden]-1-yl)cyclobutanecarboxylic acid;
3-(5′-((4-methylcyclohexyl)ethynyl)-1′,3′-dihydrospiro[azetidine-3,2′-inden]-1-yl)cyclobutanecarboxylic acid;
3-(5′-((6-((1,1,1-trifluoropropan-2-yl)oxy)pyridin-3-yl)ethynyl)-1′,3′-dihydrospiro[azetidine-3,2′-inden]-1-yl)cyclobutanecarboxylic acid;
3-(5′-((2,3-dihydro-1H-inden-1-yl)oxy)-1′,3′-dihydrospiro[azetidine-3,2′-inden]-1-yl)cyclobutanecarboxylic acid;
3-(5′-(2-(trifluoromethyl)phenethoxy)-1′,3′-dihydrospiro[azetidine-3,2′-inden]-1-yl)cyclobutanecarboxylic acid;
3-(5′-((7-chloro-2,3-dihydro-1H-inden-1-yl)oxy)-1′,3′-dihydrospiro[azetidine-3,2′-inden]-1-yl)cyclobutanecarboxylic acid;
3-(5′-((4-ethoxy-3,5-difluorobenzyl)oxy)-1′,3′-dihydrospiro[azetidine-3,2′-inden]-1-yl)cyclobutanecarboxylic acid;
1-((5′-(4-((4-chlorobenzyl)oxy)phenyl)-1′,3′-dihydrospiro[azetidine-3,2′-inden]-1-yl)methyl)cyclopropanecarboxylic acid;
2-(5′-((2,6-dichlorobenzyl)oxy)-1′,3′-dihydrospiro[azetidine-3,2′-inden]-1-yl)acetic acid;
2-(5′-((2-cyclopropyl-6-fluorobenzyl)oxy)-1′,3′-dihydrospiro[azetidine-3,2′-inden]-1-yl)acetic acid;
2-(5′-((2-cyclopropylbenzyl)oxy)-1′,3′-dihydrospiro[azetidine-3,2′-inden]-1-yl)acetic acid;
1-((5′-((4-ethoxyphenyl)ethynyl)-1′,3′-dihydrospiro[azetidine-3,2′-inden]-1-yl)methyl)cyclopropanecarboxylic acid;
1-((5′-((4-isopropoxyphenyl)ethynyl)-1′,3′-dihydrospiro[azetidine-3,2′-inden]-1-yl)methyl)cyclopropanecarboxylic acid;
1-((5′-((6-isopropoxypyridin-3-yl)ethynyl)-1′,3′-dihydrospiro[azetidine-3,2′-inden]-1-yl)methyl)cyclopropanecarboxylic acid;
1-((5′-((4-methylcyclohexyl)ethynyl)-1′,3′-dihydrospiro[azetidine-3,2′-inden]-1-yl)methyl)cyclopropanecarboxylic acid;
1-((5′-((6-(cyclopentyloxy)pyridin-3-yl)ethynyl)-1′,3′-dihydrospiro[azetidine-3,2′-inden]-1-yl)methyl)cyclopropanecarboxylic acid;
1-((5′-((6-((1,1,1-trifluoropropan-2-yl)oxy)pyridin-3-yl)ethynyl)-1′,3′-dihydrospiro[azetidine-3,2′-inden]-1-yl)methyl)cyclopropanecarboxylic acid;
1-((5′-((4-methoxy-2-methylphenyl)ethynyl)-1′,3′-dihydrospiro[azetidine-3,2′-inden]-1-yl)methyl)cyclopropanecarboxylic acid;
1-((5′-(2-(6-isopropoxypyridin-3-yl)ethyl)-1′,3′-dihydrospiro[azetidine-3,2′-inden]-1-yl)methyl)cyclopropanecarboxylic acid;
1-((5′-((4-ethoxy-3,5-difluorobenzyl)oxy)-1′,3′-dihydrospiro[azetidine-3,2′-inden]-1-yl)methyl)cyclopropanecarboxylic acid;
1-((5′-((6-methoxy-2-methylpyridin-3-yl)methoxy)-1′,3′-dihydrospiro[azetidine-3,2′-inden]-1-yl)methyl)cyclopropanecarboxylic acid;
1-((5′-((4-ethoxy-2,6-difluorobenzyl)oxy)-1′,3′-dihydrospiro[azetidine-3,2′-inden]-1-yl)methyl)cyclopropanecarboxylic acid
1-((5′-((4-ethoxy-2,3-difluorobenzyl)oxy)-1′,3′-dihydrospiro[azetidine-3,2′-inden]-1-yl)methyl)cyclopropanecarboxylic acid;
1-((5′-((2,5-dichloropyridin-3-yl)methoxy)-1′,3′-dihydrospiro[azetidine-3,2′-inden]-1-yl)methyl)cyclopropanecarboxylic acid;
1-((5′-((2,3-dihydro-1H-inden-4-yl)methoxy)-1′,3′-dihydrospiro[azetidine-3,2′-inden]-1-yl)methyl)cyclopropanecarboxylic acid;
1-((5′-(cyclohexylmethoxy)-1′,3′-dihydrospiro[azetidine-3,2′-inden]-1-yl)methyl)cyclopropanecarboxylic acid;
1-((5′-((2,3-dihydro-1H-inden-2-yl)oxy)-1′,3′-dihydrospiro[azetidine-3,2′-inden]-1-yl)methyl)cyclopropanecarboxylic acid;
1-((5′-((4-(cyclopropylmethoxy)benzyl)oxy)-1′,3′-dihydrospiro[azetidine-3,2′-inden]-1-yl)methyl)cyclopropanecarboxylic acid;
1-((5′-((7-chloro-2,3-dihydro-1H-inden-1-yl)oxy)-1′,3′-dihydrospiro[azetidine-3,2′-inden]-1-yl)methyl)cyclopropanecarboxylic acid;
1-((5′-((2,3-dihydro-1H-inden-1-yl)oxy)-1′,3′-dihydrospiro[azetidine-3,2′-inden]-1-yl)methyl)cyclopropanecarboxylic acid;
1-((5′-((2-cyclopropyl-6-fluorobenzyl)oxy)-1′,3′-dihydrospiro[azetidine-3,2′-inden]-1-yl)methyl)cyclopropanecarboxylic acid;
1-((5′-(2-cyclohexylethoxy)-1′,3′-dihydrospiro[azetidine-3,2′-inden]-1-yl)methyl)cyclopropanecarboxylic acid;
1-((5′-((4-methylbenzyl)oxy)-1′,3′-dihydrospiro[azetidine-3,2′-inden]-1-yl)methyl)cyclopropanecarboxylic acid;
1-((5′-(2-methylphenethoxy)-1′,3′-dihydrospiro[azetidine-3,2′-inden]-1-yl)methyl)cyclopropanecarboxylic acid;
1-((5′-(4-methoxyphenethoxy)-1′,3′-dihydrospiro[azetidine-3,2′-inden]-1-yl)methyl)cyclopropanecarboxylic acid;
1-((5′-(2-fluorophenethoxy)-1′,3′-dihydrospiro[azetidine-3,2′-inden]-1-yl)methyl)cyclopropanecarboxylic acid;
1-((5′-(4-fluorophenethoxy)-1′,3′-dihydrospiro[azetidine-3,2′-inden]-1-yl)methyl)cyclopropanecarboxylic acid;
1-((5′-((2-methylbenzyl)oxy)-1′,3′-dihydrospiro[azetidine-3,2′-inden]-1-yl)methyl)cyclopropanecarboxylic acid;
1-((5′-((4-methoxybenzyl)oxy)-1′,3′-dihydrospiro[azetidine-3,2′-inden]-1-yl)methyl)cyclopropanecarboxylic acid;
1-((5′-((2-fluorobenzyl)oxy)-1′,3′-dihydrospiro[azetidine-3,2′-inden]-1-yl)methyl)cyclopropanecarboxylic acid;
1-((5′-((2-chlorobenzyl)oxy)-1′,3′-dihydrospiro[azetidine-3,2′-inden]-1-yl)methyl)cyclopropanecarboxylic acid;
1-((5′-((4-cyanobenzyl)oxy)-1′,3′-dihydrospiro[azetidine-3,2′-inden]-1-yl)methyl)cyclopropanecarboxylic acid;
1-((5′-(benzo[d][1,3]dioxol-5-ylmethoxy)-1′,3′-dihydrospiro[azetidine-3,2′-inden]-1-yl)methyl)cyclopropanecarboxylic acid;
1-((5′-(2-(1H-indol-3-yl)ethoxy)-1′,3′-dihydrospiro[azetidine-3,2′-inden]-1-yl)methyl)cyclopropanecarboxylic acid;
1-((5′-(2-(2-chlorophenoxy)ethoxy)-1′,3′-dihydrospiro[azetidine-3,2′-inden]-1-yl)methyl)cyclopropanecarboxylic acid;
1-((5′-(3-(6-methylpyridin-2-yl)propoxy)-1′,3′-dihydrospiro[azetidine-3,2′-inden]-1-yl)methyl)cyclopropanecarboxylic acid;
1-((5′-((3-cyanobenzyl)oxy)-1′,3′-dihydrospiro[azetidine-3,2′-inden]-1-yl)methyl)cyclopropanecarboxylic acid;
1-((5′-(3-fluorophenethoxy)-1′,3′-dihydrospiro[azetidine-3,2′-inden]-1-yl)methyl)cyclopropanecarboxylic acid;
2-(6′-((4-chlorophenyl)ethynyl)-3′H-spiro[azetidine-3,2′-benzofuran]-1-yl)acetic acid;
2-(6′-(cyclohexylethynyl)-3′H-spiro[azetidine-3,2′-benzofuran]-1-yl)acetic acid;
2-(6′-(phenylethynyl)-3′H-spiro[azetidine-3,2′-benzofuran]-1-yl)acetic acid;
2-(6′-((3-chlorophenyl)ethynyl)-3′H-spiro[azetidine-3,2′-benzofuran]-1-yl)acetic acid;
2-(6′-((2-chlorophenyl)ethynyl)-3′H-spiro[azetidine-3,2′-benzofuran]-1-yl)acetic acid;
2-(6′-((4-ethoxyphenyl)ethynyl)-3′H-spiro[azetidine-3,2′-benzofuran]-1-yl)acetic acid;
2-(6′-((4-methylcyclohexyl)ethynyl)-3′H-spiro[azetidine-3,2′-benzofuran]-1-yl)acetic acid;
1-((6′-((2-chloro-6-ethylbenzyl)oxy)-3′H-spiro[azetidine-3,2′-benzofuran]-1-yl)methyl)cyclopropanecarboxylic acid;
4-(6′-((2-chloro-6-ethylbenzyl)oxy)-3′H-spiro[azetidine-3,2′-benzofuran]-1-yl)-3-methylbutanoic acid;
2-(6′-((2-chloro-6-ethylbenzyl)oxy)-3′H-spiro[azetidine-3,2′-benzofuran]-1-yl)acetic acid;
2-(5-((2-ethyl-6-fluorobenzyl)oxy)-1,3-dihydrospiro[indene-2,4′-piperidin]-1′-yl)acetic acid;
2-(5-((2,6-dichlorobenzyl)oxy)-1,3-dihydrospiro[indene-2,4′-piperidin]-1′-yl)acetic acid and
2-(5-((2-chloro-6-ethylbenzyl)oxy)-1,3-dihydrospiro[indene-2,4′-piperidin]-1′-yl)acetic acid
or a pharmaceutically acceptable salt thereof.

13-18. (canceled)
